SEQUENCE LISTING (2 sequences)

SEQ ID NO: 1 - DNA, 5487 bp, homo sapiens
  misc_feature (377)..(377): n is a, c, g, or t
ggggcttggcttcctcgcgtatttgaggacagcccatctcccttcaagaaccctacggagagtcggactgcatctccgcagcgagctcttggagcgccgccggccgggaggcgaaggatgcaggcggctccgcgcgccggctgcggggcagcgctcctgctgtggattgtcagcagctgcctctgcagagcctggacggctccctccacgtcccgtaagtagccgtctcctcgctctgctctggagcagtttcagtgcggcattgatgtttggcaccagggtatcaactccgaagtgcatcgcagtgctggcaccctgatgtgtttgtgtctccgctgatagggaagaaaacttctgaaggaactgcttcaaatagnctnttgtttcacatattattcaagcgaccaggttgactttgttgaacttctgattccatgacancagcaaactntgcatgtgtgctcagggtttgacatgctgtggtgtactctctacctaaaagagtacgcagcataatggttgtgaatctctggaaatcagggtgtcgcttctgttttccagaagatgaagatgccagaagggcttctgagcaatgtgctatgagaccacctatggaagagagagaacaactctttaactgtggctttacttagtaaacttccaaacgattactgaaatgtcatcattagacaacgtaagcaactagcagaaaattcatatgtctaatgcagcctcctcattctttttctgtctttccctgtgatccaggaggaaggatgctggtttcttatcatacaaagatcacctgccagtgagccaagtggtggttggagatactgaccgtcaaggctcagaagccaaattgagcgtaggtcctctgcgctgccaaggagacagtaagtttgcatagcagctatggcttgcactttcttatcccatttaaatattgtttccaggctcctcagaagatgcctgcaatcagacttgctctcctgtagctcctacagaaacatcacacaacccaagcagagataacttagaggaggggaacagccataaagcctccagtacaggagccgagatttgggcataaagtggggatatccatctttcctctatattcatatgtaacaagtttgactacctaagtttatgatgatattaatagcaatgctaatagcataaatgcagtactcaacagctgtgattccacggagtgctgctgttaatgccattaggatgactatttatatcaccataaaccgagtttatggtgatataaatagtagcattatggcttaatgtatcattgtaaacttggattatggccatataaagtttgacattattgctatcaaacttcatcatcatcagagcttgatggtcccttgtaagccaaagattgtttctgttaactatgtcttgctttcaagattagtcaccccagagaggaatttggggctgctagcttggacagtgggaggtaagggagttggagtgatggtccctcctaattaaggcaccattaggatttatgttgttgatacccttcgtgtgcttatgtgcagtcagaggggctctgtccccacccaaatttcaaattgtagttcccataatccccacatgttgtggaagggacctggttggaagtaatttaatcacaggaggaggtctttcttgtgctgttcntatgataatgaataagtctcacaagagctgatggttttataaatgggagttcccctgcacatgtgctctctcttgcctgccgccatgtaagacatgactttgctcctcctttgccttccaccattattgtgaggcctccccagccatatggagctgtgagtccactaaacttctttctctttataaatcacccagtctctggtatgtctttattagcagcgtgggaacagantaatacatcataccctgttacatgctttttttttttttgataagagtatttgtgttgatgcaatagcatctttctccatagaacttactcagatgcccttcctggagccagtgcctgcactccatgaactgctggagaggtcattgtagggtatcggcatcagacctctcctcatttttcaantctctctctcctttataacagatgttggtgcattttttgaagaagggatgtggctacgatataactttcaggcaccagcaacaaatgccagagactccagcagcagagtagacaacgctcccgaccagcagaactcccacccggacctggcacaggaggagatccgcttcagcttcagcaccaccaaggcgccctgcattctcctctacatcagctccttcaccacagacttcttggcagtcctcgtcaaacccactggtaaggacaaggatacccagcctctgccatttaacatttgggcagacagaatgttctagcaagagtctatagattgttcttggtttgttatttattccccataggcttttttaagcaagtcacataacttctttgaaccccatttctctccatgaatatcaggaaaacaacttgccagctgggtgtgttagagcaaggtgttagagtaataatgttggtgaggcatgttgcgttctttacaagaatgctgttgcatgaataagagatatgtttgcatgtgtaatgattattttttctccctactaaataaacacatttggctggacactgtggctcacgcctgtaatcccagcactttgggaggccaaggtgggcggatcacttgagctcaagaatttgagaccagcctggccaatatggcgaaaccccatctctacaaaaaatacaaaaattagctgggtgtgatggcacatgcctgtagtcccagctactcaggaggccaagatgggcataatcccaacttggatcatttaaactatttgcatcttcaactttgagacgacagtcactgaagttcactgtgggcagattgtaaatatgtgcgagcctggtgctgtcagccaatatgggtcagtaggcctctgagctcgtgaatccaaattcaaatattgaatgttatctgtggacatcaagggagggagtaaagtttaacagaaattggatgggcatggtggctcacgcctgtaatcccagcactttgggaggctgaggcgaacggatagcctgaggtcagaatttcgagaccagcctggccagcatggtgaaacccccatcactactgaaaatacaaaaattagccaggcgtggtggcaggtgcctgtaatgccagctactcaggaagctgaggcaggagaatcgcttgaacctgggaggcagaggttgcagtgagccaagatcgcaccattgcactccagcctgggcaagagcaagactccgtcaaaaaaaagaaagaaaagaaaagaaaggaagaaagaaagaatctaagagcaggaattgaggggatgtgacattaaaatgaaatttagggcaaacaaattactgagctttcttttttcttctataggaagcttacagattcgatacaacctgggtggcacccgagagccatacaatagccctacagtgtcaacatcacccgccacgagaagaccatctttctcaaggtatacatacatgtacctataaattacatataatatcgcattatagtccctgtccctataatgcttggccattggttttgttttgaggggatagaagtagaagagatgctttttacactttctcctacaagtgcataactagtgaagtagaaatatatataattctgacaaggaaagacaatgatgaagttgaagacatcttttagggggaaatagaataaatgtttattccgtatagtctttctggttttcccagttctagatccacatatctatcaatttactgcttttatccattgcaccatctttcttcgccaagctctcagacatctgaaacttaccgtgtttcccagcaaaggttattttggtttctccgacctacttctttcactgctccaacacaatacgaagagctgtatttttctctctctctctgcagagcctttgtcttaaaaatcctcaaaacagtctttccccagaatctcagactctgactgtgggctttcggcacacaaagggatggttttattatgacactcaaattttacttcaggttcattgacgaccccggccctcaccactgatttttacagtgaagaaaccagcttgggaaacaccatgaataaaacataatgtgaggaggtctccctgggagctaggccacagcttcatttctctccctntgctgccaactcgcccaaccacccctgcctgtggcaggagaaatcattctcattgggataaccgagaaaactacaactgtttaccctacttgaccctcagcatttgagtgtgtgtgtgtgtatgtgtgtgtgtgtgatcatgtcacaaaataaaatgtttccgcaaaacttcattgctgtagatgtggcatattccctatcagaggctgacactggagattggatcaagcagattttactccattaaaaagaacatggatcttaatcctgtaggtaaatcatccacagaaaatgtacatcacctggagagccgttcataaaggatcttcacttaccatgctcagattttttcaaagaaccccagcaaaggaagacatgggtttccattttctcgggctggtgttttagagtcagtgctgatgaaataaggtatcagaaaaccagggttcaaagagtgatgtcatccccacagggaagatttggttccacacagggtagagacgtacttctaaaagtgtctcttgttttcctcctgcagctcgatcattatccttctgtgagttaccacgctctttctgggaaaagttataggtaagaatgtggttcgttaggtataaatgcgtgctacaaatacatttgggtaatgtgagtttggattttaaaactatcaggtaaatttctcttactggggccagtcacgaatatcttctctgtacaggaaagttacgtggttgttctcggtgttgcacgagcaaagcgtcaacaaattatctttgggggaaattgctcatgtaactcattctaaagctaattcgcctttgaaatacattgtgattaggctgggcatggtgggtcacgcctgtaatcccagcactttgggaggctaaggctggtggatcacttgaggtcaggagttcgagaccagcctggccaacatggcaaaaccccgtctctactaaaaatacaaaaattagccatgcatggtggcaggtgcctgtaatcccagctactcgggaggctgaggcaggagaattgcttgaaccccggaggtggaggttgcagtgagccaagatcgtgccaccacactccagcctgggtggcagancgagactctgtctcaaaaaaaaaaaaaaaaaaaaaccaaacaaacaaaaaacacattgtgattggtaatgggggcttctaagtcancgatggtaatggaacaaacnttgtttgg

SEQ ID NO: 2 - DNA, 421458 bp, Homo sapiens
agaaaatctgggagctatgtatgtacagctgttggaggaggactgtaaaggggctggtatagacatctacagaaggttgatggttctgtgtctacttcctctgtgaatgtgcagacaagcatctcatagggagcctggggctgctattggtccatagagctagtagttgaggaggagacctgggcatggaatagggagagacagggcaaactggaaaccaccagcacctttgcatctgtctctgactgtctccaaccaacagtaaccttagaacaaaatgactactgctcactgccacctcccaaatattattcttttggccaagtgtagctgggatccattcagggaaggtgattctgcaaaacatagttcccagaataatcaaggtgaaaatagaataatcttgcacacaggtctttgataagactaggaatatataatacataacagctaggaaaaaatatataaattttccccaagtgcttataatgaacaaacatatttgggaaccatatccacctagcctagtcaactaaattaaaagctggagtcatcttagatgctttcctctcaattaccctcaacatctagttaccaagtttcatataattttacttcctaagtgtctcttgaatatggccccattttctccctttttctctcactctctctcctctcattttgacacaaggtcttactttgtcatctaggctggagtgcattggcgtgaacatggcttactgcagcctcaacctcccagactcaagtgatcctcctgcctcagccccccaagtagctggaactacaggtgcatgctaccatacccagctaattttttttttgtagagatagggtttcaccatgtcacccatactggtcttgaactcctagactcaagcgttccacttgtgttggcttcccaaagtgctgggattacaggtgtgagccaccgcgctcagccagccatatcttctctatccttgttacaaatgcttatggtgtcttacctggaactcctacctgtttcctaatcagtctttctgcatctaccacaaatctgtccatactaccactacagaagggggtcccaaccctcactgtgctttagaatcacctggagggcttaaaaaaatccattgcctgggccccactctagaccatatatcggaccctctcacagcaaccttctatggtaccaccattacctttgttttgcagatgagcagactgagacttagcagagggttaaataacttctcaaagtccacagctaagaagtagtggagcttggatatagtgccgttatctgagtgcagagcgtatcctcctaaccacatagtatgatgccttccagactatgccacagggctaatgagatggttggtaggaccagtagtggaggtatggaacacaattagaagtacagaattgaggggaaaagaatgggaatggttttcaatttggttggtttgaatggattcttagacaaccaagtggtgctgttgagggggcagttggaaatacaatgaagctcagtctaatttagggctagtgagggatggacaggaggaaatggatacaaaagacaattctgaggtggaaaggacaggatccaatggctgaatgaaagggcgaaggtgagcggggatgtaggattggctgggaggagaagaagagagtaaagctatcttgaagaaaatttaatacaggaagggcagcagttttgggaatgtgctagagaggactatgagaataattttggtgatattaactttgagaacccagcagaatctgtaagttggaaatatgaatctggagtttgggagagaggttgcagtcagggattctgattgaggttgcatcgggtggttgaaaccctggaagtggatgagagggatgagtaaggtcaagtatgagggatgctcaagaagagaagcaagccatggattaaattctggggagatccaagataaggggtggggagaaggggagaaactaatgaaggggacccaaagaaaggagtgagcaagtgggaggaggacaggaagacagatagatagatagacagatagatagagaagacagaagagaaagagagagagaggagaaagagagaaacggagagagataaagagaggagaaagagatagagagaagaaaaagatagagaaagaggcaaaagagagaaagaagagaaagatttattttaaggaattggctcacttgattttaaggtctagcaagtttgaaatttgaagggcaggctgctaggctaaaaattcaggcaggatttgaagcttctctctctctgtttctctctctctttaggcagggtctcactctattagcctggtctcactctattaggctggagggaagtggtgcaatcactgctcagtgcagcctcgacctcctgagctagagtgatcctcctgcttcagcctcccaagtagctgggactacaggtacaggccactgtgctggctaattaattttttttttctttatagaaagggttttgacaagcaatgggtaaagaatttcctaaatggtgatgggataactggctagtcatatgcagaagattgaaactggacctctaccttacagtttatacaaagttaactaaagatgtattaaagacttaattgtaaaacccaaaactataaaatccctgcaagacaacctaggcaataccattcaggacataggcatgggcaaagatttcacgacaaagatgccaaaagcaaaaattgacaactgggatctaattaaactaaagagcttctgaacagcaaaagaaactatcaacagagtaaacagacaacctacagaatgggaaaacaattttgcaaactgtgatctaacaaagatctaatatccagcatcttataaggaacttatatttacaagaaaaaaccaaacaaccccattaaaaagtgagcaagggacatgaacagacacttttcaaaagaagacatatatgtggacaacattcatataaaaaaagctcaacatcactgattatcagataaatgcaaatcaaaaccacaatgaaataccacctaacaccggtcagaatgggtattactaaaaagtcaaaaaataacagatgctggagaagttgtggagaaaaaggaacacttttacactgttggtgggagtgtaaattagttcagctattgtggaagacagtgtggtgattcctcaaagacccaaagacagaaatattgtttgacccaacaatcgtatgactgggtatacacccaaaggaatataaataattctattataaagacacatgcacacacatgttcactgcagcactattcacaacggcaaatatatggaatcaatctaaatgcccatcaatgataggctggataaaaaaatatggtacatatataccatagaatactatgcagccataaaaaagaatgagatcatgtcttttgcagggacatggatggagctggaggccattattcttagcaaactaacacaagaacagaaaaccaaacactgcatgttctcacttataagtgggagataaatgatgagaacacatggacgcatagaggggaacagcacacactggtgccttttggagggcggagggtaggagaagggcgaggatcaagaaaaacaactaatgagcactaggcttaatacctgggtgacaaaatactctgtacaacaaacacccatgacacaagtttacctatgtaacaaactgaacttaaaagttaaaaaaaaaaaagaggttcttattatgttgcccaggctgatctcaaacttctggactcaagtgaacttccaccttggcctcccaaagtgcctggccttagatattcatacatttatttatttttagagatgtggtgggggtgtggggggtctctctctgtcacctgggctggagtgcagtggccccatcatagctcactgaagccttgaactcctggactcaagcaatcctcccacctaagcctgccgagtagctgggactacaggagtgcactaccatgccgggcaaacacactgattttaagtacaactgtgtgctttcagtcttaagatcttcagcttttgaaggacctcagccttcctctagttctaactcctctcgttctaaggagatccttcccattcctgcagaacgtctccccaacccttcttcttcacagttctccctcctatccctgaaccattgcaagaggttttgtgccaagcctctggcacatggcacattctatctcatgttgtgatttcgttttctatgcacgtgctctccttctagcctggaggtgagctgcttaagggcaaagagctcatgtgtagcccttccttgtacccattgtcctatagcaggtgctacctagggggtgctccagtagcttccttgaatgaaccaaagagaaggggtgaaggggggagtcagttgcctaggagcttgcatggacttctgtgtcctggaaccagcccggagctgctggaagatgctagaagttggaatcctatctcatttctaaacatctgctcccactcatctctttccagggtcatcagttatccccactcctcccccccgcccccactttgtataaatgagcccctcacatccctccatcttctccatcactgtgtggtcacttcttaaatcatgcgtctggaaggcgccctcctgtaccccatcctggaggcttcctgcacttcccctcttgtttcttccagaaatgagtcagagtgaaagcaaactttctgaggtcctgagatcccggagacaggctgtgagggccgctggccccgcccgctccagcagcgtgtgtgtgtatgtctgtgtgtgtgtgtgtgcgcgcgcgcgcgcgcgtcaggggccaatgctggaaaggaaacagcggactcgctgaggagaagaaaggagccgcccccttcgtcgccagccccctccccacttcccgtccagcgcgctgctcccttccccccgcctctggcctcggggaaggagcaggaggcagcccaggcggagcgggaggagctggcagcggggcgcatgccaggcgggggtcctcagggcgccccagccgccgccggcggcggcggcgtgagccaccgcgcaggtaggggagctgccacagagtcctagcacgtcctgggggggtcctggcaaggagggcgcgctctggcccccaggaagtcgcgtaggtcagggcgtgcctctgttaacaacccctttcgaggtgagggaaactgaggcaggacgcgaggcgtgcgaggttgagagaagagcgagtcttgggctgccagcccggcaccagcgccgagccgggccaggtccctgacccaggactgtgcagcgatccccgggacccgcagtttgcgaaactgtaggagtctgacccgcgcccccgctgcgtgtccaggtgaccgggaaaggttgcagaggcgtgcttggggtttggatccgtccccgtcctgggacacctgcgccgcctgcactctcgttgcggtgcgccccagcctgcgccccctcgggacaggggacgtcccgcccgccttaaccccaagacccagctggatctcgggttccaccccttcggggacctggagggaggaggctgggagcgggaccaggaactggtgcctgtgccttccctaggggctgctctgaaatcagtgtcaccgcccacttcctacttggctacagtaacggggatggcccaagccggagactgctcggtttccaccgcctggagcgcgggtctcgctgtcaactcttagtgatgattgtggatggttgtttttcaacaatgacaggacttctaccttctcactcttaaaatgtgaggttttccaaagacagtgagatttggagttagggagtgctgccttgtgttcttgctgggtgaggacagggctgggtcagtcgcttaacctctcagagcctcagtctcctgaaatgtaaaggagaacactcatagcctggcctccatgtgtcgttaggtttgttgtatgggaaatcaatttgcaaagtgttatcatcaactggacaaatttgaattggacagcactaggaccataggaactggggtttggagacagtctgcctgcatgtcaatcctggacatgcaggagactatgacagtacctgcttcatagggttgttatgcggtttaactgtgatggtggaagtgaagcacctcgcccaatgcctgaaatacccagtgcactctcaaaaaaccttagttgctatttttcctaggcaaaatctaaataattgggagtaatgtgagaataccagtttgatttgattaatgcaggaatagtagggcatttaaaaacacatgtagttctcctgtgctcggcatggtacatgaagtcctctgaggttcagttctgtgccacagagcaaaaagaaccgtggacaagatacccgcttttcgggctctttgactttgagcctgttttctcatctgtttacaaaaaagataaaagaaaaatgggggtagtaattactgccttttgtactgatcgcattgcctgaggattaaatgaggcaaaagatctgaaagcaccttgtggagtatgagtcactatgcacatataagacatgatagtactaggagggaatggtaacatctgcaggtttttagaacttacaatgtgccagacattttattatgaattcctttgatgaaatcctccctgcatctttatgggactgggattacagtcctcatttccctatgacaaagctgaggttcagggtcaccctgcttggttcccaagctcacagagctcttaagtgtaaagccaggattagaaccctggtgtgggtgccactcacaactgtgttcttggccgcttgccaaactgaacttatagagtggagctgtgattcagaacagacctatattagaatttaggtaatttttttttctttaggagacagagtcttgctctgtcacccaggatggagtgcagtggcacaatcatggctcactgtagccttgaactcctgggctcaagagatcctcctgttccagctttttgagtagttgggattacaggcgcatgccaccatgcccagctaatttttttttttagagttggagtcttgctatgttgctcaggctggtctcgacctcctggcttcaatcattcctcccactttggcctcccaaagcgctaggattacaggcatgagccaccaggcccaacctccatcctgcatatctatagacccaaaatgtaagagtgaactgttaatcctggcaaaagaggctttggcataattttaatttccctcagacttattttataaaatatactttaaactatcttcttgtaaaacaaatgatgacaatttgcagaaaagttattaaaacattgaaagaacaggttgcaagatctcatcacaaatctctaagtggtatctggattgttttgttttgtttcaacactcatttgccagaacccttaggatagcacatgaggtgtaaccggaaatcgaggcgttgggtgtcatggttactttgcagggctgggccccgggacttttctgcaggaagttcattgctcacccaagatgcctccagaagaaagcagaaacttccgagaggagcgtgaatgctctgtttctgtgctgcagaaacaattcctagtttatttgtttcctaaattaagtttatcttattggtttttgtttttccaaaagcaatgcatgctcattacaggaagcacagaaagctacaaacaagtaagtgaacactaaaaaatactgtaatctcaccaaggtagacaaatagcttggtgtctctgtccttgtaacccatttctatgctgcatgcatccatccaaaagtgagaggcaaactattattgttttttgttaaatgacaaaaatagtacttgctaattgctcaaacattatgacattaatgtagaacaaagccaatttattatgctcattaaaactttttctttttttaaaatctaaaaattttgattggagaaggattagagactaaattgttgatatttgaattttattgtggttttggttaaagtttgatgcaggaccttcagagcaaagagagggtacaatggtgacctttaaacaatatatatattttttgagacggagtctcgttctgtcgccaggctggagtgcagtggcacgatctcggctcactgcaacctctgcctcctgggttcaagcgattctcctgcctcagcctctcgagtagtgggattacaggcgcccaccaccacatccagctaatttttgtatttttaatacagacagggtttcaccatgttgatcagcatggtctcgatctcctgaccttgtaatctgcccgcctcagcctcccaaagtgctgggattacaggcatgagccactgcgccgggccctttaaacaagatttaaagaccagcacacaaagtttctgagtacaaaaaaaggtaatattttaggttagggtcattagaccagaattagaatcattctttttttttctttttttaattaaaaaagatggggtctccttattttggccagattgaccttgagcctcaagcaacttctgcctcgacctcccaaagtaccaggattacagatgtgaaccattgcacctggccagaatcattcttttgataataatgatggtaattgagtttatatgctgtgttctcatgaatccaagcagagcccactgtttccagagattcccttggcagccaccctgggttgaggagggtcccctcctggcacagctggaaccaccggtgaagagccatttttcagtcagcaccgtttagtaatataagacttttagcctcgaggagtcttccgttgttagttcctaactttaatgactttgtggtaaaaattccttagcgaatgacttctgtgcaacaatcatgtaatttttaaaattccagatgctttagccgttcagaatccattttttctttcaaaaccagagttcaccagctgtttctcttgagcgaagactttgcaatgagctttcaaatgttcacaccaagtagaagcaggaagggagaactgtggctgggcaatgaatgaaatactgacatttagctgtagaaggtaggcaggggctcacatggtgtgcacagccccttatatgactactggtcctgtatctgcttcctttccctgccccatttcccctttggctttttgtcatgactcagaaatggatgctagacattatggggctccctccagtcactcgggtcactcgtcacagacttataaacagaatcacagaacttattgtcagatgaagcttcaaaacaccagttggctaaactccttgtgttctggcaggtaagatgtcggcatccttatttaacaaatgatgctactgagaactatgtatgcagagacatagtctcaaaataagtgcagtcatgcctcgcataatgacgttttgtcaatgtcacatatgtgagagtggtcccataagattatactgggctgtcaatttcctaatgcctagtatttactatacattttaatgttattttgtgtacttctacttagtaaaaaaaagttaactgtaaacccgcctcaggcaggtccttcaggaggtatcagaagaaggcattgttgtcataggagatgatggctccatgcgtgtttttgccttgaaggccttccaacgggacaaggtgtggaggtggaaggcagggatagtgatgatcctgaccctgggtaggcccaggctagtgtgtgtgtttgtgtcttagtttttaacaaaaagtttgaaaagtaaaaaaataaaaattaaaaataggagcttataggataataaggatataaagaaaaaatatttttgtacagctgcacaatgtatttgtgttttaagctgtgtttttataaaaaagtcaaaaaattaaaaaaatttaaaaattattatatataaagtaaaaaaagttatggtacgctaaagataatttattgttgagaaaaaaatttaaatgaatttagtatagcctaagtgtacagcgtttataaatacagtagcatgaaattatgtcctcaaccctcacgtgcactcaccactcactcactgactcacccagagcaacttccagtcctgcaagctccatgcatgataagcctcctatagaggtgtaccatttttcatcttttatagtgtatttttactgtaccttgtctatgtttagatatgtttagatggaaaagtacttaccattgtgttacggctgcctacagtattcagtacagtaacatgctgcacaggtttgtagcctaggaacagtagctatacaatatagcttaggtgtgtcatagcctataccatctaggtttttgtgagtacactctatggtgtttgcatgacaaaatgacctaatgacacatttctcagaaagtgtccctgtcaataagtgatgcatgactgtgattgaaataagtagccccacattatttttctcatgtcaacttcaggttaattttccagccacgtggccttgtgcttgctcatcataaaaaaatgtggataaaattctacctgttaagccacaacatttgcctgtcatgggtggacatttaccgtttattttcagagttcaaagtttgaactctcagagaaatgcaaataagacagtaaactcctcgaggtgaggaatggcaatctctgaagataaatcatatatttattgtacagtattgaatgttgtaagtggcagtagagatcatgtaatctggccttcttcttttacaggtgagaaaagtgagaaacagcagcagagtgattgttagaacctggctcctcttcttttacttagaactgttttcattaagttgtgtttcatttcatagttctgtataattcatccctgcttaagatacattaaatgaattgaactgaatcccttttttttgagctatgatggctcttaaaaacttagattttcagccaggcgcagtggctcatgcctgtaattccagcagtttgggaggccaaggtgggcagatgacctgaggtcaggagttcgagaccatcctggccaacatggcagaaacccatctctacttaaaatacaaaaatcagctgagcatggtggcgtgtgcctgtaatcccagctacttgggaggctgaggctggagaatcgcttgaacctgggaggcagaggttgcagtgagccaagatcatgtcactgcactccagcctgggctacagagagagactccgtcaaaaaaacaaacaaacaaaaaaccacgtagattttcaagtaattactattgttacactaaatgtcaatggatccttaagaatctatttgttcatgaattcattatttattcattcaacgtttattgagtggctacttggttcagaattagaattagcatgacagccacacattagaaaggtatcactttttgactaaattaaaaacttgctaaaaatagttatccattgatggaataggctaccccgaaaggcagagagcattttgtcattggaagtatgtagggcagtgtggatgactctagggtgactgtcatgagggttctgcactggggagggggctgcgttggatgatgaatgggaacccttccagtgctaagatgctgtgatcgttgtcctgcaggattgggattgtgcaggatttgagctactgtgtaaatttcctattccctgctcattgggcttccacattttgcttccttcccttgcccaggtggttccatttcaggaagcctagatagcttctttgcagtcatctgcccgaatgtcgtagctactttaactccccagagctgtacatgtctaggattgatattgatcattaacacaattatgttcgcccagatgaacaaatctaccaggagtagaaacgtgcatttgacaggtggccttgcatggagactaatcgctttcacaggttgaaaggtgaaatatctaaagtttaatgagtaagttgtgtcctgaatgtggaatctgagttgaaattgcattttgtgacatgtacgcctacctaacttttctttttcatacctgtcatctttttttttttttttttgaaacggagtcttgctctgtagcccaggctggagtgcagtggcgcagtcttggctcactgcaacctccgcctcccaggttcaagcaattcttctgcctcagcctcccaagtacctgggattacaggcatgccccaccacgtgcagctaatttttgtatttttagtagagacagagtttcaccatgtcggccaggctggtcttgagctcctaaactcaagtgatccgcctgcctcggcttctcaaagtgctgggattacaggagtgagccaccgcgcctggcctgtacctgtcatctcttggtttgtgagtggattgattattggactttatttttaacacaggagaatgtgcgtgttttgcatttgtagtgaaagcatgcctcatcttactccaacactgataggaaaaggaagattattttacaatggtcaagtgaaaaaaaaagtaaatacagagataaaacggggttctaatatttctgctacactggaaacttagagttgcaagagctgttttgggatcacaaatcttctgaagaaaaaaagtttgcgtatttcagtacaagtcttaatgtttccctagcgtcttgtgttatagctgcaggccaagcaaaatgaaatgtgagacactgtccttggggatagagcatctgtattcaatttaaacagtagatttgggggccttaaattgcctcgactctattaatcattttgtgttccttagtttatagaattatctcctagagacattaacttatccattaatttcttccattgtgtttatttattcattcacaaacactagcacctactgtatacacagtacagtgttgtacattgcagggtacttaaaaatgtagagtgccaggcatggtggtgcatgcctatagtcccagctccttggaaggctggggcgggaggattgcttgaggccaggagttcaaggtggcagtgcgctatgcttgtgcctacaaatagccactgcactccagcctggacaacatagcaagaccctgtctcaaaaaaaaaatgaaaaaactcccccaaaaccaaaaatgtagattgttctcaagggccatccagtctagagagaactaagggagcctggtattcttaggccttagagcttaacagccacacctaggatgcctcttcagaaggtgttccagcccttcctgtgtcttcccagttctgatactggattatgtcattatgtctctgttgagtaactccttccaatcaggcctgcacactgctgccaggacagtcctcacaaagcacagatctgattgggttattgttcctctgtggaaggggctttcagagactcattatctccccccaagcaaattcttagcatctcttttcaagctttccatacactggcccttaaacacttaaaacggacactctggcctttttattatgcacattattctgcccaccgagaatattacagaatccgtctttcttgctcccaatctgaacgcatcaaaatgtcatccctcatttaccacctagcaaatcaataatgcttctcagccttccctggttctctggaatcgtctgctctctccttcttctgttatagtttgttatggatataacctgtttttctgactccaatgtagacttcttcaactcaaggtttcagttctggtttatctccttactccctcatctatgtagtacacttatggtttactctgttctcactggattagaccaaactcttaattatatttaggatgtattaatccaatgcatagattaaccaagtctattatttttatatcttttgaaaccaatcaggagtttcagaagttcagttcagtttaaatatttattgagatgcatacactatatagtcagatactatgatagctcttgaatataaaaaatatatatacttatgtttgtatatatatgtgtaaattgatatgtaaattcaaatagatagtgttgtctacaccattgagaagcctatagtcccgtggaaaaaagagaattcattctcatgaagtttccggatattgtttcatcagaaggactgggcacatcggggtgtactaaaatcattaagaagtatttagcaccagtagatgcctaataaataccttacagtctagatggatggatagatggaggaagaaagaagggctcctttaatttataatttacctattgctgggtttactcctaggtatgacaattttgaaaacaatttactcaatgtttatttccacactgtgtatgaaatatgctgctcttcattcagaaagagatatataattctccatttgtagaacactgagttaatttcatggttgtgatgttgcttttgcttactttcacccacattttctcctctgtagggtggcagctccgtagggtggacacacgctttgggagacctgcagctgtttgtgtctgggggcaatggtgaagggcagtgactcattggcccagaactcatgatctcatctctcacccagctggctaatcggccttggggacaggaggatgagcgctgcactccctcctaatcatgcagcttttgtatgtctttctttacttgacttaaattgtaatttgaatggaaaaatggacaaattctatcatttgaatttaacttttatatattttcttgactctgatttgggattatctaagtgcatttattcaaatatttattgtgtttttgaaactttttttgaaaagcataaggtaatacatgataaaagatattgttattacccctgtttctgccatttcaaggagtcagaacaggctcagtggaaacaaaatgagatgtcttatgggagttactttcccttcagctaatcttggtatgaatttatattgacactacctttaaaattttattctgctttttaaattttactttaagttctgggatacatgtgcagaacatgcaggtttgttacataggtatacatgtgccatggtggtttgctgcacctatcaacttgtcatctaggttttaagccccacatgcattaggtatttgtcctgatgctctccctccccttgacccctaccccccagcaggccctggtatgtgatgttcccctccctgtgtccatgtgttctcattgttcaattcccacttatgagtgagaacatggggtgtttggttttctgttcctgtgttagtttgctgagaatgatggtttccagcttcatccatgtccctgcaaaggacatgaactcatccttttttatggttgcatagtattccttggtgtatatgtgccacattttctttacccggtctatcattgatgggcatttgggttggttctaagtctttgctattgtacatagtgctgcagtaaacatacgtgtgcatgtatctttatagtagaatgatttacaagcctttgggtatacacccagtaatgggattgctcggtcaaatgattagttttttatttatataaatttatggggtacagtgcaatttcattacatgcatagtttgcctaatggtcaagtcggggcttttaggatgtccatcacccaaataatgtacattgtacccattgagtaatttctcatccttcactcctcctatcctcacccttctgagtctccattgtctgttattccactttctacatccacgtgtggacatgatttagctcccacttgtaggtgagaccatgtgatatttgtgtttctgtgtctgacttgtttcatttaagataacggtttccagttccatcgatgttgttggaaaaggcatgagttcattttgtatggctgaatagttttcccttttgtatgtataccgtattttctttatccagttctctgttgatgaacatctaggttgattccatatctttgctgttgtgaatattgctgtgataaacatgtgagtccaggtatttttctgatacattgatttattttcctttgggtagatacacaatagtaggattgctatattaaatggtagttttatttttagttctttgagaaatctccatacttttttccatagagattttaataatttacattctcaccaatagtgtttaaaatttcccttttcacttttcactgcattctcttcaatatctgttattttttgactttttaataatggccattctgactggcataagatgacatctcattgtggtttcaatttgcatttctttgattagtagtaatgttgaacatgttttccatttgtatgtcttcttttgaaaaatgtctattcatgtccttttcacactttttaatggaattattttgttgttgttgttgaattgtttgggttccttgcatattctggatattgttggatgcatagtttgcaaatattttctcacattctgcaggttgtctcttaagtctgttgattatttcctttgctctgcagaagctttttagtttaactaagtaccatttccctatgcttttttgttgttgttgttgttgcctgtgcttttgaggtcttagtcatgaattctttgcatagaccaatgttcagaagagttttccctcagttttcttctagtatgtttatagttttgggtcttatttataagactttaattcatctttagttgaatttagttttgtatatgatgaggtataggggtccagtttcattcttctgcatatggccatccaatttccccagtatcatttattgaaaggggtgtcctttgcccagtgtatgttcttgtcaatgttgtcaaagatcagttggttgtaaatatgtggctttatttctgggttttttattctgtttcattgatctatgtgtctatttttataccagcctatttgttacctattgctgtgtttactcgttattctcttttttaatgagtaaaataccctttttaaaagacaacatccaaattaggagactgcctttgattaaaatgaataactattaatcatcacagagcagggaaagtgtgaatcatcaggaatttagtggagagaaatccactgttttgggtatgtctttgtattagtctggggtaaatttcttgaggacaaaccttgtctcatttgccttcttataattcatgttagacttatagtaacagtaaagacttactgaattgggttgctttaaaacagcatgtgtaatatagccaccatggcatgcaggagtaggtagaattgcccccaaccaggagtggctcttgaaacatcacagggagaggattaggtaacggttgggttagaaagaggagcacaaggcacttgtcttcatgttgcttgggctcacaagcacacttttcatgtagattgcgtgttcaagatcaacatgccaaaagtggcaaaaatctctaaaggagcctccatctacacatttcgtgagagaaaaatgtccattctccatatcaaagcacattatcatgattttaatttggagtggatttaggggactgatgaagcatggggcactgcagcatccccctagccaacccttacacctgtggtgctcccaacacctcctgcgttcatcatttatcatgaagtatggtgcattcctgaaacactggacaagtgcatcttctcctagaagcacggaggtacattccaaaggcaaactcacttaaattgtgatcagaatacttcaacttgcttgctattgttgtggataagtgatagttaaccacaaccggctgagtggccaggtggaaaggacatggatggggaaggaggttcagggtcctgcataagtcctctagtgtctgtagatctcagtgtcctcccttgtgaaaatggggataatatcagccctcaatttacacagttgtagtgaaggtccaatgtgatcagtgtttgtcacaacctgtcatcatttatgtgtatagaactgcttttactataaacttgcttgtattgtccccagcagccagtcctgcctataatctgttggcttcattgcttgtccagtgatctgtggctccctcctgtcctaatccaggtccaagtggattaatgttatctgattcacgttagtgtttctacagatatgtaatgttatctgaatcacattagtgtttctacagcaaaacagatgcttgttcccactaagtaggataaatgaagactctaaattgcttcacatctgtgcacatgaggttctgccaccatgtgagttcagtcaggtcagattactgccagatgaattagagaaggcttgaggttctcagagaattttggattttggaattagagatgatgcatcatggatttgtgcttaatagctgttttgctactttccctccccttcccctctctccctcctcctccacttccagttcttagcctcagttactattgtactgcctccgtttcttgagacaggctgctgatatgctgcctttggtctctgagccaccaggtatagcgctgcataatggaagaacagatgaacataagagtgtcttggggaagatgagcacaagacatcatggggttgtattgttacccccagcttcagcacattcccggagtgtaagtgcaggctggcttctccataaagcaacacagcagtatgctctgagtgggagtacctccgaaactcagcttccaaaactgtgggctcaccagacctccttggtattttttttttttttttttgagacaaggtctccctctgttgcccatgctggagtgcagtggcatgatcacagctcactgcagcctctacttgctgggctcaaatgatcctcccatctctgctctgcaagtagctgggactataggagtgtgtcacaatgcctggctatttttatttttatttattttttgtagagacaggatctccctatgttacccgggctggtctcaaactgctgggctcaagaaaacctcctgcctctgtctcccaaagggctgggattataggcatgagccaccacgctgtgcccctccctggtttttgatccttggtctgacccctggattctcctggttgacttacactctttggagtgctgactagtatagactctcccaatgctgccttttggatgaatcctgtgatgccacattttccagccttaggacaaatgataacctaccctgtagtctgtccatgctctggagaccctgctggtcttacaagatgaactggacaactgctttcactgataggcctgaaggaaaagggttggaactgtcaaggttccccattaaataccaaccagcaatgacaatcaaggtgagctattcttgtgctgggtaggatgctatcttgggctctgagtgtgtccggaattggtgggttcttggtctcactgacttcaaaaatgaagccgcggaccctcgcggtgagtgttacagttcttaaaggcggcgtgtctggagtttgttccttctgatgttcggatgtgttcggagtttcttctttctagtgggttcgtggtctcgctggctcaagagtgaagctgcggaccttcgcggtgagtgttacagctcttaaggcggcgcgtctggagtcgttcgttcctccttgtaggttcgtggtctcgttggcttcaggagtgaagctgcagaccttcgtggtgactgttacagctcataaaggcagtgtggacccaaagagtgagcagcagcaaggtttgttgcaaacagcgaaagaacaaagcttccacaatgtgcaaagggaccggagagggttgccgctgctggcgcctgcagcctgcttttattctcttatctggccccacccacatcctgctgattggtacagcccagtcgtctgttttgacagggcgctgattggtacgtttacaatccctgagctagacacaaatgttcgccacctccccaccagattagctagatacagagtgtccattggtgcattcacaaaccctgagttagacacagggtgctgactggtgtttttacaatccctgagctagacataaaggttctccaaggccccaccagactcaggagcccagctggcttcacccagtggatcccgcaccggggctacaggtggagctgcctgccagtcccctgccgtgtgcctgcactcctcaggccttgggtggtcgatgggactgggcgctgtggagcagggggcggcgcttgtcggggaggctcgggctgcacaggagcccacgaagggcggggaggctcaggcatggcgggctgcaggtccccagccctgccccgcgggaaggcagccaaggccttgcgagaaattgaacacagcagctgctggcccaggtgctaagcccttcactgcccggggccggcggggccggccggccactccgagtgcggacccgcggagcccatgctcacccggaactcgcgctggcccgcaagcaccgcgcgcagcccccggttcccgcccgcgcctctccctccacacctgcctgcaagctgagggagccggctccggccttggccaacccagaaaggggctcccacagtgcagcggcgggcggaagggctcctcaagtgccgccaaagtgggagcccaggcagaggaggtgccgagagcgagcgcgggctgcgaggactgccagcacgctgtcacctctcgtgagtatctctagtgctgctggcgatgagatgattcagtgtggatgattgataccaaatgtatcatggacagtgaaggctgtgtaacaacacatataggtaggctctgttacaggagttacagttctaggcccagtaaatcttttttttttcactgtgcaattgaattttggttttacctatataacattttcatctgtatgaaatgtaagctctatacaattctccattaggacattgtgtggattttggtgtttctgaatctcttctggctcagaaaattccctgctcttgctggtataatttcatacccaaagagggcattctaaccttgaaagaagttcagctcagcagagcgtctgtcattgcctttctctgtaaacacagttttccttcttccttggcttctcaggtgttttagctccagggaggaaatagtcacagtgaaaatctatttaataaaaccgttatgaaaatgtactcaacctgagcatgcactggaacagcagaagatgaggacttggtctactgccttcccaccaaaccttgccggctctgtaaattttcagagctgtcacttgtgagctatttccaccaagggagagcttctgtaaagttggggacttggcaggccatttcgctaattctttgggtgttcttttagcattgtctgcagtgagtgtgggagtcaaaggcccttcagtgcagagataatgccatcttgttccctggaaccccgaaagagccagagtcaaaggagctttttgtttcactcatagaaaaacccacaggaaatcccagcttcagatattcctattcagtttccttgtcccttcagatctcactgtgtttaggcctcctttgagtgaggcaataactagtaaaaaaaaataaaataaaataaatgaaagaatgccattcatggtggcttggggactgggcacacacactgtatacacggtgtgggggccttccccgggacgctgatgtccttgttctcctagcagtcaaaaattggccagtgaaatacagagaatactagctacctcatactgttaagagaattgactgcggtcagatgtgtagagtttcaagtacatacagtttctgcctcactgtaggcgctcaatattattattgaggagctattattagatttcacatctctgccagctcttgtgtcacttattaagcatatgactgggaataagctatttaattctcttcgtctgtaagataggaagtaataatgcttattttgtgggcctattgtgaggatcatgtaagatgagagataaaagggcttagcaaacggtagcatattacaggtggcctgcagcctgccgctgtgcaggtgacgtcccacacaatggtactggccgaggggtgaatggagccctggtgctgaggatggcgagcctttttctaattcatcagctcagaggtgccacctttttctaatttaccctccctaagaggacatctttttatattttaccagcccacagaagataccttttaaaaatttgcacaatggtaccatatgtgctagctgcaattctgtcaacaagagaaagatgttatttttagtgtagcagacagagaattctggagagggcacatggcatgctttatctggctccaacttggtcatttctgtttaatttcaatgatctgattgccatagagtcagaggagcaattttgctattgcccaaattctacattttcactaggattttccttgaaggcaaggactataacccccaccctgcccatcaccttgcttgctcaccactgccctgtacatatccttcagaatggacatttgttaacaatgggtaagagtgtaggctggcctttcacttaccttcctgtgctttgctctttactgctagggctagcagccggaaagctacatatcccaggctttctcgccagctcatttgctgttaaattctgttaataggaggcactggtgggagaccagtaagacggagagaaaaacttcctgattctgggttttactgtgaggccattgcaagcagttgctgtagtgatagtaaggaaggggtgagttggaacatcagcaaccctgcaggtaacccccttattggggctccaatcttagttgatgtagcacttccttggtggcttcagagccaaatggcacaacctcctcaacagtacagcagattgagaaggctgagggacatcgtttaggagcattaatgatgggtgtctagctatgacccccttttccttttcacttctgtcctctccttctcctttgttctcttctagttctttctatatctttgtaacaaattccctatattaaatttcttctgcttgagatacttgaagtggtcttagtttccctgcatttagcacgtggactctctgaatctgttgggtgcttagggcctgggtttgtgccctaggatcctgtgactgaattcttcctttctctttccatcctcatcatgcatgcaggattgtcgcagtagcctttaaatagattttcagccaccagcctctttctgttcactgtggacattattggtagtttaatttttctaaaaatctattttctggatccacatttctgctcaaaactctatttttatgataactatttaagcataggtatagtcatctatgacctaaaccccaaaggcaggatcgtcaccatggccaccttttctgtgatcctaataacagagagatctgtgtcagaagaggattggttgttctttcaccctggctaactggaaggcagggagctcagtggtagacacaccggtcatcatgctggtgtgggaaagatttcaaagcctggccatagctcccactgaagctcctttctgcaccttactgcttccttgtacaagtcaaaccagggcctttagagcagggaccttcattgcactgccctctcctcgcctcccaaatgataatatccagctttcaaggtctgcctgatgtcccagggttaggagggcagagaccaggcagtttatgtcatgtcacttcttttttttttttgtcttttaaaaaatttattatactttaaggtctggggtacatgtgcacaacatgcaggtttgttacataggtatacatgtgctatgttggtttgctgcacccactaacttgtcatttacattaggtatttctcctaatgctatccctccccgcaccccacgacaggccccagtgtgcgatgttccctgtcctgtgtccaagtgttctcattgctcaattcccacctatgagtgagaacatgcactgttcggttttctgtccttgcgatagtttgctgagaatgatggtttccagcttcatccatgtccttgcaaaggacatgaactcatccttttttatggctgcgtagtattccatggtgtatatgtgccacattttcttttctttttttttttttagagataacaatactcactttcaacaatttctagaactacagagaagaaaaacaaggaaatagaagatttgagcaacacaattaactagacctaacacacatctacagaacagtctatccaacaatagcagaatatgtacttttctttttttttttttacaataacagtgttatcatttattattttgattaactcgtcatttagcattaggtatatctcctaatgctatccctccccactccccccatgccacaacagtccctggtgtgtgatgctccccttcctgtgtccatgtgttctcattgttcaattcccacctatgagtgagaacatgcggtgtttgattttttgtccttgcgatagtttgctgagaatgatggtttccagcttcatccatgtccctacaaaggacatgaactcatccttttttatggctgcatagtattccatggtgtatatgtgccacattttcttaatccagtctatcattgacggacatttgggttggttccaagtttttgctattgtgaatagtgctgcatataaacataagcgtgcatgtgtctttctagtagcatgatttataatcctttgggtatataaatagcaattttgctattgccccaattctaaatttttggggcaatttagatcgctgggtcaaatgggatcactgggccaaatggtatttctagttctagatccttgatgaatcgccgcactgtcttccacaatggttgaactagtttacactcccaccaacagtgtaaaagcattcctgtttctccatatcctctccagcatctgttgtttcctgactttttaatgatcaccattctaactgatgtgatatggtatctcattgtggttttgatttgcatttctctgatgaccagtgatgatgagcattttttcatgtgtctgttggctgcataaatgtcttcttttgagaagtttctgttcatatcctttgcccactttttgatgggcttgtttatttgttttttcttgtacatttgtttaagttctttgtagattctggatattagccctttgtcagatgggtagattgcaaaaattttctcccattctataggttgcctgttcactctgatggtagtttctgttgctgtgcagaagctctttagtttaattagatcccatttgtctattttggcttgtgttgccattgcttttggtgtttcagttattaagtccttgcccatgcctatgtcctgaaaggtattgcctaggttttcttctagggtttttatggttttaggtctaacatttaagtctttaattcatcttgaattaatttttgtataaggtgtaaggaggggatccagtttcagctttctacatatggctagccagttttctcagcaccatttattaaatagggaatcctttccccatttcttgtttttgtcagctttgtcaaagatcagatggttgtagatgtgtggtgttatttctgagggctctgttctgttccattggtctatctctctgttttggtaccagcaccatgctgttttggttactgtagccttgtagtatagtttgaagtagtgtgttgcctccagctttgttcttcttgcttaggattgttttggcaatgcaggctcttttttggttccatatgaactttaaagtagttttttccaattctgtgaagaaagtcattggtagcttgatggggatggcattgaatctataaattaccttgggcagtatggccattttcacgatattgattcttcctatccatgagcatggaatgttcttccatttgtttgtgtcctcttttatttcgttgagcagtagtttgtagttctccttgaagaggttctttacatcccttgtaagttggattcctcggtgttttattctctttgtagcaattgtgaatgggagtttgctcatgatttgggtctctgtttgtctgttattggtgtataggaatgcctgtgatttttgcacattgattttgtatcctgagacattactgaagttgcttatctgcttaaggagattttgggctgagatgatggggttttctaaatatacaatcatatcatctgccaacagggacaatttaacttcctcttttcctaatggaatactctttatttctttctcttgtctgatggccctggccagaacttccaacactatgttgaataggagtgctgagagagggcatccctgtcttatgccagttttcaaaggtaatgcttccagtttttgctcattcagtgtgatattggctatgggtttgtcataaatagctcttattattttgagatacattccatcaatacctagtttattgagtttttagcatgaagtgctgttgaattttgttgaaggccttttctgcatctgttgagataatcatgtgttttttttcattggttctgtttatgtgatggattacgtttattgacttgcatatgttgaaccagccttgcatcccagggatgaagctgacctgatcatggtggataagctttttgatgtgctgctggattcggtttgccggtattttattcaggatttttgcatcgacgttcatcagggatattggtctaaaattctcttttttgttgtgtctctgccaggctttggtatcaggatgatgctggcctcataaaatgagttagggaggattccctctttttctattgattggaatagtttcagaaggaatggtaccagctcctctttgtacttctggtagaattcggctgtgaacccatctggtcctggactttttttggttggtaggctattaattattgcctcaatttcagatcctgttattggtctattcagagattcaacttcttcctggtttagtcttgggagggtgtatgtgtccaggaatttatccatttctcctcgatttttctagtttatttgtgtagaggtatttatagcattctctgatagtagtttgtatttctgtgggatcagtggtgatatcccctttatcattttttattgcgtctgttttattcttctctcttttcttctttattagtcttgctagcggtctatcaattttgttgatcttttcaaaaaaccagctcttggattcattgattttttgaagggttttttgtctctatctccttcagttctgctctgatcttagttatttcttgccttctactagcttttgaatgtgtttgctcttgcttctctagttcttttaattgtgatgttaggatgtcaattttagatctttcctgctttctcttatggacatttagtgctgtaaatttccctctacacactgctttaaatgtgtcccagagattctggtatgttgtgtctttgttcgcattagtttcgaagaacacctttatttctgccttcattttgttatttacccagtagtcattcaggagcaggttgttcagtttccatgtagttgtgcggttttgagtgagtttcttaatcctgagttctagtttgattgcactgtggtctgagagacagtttgttatgatttctgttcttttacattttctgaggagtgctttacttccaattatgtggtcagttttggaataagtgcaatgtggtgctgagaagaatgtatattctgttgatttggggtggagagttctgtagatgtctattaggtctgcttggtgcagagctgagttgaagtcctggatatccttttaaccttctgtcttgttgatctgtctaatattgacagcggggtgttaaaagtctcccattattattgtgtgggagtctaagtctctttgcagatctctaaagacttgctttatgaatctgggtgctcctgtgttgggtgcatatatatttaggatagttagctcttcttgttgaattgatccctttgccattatgtaatggccttctttgtctcttttgatctttgctggtttaaagtctgttttatcagagaccaggattgcaacccctgcttttttcttttttgctttccatttgcttggtagatcttcctccatccctttattttgagcctatgtgtgtctctgcacgtgagattggtctcctgaatacagcacactgatgggtcttgactctttgtccaatttgccagtctgtgtcttttaattggggcatttagcctatttagatttaaggttaatattgttatgtgtgaatttgatcccatcattatgatgttagctggttattttacctgttagttgatgcagtttcttcctagcattgatggtctttacagtttggcatgtttttgcagtggctggtacccgttgttcctttccatgtttagtgcttccttcaggagctcttgtaaggcagacctagtggtaacaaaatctctgagcatttgcttgtctgtaaaggattctatttctccttcacttatgaagcttagtttggctggatatgaaattctgggctgaaaattcatttctttaagaatattgaatattggcccccactctcttctggcttgtagagtttctgctgagatatccactgttagtcatcagacttccctttgtgggtaacccgacctttctctctggctgcacttaacattttttccttcatttcaaccttggtgaatctgacaattatatgtcttggggttgctcttcttgaggagtatctttgtggtgttctctgtatttcctgaatttgaatgttggcctgccttgctaggttggggaagttctcctggatagtatcctgcagagtgttttccaacttggttccattctccccgtcactttcaggtacaccagtcaaacgtagatttggtcttttcacatagtctcatatttcttggaggctttatttgtttctttttactcttttttctctaaacttctcttctcgccttatttcattaatttgatcttcagtcgctgataccctttcttccacttggtcaaatcagctattgaagcttgtgcatgcgtcatgtagttcttgtgccatggttttcagcttcatcagttcatttatggtcttctctacactgtttattctagttagccatttgtctaattttttttcaagggttttagcttcctagcaatgggttcaaatgtcctcctttagctcggagaagtttgttattactgaccttctgaagtctacttgtgtcagctcgtcaaagtcattctctgtccagctttgttctgttgctggcgaggagctgtgatcctttggaggagaagaggtgctctggtttttaggattttcagcttttctgctctggtttctccccatctttgtggttttatctacctttggtctttgaagatggtgacctacagatggggttttggtgtggatgtcctttttgttgatgttgatgctattcctttctgtttgttagttttccttctaacaatcaggtctctcagctgcaggtctgttggagtttgctggaggtccactccagaccctgtttgcctgggtatcaccagtggaggctgcagaacagcaaatattgcagaacagtaaatattgctgcctgatccttcctctggaggcgttgtcccagagaggtgcccgcctgtatgaggtgtcagtcggcccctacttggaggtgtctctcagttaggctacacaggggtcagggagccacttgaggaggcagtctgtctgttctcatagctcaaacaccatgctgggagaaccactgctctcttcagagctgtcagatagggacgtttaagtctgcagaagtttctgctgccttttgttcagctatgccctgcccctagagctggggtctacagaggcagcaggccttggagagctgcggtgggctccgcccagttcaagcttctccagccgctttgtttacctactcaagcctcagcaatggcagatgcccctccctctgccaggctgttgcctcgcaggtcgatctcagactgctgagctagcagtgagcaaggctccatgggtgtgggacctgctgagccaggcacaggatgtaatctcttggtgtgctgtttgctaagaccattggaaaaacgcagtatttgggtgagagtgtcctaattttccaggtacagtctgtcacagcttgccttggctaggaaagggaaatcccctgaccccttgtgcttcctgggtgagataatgccccgccctgcttcggctcaccctgtgtgggctgcacccactctccaaccagtcccaatgagataaacaggtacctcagttggaaatgcagaaatcaccatcttctgcatcgatcatgctgggagctgcagaccagagctgttcctatttggccatcttggaacagaatgcatcatgtcacttcttatacatgaatgagaataactgtgaaatgtgcacatgtgctctgagcctagataagattgttactatcatgttatattgttattattgtgttattatcgttattatcatgttattttcttacccataggttttccaacttgcttgaagtaatgctttcaggatactcaatattgaatattcaatactgatgggaaaactttcttaaaggaaatggacattacagggtcaactggaggaaaaataacacatttactgctcaaacatagcttgttgtttttatatttaaaaacataggcattgtaaaaagtcaaacaagcctgaaaaatgtatgggagaagatgaaatgatcactgaagtcacatattcctcactgttaatgatttagggaacatccttctagaaggttcacagagccatacaactttaccgaaatgggattatgttgcatcttgttttccaacctaattttttcccacttagtagtatagcacagatatgttttcatgtcaaccagtatgactttacactgttatttctttcttcttttttttctgagatggaggctcgctctgtcgcccaggctggagtgcagtggcacgattttggctcactgcaacctccacctacctggttcaagcgattgttctgtctcagcctcctgagtagctaggattacaggtgcctgccaccacacctggctaatttttgtatttttaatagagacggggtttcaccatgttggccaggctggtcttgaactcctgacatccggtgatctgcccgccttggcctcccaaagggagggattacaggtgtgagccactgtgcccggcctaatggttacacagtattccatgctgacaccataatttactcatccagttacattaaattggaatgttcatagttgttgacctttagaacatgccagtattaagtttttgtatttaaggtgatgctagttctaaagcatatattctgaaatctcagtggcttaacacctcagaagtttatttctccttatgtaaagtttaggttctctccatttgctgatggttaccttgaggtaccaggcttgcgtggaggggctgctgtgccttgcatggccatgcagggactcaggccctctccagcgtgtggtttgcaaggttgccctggcccttgctgcttagctggctgatagtggaagagaaagtgaagaagatgtcacaggaggttttaatggatcagaactggaagtggcagatactgcttctgcccccattccattggccagaatgtcacatggccatacaaaactgcaagggaggctgggaaatgccactcatctgtgtgtgtgggaggaaaggaaatacatttggtgaatactagctagtctctaacagagcttcccctactgtttaccaaattcctgttttgcttttcccctcacacacagaacacacccctcccagagtgagggaccctaagtcccatccagccctggcaccttgcttagtcactgttcaggatctctgggtgatgtgctgtcttccccatcacatctaaatgaggatccttgtggtttgatgacttatgaactagaagagaataaagttatttgcctcctccctagcgcctcccaattttcaatgaagcagggaagcattgtaacattagaagctcccatttagaacagagtaaagagcgggcagcacagtcatcacaggcccaaagcaacaatggaatccagttgggtgggcaaggtggaggttctcaaccagagggtgggagaactttcttgattagaccttgattttgccagaaaaggcaatctcttctcagttggtctccatggtccgagctctgtcctggcaagttccttcttgcactttattctcaatggctacgtttgaagtgattgcaggggagtccgccatcttaggagggtacacagctttgcagccctctagtgccctgctgttgtttcaaggctcaaatagacaaacgtctttaaaaaattttatttttagtccaggctcatggtttctttggccatacatttatgtcaaaaatttgataggattctgttttttttttaaattaattacttttatttttgacaaataatttacatatttatggggtacaatgtgaagctttcatatatttattcactatgaaatgattaaaccaagctaattaacatatccatcatttcacgtgtgtgtgtggtatgaaatttttgtgtgtgtgtggtatgaaattcttagcaattttgaaatatattgcattattattaactatagtcaccatgctgtggaataaatcttaagaacttattccttctgtctaactgaagctttgtatactttgactaacatcttcccatccacttctacccttgccccagcccctgctaaccaccattctattctctacttccgactttttttagattacacctgtaactgagatcatgcagtatttttctttctgtgcttggattacttgtttctgttctgttttcttcccttaagttttttatgccaatagcaaatccctaagtttttttcctagatattattaccaagcttgaattattatattactttctcaccctaatacccttttctgtcaatgagatggtgacttccttgaggccttcagggtcagatgggaaggcaaaattcttaattagctctttgctacaaggctgaatcttttacctgtgtcactcaaaagccttttcaatcttgtttcttactcctcagactttagaagcagtcatttcttccaaggctttacactcagaagtttctggacaaacatctggactctattccctttcattttatgcttgcaaaccaacaaattctttttttaaaaaagtattttaatagctttaggagtacaagtggtttttgcttacatggatgaatggtatagtcctgaagtctgaaattttagtgtacccaagttgcgtacattgtgcccaatatgtaacttttaaagtcgcccctcccacactcccctattctgagtctctaaagtccattatatcactttgtgtgcctttgtgtatccatagcttagctcccatttaccaatgagaaagtacagtgtttggttttccactcctgagttagttcacttagaataatggcattcagttccatctgagttgctgtaaaatacattatttcattcttttttatggctaactagtattccatggtgtatatataccacattttcttcatccactcattgattgatgggcacttaggttggttccatagctttgcaattgtgaattgtgctgtgataatcatatgcatgcaggtgtctttttgatgtaatgacttcttttcctttgggtacctagtccagtagtgggattgctggattgaatggtagatctacttttagttctttgagaaatctccatactgttttccatagaggttatactgatttatgttaccaccagaagtatataagcttttccttttcaccatatccatgccaacatctcttgttttttgactttttaataaagggccattctgtttgggataaggtggtttctcattgtggttttaatttgcattccctgatgattagtgatggtgagcagtttttcgtatgtttcttggccatttgtatatgttattttgagaaatgtatattcatgtcatttgcccactttttaaagggattttttccccttgctgatttgtttgagttctttgtagattctgggtattagtctgttgtcggatgcatagtttgcaaatattttctcccattctgtgagttgtgtgtttactctaatgattatttcttttgctgtgtggagcattttagtttaagtcttatttatttatctttgtgttggttgtttttggggtcttagttataaattctttgcctaggctactatccaaaataatttttcctaggttttgttctagaatttttatggtttcaagtcttagatttaagtctgtaatccatcttgagttgatttttgtgtatagtgagagatagagatccagtttcattcttctacatgttgctatttagttttcccagcaccatttattgaatagcatgtcctttcttcaatttatgtttgtatatgctttgttgacgatcagttggttgtaactatttggctttatttctgggttctctattctattctatttgtctatgtatctacttttataccaataccatgctattctgattacaatagccttgcagtataatttggagtcagttattgtgatgcatccagatttgttctttttgctcaggattactttggctgtctgtgctctttttttggttctatatgaattttaggattgttcgttctttctttttcttttcttcaacttttattttaagttcatgggtacatgtggaggatgtgcaggtttgttacacaagtaaacgtgtgccatggtggtttgctgcacagatcatcctatcaactaggtattaagcccagcatcaattagctattcttcctgatgctctccctcccacccactgcccactgatagttcccagtgtgtgctgttcccctctatgtgtccatgtattttcatcatttagttcccacttatagatgagagcatgcattctttggttttctgttcctgtgttagtttctggaggataatggcttccaactccgactccatccatatccctgcaaaggacatgatcttgttcctttttatggctgcatagtattccatagtgtatatgcaccacatttatttatccagtccatcattgatgggcatttagattgattttatatctttggtattgtgaaaaatgctgcaatgaacatacacatgcatgtatctttataacacaatgatttatattttttggggtgtataccccgtaatgggattgatgagtcaaatggtacgcctctaggtctttgaggaatcactacactgtcttccacaatggttgaactaatttacactcccaccaacaatgtaaaagcatttctttttctccacaacctcaccagcatctgttgttttttgacatcttagtaacagccattctgactggtgttagatcgtatcttattgtggtttttatttgcatttctctaatgatcagttatgttgagcgttttgtcatgtttgttggctggatgtatgtcttcttttgaaaagtgtctgatgatagtttcttttgctgtgcagaagctctttagtttaattagataccatttgtaggtttttgcttttgttgcagttgctttggtgttttcatcatgaaatctttgtccatgcctatgtcctgaatggtattgccttggttgtcttgcagcatttttatagttttgggttttacacttaagtctttaatccatcttgaattgatttttgcttaaggtgtaagggatgggtccagtttcaattttctgcatatggctagccagctctctcagcaccatttattaaatatggagtcctttccccattgcttgtttttgtctagtttgttgaagatcagatggttgtaggtgttgggtcttatttctgagttctgtattctgttccattggtctatgtgtctgttcctgtaccaataccctgctgttttggttactgtagccttgcagtatagtttgaagttgggtagccatgatgcctccagctttgttctttttgattatgattgtcttggccatttgggatttgttggttccacatgaattttaaaatagttttttctaattctgtgaagaatgtcaatggtagtttaatgggaatagcattgaatctataaattgctttgggcagtatggctattttcatgatattgattcttcctgttcatgagcatggaatgtttctccatttgtttgtgtcctgtacgatttctttgagcaatggtttgtagttctccttgaagaggtccttcacttcccttgttagctgtattcctagttattttattctttttgtagtaattgtgaatgggagttcatttatgatttggctctttgattgcctgttgttggtgtgtaggaatgctagcaatttttgcacattgattttgtatcctgagactgctgaagttgcttatcagcttaaaaagcttttgggctgagatgatggggttttctagatataggatcatgtcatctgcaaacaaagataatttgacttcctctcttcctatttgaacaccctttattttgttctcttatctgattgccctggccagaacttccaatactatgttgaataggagtggcgagagaggacatcttgttttgggctgtttttcaaggggaatgcttccagcttttgcccagtcagtatgatattggctgtgggtttgtcatatatggctcttattattttgaggtatgttccttcaatacctagtttattgagaatttttaacatgaagggatgccagattttatcgaaggccttttctgcatatattgagataatcatgtggtttttgtctttagttctgtttatgtgatgaatcacatttattgatttgcatatgttgaaccaactttgtatcctggagatgaagccaacttgattctagtggataagctttttgatgtgctgctgcattcagtttgtcagtattttattgaaggtttttgcattgatgttcattaaggatatcggcctgaggttttctttttttttgttgtatctctgccaggttttggtatcaggatgatgctggcctcatagaataaattggggatgagtccctccttttcaattttttggaataatttcagtcaaaatgatacccctttttctttgtacctctggtagaattcagctgtgaatctgtctgttcctgggctttttttggttggtaagctatttattactgtctcaatttcagaactcattattggtctattcagagattcaatttcttcctggttcagtcttgggagggtgtatgtgtctaggaatttatccatttcttctagattttttagtctatgtgcatagaggtgtttacagtattttctgatggttgtttgtatttctgtggggttagtggtgatatctcccttaacattttctattgtatctatttgattcttctctcttttcctctttgttagtctagcaagtgatctattttattatttttttcaaaaaaccagtttctggatttgttgatttttttgaagggttttttgtgtctctgtcttcttcacttattatgccctcatcctggttatttcttgtcttctagctttggagtttgtttgctcttggttctctagttctttagttgagatgttaggttgttaacttgagatctttctagcttttgatgtgggcatttaatgctctaaatttccctcttaacaccactttacctgtgtcccagagattctaggacattgtctctttgttctcattagtttcaaagtacttattgatttctgccttaatttcattatttacccaagagtcattcaggagcaggttgttcagtttccatgtagttgtgtggttttgagtgaatttcttaatcttgagttctaatttgatggtgctgtggtctgagagactgttatgatttcagctcttttgcatttgctgaggagtgttttatttccaattatgtgatcagttttagagtaagtgccatatgtcaatgaggagaatgtatattctgctgttttgtggtggagaattctgtagatatctatcagatccacttgatccagggctgagttcaggtccttactatctttattaattttctgtcttgatttgtctaatattgtcagtgggttgttaaagtctcccactattattgtgtgggagtctaagtagtctaagtgtgtcattcttgaagaacttgctttatgaatctgggtgctcctatattggatgcatatatatttaggatagttagctcttgttgaattgaactctttaccattattaatgcccttctttatattttttgatctttgttggcttaaagtcttgttttgtcagaaattaggattgcaacccctacttttttctgttttccatttgcttggtaaattttccttcatccttttattttgaacctgtgtatgtcttgcatgtgagatgggtctcttgaagacagcacaccagtgggtcttggctctgtccagcttgccattctgtgtcttttaattggggcatttagcccaaaaaaaaaatatatatattttatattttttgagatggaatttcactctgtgtcacccaggctggagtgcaatggtgcagtcccagctcacggcaacctctgcctcccgggtgaagcgattctcctgcctcagcctcccgagtagctgggattacaggcacctgccatcttttagtagagatggggtttcaccatgttggccaggctggtctcgaactcttgacctcaggtgatttgcctgcctcaggcttccaaagtgctgggattacaggtgtgagccactgtgcctggcctagcccatttatatttaaggttagtattattatatgtgaatttgatcctgtcatcatgatgctagctggttattttgcagacttgtttatgtagttgcttcatagtgtcactggtctgtgtagttcagtgtgtttttgtagtggctggtaacagtttttcgtttccatatttagtgcttcctggtggtgatgaattctctcagcctttgcttgtctgaaaaggatcttatttctccttcatttatgaaccttagtttggtcagatatgaaatcttgggttggaaattcttttctttaagactgtagaatattggcccacaatctcttctggcttgtagggtttccattgagaggtcctcagttagtctgattggcttcccgttgtaggtgacctggcctttctttctggctgcccttcactttttttttttcatttcgacctggagaatctgatgattatgtgtctgggggttgatcttctcatggaggatcctactggggttctctgcacttcctgaattgaatattggcctatcttgccaggttgggtgagttctcttgaatgatatcctgaagtgtgatttccaacttggttccattctccctgtccctttcaggtatcccaatcagttggaggtttggtctcttcatagaatcccatatttcttggaggttttgttcattccttttcattcttttttctctattcttgtctgcctgtcttatttcagaaagatagtctttaagctctgaaattctttcctccacttggtctattctgctattgatacttgtgattgcattgtgaggttctcgtgttgtgtgttttcagctccatgagcttggttatgttcctctctaaactggctattctggctatcagcttctgtattgttttatcatgattcttagcttctttgcattgggttacaacatgctccttttactcagtgaagtttgttattacccaccttctgaagtctacttctgtcagttcagtcatctcagcctcagcccagttctgttcccttgctggagaggtgttactgtcatttggaggagaagagccactctggctttttgagttttcagcactgattctttgcttatctttgtgggcttatctaccttcaatctttgaggttgctggcctttcaatagggttttgtgggattctttttgttgatctttttgttgttgttgttttctgtttgtttttcttttcatagtcaggccactctaccgtagggctgctgcagtttgctgggggtctgctccagaccttggttgccttggtttttcctgtacctggagatatcaccagtgaaggctgcaaaacagcaaagatggctgccagctccttcctgtggaagctctgtgccagggggatactgacctgttgctggccagaacatatctgtaagaggtggctggagacccctgtttggaggtctcacccagtcaggtggaatggtatcagggacccatttaaagatgagtcttgctgctttttgtctgaggaggtgtgctgcattgtggcagacccttcctcatctggactgtttgcattctccaaagccagcaggctggaatggttgagtccactgaaccacagagatggcagccacctctccccctgggagctgcatccgagggagaggtcagagttctgttaatagaacccttgttggcgtggctgaagcccccacagggaggtccctcccagtaaggaggaatgaatcggggtcccacttaacgaagcaatctggccacagtgtggcaaagcagctgtgctgcattgtggggggcccttcctcatctggaccatttgtattctccaaagctggcaggctggaaaggccaagtctactggaccaaagagatggtggctgcccctctccctggaaactcggacctatctcaggcagattccagcctgttgctgttgtatgcctaagttaagttggtataaattcaaaatagattgttataactataggatattatatgtaattcccatggtagccacaaagaaaatatctatagaatgtacacaaaaggaaatgagaaaggcatcaaaatgtgcctctgtaaaaaaaaggtcaacaaaacacaaagaaaactggtcatggagaaaatgagggtcaaaaatactgtaagatataaaggaggcaaaatggcaaaagtaagtccttccatattagtaatcgagactctgtcttaaacaaaacaaaacaaaacaaaagaaacaaacaagcaaacgaaaaacgtccagtcaaaagcatagattggcagaatgaattaaagaaaaacatgatctaactgcatgctatgagagacccactttagatctaaaggttgaaagtgaaagcatagaacaagatgtaaatagtaacaaaaagagagctttggtgtctacactaatatcataaaatagaatttaagtcaaaaatttttacaagaaaaaaggacattatatgttgataaaagggtcaatttacctagaagatacaacaattcaaaacatatatgcaccaaacatcagagctccacaatatacgaagcaaatgttgacagaaatgaagggagtaatagacaactctgcaatcaaatgcctgaataatgcagtatgtgggctagtccagcctctctcatgatttccacatcactttgatgtggacatcctggacatctgcagttttcagttacttcctgtttcctgctggcatttaagtcccttgtactttatgtgaattcttacttcagttatactgcccctacaacttgcaatgaacattctcactttggttgtatcactttccaccctcctgcctgaaatctgatgctttccttcaagctccatccccactaggaagccttttgtatgagctaagattaacaacttgcatcctttaccctccacgtgttgcatatacctataccactcatttagcacatcacatttgatacttatttatttgacacatgtagctatatttaatctatttgtttggtctcctccataaccagagctttatttccatgggggctaatgctcctttaagtttctggcaatgattactgttatatagatgcagaatcttatttttttaatgaattaacagatgtctccctaaattaggcccaaatactgatcttaatcctaaggattagctaatagaagcatcataggtattaagttggacagatatggccaaactgtttaattttgtgagcttcagtttcatgtgctaatgccatatctatctatctatccatctatctatccatccatccatccatccatccatccatccatccatccgtctatctatgtctagcttgaccatcacaacagtctaaggagaagctatgcttatccctgtttttcagatgaggaaactgagacccagagacattaagtaatttgccaaaattacccaactcacacatgaaagagtcaaggtttgaacctagatctccctgactccactgcttgttattttaaacatctacaatatttacctttcagttcttgaacttaatcttgcctgagatctaagttcaagcctttcattatccacatccccccatatctaatgatagttgtggtaggcagaataatatccccccaaagatgtctgcatcccaatcctggaacctatgaatattttaccttgcatgacaaaaagggactttgcagatgagattaagttaaggatctttagatggagagactatctttgctatccatgtgaacccaatgtaatcatagggtgctcataaaggaaagagggaggcaggagtcaaagtcagagagagatttgaagatgctcaactgcttttgaagatggagaaagggaccaaagccaaggaattcactgacaccttgattttagcccagtgagactcactttgacctccagaactgtaaaataatagatttgtgttgcttgaagccactaaatttatagtaatttgttatggcagcaataggagactaatacagttgaccctcgaacgacacaggtttgaactgtgagtccacttctacatgaattttttttttagtaaatatattagaaatatttttggagatttgtggcaacttgaaaaaactcgcagacaaaccatgtatcctagaaatataaaaaaaaaataagaaaaagctatgttatgaatgcataaaatatatgtagatactagtctattttatcatttactaccataaaatgatacacacatctattctaaaaagtaaaaatttatcaaaacgtatgcaaacagagtgtacatggcatcctttgcagtggagagaaatagaaacaacataaaaaatgcagtattaaatcataattgcatacaattaactgtatacataaggtactgtggtaataatcgctgcattgccacctacattgctattgtggtgagctcaagtgtgggaagtatccacttaaaatgccatgtgatgctaaccgtctgcacatgagcagtttctctctccagtaaattgcatattgcaatgaaagttatctctttcagttctcacatttttttttgtgtttggagcaatactataaactttgagtaacacagcgggactcatacgaagtgccactactgatgctggaagtgctcccaaaaagcagagaaaagtcatgacattacaagtaaaagctgaattgcttgatgtgtgctataaattgagatctgcactgtggttgcccaccatttcggacagatgattcagcttgtaagcagacaatgtaaacttacaatatcaataagtaaagtacagtagtgtaaatacattttctcttccttatgattttcttaatattttcttttccctagcttaccttaattgtaagaatataatacatatacagaatatgtgttaattgaccttttctgttatcaataaggcttctggtcaacaatagcctattagtaggtaagattttggagattcaaaagttacagtgaattttaactgtgcaggtgattgatgcctaattcccatgttgttcaagggtcaactgtacaataggttttctgctttccttaccctcctgatgatttctgaactagttctacagagtttcctcttttcctttgcatctctccttcccactgccaccatccagattgttttcagagcggcacttcatcagctgcatcgtcaccctctgagggtgtggaagggctctcacttccacttaaggccatgccatgctaattgcctttctggctcttttctcttaaaaggtggtgcttagaaaggaaaggaatagtccatatgtggtcttagtaggacagggaacagtaggtctttacctcccttaatccgaatgttagatctcttcaatgcaatcaaggtaatgcttatttttttttggcacccacatcacatccactgctcactcaggttgagtttattgataactacaaccccaagcttttcatgtcatattaaagcaaaagccattatgagctgttcttatatctcgtagaatgtctgccatcttgtgtcatgtagcttttgtctcaggaaaaaaaaaaaaaaaaaaaagcacagtacgaattaatctctccagggaaagatttaaagactaggataataaggagagacagatggaattctgagagaaatatggcaaattctagagctaatctaattaaagggccatgggaacttctccctggcgcataaataaaatatgacaatcagtgtcagctaagaaacccttagcaaatacctaatctcatttttaaatttatcattacatagaactgttatcttttaacaaataatggtatgatgcaaaataagctgagagaagccaacagacacaggccaggcagctgcccagcctctctgcccacccctccctacatacacttcaggtggcatgggctctcccagccatttcgatacatgacacctctgtgacctcgccagcttgaaggataaaatgaaaggttcttagtgaaggaaaaaggcgttcatcttcaccccatcgtaatgggctctgaggaggactggcaacatgtagaatctggagaggagaggtcaggactggctgcagaggaggaagctgctgaggtgattagtagttctcagagagggccttgttctttccctttaaattcaaaatggcagtgcctcaaaccctctgggcttcctggaatttgtgtttaaatgtttatgtatttccaggcgaatattcaaacaatgtagctaagatgtggtaccttcaaagcaagtaaaccagctggcttccctgctatgatccccttgcacagcctggcccttcagacaggagcctgtattctgaaggaagtccttacagagcagcttggatctactaccccttaaaagaaggaggaactggtgaaattatttccattaactttaaggtactcaagaggaccacagaggtcttattcttttcagtttggataaatctggggcagtccaaaggttgccaagggaggtaggaagaaaagtttaatttgcacacttgagctttacagtctgtctgacttcattggagtttacttctttttaaattgcagactcagggcttaaatacttccttaccttcagtgacctttcctgagccctcctgttcctgataagctgcagggcagtgctttctttcctatgatgtgttgtcatattaacacacacacccagagtgattactgtgttcacttcccctggatgtgcttcatctttgttgagttaaaatcaaattgctttctacttcagtaaatccaattcagccttcttactgaccatgggttaaagttttaatttaaaattccctttcttttaaaaattagcaattcaaattcaaggtggtaaagcagcccacattctgagattgttggtgttggaaagggccttagctatcgtctgtcctgcgtgatgcttctgttgaattatctcaaggacagaatagttactactttgaacagattaggtggaatgatgtaaatgaaagtgctttgtaacttgtaggacattaaaaaataaatattattagcatttataaagatgctttttccattgttgccaatgttacttattttttcaaaactactttattgaggtcttattgacataaaaaaagctgtacatacttagctgggcgtggtggggggtgcctgtaatgcaagctacttgggaggtcaaggctggagaattacttgaacctgggagatggaggttgcagtgagctgaggttgcactattgcactccagcctgggtgacagagcaagactctgtcaaaaaaaaaaaaaaagctgtgcatacttaatacatacatcttgataagtttcaggataagtacaacacacttgtgaaaaccacctgtgaatcccacttgtgaaaccaatcattaaacatgtccctcaactcccaaagtttcttctttattattactatgattaattttttttggtgaaagcatataacataaaatctaccctaggcaaattttaagtatacaatgcagtgtttgttagctataggcattatgctgtatagtacagcatactgtacttactagcttaagctttagatattataacatgtgttttcctgttgaactcctgtcttccttcttgtgggtataaactagaataactttatagatcgcttgggaacatttttcacctcaagcggccattcctgtccaggtgtgggatgtgcaacacagtggtccaagagatgattttcagagtcacaaggacatggcatggggtaccattgactcacgttctcctgctaaggtgaaagtctcaatttgctgctagtgcatctttaaccttctctaactctcatttccctttgttataaagacagagctcagcagacttctgtcccaaaatctttggccaacagcaaaccatatttgtatggttatcttctaattatggggagtgattttcatatcccagtgatagtgataccaaaaaagaataaaagtatcctttaaattgacaagcaccatgaagtggtttgagactggctggtgtttgggacatggctttccacggagcttgagagatgggccatggatcagattactgcccactgtgacagacaacacacaatttgccatttcctatggcagtctttgacatatctgaagtcagtgctcatgtgtcctttcagccttcccttctttaggtctcacccttccagtttcttcacatgttccttatttgatgttctcctgcaatggtgactggagtctatcagtttttcttttgaggaagctccctgttcatcatttttatagaaggagcccttgttctgatatcagagagcagttcctcaagttggttgagaaagggaagtcagggagcaaaggagagttaatagggatgacatggaagaattgcagtgatagggatgcctgggaattgttaggagaacacttaggtccttagatgtctttatgttggtgtacaaaataggggttatcaaaactataaaaaccctggaagataactgaggcaataccattcaggacatagacatgggccaagatttcatgatgaagatgccaaagcaattgcaacaaaagcaaaaattgacaaattggatctaattaaacttaagagcttttgcacagcagaaaaaactatcaacacagtaaacagacaacctacagaatgggagaaaatttttacaaactgcatccaacgaaggtctaatatccagcatctatagggaacttaaacaaatttacaagaaaaaaaacaaacaaccccattaaaaagtgggcaaaggacatgaacagacacttttcaaaagaagacatacatgcagacaacaatcatatgaaaaaagctccacatcactgatcatgagagaggtgtaaatcaaaaccacagtgagataccatctaacaccagtcagaatggatattattaaaaagtaaaagaataacagatactggcgaggttgtggagaaaaaaacgcttatatgctgtcggtgggagtgtaaattagttcagccattgtggaagacactgtggtgattcctcaaagacctaaagacaggaatatcatttgacccaacaattctattgtggggtatatacccaaaggaatataaatctttctattataaagacacatgcatgtgtatgttcactgcagcactagtcacaacagcaaagatatggaatcaatctaaatgccaatcaatgatagactggataaagaaaatgtggtacatatataccatggaatactatgcagccataaaaaagaatgcgatagggacctggatggagcgggaggccattatccttagcaaactaacacagaaacagaaaaccaaacaccgcatgttctcacttataagtgggagctaaatggtgagaacacatggacatatagaggggaacaatgcacactagggcctattgaagaattggagggttgaaggaggaagaagactaggaaaaataactaatgggtactaggcttaatacctgggtgatgaaatagtttgcacaacacacctccatgacacaagtttacctacatgcccctgaacttaaaagttaaaaaaacacaaaaataaaataggggttataagcaaaatgtgtgcaacaggtggtacatgtgtaaagactgtcaagattgggttgaaaagtctgtgagagtgcctttgcagcttgcttctctgagccttaaggtgttccacctctaaaattggttgaaatgaggtattgtatggaaaaggctttggtaaacgaggaagtgctatacaaatgtcaaatattagttttttcattagttatattgttattttatgtcactgttataacattgacattctgttcagtgtttatcctggtttctcttcagatcatataaatcttgtgacttttacgaaagattcccatgaagaggaacaactctgagacttaaaccaatttttcagccttggtgctttgtataaaggatccagcactccccagtagtcctttcataaaagagccctgccattcccaagtccattgtcatgatttattgcttgcttgttgggatttcaccatctagaggtttttaatcaagaaagcctatggttgttttagtatccaaatccatgcatgttaaataatgactttttattgtctttataatgtctgccaacctggctgggtataaaattcttgagtatgccctgctccccacctccatccaaactgtatagggagtgttacttggcgttgaatattgccttggaagccagctttaattttgccctttgtacatgacttgagttttttcagagaatcagtcaggattcattcttttttctttggatttcagtgattttaccaggacatcttggtgttggtcataatatttcaatttgttttttaggagctgtgtgatcttttatttgtaggtttagtttggtttcctatttattttttcttctcaagggacatcaattgtccatatttttgtttgccttgtgtatatatcatcttccttttcattattttttttttaccttttcctttgcattttgctattttctcaagttgatattccataccattgactctattttcagctgtaactgtgcctcttgctgcttctgagatggcttcatattatgaatgaactgagcagaacacccaggcaggcatcagttggcctatggtaaatcttcctaggttatcataaaaaaattaacattaccatagctcagattaatagttggccaatagctgccttcttctccccagctcaacacatgcttgaactctccattctcctgagtaaaggaccttggaaaaacactccttaagaactggagaaggttgtattggttttagtatctgccattaattttatccacattttacagtatttggcaggtattctcatggttttaagaagcaatatagtgttaagagcatagttatggagaaaactgcctgggttttaattaaaatttttccagttgctagccatgtgaggttgggcaagctacttaacctcttgattcctcagttttcctcatttgtgaaggaggttagtaatagcactgctatgagatgaaatgaattaatatttgtaaaatgcttataaccctgctgggtacaaagtaggtgtcttgaagtgatcataagatgcattgtcaataaactttcctccccaatttctcaatcactttgctcatttcagtgggtttcacagaggtgtcattaactgtctttattccattatattgtgccagaggttgcctgatttgttttgctcataacgggattgttttaattattcatttagaattgcatccagaatatataactggctggctagtctgtgcttcttagattctgttttttttctcttcttgaaacgagaatacttgtcaatctttgatcttcagtaagtccatgatttctcaaggtctattgagtatatttctgtcatcatgtctgtcagttcttttagtactagatcctgactgatttgacctcagttggggacatgaactcatttgaaatggctaggtactctctttctatctttttacctctcttgggcttcaattcctcctaaattatgctaccactatctttttcaatctgaagatcattttctttaatagagatgaaggctgagaatttgagctatgtcattctgccctgtcttctctcttatctattatcactgtgccctagatactgggcacttccctgcctggttgactttcttgtcctgctccatttgtttgtgtgtttgtttgaagcacacctttgtgctgtccttagtatatttacaggaagtggagctcattctggcctttggtcttcttgacatttttcttattgctttgtgacccccttgggcttactcctggttgcttcccttctgtggatctttgtacatgtcctttgaaaatttctgcctttcagacagcttccttttcaatctcagaagtacttgtagatactttacccaccttgtctgccttggcattattgtactaattaatgtcttggggcacttgggaaagatacctgttgttccttaactccctgacctgggggtttggtttgggagaggtgagaagaggcccagaaatctgcatttccacaaacctcccaggtgattttgatgcacgtgacccccagatcacactcatgcctaactagtttggtgcagaagctcctaacctcttttttctgatcaagctgtccatgaagctgagttgatgagtgacctctggtgagtgcagggggactccctggggcttaccccagggtcaggtgcaatggttcttggcttgaagccagctctaattctgtgccttcctttgccactcaagaaagacaactgaatatctgaacttgaatctgttttcattataaagaattaaataactcgcagttttagtgctttattacttcctccaacatatcttccactccattactggtgtgctgtcccctggaggtgggaaaatgactcactcagcatcctttccttggctatttctttcttttcttttctttttttaagaaactggggtctctctctgttgtccaggctggagtgcagtggctattcataggtttatcataatgcactatacccttgaactcctgggctcaaggaggctgggctactcctcagcctcttgagtagctgggactataggtaggcgccaccacatctggctttcttcttggctatttttaactctagtttgacctggtcactttctccagagatttctttccttccacataatctgtaaattctgaatggctgtcagattaagtctgatgccattcactagaaaagatctttcattaggaggctctacttgaaaccaaacggactacagcacattttcaacttggaaagatttgaagaactgggaagcatgaagatctttgtacctaattcaaggcagtgatgtttggacacttcgactgtgtcgtgtttctcttagagctcttagtaggctggttattctgaaaggttggagtgtctcatagataaacaacaaacatcattgagtaattatacttttggggctcacgtttgagtattgacaactgtgaaaagaggaggtatagactccttacgagtttagtcctatgctctaagacatgtgcatttatcccagactctcgtataaaaagccagtgcccattgtgtgcagaatagggtaggactgaggccagacttcctgggatctagtcctggagctagcactaaccagctgtgttacttatctcctataaacctcagtgatcttatcttaaaaataaggtgataatgatccttacctcattcagttttggcgaggattaaatgacataaaatatgttaagagcttagaatatgtctggcacttagtaagtgctcagtgaatttatgatgcgtaaatggacatcagattatgtcagatttgtcaagctagtttttgatttttttttttccaatccctctctgatttttttctgcgtcacatctgttatatgtgccattttgaagctttgtaattttgggaggaagagtaatgggaattgaataatgtgggcagtcactgagctgttactataacaatatttgatccatagccattgggataagaccatgctatcaattgtagaaagtagcattagtaaaacatttcatttctatggcactttggagtttacaatgtattttctaatgtcctgtctaatttgaatacaaagaaaatatgaagtgcagagaaataactgattttctttttgtctcatcaccaggctaggtgtgacccgggtgtgcagcctcagcttggtgatctcacacatcatcaaagcagttctctgcagtacattgaagcacttgttgggtacacagagtacctgttgttaggttaagcattgtaggaagttgtaagaaaagcagctttgtatggccttcgtggccatgtgtatgtagttctgcctaagagcaaagttaaataaacttgtagccaatatgacaacacagcctgatgacagaaaaaaattccccagataaccttaaaactcacctagaaggagcgaggatttgtgtagtgttccggatgtccctttgttttcaccccattcctacgcccgcccagacttacctttgctgtcactggggctcttttggaggctgggaaggattttaatcctgtctggtcctttcccaagaatagcagtatgattgaaaatgtgcccacaagcataatctctgaattggtggatttaaaacaaagttgatgagagacttccaacggcaagtgtgtaaaaagaaaatatctctttaaaagatgttattgataatgaaaagctgagacccttgatactctgcacacggctagtatgggctttatacagattcagagacatccatgtgtgtcctgcttattggtccttaggctactcccgcccgcaaagtgtagcgctgtcttggtggatcctgccggaatgacacatctttttgctttgggatgattgctgggtcctggggtctctctccctgcctcttccccagccctaccctattgcacattttagtgtggcttagctctgttgcctttgtggtggttaacactcaacctcagagacaaagatctaagcaggaaggaggatctattgtgaaccggttggaagttgatttttgaccctaagaaattagttcaccttatcatcagatagagatgctatttgtacatttctccctcatacatgtacatttactctcaaaatttcctgggctagtaggtctctcaaggtggctgagagaccctagaaggtaccagtgtaccactgggccgcatccttaaagatggtgctgtgagggacagccatggaggcccatgtgcgccattgcccttatcccttgggggctctctggatgagggctttctctgtctttgtcttctcttgtcttcgaggtccttcttactcccttgtccaatgtgtgacatcttatcttttcctccatgagctccatttttctgttccaaatagctctagtctgctggtatattgtatttgttttttcttttcttttttttttttttttgacacagaattcccactgtcgcccaggctggaatgcaatgatgtgatctcgggtcactgcaacctccgcctcccgggttcaagcaattctcctgcctcagcctcctgagtagctgggattacaggcgtgcgcctccacacccagctaatttttgtatttttagtagagacagggtttcactgtgttggtcaggccggtctcaaactcctgacctgatgatccatctgccttgtcctcccaaaatgctgggattacaggcatgggccaccgcccccatcgtctgctggtatattatactcctttctctttgtttagttaaattatttctgctttgttttgatacataacagtggtatattagtctcaaccctttggaacatttttaggtcccgatgctatcatccattatgttgaagaggacaggtcaaaggaaagagccctgtgggaccttccctgtgaccatttaggatttagagtcatttggcctggaactgttatttcccagctacgaatccacctacctgtcccaccggtctgtgtctccagcctccacttctacacatggaaatcaggagaggctgcgtcttaaatccttttggtaaaatccagacatttatgacctctacctctcccatagtttaccatgcctgacccacagtagagttcttaagaaatactattgtcttttccccatttcttatttcctgtttaaataaaataaattcagtatttgtctttctagtgtgttgacatgtctcctattctcccaaattcctcaaagaccagtgcagcaatttaccaatctcattttcacattccttcagtgtaataagtgaattaaagccaatttattatccaggattgcctcatttatttcaaaataagctgtgtaccaagaaagctctattccctcagcaagagctaactccagttttaaagctcattctagagaaaaacagacttttatagtttgttttagctattaaagaccccttttaaaaaagaaataggcctttggatgttgccaggggttagaaagctagtccagagactgagaaggtaaacaaagaagcaggggaaaaactgattttgagtgtttagagtgtgtgaaatgtcacttttccccttaacctcttccttaaggaaaaggagagattgttaaagtagtgtctggtcagggcgcacacatgtagaaggtttctgtaactctagggtaaaatacagatctgttaaaattttcatgagtttcttttacttgtcagaagttgcctacagcttagatgatattttcctgatagtttgtaaacttctattgacttctctgtttttctattcgcccatacccatttattttctctttactgcattttactgtcttaggaagataaaacagaaaagcaaaacaaaacaaaaaccccaaaaaactaagtctgaagcagactgttgaataatattactttaactgatgaacagattttcctcccacaaagaaggaaaaagggagaaaattcgtagctcttctctctttttaaagactgtgctggaaaattccaaaatgttatcggaataaaggaatgacttcgggggctcagaacaactcggggaaagacactaaaatttattgagcatttaacgtcacctgccagaaatttaaaatgtgttttttaaagaacaaacatttcatgattttattgtatccttacgacagcctttcagggtatggggattatccccattttacagatgagcaaactgagacctggagaggctaagtaacttgttacggatcacccaggtgggaactgaccgaattaaaatccaacacttcatgccaggtgcggtggctcacgcctgtaatccaagcactttgggaggccaaggcgggtggatcgcctgaggtcaggagtttgagaccagcctgaccaatatggtgaaaccctgtctctactaataatgcaaaaattagccagacatggtggcacatgcctgtaatcccagctactcaggaggctgacaggaggatcgcttgaacctgcgaggtggtggttgcagtgagccaagagcgtgccattgcactccagcctggagtgcaagactgtgtctcaaagaaaaaaaaaaccaaccaaaaaacaacactttgatttgctgtaagaccctctctgtgaatattaaactggtaaacctcatacataatgatgagattagtcatctacagaagtaactcagggcttgtctgtaaacatcctatgctccaaagtgactgcagtgtaatctcaggaaggcaagactggaatgagatcgtaaataaaaaaatggacatctaatacatacattggtctcgtctgaagaaagactgttaacagagggggatggtcagtatggaattgtaaacaggaggtcaaagaggacgaatgtgttatttacacccaccagaacccagggaagtgtggtgagaagcagcaggaactcttctgtctgctcggcaggcgggcagctctgtgtggcggagacatttgcttccactattatggttgaagtcagcggccataagatcatctatagactgagagaaggcagtgtggacaagggcctcagagcacaggctctctaggggagcatgagttcatgttctccaatctttccaactaccatgcatggaagatattagaatgaacttttccccagatgagaaaacaggcatggagaagttacacttgggcaagtcaaagaggggtagagataggcttaaggccagtctaaattcagaactctcttgactacgttgctctatctctctctctctctcttttttttccttgagacggagtctcactctgttgcccaggctggagtacagtggcctgatcggctcactgcaacttccgcctcccgagttcaagccattctcctgcctcagcctcccaagtagctgggactacaggcgcccgccaccacgcctggctaattgttttttatttttttgtatttttagtagagacagggtttcaccgtgttagccaggatggtctcgatctcctgacctcgtgatccacccgcctcagcctcccaaagtgctgggattacaggcatgagccaccgcgcccggccacgttgctctgtctcttaatagtcttgtcattcctgggatgctgccatttgttttcttttctaatggatagtggatcatgaagatgggttcacatgggcgacttgaattctagagagccaggcaatgtcacagcttcctggccagtttgtgcctgttgggggattccccatctttgatgcctttgacttccagtccttgagggacactcacttccctggcaagcactgtcttggactgcatgagtgattttctattggttggtttctgaccgggcgggcacttagctccatgctggtctgtgcctggctgagatatctaagagcagctcattgccatattctgaatcccagccctcaggggagcagccagcacctctgcttcctgcctcctctggcagatggccctgatctgagagtttaagtgatagctatctgagaggagtgattggtagagacagtagcaccttattttaataatttaaaaatcataaaatatgccactttggatacaaaagcttttaagaggcacttaataaaaatgtttagttgctttccacaatgagaaaacattgttataactaaagcacacttcagatatttaagcattaaagcatccccagagacttattcttaacagtaaaatgtattgctttgttcaagatgattctgtaatggagaggcattacagaggcaagaagagacgagaattctttgtaattagtgtgcactgtttgaatgtaaatgagtacttgaaaacagtttgctctcttaaacatcctaagttttcccactgtcgtcttccataaggtattaattggtttaaacagcctctttatttgtaggcagcacaaaggttaacttcagtccagcccctgtctagttatcatcaactccaaatgctaatgctgtttgaattcatcagacagaattgagctttttgtatgtagtcatggcctcttaaaacttacaaggccaggcgcagtggttcatgcctgtaatcccagcactttgggaggccgaggtggatagattacctaaggtcaggagtttgagaccagcctggccaacatggggaaaccccgtctctactaaaaatacagaagttagccgggcatggtggtgcatgcctgtaatcccagctacttgggaggctgagtcaggagaattgcttgaacacagggggcggaggctgtagtgagccgagatcgtgccactgcactccagcctgggtgacagagtgagactcttgtcaaaaaaacaaacaaacaaaaaaccaaaacttggaaagtatatatagtgtctctatatgaaatgagactagagaaattttataaaagaagggtcctcaagtatagataaaagacaactgtattttattataaatgctttcttatatctcagacccaggtaagttacaggtcatgctcagcacctgcccccgtgcatatagcagcaggttgttgtaacatcagcttattgagaatgtggatcactataacataggagtcgtgttatgctgttcttttaattccctcataccagtgcactacattccaatactaacatgtggaaaaaaaatactgaatatccccttgttttttagtaagaccatagagcagaacatttgcaattttataatggttttatttttaaggtcaggcatttaaagtggcacaaagctttgatgtgggcttacatctgagtattataaacatttagcacaaagtaagaacttacttaaaagatgctccagcttccagtgtgctgtttctgtgtgaaatactgaaaatttgggatcattttaaaaataaacacttatctaagttggcatgcaaaatggagcatttagaaagccatgaaaataagcccatgcataaaatatggcattaaaaaggtaactgtgaactttacagatggactagcatgaaagaagctgtcttttgctagggtggatacgagagtttgcagctctttgctgcaaaaaaataagagcagaaacttaagttgtttagttgttttgaaataacagctacacagagggctccttctggttcatccatattttattagggcagcagttaaaaaattcattagagacacccatctgtagctagcagcacccttgaggggggctcagatggtgatcagattttggagtagcactttacactaaaagaaaaagacaatcgtttaggagaattaatagcacaatggagaggggctgcttgatgcatttgaagcatacccttccacatggtttatgaatcttttgctactgaattcctcagccacctgcttcagttgggaaagagagatccaggcataacaaggagagaaaaattggatcagagagttgagactgtattggtcagtggagaacatagatagagtgtctatgtgatccgtgttgggctctcactcgtgtgtgtttgtgtgtgtgtgtgtgtgtgtaaatcacgttggcctcctggggaacttcatcagtacaaaaaaaagcttgtggtcagagctgaggctgccctggtttaccttggccttgaactccacctccctggtggtcaggtccaggtgcctttgcctcatctttattcatgaattgaaaataactctagttattgaatggcaatggaggggaagtgacctctcctctaccctcctagattccctagctgggtctatgaaattgacaacagatagattaacaggagaataggcatagacatttattttttttttttagaaggagttttactcttgttgcccaggctggagtgcaatggcacgatcttggctcactgcaacctccgcctcctgggttcaagcgattctcctgcctcagcctcccaagtagctaggattacaagtgccctctgccatcataccctgctaattttttgtacttttagtaaagacagggtttcaccatgttggccaggctggtctcgaactcctgacctcaggtaatccgcctttcttggcctctcaaagtgctgggattacaggcatgagccactgtgcccagcccatttattaatttttaatattatgtgcatggggggcatcacaggaaaaaatagtaaataccccaaagagcggtgagatttgagagcttatatactgttttcataggggaaggggaggaggatataggttgtgatttttagaaatgtgctgttagaacagcagatggaagataggagagtttgtgacaaagtttgtctgggtgtggtgtcgactcctagtttcatctcctgcaatacaaatctgtcttccctggttgatgaaacttccaggaagggaattcatgacaactgagttccctttggcacatctgttttgggcagataaagggagtttagagaaaacctttccctgcacttgctgtttttcaagtgccttcagctcaaaataatcaatattccaaagcagcatagtttggagtggcttattttggtttccttcagtgactctgtgccaggcatggtatagcacggttaaagtcataatctcatctatccttccagcaatcctaggggtggagtcccatttcacagatgagataatggaagcaacagagaggttagtcacctgaccatgctcactcataagaggcagagctgaatttcatcttctgctattaagtccaggtccataaactgtgtgcatatacatgttttttttagataaactttctattttagaataattatagatttatcgcaaagctgcaaagacagcacaaagttcctttcacccagcttcctctaacggtaagggcttatataatcatggtgcatgtattaagactcaaaaatgaatattggtgcaatgaactaaactacagatcttattcagatttcaccagtatccttttcctgttccccgatccaatccaggatcccacactgtatgtcacatgcatggtttttgacccttaaacacgaagtgggctggatgtcaaactgctgggtggcctgtttctcctcattactgatgaagaaagtgagacgctggtgccatctgtcaccttccttgtggggcccaggactctcattgtctaatggaaaggggaaatcctatgtagggtagaagatttagaacagaggcatggggacaaattttaaatatagaccatactttttctacttgtagtagtttgtagctaaaaccacattcaagaaatagaaaaagattgggggagagagagtattctgcaaagcaacaacctctctagcatcattaaagtttatgatgcatttgaatcccaagtgtactaggatgttaaataggacttttccttaaaacaaagccttccagtcctgactgcctcctagaatcacctggatagtgtcaagaagcaccaataccggaggcccatccctcgatgttgtaatttcatcagtcttggttgtgatctgaacctcagaatttttaaaagataccaccatgattttttttttttttttttgagacagagtcttgctctgttgcccaggctggagtgcaatggcgtgactgtggctcactgccgccttgccttcccgggatcaagctatcctcctgggcacatctaggatgcatgtaatttgcctgtattctgtgaccaacccttctgtgaacaccccctctgtgaccaacccctcctggaatcccagtgtggtttccttggatggtttctggtcacttttttcgcaagccctcttcttctttcaggatttaccttcttaggcggtctcatttaaccctcctggctttcccacttgcatggcatgtgtttccacctctctgtggcccaggtcctgccttggccctgtcctctcggtccccacctggaagccaccctgggcctattcccatcctgtcttctgcctcttcctccacagctgccacactgtctgtacccaagtcccagtggttctgaacttcttcaagctggctgcactcaacactgcctttcacactgtgcttgctgttctctccaccggcaatgacctcaccctcctttttggcttgggaagcctcagccttccagtttactttcctggcctcctgcagtggagttgctcacagcctggtctgcatctgcttgttgacatgtctgtcttccccagggagccatgagctccattgaggacagggaccatgactttgaaaaattaataattaatttttagagatggggttttgctaacttgcccaggctggagtgcagtggctattcataggaggaatcatagctcattgcagccctgaactcctgggctcaagtgatcctcccagctcagtcttctgagtagctgggactacgggggcatatatgcacccaacttgaaaaagtattatcaaatatttcacggttataaaaactgtatttatatataattatgtgtttaatatataattacacataggttttacatgtgctgtttgaaaataatacaatcagttcctgtgtaaccaccacttaagaaatggaacattacccttgaattactatataattgcatgcattttttcctatgtattgtttgagaataatacagtaggcacctgtgtaaataccagcaggcttaaaaatggaacattaccctcgaggtcccttgcacatcacgtcctgttccctgttaagactggtttatcattcttttgcttttgtttatacttttagcacgtatctatctcttgtgctaaaagactgttacaagcagaacagtcattccagttttagtgattctgggctgagactgtagacaattaaataaaaagccaattcagtgtaggattcatgccttccgactgtgaggctgttgtaaggaagtagcgtttgtgggaatgccacccaggagtcaaatctctagcctttgggtgatcaggaggctgacaggaaaacagcgacagagctttgcctctggtggaactcctggtggcctgcagacctgttggcgtctgtacctgcagtccactcagctttccagatgactgaaatgccccaacagggtagagtggctcgcctctccaagaaagagcagttggtgaataatcatgcctgggaaaacaatccgcctggcaaagagatggatggtatccgttggtgaatgtgatggcagcaagcccagtgcaagattctgtggcgattccttgattgacctgctcggttggtggattttgaaagggagtgggaaggactgcttcacttgcgtagaactctccctctctcttgcgctatcttcatatcgatgtctacctttctctttgtgtatatgtgtttataaatatttcatatgtatatttgtgtacagacatatacacatttggatttatatttgtatacattttatattagtcagttttcatcctgctgatgaagatatacccgagacttggaagaaaaagaggcttaattagacttacagttccacatggctggggaggcctcaaaatcatggcaggaggtgaaaggcacttctttattttatcttattgtattttttttttgagacagagtctcgctctgttgcccaggctggaatgcaatggcgtgatcttggctcactgcaacctctgcctcctgggttcaagcgattcttctgcctcagcctcccgagtagctgggactgtaggcgcctgccaccatgcccagctaatttttgtatttttagtagagacagagtttcaccatattggccaggctggtctcgaactcctgacctcgtgatctgcctgcctcggcctcccaaagagctgggattacaggcataagccaccacacccggactggcaaaaggcacttctaacccaaaagtccacagtccaaagtctcatctgagacaaggcaattcccatctgcccatgagcctgtaaaatcaaaagcaagttagttacttcctagatacaatgggggtaccagtattgggtaaatacagctgttccaaataggagaaattggccaaaacaaaggggttacagggcccatgcaagtccaaaatccagtggggcagtcaaattttaaaactccaaaatgacctccagatctcacatccaggtcacactgatccaagaggtgggttcccatggtctggggcagctccacccctgtcgctttgcagggtacagcctccctcccagctgctttcacaggttggcactgagtgtctgcagcttttccaggtggatagtgcaagctgtaggtggatctaccatcctggggtctggaggatggtggccctcttctcacagctccactaggcggtaccccagtagggactctgtgggggctccaaccccatatttcccttcctctgccctagcagagattctccatgagtgcccagcacctgcaacaaacttctgcctgggcatccagccgtttccatacgtgttctgaaatctaggtggtggttcccaaaccccagttcttgacttctgtgcacttgcaggctcaacaccaagtggaagctgccaaggcttagggcttggaccctctgaagccacggcccgagctctatgttggcccctttcagccatggctggagcagctgggacacagggtactaagtccccatgcacacagcatggggaccctgggcccggcccacaaaacctttttttcctcctgggcctccaggcctgtgatgggaggggctgttgtgaagacctttgacatgccgaggagatgtcttccccattgtcctggggcttaacatttggcttgttgttacttatgcaaattcctgcagctggcttgaatttctcctcagaaaatgggtttttcttttctgtcacattgttatgctgcaaattttctgaacttttatgttctgcttcccttatatatctgaatgcctttaacagcacccatgtcacctcttgaaggctttgctgcttggaaatttcttccgccagataccctaaatcatccctctcaagttcaaagttccacaaatctctagggcaggggcaaaatgctgcccatctctttgctaaaacataacaagagtcacctttgctccagttcccaacaagttcctcatctccatctgagaccacctcagcctggaccttattgttcctatcactatcaggcttttggtcaaagccattcaacaagtctctagggagttccaaactttctcacattttcctgtcttcttctgagccctccagactgttccagcctctgcctattacccagttccaaagttgcttccacatttttgggtatcttttcaggaatgccccactctactggtaccaatttactgtattagtccatttttatgctgctgataatgacatacctgagactgggaagaaaaagaggtttaattggacttacagttccacatggctggggaggcctcagaatcatggcgggaggtgaaagacacttcttacatggtggcagcaagagaaaatgaggaagaagcaaaagcagaagcccttgataaacccatcagatctcatgagacttactcactaccatgagaacagtatgggggaaattgcccccatcattccattgatgtcccactaggtcccctcccacacaacatgggaattataggagtacaattcaagatgagatttgggtggggacacagccaaaccgtataagatttgtattatttttatatcacgcatgtgtgctcccccaacagtgttccttcatgcacatggatggtggtgttgaaaatgcactttctgttggacttccaattttaggccaagcttaggcccaggttgttgaattaataatgaataaatctagataaaaatagaatggtggttaccagggactggggcagggggaaatggggagttaatattcaatgggtaccatgtttcagtttgggaaggtgaaacagttcttcaggtgcatgatggtgatgattgtacaacaatgtgaatgtacttaatgccactgaatgatacattgaaacacggtaaaaatggtaattttatgttacgtatatgttaccacaattaaagaaaagtaaaaaaaaaaatcatagggaaaattcaatggaagcctcaggaaatggtaggggaggtcaattccaaaaccacatacattctaaaatgtcagaaatggtcagttagcaaagtaaggattaattagattttacacttgtatgtctttgttgacatttctgggaataaagagagaagagtgtgataattcctcttctatgtcagttctagaaccttctctttgttccttaccctggtgcttgtatgagtatgtacaaatgtgtgtgtgtgtgtgttgtgtttgcaaacatggacaatggcgtgtgggtttgctactctatttctagtatttctgtgccaccttattaataggagagctgtgggatcaggcctctggatgtaaccctgtccttccttagtgcttctgagttcaaactgactaactcacaaaatgcgtctgcatcgggactgacttgtggcattcattttctcaaagggcaaaaaaccttttgtttctggtagaaattaaagatgtgtttgtgacggccctcacttctttttcaccaaatgcagcacccttcttccctttaatccgatgaggtggttttatggggtggcacataacttggagctcatgggaggccttagaaccattaaccaactgagctaaccaggttcctccattagtttaatttcaaattgaattctccttcctccatgattgaaagagtacattcaattcctgtaacagtttttatatgcctgactgacatcccttcctaaagcataagcagtttgagggcagggatggtgtcttgtttatgtctgtaccccatgtatcatctagtgttcaataaggaattgttttatggaaatggattacatgattaaattggaaatagaatacaggttgatttattccacccccagcttgccttgctcactggatgcttattacagtgacctgaaggcacttcagaagacttaagtgctctctgaattattgaaggaagatggactttggctcagtagagtgtttgtgcttagtgcctattactggattctgattctttggaaacaaattaatccagagggatatattgtttctttatggttctgaatatttggttagcctgaatgtattaagatcttacaccccaaccatgacagatgggattaaaattccttccctgaaaagaatatattcctttatgcttccactataaatttatatgcaagtcatgttttgtaaagtcgtgttcttctcttgctatttggtgcattccctgcagcattctcgaggtcccagcattttcccttcaggagaccttagaattattggggtacggggcagccaagtgacctgcccaagcccacgtgtcttttttttttgttgtttttttgagacaagagtcttgctctgtcacccagactggagtgcagtggtgtggtctcggctcactgcaacctccgtctcccaggttccagtgattctcctgcctcagcctcccgagtagctgggattacaggtgtgcaccaccacgccaggctaatttttgtatttttagtagagatggggcctcgccatgttgcccaggctggtcttgaactcctgacctcaagtgatccacccacctccgcctcccaaagtgctgggattacaggcatgagccactgctccaggaaaaaaactgagtatcatgttttcttaaactgcatgtttaagatttcatatttaaaaatgaatattgtctggccaatgtgtattttaatttcttctctgatatactgtaaactaggattgttttaggaattataaggacctcaggaaacttctgtgtagtataaactggaaacaattcatatccaccacagaaaataattaacagtgctgggtgttaacaatacttaggggggaatgagtgatggagggaatgagtgatatggacaattggtgaagccctgttgactgggtgatcaggagaagggagctgatgggggagggattggaagctgaaggctgagaaggatttggatagatgaagggagaacataagagggagggctgacaggattcttgctgatccggctggttaatattggcttgccgcaattttccttctaagctaggtaaacatatatttccaggcatcctatttggactgtgtatagagacccattgacatctgggtctgttctaagctgctttcacttccctccaatcagagcttgttgggctcaataaggtggagatgggggtcatatattaccagggcaagcaggtctgtgcttcagagaggaagtaaggcatcagcgcttccctctagaattgatctgttgcggaacaagagtcctcgagcagcagagaaagctgcgccaggctccctttccctgctcagtcgttcgttcctggactagatgggtggaacgaggcatctctgggtttcacagagcacagagggcacggcttgtttcctaaccgggtctcaaaacctgcctgcactttgggctttttcttcaccttgccatatgttgcttcctgcccttttgaacctccttccggagctgattgcacttatgtgctaggaaaggagtatttgtcttcaaggtctgaatgcatcctaatccttattgaactgcaaggtgtgatggcacgacttggatatgtaagtgagaattgattctggtcacaggggatgtcataagtttcctcaaaaaataaaaaagaggaaaaaaatagtgagaaaaaatggcctttcgttatgggaaactgggagtttttcctacagatttcattcttgccttatatgggtttcctggaattcttctagcagattccagcagtgccctccggatctgccctctgccctctgagggttaggattcatagtgtgtgacagtctttgctattcaggccaccttctgggaaggccagtggccaggggatggttttcatgagccctcagcagggtgcagaagccaggtggttcccatatctcattcccacttttggtaaacacctctcttatgtttgccctcctgggcttgttgcttctggtccccggctcatcacagcttcccagggccccggtagaaacaagggttcacagtgggtaacctcactgtgctgcaactggaacctggcttgcaggatttcaggacctttggttgagtggttttagaaaaggctactcaggggctgggcgcggtggctgtggctcacaactgtaatgccagcactttgagaggcctaggcggggggatcacctgagatcaggagttcgagaccagcctggccaatatggtgaaaccccctctctactaaaaatacaaaaattagctgggcgtggtggcaggcacccgcaatcccagctacttgggaggctgaggcaggagaatcacttgaacctgggaggtggaggttgcagtggggtgagattgtgccactgcactccagcctgggcaacaggagcgagactgtctcaaaaaaaaaacagaaagaataaagaaaaaaagaaaaggctattcaggctgaagtgccctcaggccttggaaaagaaatgaaatttcaaacaaattattttctacctagcctttttcttttaacttcagaagggttatctggatgactggtatttacatacaaaaaatagttaaaagtctttttaaaccacagacagtataggtttcactggactcatttttcaagatacgaaattgttgaattgttttcctttggtaattgaagccagaattcagtgttttctctaacttcttttcggattctttgctctctggagaacgttttgcctgctttgcaagggggtatgagtagtgcgtgtgcctgagggcctctctctgccccctcctaacgcctggccaccctgttccgactgctgcccctgccggtcgggcccggacactgcagggagcctcacatcctcggatgacggattccttctgtacatctcatcacatccttttctactggcttcaacttgcatggcctctgtcttgaagaaagaaagaaaaaaatgaaatgtttcttctgccttcctgtcattttacgcattttacgctgatatacttgtaggcaaagggaattgaaagggcatgcactttggaacgagacaagcctcagttcaaatccgggctttgggaaaccatgatccgggagttctggggcaaagtaaggctttctgagctttagctgtattattggtaaaatgggaatagtaccgatctcctagggttatagtgttaaatgcctcccttcctttctttactctgacttcttccaccaccacctcatggtccctgcttccattcctcccttccttgcaagcttccggaattctttgtaccccgggcagcaatagcccttttggagctccccgcaattggcaagagcaagtcactgtgtgagcaagtcttgggctctgttatcttcagatgctcaagtcaggaaatagggggctagaggtcattgtgctcatttccccagagattatcaatgccacccccaccccttttaggagatggcatgtgccatgatcgtagctcagtgggatccttaatgcctagtgatctcggcttaacaggacaaagattccaagcccatgtctgttctgggaagcaaagcaggatgtgtgggcctggagtgaagtttgttccatttctcgccataggccatggtatgattacagctgtgcctagtgctgcctagttttactttcatgcctgatttgcaatgtggcagggtcctgagatgagagctctgtgtgggccagtagagtcaagagactttgtcctggtcacttggctacttgcagggggttctggaaagtcaagttctctgaatctcagttgcctttaatataaaaggaaagctatagtttcgggtcggattgattcacatggttttggtcgggagtgaatgaaattaatgtaaattaaaacacattatgagtagcaaagtgctatgcaatctaaaaataataacattttaatcactcttatcatcatatgtgccaggcactgttctaagctgtctacaaatagtaactcatttagttgaatgctcacaaccaccttatgagagagctactgttaatatcaccactttactgatggggaaactgaggcacagagaggttaagtaacttgtggaaggtcacacaaccagcaagtggtacaatcagggtttgaaccagtcaatgaggagtcagcatttgttattgttcctattcctatagcatagctgggaagggaatgcaatgctggtttggagaacacatcaagcaggcctcctgtcatccactgagtgagatgagatgatggccatacattgcggcacagccagctatccatggtctgcagcaacagatcagtcttgcagggacatggatagctccgctagtgaacattcacatgttcaacgaatagctagagtaaatagttggatctcatttagaggcatgctctggcattcctctttcacgtcatttgctttccaggcccactccaggatgagagtactgaggtgccataaaagaccaacaggcaagatgttggagggttcaggagctgccaaggttgtgttcatgctgacctgttggcatagcgtacagcagtgccagctgctctgctaaaccgaggagctgagatgctgtgttgatttctacatgttccaggcaaatacttcctgaggattgtcaggatttacataatggattggccctgggaccactgagccagcatctatctttgacaggtgtactgagagatgacctttaaacctagggatgttctccagatgcagttccagctgggccagtaagatctgcatgtttatggtgctgttttttcttttcttcccaggcagccgggattgcttaccacctgcagcgtgctttcggaggcggcggctggcacggaggccgggctacatgagaagctcgacagggcctgggatcgggttcctttccccagcagtgggcacactgttccggttcccaggaggggtgtctggcgaggagtcccaccactcggagtccagggccagacagtgtggccttgactcgagaggcctcttggtccggagccctgtttccaagagtgcagcagcccctactgtgacctctgtgagaggaacctcggcgcactttgggattcagctcagaggtggcaccagattgcctgacaggcttagctggccgtgtggccctgggagtgctgggtggcagcaagagtttgcagccatggatagttctgagaccctggacgccagctgggaggcagcctgcagcgatggagcaaggcgtgtccgggcagcaggctctctgccatcagcagagttgagtagcaacagctgcagccctggctgtggccctgaggtccccccaacccctcctggctctcacagtgcctttacctcaagctttagctttattcggctctcgcttggctctgccggggaacgtggagaagcagaaggctgcccaccatccagagaggctgagtcccattgccagagcccccaggagatgggagccaaagctgccagcttggacgggcctcacgaggacccgcgatgtctctctcggcccttcagtctcttggctacacgggtctctgcagacttggcccaggccgcaaggaacagctccaggccagagcgtgacatgcattctttaccagacatggaccctggctcctccagttctctggatccctcactggctggctgtggtggtgatgggagcagcggctcaggggatgcccactcttgggacaccctgctcaggaaatgggagccagtgctgcgggactgcctgctgagaaaccggaggcagatggaggtcagtgtctcttccacctctgtggcccgagattgtcgtgagctcagattagcactgggcagacggcaggaaacgaggggttctgggctcaactgacttcctcctggaagctgcagcagctccttttggagccggctgcacaggatgttgctgcacttctgtctctttgaattggacacagatctactctcctttttgtcttctctgtcacttaggtgctcagatttgctctccattgggccgcaggctcctctgcaggccccggcttaggtgagacgtggttcatgcagcccagctctgctttgagacaggaaactgggggcaggggagattgagctgggtgatgtttggaaggacttgagagatacaaaaatggggatgccctctatcagggcgatcctgctcttcatccgcttcactcattcccttccatcctgctgcagtggctgctgccttccacagggtcgggagacttcgcttctggtcattgaacctctttggctctcagtttccgtatttagagataacaccaactcataggattgttctaaggctcaaatgagaatgtagctgctttggaaataagtacaaactggagacatctaaggcccagccttatgatgatgctgtccatccagttatctgagaggtgggtatagccggaagtaggtagggttcctctacccacaaaagttcccagagaagcccaggtagagatgctctgtggctcatctgttgccagactgtattttaatcctggtgtgtgtgtgcatatgtgtttgtgtgtgtatgtgtgtgcatgtaggtgtgcctgtgtgtgcgtgtgtgtgtgtgcatgtttgtgctgtgtgttcatgtgtgtgtgtgcatgcatgtgcgagtgtatttgtgtgtgtgcactcatgtgtttctgcgtgtggtgtgtggatatgttcatgtgggtagggaagtggagcacccactcatcaggcatttgggaggagtaatctgcagaagttcatgctcattccccgaaagagcatctcccatctgttctttatctatagtctgatgtttctagccctgtccctgtttgccctgtttcagccctcagggacctcagcccagccctctgatttattatgcagagagggagtgaggtcagagcacagatgcaggcttgtattctgggtgatgtgtggagtcttccagttagtcattgcctaaaccagtgccccttgtctttccccgaaacccgttctgaagggcctccaatattggttgatttgaatgcatagaatctatccagtttcttaggccagaaaccacaaccatcttccacacctccctctcccttgttccacatctggctggcctccaagccctctcagtcctactgctaatctgtctctcatcaccatggccgctgccctggctcagatccttgttatctttcacctgggttttgcaagagggtctaccttcctgcggtgtttctcttctccagttaattaaactaaatgtgatcatatgtgcaaagtgcttcagacagggcctggcatctagcaagtacccagaaattaaccattgtcatcattagagaaatagctccaatttagagagtttgagtcaaaagcaaatgaagctctcccttctgtccatagctcaaattaaattcctatcttatctcaaatggtagaatctggacacactttttctttagtgtaagtattgttgtctatggcagtcccacagctgggcagggaaaaacatgacactctcttcaattacactcatgtactgcataatgacattttggccaatgacggatcacatatatgatgtgacagtggtcctgtgagattatcgtactgtgtttttgttgtaccctttctatgttcagatatgtttagatacaacaataccttaccattgtgtgacacttgtctgcagtattcagtacagtcacatgctgcacaggtttgtagccaaggagcaatagactattccatctagcctaggtgtgtaatgggctacaccatccagatttgtgtaagcacactctacgatgttcacacaatgatgaagtcacctaataatgcatttctcagaatgcatccctgtcattaagtaacgctaggctgtacttgagtatctgttgtgtgacagcggaattaacagagtagtgggaatgacagaaataacacagtccagggcataggacttacaggaaggcagagtttgcctcaataggaggaagaacctcttgacagtgggaattgtctgtgatgaggtgggctggtcagggacttgttttcccatggtgcttacacagaggccccagactgctcactggggttgcggctgaggccttcgggacagggcagaaggtcggtgaaggatctccaaaaagatcctttgaaagagtaggatttctagtgtttgggggccttattgatattatattttaattccttaacctcctctcgcttctacttaagtaaaaatgagaatctcaggaatttaagtttcagcttcttctaaaagatggttgatcctcagtgtttgtggattctatatgtgtgaattcgtctacttgctaaaatttgttgttaactccaaaatcagtactcacagcacttttatggtcatttttcatttttattttattttttaagagacggggtctctgtcttccaggctggggtgcagtggtgtgatcataactcactgcaaccttgaactcctgggctcaagcgattctcttgcctcagcctccctagtggctgggactacaggcgcatgccaccatgcctggctaatttttttttttttttttttttgggtagagatgagggtcttgccacgttgcccaaactggtcttgaactcttggcctcaaatgatcctcctgcctcagcctccaaggtactgggattacaggcatgagccaccatgcctggtggtaattttgaacctgcacagagtggcaaaatatttgggtcacctgatgtacacgctttctacttaggtcaaacaagttcagaccagatgacattctaccttctctctcatactacaaactagtatcctttttgtggtctatttaatgccatctttttttacatttttgtgctctttattggtgagttaaccattgcaaaagctccctaagcatagtgctaatgtcatctagtgctcctaagcatgagaaggctgtgatgggcctgatggagaagatacaggtgctccagaaactttattcaggcatgagttagagtgctgttggccagagtacaatgcttatggatcaataatacatattaaataagatgtttttaaacagaaacacacatagaacaaggtcatgtgttgaccgatggaggaaggtgttgtgaccaggggttcacaggaacctaccctgtatttctcttaggagcaatgcttcattattccctaattcagtattcacagaaactttatagaacataactacagtactgtgaataaggagaatcgactgtgaatattttatgctgtttagaaatcgctgcttagcgagatttccaggggttgcatgggactgagaagatggtaataaaggtagttggtgaccaggcatgcagaggaggggaagtggagtgggccctgcaaggtaggctccacctaaggccatggaaactggtgaaaaatgaaggactctgcctgtagagtattataggacgttccacaggcagggttgtggcacagtgcaagacatggccactggggctgtgcctccagcaaagacgggtattcagacatatctcagggcagtccattcagggccttatagagatcactggacgaggtgggcatcgtggggtgggctttggctgtaaatgaatttttttcctgaccttgtttcctgcatttcttcagcttctgttctaataaatgtttaatgataccatcatttgggtgtggcgatggtaatgctatagctttatgtggccctcaaaccattcacacagcctttcttctgtccctaatatataggactaagttactggtaaactttgcagttgtaatatagaacaattcaaagacaaaggggttaatatgattattcattcattctttcattcattcagaaagcatttatcaatgacaacgagtacaatgccttatcctaggctttggagattgtattaggacaggctagtaacaggcactacgttagaggctcaacacaacaaaattgtgttttttgcgtatgcttcatgttccacagagatttttggggggcaagaacccagcctgcaccatctgaacatatgatccccttagtcacagacttgaggaagagaatgttagggagtcctacaagagcgatcaaatgcttcagcgtagaggtaaccccatcactgccactcataccctgttcagcagaacaaggagagaggcacttgtactctttccatgtgcccaaagcagtggaaaatcaagtcagtgtaagtactagcatcctatacatggatgcaaaatagttcttgccccccagatgctcaaggttgaatgatgtgcacagataaaaccaaacaaaggaaaatgatggctataacctggtgcaataagtactccattcttccagtgggtcagaaaaaaaaacccaaaaaacaaaaaacaaaacaaaccttggcatcatcctggtgcattttcctcagaccccacctgtgatgtattagcaaatcctgttggctctatatttaaccagcggtcttctgaatataaccggaatcagatcacatctcaccatcttcaccattgccaccatcctcactatcatccaagctcctgtcatctccttcctgaatgttcccatagcctcttacctggcctcccttcttctgctcttactaacagcagccagagtgaaactgtttaaaacataagtcagatcatgtgctcctctgccccaaactctccagtggcgccccatgaacttcagagtaaaatcagaacttttaaatggtcttcatgtctcttcttgacacagtcctcattacctgtgtgacctcattctctacagttttcccttacatcaatctgccccagatccagtggcattcttgctccctcttgcttttcatttcactttcacttcttgctttcttaccagaaacacttctgcctgagcctttgtactggctcttccctctgtgggaatatttttctcccagacagcctcgtggttcactcccttaccacttttaaatctttgtgtaaatgtcaccatctcaaggagatcagatttcaaattgcaacctctaccctctaccagcatccctgatccccataacgtggttctcccttttcctttttcatagcattaatcacttgctaaggtgccataaaacttacagttgacccttgaacaacacgggtttgagctgcaagggtcacttatatgcagattctcttccacctctgccacctctgagacagcaagaacaacctctcttcctcctcctcctcagcctactcaagatgaagatgatccactttcacttaatgaataggaaatttattttctgttttttatgatgtttttcgtcatctagtcaatgctgaatttttttctcaatgatttttaaataacattttcttttttccactttacttttattgtaagaatgcatatataatacatataacaaacaaaatatgtgttaatcaactgtttatgtttttggtaaggtctctggtcaacagtagactattagtagttaagttttggggaagtcaaaagttatacacagattttcaactgtatagggccggggttggttcccataatccccgttgttcaagggccgactgtattatgtttttgcctagaatgtaagtctggagggctaagcattttatgatgtatccttgttacatcaacacaatacaaacagggcctgataactaagaggcactcagtaaatatttgttgaatgggtctatgaatagaagaagcaagtacaggtcacaatggaggtatagaggagggacatttaacacagactggaggagacgacacttgtgctaggggttgaaggaaatgtaggtgtgagccaggcaaagaatgggaggaagatgtttttcagggatcaagcccaaatccattcattggtaggccaagaattcattgtcagtaaatctttgggtctcctgttgagaagaacaatgattgtattagtcagttctcatgctgctaataaagacgtacccaagtctgggtaatttataaagaaaaagaggtttaacggactcacagttctacatggctggggaagcctcacaatcatggtggaaggcaaaggaggagaaaggcacatcttacacagtggcaggcaagagggcgcttgtgcaggggaactcccttttataaaaccatcagatctcatgagacatattcattatcatgagaacagcatgggaaagacccacccccatgattcaattacctcccacgacatgcgggaattatgggagctacaattcaagatgagatttgggtggggacacagccaaaccatatcaatgatgctaaatcaacccattggtaacactcagagaagtaactggatcactgagtctccatgtgggttaaataagactgcccttttctgtaatggaaaatatttgaaccttagggcttctcagaagggttttgtgagagctttgccatcattcttactcaagccttcaggacagcctgctgagcttaagttcatgctctttttctcttggtttctctgccaattttggtgtgtttaccttcataatttaaatgtgaggtttagaacagagaccatttggtcaggaaatttgtattccttctcattatgcatttcttcatgcagatgagggcacagtgtgatgtttttgtttaaaataaaataacattttaactttttatgtggatgttatgctcaaattacactttttctttacatagattttttttggagaaactaatgcatctggcattgaaaaaatgtgcatttttgttttttttttaaaaagagaatgaagaagttcagttttgaaacagtttctaaatgttcttagttttcacaaaaatgtttgcttgaattctattgtaattttttattttttcccctttaaaccaacataggtaatatccttaagattaaaacttcagaaacttcaggaagatgcagttgagaatgatgattatgataaaggtgagttttaatttgtttattgattgttttgtcatcatgtcccaattttctttccatctttactcatatctaccttttgaatcccaaaagaattgtacaatctgttcctctgatcatctctaccagggaatagttgactcttttacagcattattgttttgtagatttcaaagtacttcatgaacattaatcctttgggttataaatataaccttatcaattgtccagaaacacttagcatactcacacaataaaaattatattagcttgccacctgtctgcccatggtgtgatgtatctataatccacttgttcataaaaaatattcgtctgacacctatgatatgctagggaatatgggagacaataggaaagtaagacagacatggtatctgcccttgtgatgccagtaaacttaagccttcatggggctctctgacttcataatttccagaaccagagataggaaatgaggtgaatttgagaaatgtcagactgtgccaaagggggtcacatgcatcaaatttccacatacatggcctaaaatacccaagagaaaaagaagttacaacattagaccagagatagagggactgatctgaggccaggaaaacaaacaaacaaaaaaacaaaaaaaccaattaaaatgaggcaccaaaatagaagttgggctgcaagcagatgagggctgacatgggggttgaatgggcacagggtagactgagtcaaaggaaagctttcaggtatggttgtcctgggaacaatcctgaaaactttagatgctcattgcaagatctcacacccatttctgctgggtgtgggaaagagaaagggctgaaaatgacatgtagattcccctaggcacctactaacttgatagatactcctgctattgagcaagttaggacacccaggattggtatggaaatggaatgtaggtttaaggattcttctggaagctggttagctttaaggcatgggtcttcagcatttagcccatgtgacattctcattgtatgtgtcatagactgttggaattagggcactatgggaataagataagtgatttccaaaaggaggtagaaaatcctgttctaggaaatgctgttgcatgttctcattacctatccattgcagttgggtttagaaagtcacggggcatgggaggaagggagaaaggactggagacagaggagggactcttgacattctcccagtcctccttctacaggccacttgatttcataataacactgggtggaggatgagttgcctgagagaggaggcaagtcggaggcaggggaaggcacccctcaagcccagcgtcagtggcttcagcccgggcacagctgtaggcagggagaaaatgagctattcctgggccagggctcacctgctggatttcatctatgagagataattacagctagttttgaagctgcccactattgcacagatttatttttattaccatgaattgagtcctggactctctacctttaatgacactgtaacacataaagcttacaatgcagtgaaaaagtaggccaatcaggccctagtttgtcccctgctcctgcttaggacctggtgaactttctagacttggttctctcaatggcttcattaaatggagacctcacttctttaatcttttctccaaacattccgcaacacctaagggaatggggtttgagaggatacacctcatcagggaagctcaaagaatacaggcatcttctttagtgtttctgcctttttctgttcctgttggtacctcactacttactacatcagaagtttcagggtgggctcagcattctgtgttttcccaagccttcctggtgactggggtgtgtcctgaggcttgagaaaacactggtgcagagtttatgctagtgagcactaggcctggggggcagcagccagccaagccctggaagaggggttgagtctatgggttgtgtccttcctgatccaccagaaatctgtgcagcagacaagcatcctggtaggcacaggacacacgatttacatgccactccccttgtcttcttcccaatcccacagacccgattcagtgtgttgagaggtaaatgttcttgtggaatatgcacaatgcctggcatataggaggcaggccctcagtaaatgtggtgtgagggaattaattaataaatgacttgccctctgaatgcatggaggctatggatggcaagctagagtgaatgaggggaaagcactcttgccacctgaaggtgggcttagttctcagctcatctgcagtgcccaggatggggggtgggagtgggtgaagggaaagatctttgcatagaaattggcttatgtgaaaaatatctattaagtatccatcgtatacaacagcacttctcagactttaatgtactgtgaataacctgggttggtaatggattaaaatgcagattctgggctgggcgcggtggctcatgcctataatcccagcactttgggagtccgaggcgggtggatcacgaggtcaggagatcgataccatcctggctaacgcagtgaaaccccatctctactaaaaatacaaaaacaaaattagccgggcgtggtggcaggcgcctgtagtcccagctactcgggaggctgaggcaggagaatggcgtgaacccagggggcggagcttgcagtgagccgagatggcgccactgcactccagcctgggcgacagagcgagactccgtctcaaaaaaaaaaaaaaaaaaaaaaaaaagcagattctgattaagtaggactggggtggggtgggagactccatttctaatgatctcccaaatgatgcgcatactgcgagcctgtgagccacacttagagtaataatggcatataagaaatcacaacaacgccgtgtttaaggcatgctgaatgccgtcctcaagatctgcatctcaaggagtatgcagtccaggaggggaaactggataatacacagtaagaataataaagctatacataatatgtcccttaaaatacagacagaccaaggcgggtggatcatgaggtcaggagtttgagaccagcctggccaacatagtgaaaccccgtctctactaaaaatacaaaaaattagccgggcatggtaacaggcacctgtaatcccagctactcgggaggctgaggcaggagaatcgcttgaacccgggaggcggaggttgcagtgagcagagattgcaccattgcactccagcccgggcaacagtgggagcgagactccatctcaaaaaaaaaaaaaaaaaaaaaaaaatcattaaaaaaaaaaaaataaaggcagacaaagacagtcccaaaaagggacaaatgacttctgccagagatcttggggtgggtgaagaatcaaagtagtggaggaggaggcagtttagatgggtcctgaaggatgggtttgatgcaacagacgggggattccggcaaagagaagagggagaaagggcactgttaccatgagccaccatcccaaagttaggcttgaaaggagacttgcggctgactatggtttgaaatggatcacaaaagggctcagtacttggacttgtcagtgattggtaaattattttaaattgatcactaagtcattaggaaagtttggaagacataaaggtagtgaatttgttatgagatttgttcaaataagatttgaatacaatttcttctctcatatagtttattgagggtaactattaaacattttaagaattgttgagttttttttcatagtttttacttgaatttgctcctttttctctgttaggagcagctgtgcagccctgttctgtctcaggggaaggggtctgccggaatctgagaggtttgccagaagaaatgagagggtgaccttatctcccttggtttttgctgcttttttcttttgctctttctttctttctagtttcactcataaaaatgactttgtgttccaaagaaagaagcagttggcacagagtagagagttccatgttttatctcatcaaagtgctggcaaatttccccactaatgccttgtgggaaaaccttctcaggctctaattacatctctccatcactagggaagtaaagttaatatttccttatattcttgcataaggtgggagggcttgggagaaaaagaataaggctttcattaatagcatttacatagcgtccttctccaagaggattcattagattcattgcattaatgcctatcccgtgcttatggtcaggtgaggggcaagtttgagacctcagagtcctgcagagagaggaactgtggtcagtactggagataaggattccagctctgacatgggtggggcagagaaaaggtgggcgtgggcaacaagtttctatgtggttactgttctagttgttcctgcacctgtttgtgctccataatatccccctggctgctcgtgtcccatgtccaccagtgaccatgctcacttccacttccgatgtgagcaaggagagattgtgaaaaggaaggaacacaattttgggaactcagcatttcagtgacttatatcaatgtgacatatagagcagctggcttcactctgcttgtttagaatttcctttttggagtaaaagtagccagcatcatattactttctctatcctttgtctcaactcatatccattatgacagaactctagtataataaacatttatatacaatgacagacattgaaagaaaaatgatggtggcagaggccaagcataaaaacactctagggtgagtcttttgtctaaattaccataatgtccctttgtctgaattatgtcttgagcaagatttctctatcctcagtttctcgtcactgatggtgtgcaaattacccaaccagatacccctttccttcacagggaacctgccttacacctcccatagggtttggtgtctcagtggcttccaggagcctggcagttagaaccagaatccctgccacataagcgttgatgtgactgaacacagcacaaccccagagatttccttttccctgcgtttcattccagtctagaaagaagaaaacaagattgaatagaatcggccattcttatatcctcaggactgccagaatcaatgggatgtggatgccatctgggggcccatggaacataaatgcttttcttggcagcctgtatcattttgctttctgacttaaaaagtggactgcaattgaaatctgctcagagagacaggtccagcaaaactgtcactttataatgtctgcacagaaaaaaagcaaacatgaccaccagtgcaatttccacctgcagctcccttgaacaaggcacttctctccctgcctgcacaccaggactttggagtgttcttgtctgtgctctatagagctttcccttcctttgctgtgagatctgcttgctatatcaccttgtcctggtatactaacaaaatctgctgattaaaacccaaatcaacagcacaaatctcaagtatgtgtcatgagactcactacatccttagcacctatgaaaaatgccaacacttagacctgcaaacacaaatgggaaatgctcaaagtcaccattggctatgatctctgcccttaatttacagaattgtaaattctcctgcaaacaagaaaagaagctaagatagtgcaaccaagtaaaagaaatgcattttatgtttcctctttttatgatatgtttgttttattagatagctgctgctctggtgccaatcataacatcaaaagacacaatcctgaatgccatcatcctgaatgttgaaatctcccaaaattaaatccctaaaaccccaaattcctaaagtctaaatccctaaagtctaaaatccctaacatctgcaatcctgaaaaatcacagtcctgaaagattaaaatcccacatgctggaattctgaaaaccaaatgtggggaaaggatttagtgagttttcagttgtgtcatgttagttgcatcatggtaggtggaactattacctttttattgtctttatttggaaattaggtatggtttaagaagatgcatataggtgcccagctgacaaggggtggacttgtggacttaattttaagtgtcaacttgattggattaaggaataccttgaaacctggtaaagcattattttgggtgtttctttgagggtatttccgcggagattagtatgagtctgcgtgtactaggtagggaagatctgtgtttaatgttggcaggcaccatccaattgtcaagggcccagagaaaacaaatgtagaaggcaaactggtctctctctgagaaatgagacagactttttcctgactccatggacatcagaaatttgactgcatgaaagtgcattataacaacgttgactttgtgtgtaagcactgtgcgtatacataacaatgttgggatttcctcaatagatgaagagatgtttggacatcggcatttgtgaaaaataaaatttcttgagatctctgctctctgggtggctgcatacgaccccctttgcaggtctgttccatggtgggcacattctcattcgttgcctgattacatcacgtgattactgtgggaagaattgtcactatcattggttttcaattgaggtcacggaatctgctgagagggaggaccagctgtgtgctctggccactctgtcaatttgtctcccatccctactgggtctagagcattttgtgtcccactgtggaagactgagctttgtagacagttgaggacaaaacttagctaggaagtttctgacttctggaagctcttatttacatgacagtgaggaaaggaacagatgagcgctctacacttggaggtagcaggaaaattttccacaattttatctaaaaaattagtctatttttatctaaaaaacaaatgctaattaggaaattagctcttgtgttaatcttcagcattgttctcagaagagcataagcttgtatatctgcttcctcttactttaagtaaaaacttaaaaattgggagatgggctatacattaagacaaaaataccataggaagagaatatgaacaaaatgtcatttatttagtttggttgcattgtcttagcttctcttcctgtttacaggagaatttacaattctgtaagttaaaggtagagatcatagccaatagtgactttgagcattgcccatttgtgtttgcaggtctaagtgttggcatttttcatgggttctaagggtgcatgcagtgagtcaagtgccacctacttgagattttagctttgtgcacagatgcgaaagcttattggcacctgatgtacttccctctcattcacagcataaaatgtgatttcacggtgatgccttttctccttggctttgcatctgggtacacgcttggctctttcttaggtcttggtaggatttcttggaacccctgcagctctggaggtagaagcacagtggcatgaacattccttcctaactgccatgtcgtggtgtgtttctctctgggaatgggaccatgctgctttgccttccctgcctatgaggacatcagtctgtcctataaggatcacgtggaatgttgtcatggtgcattattcatgataaactctgacagcttcaaggttaggtgtgatgctgaatacaaaacaccaagccaagccaaaagaattttttttctctcctctatgtcctctccctaaatggttcttagttgaaacttgtacatgatttcctactagctgagctgtagcaggaagttgtgccaacagtggacacatcttattcacgttttatgcatctgcctatgagaatctgtaagagacacaagctgttcatgttgtagagtccggttgatgaagcagaatgtggttcaggaagtctctcttgcgcttagaagactgattttgaagctgctctcataggttttctgcttcttctgccctcttagagtggctaggaggctgcctgggattccaggtaggtcttggccctaagacctcagctgctccatagaaggtggtcagacaggatgaggcagtgacttcgatcatctggagcctcacacctgtaatcctagcactttgaaaggctaaggcaggagaattgcttgagcccaggagtttgagaccagcctcggcaacactgtgaggtcccatctctacaaaaaaatgacaaaaacaattagccaagcacaatgtcatgcacttgtgttcccagctactcggaaggtggaggtgggaacactgttttagcttgggaggccgaggctgcagtgagctatgattgtgccactgcactgcactcctgcctgggtacagagcaagaccctgtctcaaaaacaaaaacaaacaaacaaacaaacaactcaatagaaatgaaaaggatgtgctaatgatgagatgcaaacccccaaattgacatgtaccttcccattacaacaattttaggtccaaactatgcatttggtgcaaatggttgatttatccttaacaggaaggatgtcttagtctgcttggactgccatggcaaagtactactgactgtgtggcttaaacaatagaaacttattgtctcactgctctggagattggaattctatgtcaagttgtcagtagggttggtctcttctgaggcctgtctctttggcttgtagacagctattttctccttgtgtcttcacacagtcttccctctgtatgcattggtgccttaatctctttttataaggacaccagtcatattggaataggatgcatccttgtgacctcattgaaactgaatggacctctttaaaggtccagtctccaaatacaattaaattctgaggtcctgggggttagaactccaacatatgaatattgcagggggatataatttagcccaaaacaggaagcctaaagggctcaggacagagctaaatactaaacacattcaggtatttccacaaaatttgaataatttagtttgactatctgtggtcattgtggtcatatgtagacatttgattgaaacttttttttttttaaagcagactttttgacacaagtaagttgattcttcccacaagtgaacttagctgtttttcacattttgttttattgcagagcctggctcttaggccagtatatcatggtatcccttcacacaaacaactatctactgaatatctgccagatttaccatgccatggtggacaaactatgcataaaaaaatagcccaccaccaaggagtatacagcctagttggaataaaacataaatagtacaaatatgtgaaacttcagctaccattagaattgattttttttttagaattgatgtttatttttgtatgaggacatatttctttttttttttttttttttttgagacagagtcttgctctgttgcccaggtggagtgcggtggcgtgatcttggctcactgcaagctccgcctcccgggttcatgccactctcctgcctcagcctcccaagtagctgggactacaggtgcatgctgccatgcctggctaattttttttttttttggtatttttaatagagacggggtttcaccgtgttagccaggacagtctcaatctcctgacctcgtgatccaccgaccttggcttcccaaagtgctgagattacaggcgtgagccaccacacccggccatgaggacatatttcaataggcagagacacaaattaacctagtaccagctgaaaagaggacttatgatctggtgtgaccagaggtggcaagagtgatcagaactaacatgttcttcacttttttctctgtattgtggcttattctttcaaacctcttgttttagttatttattgctgcatgtggtaggctgaatgatacccatccctccccaaagatacccacatcctactccttggaacctgtaaatgttaccatatttggaaaaggggccctgcagatgtgattgaattagagaccttgatgagggcatcatcccagattatctgaggtgggtcctatatgacatcagatcatgtctgagagacgcagtgggagattggaccaattgaagaggaggtggtgatgtgaccacagaggcagggattggagtggtagctccaagctaaggaattccagcagccactggaagctggaagagacaaggacaggattttccccaagaatgttgggagggaatacagtctgtcaataccttgattttagctcagtaaaactgatttcagacttctggcttctaaaactatgagagaataaatttatgttgttttaagcctgtaagtttgtggtaattagttatagcagccacatccaatttatatacggcataaccaccaaaaaacttaaagcagtgtcaaagagttacgttagcaaaatggtagagtttgcagctccaactttgtttccccacagaaacatcaaaaaacaaacagaaaccattagaacaaacatgatcagaactctggaaaataggcaaaagtttaagcaaccaagcaaacactgaataaagaaaaaggccacttgaaaatggtgggaaactttgtagcatttttatttgctcttgtcccagccttctctctagcaagacagttctcttgatgatgacagccttcattcccagtgtaggaccctggtccctgattacagagggactagagaagaccttacttattgtggatgtctgttctaacctgtctgggggctacgttaagtactgacataggcatgtatctctgcttagcctaacttagaacataggctggaaaaatggcaggcattgcttgcaaatgctcaaagtgagctaacaaactgcaaaaacctggggcaaaagattactggttgaggtgtacaatagaccatctaaagtccaggtgcaaaagctggaagagattctgtgggaaattaggacattcaaaagcaccatgtatacaggaaaatgtagaaatccatgcatatacttggggcaagatgcatgctcagaaaagaggagagaagaccctaagctttcatttcaggctgatccttgagttaagtgtaaggttgggtaagtattgtaggagttccccagcacagagccaatctgcaaagactgggaaaggaagtttggaaaagtatctacaaatggactattatagccatcaaaaataaaaaacagcaaaccctggggaagggtgagagtccgatagccagagtttacattataatatttaagtgcccaatgttcaacaaaatatcacaaggcatacaaaggaaacgagaacacatggcccattcaaagaggcaaaataagtggacagaaactatccctgagaaagcccagccatcaaatttattagacaaatactttaaaatgattgtcttaatgatgtccgaagaactaaaagaaaacatggacaaagaactaatggaaaccagaaaaacagtgtatgaacaaaatgagaaataaagtgatagaaattataaaaattgatcaaacagaaattctgaagctggaaactgaaagctgaaatgaagaatttactagagggttagacagaagatttgagcaggcagaagaaagaatcagtggacttgaagatactacaatggaaattattgagtttaaagagcagaacgcaaaagtaacgaagagaagtaaagagaactgaagggacctctaggacatcatgaagcagatatatatatatatatataggagatatatatatatatatatataggagatatatacatatatataggagatatatatataggagatatatatataggagatatatatataggagatatatatatataggagatatatatataggagagatatatataggagatatatatatatataggagatatatatatatctgctttcactgttgttattcaacattgcactgcaagttatagtcagagcaaaatagataaaaggcatcataatggaagggaacaagtaaaattatctctatttgcgattgacattatcctatatgtagaaaatcttaaagaatccacaagaaagctgctagagctaatgaacaaattctataaagttttaggtgtgaaagtcagtcatgtttttatctacctggaatgagcaatcccaaatggaaaataagaaataattctatttataatagcatttaaaataataaaatacttggaaataaatataaccaaagaagtgaaagacttttacattaaaacatgtaaaatagattttaccttgaaacatgtaaatagagtttacactgaaaaatggaaaatagcccagaaatacacccatatatctgtgtccagttgatttttgacaaggggtgccatgtccattcaatgggagaaaatagttctttcaactgatggtgctggggcaactggattttcacatccaaagaataaagttggactcttgtctcacaccttaaacaaagtttaactccaaactgtgcaatgattatggataaaagtttggtggttcttcaaaaagagaaacatagaatcaccatatgattcagcaattctactcacagatatgtatgaaacagagacagagagaaagagacagagagatggagatgggggagagagacagaaagagagaagagagagaaacagagagagaggagagagagagaaagagatagagaaagagacaaggagagacaggcagagaagagagacagaaagagagagatagggattgagagagagagaaagatatagggagagagagagagagagagacagaaggagatggaggagtctctttcacacatatgcacatacacagtatgctggaggaagctctatcatcttttatgaatctatcttaagaaattatgttgttacttccaccacatggattcatttgaaacaagtcatcaaagttgtcccatcttcaaggggagaagaattagaccatttttttgatggaagaatgtcaaagaacttgtgggcatattttaaaatgatcacaccactttattccatgtggcaattgaagacttttgaattaacaacacacgactagagaagaaaaagagacttcccctcttggatccagtatgaaaaccttcaggaaaggattgggttaggtgctgactccttggaccaatgaccataaccaaagggatgaggtactgtaattagccaagcctggctacctgtctgtctctgtgatgaggtgggtacatctgttaggagaagatgggggaactggtgcagtgcagataaaaatagtagctagtatgcttattgcctagtgaaacatgcacacacgaagcaattaggaatgattgagagcagcatatgaatttaaaaatgagggatgttaaaaatgcataaagaagattaaaatgaaaacactccttacagagttcattcatttggtcaatacagtcatttccatcccaaaacctggtcttacatgttagcttgaaggtgcctcttgcaacagattttccagagacttttgtctgatctttactgggaccaaagtaacctttgattgtctctgactgtggctgtttttccgggagctttatgtgctgggctcaggaaaagttcaacccatcaataggttatcacatatatatactctgggactgattggaccacattttctcactgaattgactgattgatgaattcagttggcagaattaactcttctatgtctacatgaagtgccatttagaaataatcaactcttaatcagcctgggatagtcagtactaaaagcaccttcatgagctgtgaaaaatttaatgcatttatttacatatttagttttaaattttagtatattgttagttgaggtatagtttccaaacaaagagccgtgaaatgtttagtaactgtctctgtacctctggatgaggacagctcagccgggaatggagggggactgggtgaggagaccagaatgtcagtgtggccacgcagcacacttttgttttgtcttctgtccttgagcactggcttgttcctggataaactaggcataataatacctatcctgctgtgtgggtggaaggttaaatgtgataatgatgtgtgtgagatgcctgcacagtgcctggaggtattgaagaattatttgctgccttttctttttctacctaccacttacccgctacccccgggtgctacatgttagaaaacactgtgtaaagtgtggatgcttctgaaaaatctccctgccagcagttagtgccaatagcgtgcagaaaataagatgcaatgatttggcttcttttctgtttggcaataagaagcttatttgcacatagcctgatttctttcaatctgcaaaaaaaaaaaaaaaaaaaaaaaattggaaatgaagtaaagcaatatatattaaaacatgcaaattataaagaagcatttaaagcttgactactcttcacattgtcgtatggcaaactattttttttatctgcagtctaaatttcatgacctagaatctctcgttttctgcctgatgctttgttgcatgacattggatgacattgagaaaatgaaattgaaaccctgcttgggctttgaaagaactggcaatcctgcttttaagagtggaaggaaaaaaaatggaaagggaaggaggaaaatgggacatacatggaatactaaagacaagactgaagaaagagaggttgaaagctctctctcttctgattatgtttctgtgggtctctactgttttgtggccttcaccctcatgggcagagaaggctaaggtgtaattctcaggtaaccatttaatctttccatttaaaatggtcatttacaaacatggagtccatttcctttgcataatggcaacgagatggcgcctgtgctccttcgtggaattccttgctctgggtgatggactggacagctaatttgtttttataaagtgtctgggcacttaattaaatcttgcatcctgtctaataaaaaatcaatatcaaatccactaccaacacgttctggatggcagagaggcatgccagaagaaaatcttgagtggcggttccttccagatttagcaagctctgcagtttgtgtttggctgttctctcatcctgttgcttggcccagctcctattcctaagtcaatgagggcttcaaagcatggtgtgaatgcatcagctgctctagaggtcctgtctttgggagatgaattgtggcccattaaaaaaaagttctctagggaaatgtgaataagtttcctgcaagtctcggaaataaaaacctttatggtatataaatatttgaaggttaacaaggtcttcagttggaggaaggcaggcccttggcagaaccaaagggcatctcacgagtgggtagaaagttataggagggattggtcttttctgctttggtgttggtagtggtagctgggggtctctaggctgattgtgtatgatctggagtctggagccttgctacagtttgaactggtcagaaaagccaaggcccgcctccctctgctgactctgtctcactgagccataggcgtggggtcctgcattggccatttcagttttgcttgttgatgaggaaaggtagatcaagtgggtcaaggtggcaagggtcatgtattcagagtagggactgagtggagttggaaaataagcagcagtgaagaagagagatcactccagcaatcttctcactgccaaaagtctccaggtcccatagactgagcaccattcaataattctatgtgttgtatgaacgcatcagacaattccacaacctccttacttcatttgcctgcttgtcatagtgcgagaattgctttcattgtccatttgtttcttgacagtcaaatctcttcattaataaaaaaaagacttgaattcatggcaccattttattttccaatactattgtatagaatgtccaaactaagatctaaagactatctttttcccaccatccctctagaagttcctgatctttgttatatctcagcccagctcctcttgccatatcccctattacaatgaatggcaagtcaagcagaaaaaagcctgtgggtcattgtggactttcagctctcccgggacatgagccactacaccttttcgattttatctcttaagtatctcttccttctgtcctttgatctcgtaagtgttgtctggacacacgcactcgcctcttaatgggtccttggtttctattttcatccccctccaaagcatccttcgatcatccttcaatccctttccaatcatccttcatgttgtaaccagtcttctttctaaaactcatttacaattgcttttctacttcatctctctgcgtgactaatgtcactatcaggacaaatgttaaatatggcctgccataaccagcttcttgccctggcccttgcttagctttctaacttcatttcttgccattccttccccacttcaagcaactttctctctctttttttttttttttttgagttgcagttttgctcttgttgcccaggctggagtgcaatggtgcaatcttggctcaccacaaactccgtctcccgggttcaagcgattatcctgcctcagcctcccgagtagctgggattacaggcatgtgccaccacacctggcaaattttctatttttagtagagacggggtttctccatgttggccaggctggtctcgaactcctgacctcaggtcatctgcccgcctcggcctcccaaagggctgagattacaggcatgagccaccgcacctggctgcaactttctctttagtcccttggtagttcccgaaggcatactgccttgtccactgtttagtacctctgcctgcacttaccacctccttctcctactaggtcatccagacttggtttctggttggtcatggtgtggctcatgccacaatcctagctttttagaaggctgaggtggaaggacttgaggccaggagttgtagaccagcctgggcaaatagcaggaccccatgtctataaaatttagccagacatggttctgtgcatcaatagactcagctacatgggaggctgaggctggaggattgtttaagcccaggaggtcgaggctgcagtgagctatgatcacaccactgcactccagcctgggtgacagagcaagaccctgtctccaaaaaaacaaaaaagacttggtttcagtgccccttcttttgagtgcttggtatattctctatcctcatacatattgtgttttagcatagctattggctgtcctcttggctaaactgttagcctcctagggaaagaaagagaccctattcatttttactctttatccccagaacttagcatagtgcttggtcaaagtagcctctcaagaagtggttgttggatgaacaagtgaattcatcttattatcacttatcatcttattatcattgttacattattaatagctgataattctactaacaatagaattataaagcattagcgcttttcaggtcaaccttgttaaaaacctgactgcaaagttcaattctgccatttgctagctgtataagcttggacaaattattctttctaagctttattttttctatctataaaatgggataatagggttgtttggagaattaaatgagatggtgcatgctaaatatttgggttatgtatcattcagaatgaatgtttgttaaattatactcactggtaaaatatttaggtattttagatctgctagagataggctttttaatctctttgtgtttttttgttcaaatgtatttgcgaactgattattttcttagataatcattttcttagataaccttagcgcacctaatttgatgcctgttgagagtagagagaaaccaaaaagctcacagcagaaggactttttcatatgtcacagtagacaaacaaagttattttccgtcacagccatgtattgggtggaatgacctttagtagctgatgacagcgatgtagcagaagggacagttttccacttgacctttatacaatgtgccagcttgtctcataacacaaaataagttgcaattgacaggcttgatagaacactaacccgacaggagacagacactgaagcagcccctgtttgctaccgagcgtggcaacttccatatacccaagggtgccttagggatgactactccctaagtcatggccagaacatttagtgtaattagttcctctaggagtttattgtgaggtgagatttgctcataatgaattttcattgccaaggacttggttactcgcaggtaccccactatagtcttttgacttttctgtctttgtaagctgtcttcctctcctctttgctctttgcttttaccataggatgttttcttctttactctttttaattttttttctttaagacagggtctcgctttgttgtccagctggggtgcagtggcatgatcatagctcactgctgcctcaaattcctgggcttaggcgatcctcctgcctcagcctctcaagaagctgggattacaggtgcatgccaccacacttggctaattttaaaaattttttataaagacggggtctcactatgttgcacaggctggtcttgaatgcctggcctcaagcgatcctcccacttcggcctctcaaagtgctgggattacaggtgtgagctgctgcactcggcctcttctcagcttttctaatgcttcatttttgccgttccctaacttgactacttttagttttctaccactgggtgatttttttcccttggggctttctgcatgagggctaaaatgtgaagttcttaagtttgttccttctcccagatagtcacagggcagaagtggttctgagcagcaggtaggaaacgtgttgtcgtttcgtttacaggtttgttggaggaagttgctaccagtcatctcactttgcatacctagttctgacattgactgcattaataaccacaggtaatttactttacctctgcaggaagtcactggaagggttacattttcatttggggtgaacagcaactgtggatacggatcatgaaaattgacaatggaaaatgggcatgccattctggaacttttcccagcttttttccagagccaggtctgtcctgatgtgagaaatgatgtcctcatctgccaaatgagtacaataacattagctctacctaccttgctgggctgatgaaagaaacagaggaaataatacatataaaaactgtgctgattgaaaggagctatgtcaacatattaattcaggctgtccctggttggagtattgtggataatgttatctgctttttattcttttccacactacagatgagaaaaaaaaagaaaaaaagtttttaggagagaaaaaattagaagaaaatgtttaaaaatactctctggaaagttagaattctaataaaatccttagtaccacagttgaggatttggcttcatgctccaagtaagtaaatctggctgagattaatatttgatgcatatttatgaaatcagatatatcaaaactgtatcaaaagatataaatcttttatttttcttaaagaccttatacaatgaaatgttcatttatatcatatggacttggggcagcaacaaaaacttggcaacctggggttgctgatatttacagctgttcaacattttttggatccatgtattctcttcagaacaccagaaggctgtgcttttcaaattgtgttctgaggagcctcagagatgccttagggaaggctgaaagggaagcttcatccagagtactttggtttccatctgttattagtccagtaagatttcatctcaaaaagggctctgttgctaaaaaaaagaaaaagaaaaacaaacaatgaaaaaaaatcaagaagtaaacaaaaattcttgaaaagcactgccttggctgggcacggtggctcacgcctggaatcccagcactttgggaggtcgaggcgggcagatcatgaggtcaggagattgagaccatcctggctaacatggtgaaactccatctctactaaaaatacaaaaaattagccgggtgtggtggcgggcacctgtaatcccggctactcgggaggctgaggcaggagaatggcatgaacctgggaggcggagcttgcagtgagccaagatcgcgccactgcactccagcctggcgacatagcaagactccatctcaaaaaaaaaaaaaaaaaaagcactgccttaaggactttcaaaaatggtatctcagatatttggaaactattgttgagagaaaggaagagattcctcccaaatctgggcttctttttcatgtttgatagaagcttgaattttgagtaaaaaacacagtatgctagagatgcttaacctggaatgaccatggtatttgatacaaatgaggttctgtagaatgcggacagctctgcctggactgctctatgaaatagatctttctacttatccacactcgaacaagtgtgtccagcataaacatcacagtgcttcaaatactgttagtcccagtcaaggatgttattctgtgtccattccactcagaggtattcattttcttcttggcagctggaaccaattgctttggatccaccatggaagcctcgacatcctgaaccaaactcctattaatgaaacttcccaaatttaaacaaagcaagacaaatagatatccacacagcgtagatcatgacttctttagacatcatgaaaagaaaaagaggacccacgtggaagaatacgctcatttatgatcagggtagcatcatattcctctcccttttaacattatttcaggatagcagctgtctggatgctggccatgggcttgaaaaaaaccaggtgggcatttctgcccactgtaccgtttctcccatgcgtttcccagtcccctgtcatggcatgaaaaaaccgctattgttctgtgttgggacaattagatttttgtaggggggagaaaccacccaaaagcaaatggaaaaatatgctatgattaatgtcagttacgggaattaaacttcggaggcctttaggacaattatgaaggaatcattttccccttggtggcaatatgtactacagttgtcccttgatatccatgggggattaattccaggacgcctgtggatacagaaatccacgtgtactcaagtcttgttttcagtggatatgaaaagtcggccctccatatctgcaggttttacacttggcgaatgctgtatttttgattcatgttggattaaggatctggaacccctgatacagactgtatttattgaaaaaaaagtctgcctatgagtagactcacatgttaaaacccgtgttgttcaagggtcaaccataactgcaggcagaacatctgggcaggaagctcctgcaggaagacctgtaaactcaggtctgtggaacggcgatgatcttcttcccttgcttccagaattattgctgtgagatcaggtgcactggctacgaccttactctttgtgcagaacttatttctggtctggtttaagtgaacgttattctttctgtgcagtcctgtggttacatcaaaggttaaatttcatatagcatgtctgttatatcagacttctctgatggcctgtccagagcaaacattttaaaatattggtcctattctgtcttataacagtctgctctggtgatttttgtctcatcaattggattatgacactgtcagccactgttagtgttattctcaaattcagtcttccattcatctctccttcccttccctggcagacagacttctgcagccactgggactgttggggtgaggaaaggcaagaaccgatgtttcattctctccctcccttcttcctccctcttttttgttggttgtggggtgggctacatcttcttacccaaacaaccaggagcatttgttggcaagtttattgtgcccagtgccgatgtctggacaattaattccctaggtcagtggctttcacagtttgtgatggtgactccaggaaggaaactcaccatgaccctgtatatacatagaacaaaaatgcagcccactctgatattgtatttgttctattctagttcattaaaagtgaatgttggtcttcatcctttaaatggatattgtcacctgctaatagattggaccagtattttgaacaatggccttggtacttcatttggctttgcatgctcaattgtcatatcctctgtgtggggaagggggcagggctgcttccctgggctgtctcctgggatgcccaggggccctgcctgcctgcgtgggtccttggggacctagaagttccggtttttcatgaaacagcggggctctagaagagccttttcccttatcctgactcttccaccctgatttcttcccgaggacttcttactatgtttaaagatggcagtgaagtggcctgtgggttctagaaccctgcaaacaccgccccaagtgtgataccagtttacccttcttgtagacatccccaaacttcaaggtgattgtcttgaagcccttcacccaggagagggaaccagcgtacaataaccgctgtgctgcaggagaggggaggtaaagagttgtcacagccttgagcctagctcttcccccaaaggcagtctctcttttcttcttccttcctttagtcatctgtgtatagacacagatgcaaacttttcctggaattcctaatcttaagcagtcttcaataggagacctggcttcagagtctgtccttaaacagtgaggtgccctgtgtcttttattcttaactgtggcctatctccactcaaattttggcatttggcgtttggttgtcagtggttattttacattctcaagtgtcactgtcatttggttagggtagttgtcagggtggttctgagagccaagtgtgtagcatagctttgttgttacaaatgaaaaggccagttcccatttaaccaggtactgctggctttctctacaggatttggtctacatccattagtgctctttgttgtccatgtcactataggagagattccccgggacctacagggtcactgtctgtttccaggatcagtgagtcactccctcgtggcctgttatggctttgtgtgaccacagagagggtcacggtggtgtggaagcagggagggggagtcagccctcgctgtgcagcaaccacagtgacgtccccagtgatcaggcttcgtggggatgctcaagtccctcttgatgatcagctctagtgattctcatgttctgcacgcacagaatacatcctggaataatggtggtgaggtaggaaactggcaagacttattttgtgttcacaagcctcctgacccaaacaggatctggtccagacagggtcaagtgaagaaactgacgcaaaccagcagatggcgacaaaagccatccctaactgcccttattgctcattagcataagacatcccaccagccccatgacagcttacaaatgccatggcaactaccggaagttactgcccctttcctaggaagttctaaataacccacccctcaatttgcactagctcaccccttaatttgcctgtaattgaaagtgggaataaacacagttccgagagcccatatgttgctgactctgcattgctgcctatgagttagccctgctctgcgaggagcagtacgcttcattaagatgattgctgtctaacaccaccaggtcgcccttgaattatttcctggttgaagtcaagaaccttcctgggataagccccagtttgggggcttgcctgtcctgcattgttggtattttccgtttgctgagaaaagttcagttagtgctgcctgagctggctgaaggcccttctgggctgggtccatatgcccaagagggtgacactggctggtctcgggggtggtgtctttgagggaatgagattggtcctacgagtgctggagaaactgcacactgaaatggtgtagcctagcattgttagggagtggcagataggagcaagaagctgaaagcagggagcagacaggaaggagcacatcccctttcctcctccagccttgcagtctctttggagctggcagcaaagtcagaaacatggttggtctacagaggtccagcctcagcatcacaaaacagaagataaaaagatatgtttagaggggagagacaatagcttaataacaggcacagccccaaaataacattttgcttttccatgttatgaaccaagtgagagataacatgaaggcatgaagaagatggaaatctaaatatcagcatcgttcctgataacataacattatggaatgtggcttgggtatatggcaacagtgggcttcctaataccaaaccccagaacaagaattgcccatgcagctgtgggcagtgatgagctgcaggcaccttttttttgaggcctggccttgtaaacctgagtttctggaggagcagagcaaatgagtgctgcccaaggtcacctggcctccaccaaggagggagagaggaagcactgagttacacatgctcagtcttaggtcccagtttaccagtggggaaattcagtctatctccctggacaggtgaaagcagcggggcaacagggaaagcccctcctcatggatacctaagggcagggaagacgggctgtattccctccctacataatgtaaaagacaatattctcaaaagcagttgacacaacaggggccagttcttataaattgatagtatcaccccagaatgtgtttgggcaaaggcagttttataagtggccacatgatctggttccatgacatgctgcttaactgctctgtcttaattcaggaagtggttttagaatatcaggaaaagagagacggatggactgcatcatctttctagaaatatcatatccataatcacatccatgatctctctcttagcctatgatggacaaataagggtctgcagtgagattttacccttccctgcaagaatctggggtgtaactattcaggattattcattaagagagggctctcaagctaatagaatcaaccttggattaaggctggcatagtaatgcagtcagtacttggtaattagcaatattctgattcttttcttaaaacaccagctcatccctgcctgctcctgcttgtcttcaggtgtctgcaagatgataaaatggatgaacatagcacatgattatccgtggaatactcagaaactatcatctctcgtgttatagcttgctttttctctatttttcatggacacctgtgcttctcactctaattttacattgcacacctataatggattcttattttctgctactgatatttttgctagtgtataagatagaatttcatatatcaacatgtcccagtattcctcagagggcttaatgacttccaggtggttcagaaccactgatgcctggggccctaaagggagactgctgcatgtcctacatgtgtgaatggaggacagagagcgagtttaggacagggggcagggatggacattttacattttgaagaagtaaacactgggcaagactggccattaattcctcctctatttgaccatcctctgggtgttgggactcactgaatataggctgagtttcagagagacgccccttgagtagaattaaatctgaatcaagaagcaattaaacccatgacagcaatctcttggaatcatttttacttttgtaacaactcttctacttgtgtttttcaaaactcttcacaggcattcttttctcaccattttacaataatctggtggcatgagttggcctagtgttatttttcatatgtgagatgagtaaagtggctcttagaggggttaaatagggcctggtgtggtggtgggtcatgccttgcaatctcagtgctttgggaggctgaggtcggaggatcgcttgagcccaggagttcggggctgcagtgagctatgattgtaccaccgcactccagcctggtgacagagtgagactctgtctaaaaaaaaaaggaggggtaggggggtggtaagaaaaaaccctaaaattatattataagattgtttaggatgaatccaggactaaaatctgggtctcctgactcttaagcagataaatcagttttaaaacttgatttttttttcgtatgctagaggatttcatacaaagatggaggaatcacgctgcatccctttggtgtttacttgatcattttattcctctttagaggtttatctttaggatcacatgttgtgccatctatgatgttaatctttaagatgctagcttaaagcattataaagccaagatttgttttttaaagtgtctaaagagaatgtctcttgttaaagtgagactgtcttctcattttacaaatcctaactgccttttacagcacacagtgcggtagatgttcgggaggcagccgccaacaaggcacaatcgcccactgtcttcaggggcctctggcttagtcctcatgctcctggatagcacctaccaggagttcccatctgggtccaggagttaggctttaatacctagtaagagtcattgagctgccaactattattttgtagctcagaggtcttggagctctatccttggagttatacacttaaccagtgaagagcctaacggccaaagatgtaactgcttgacatatgaaaccacatggtgaacttagtttgttctaccaaggacctgaggtctttcttccatctgcattctgttactattgcatttttgctctggaaaatgttcccatttaacgaaattttacttctaaaatctgcatggcatctgaggagtttgagaaagtacttctaaaatgtgtcctgcagagccttctacagtgtatctttcggcttctccttagtatttatagaggcatatatatatatttttattatgctttaagttttagggtacatgtgcacaacgtgcaggtttgttacatatgtatacgtgtgccatgttggtgtgctgcacccattaactcgtcatttaacattaggtatatctcctcatgctatccctccccctcccccaaccccaaaacaggcctggtgtgtgatgttccccttcctgtgtctatgagttctcattgttcaattcccacctacgagtgagaacatgtggtgtttggttttttgtccttgagatagtttgctgagaatgatggtttctagcttcatccatgtccctacaaaggacacaaactcaaccttttttatggctgcgtagtattccatggtgtatatgtgcctcattttcttaatccagtctatcattgttggacatttgggttggttccaagtctttgctattgtgaatagtgccgcaataaacatacgtgtgcatatgtctttatagcagcatgttttataatcctttgggtatatacccagtaatgggatggctgggtcaaatggtatttctacttctagatccctgaggaatcgtatagaggcatattggagtccagtacatacacaggttcacagtgtgctagaagcattgctctttattagttattcttatcactggctgcagttttgtgatttctctctattaacattgcctttgaattgaaggtcactgttttatttgtattgctgctcttcagccaaaagaaaatagcttcattctgctttgttccaagaaactaatattctagcacgctttatgccttctgattggatttcatccaaattggaatagctcaggctagtggaaatattttatttaatcctctagggtttttttttttttttgactagctgttgtatcttgatacctttacatcactcaaagaatttaagctgttcctgctgccatgtcattagccagagaccaattttgtacaacccccttggctttgggcctaacactggtatgttccatttgtccattccagaaagggatgtacacatcactgtggtttgaattatattttaaaaaataatgatgatggtggttgctcagtggtgctcctctgatactctgaattctattatagatgaatgagctgcaaaatattctataaaaaaacaattatgtcttaaaagattataggctgcagcaattaccactaaaagggtgagagagcctgtagtttttgagcactgacaagcagatacatccagctaattttttgtgaaaacaccagcagagcaaattactcatgaaaaatctgttcacaggaatgaattaaatgccatgtccagtttaacagtgaacactgactgcaaccaaaaaccaatagagataaataatgaatggcatttgaattcattcatttgtatgattccaaaaatagcctcagtgccaatagtgtttctaatgtccaaaacccactgtagacatggagaaggataaagaagccaatgaaaaagaaactaaaaaagtgaccatttggattttattttttctccacattcctgcagccagtcacattgaccttttatttttattttttatttttaaactttagattcaggggatacatgttgtgcaggtttgtgacaaggatgtgttgtgtgatgctgaggtttgggcttctattgatcctgtcaccccaaatgtagttagtgaacatagcacccaataaaatgtttttcaatagtgcagtttctacagttactgttaatatggaattggcccaataacaactggctgaatgtataagttggatgatagcttgccagaaatttcccatggccttgttggtaggctggggtaagatatgataatatgctataaagaattattcttttaatttctctcctagttagactatacctaataaacttcagttttcagtgcacagaattggtcatttatcagctgtgcacctagattgctaagtgagttagtcctagcctaagtaattaacccattttggtgatgactgaatatatgtctaaattcaattaaaatgaagaatatggtgtaaaattatttttatgcaggagagcctgccattacattttaaataagagtttctctttcagagtttacatatttttaaatggctaaaaaaaaaaaatcacagttttttgtgtaggcatgatctatcacatttccctgtataaccccaaatgggaaaatggaagttctaggttccaagcttgggttcctgaatttcactttaatattttaaggattaagaaagaaccttgaaaggatattctgttgtagcccagtttttaggcagtattgattcaggaaaggcaagccccaaactggggcttagcccaggagggttcttggctttgcccaggaaagaattcaagggaaagatggtggtagaagaaaacagctttattgaagtggcagtgttatagttctgtgattgctcctgcagcttaaaggcagttctgcagtaatatttatacctacttttaattacatgcaaattaagaggtagttcatgcaaaaatttctaggaagagggtggtaacttctgcattgttggatcattgccatggaaaggggcagaaactctcaggtgttgccatggcaatggtaaacatggcatgctgggcctgtctttgaaaaggtgcttccattgcctccctgttttaaccagtcctcaattaggcctggtgtctgagccccacctcctacctctgtatcactcttaatactttgttatatttttattaatatatcaaggttatgttaattttgtgcactgaaagagctgtgaattttgcaaaagtttccaaattaactgagaagtgccaccaagagatatagtctattcagtttttccaaaagctgctttcttagcacatattccatttgggcataattttagcaagttgacttataaagcgtctttaagtggaatatttcttcccctgcctcacagccaaggcaatggctctccctggcactgtgcaaaccctgagccagacgctctgctctcaacagttacaacaaagtcaccaggtagatctagtttcactgtctctttcactgtcaaggagactgagtcttagagaggttctgacttgttcaacatcttgcaactagtaaagagtaaaattaaggtttgacttgagacagcttcttcagaactcatgctcttaaatcctgaccctatagtgtgaataccttggacccctgctcagccataaagtcctctcttgccctccactgtcatgaggactctccaaggttgtattgacttgaaggctacatgagaaaggtgggtgggtgcatactttacagttgagtttgatagcatagcaaggcttgacagataatgataactagctatctatgtgcctagggtttctcatacatcgcatcatgaatttttttacaaccttgtgtccagtgtattgatcagaaaggtcacagagacaagaagtgccccagcaggaattcagacccatctctgtttggcttcataatttatgcaaatcgatcccagacagaattctacttagaaatgcctcaagataccagttggaaattatctttcatttctgaaactggaaattaaaaaattatttaaacaatgcataatcattgtaaaaatatgtaggcattgggatatacagtataaataaaagaaaaatatcctttgtagagatcctttgtagacatagccaccattcatatttcgatgtgtgtcctttttgacctttttcattgtgtggctgagtggcattttgtcttttgctatttccgttgggttctctgtattatggctacttggatatgatctaaggaatgtttctctttctccagtaagtgctttcaatgcatatatcttaataaacaggtagaaagttggatagtttgctctttctactaataagtaggagccacgatagtagtattgtgacagtggtgatagtggttggagtgctagcggaggtgatagtagcagtggtcgtggtgctgatgatagtacgtgtgagagatgagttggtggtgtaggtagtgggaacagtggcagtggtggtagtggtgatggtagaagcagtggtagtggtgataggagtggtggtggtgatgataggagtggtggtggttgtagcagtgttgttggtaatggtggtagtgttaggagtggtggtagttgttgtggtaggagtggtagtggtggtaatggtagaagcagtgggtagagttggtgatggtggtagtggtaggagtggtggtgatagtggtggtaggagtgatggtggtgatggtaggagtggtggtgatgtttacttggcaaattcttgctcaggaagagtgtaggagtgttgttggtgatggtaggtaggagtggtggtgatggtagaggtggtggtaatggtaggagtgttggtggtgatggcaagagtggtggtgatggtaggagtggtggtgatggtgggaatggtggtgattgcagtaggagtggtggtagtggtagtgagtggtggtggtgatggtaggagtactggtgactgtagaagtggtggtggttgtaggaatggtggtgatggtggtgattgtagaagtggtggtgatggtaggagtggtagtggctgtggcagtgctggtgatggtgagagtgatggtggtagttgtagtggtgatgatggtaggagtggtagtggctgtggcagtgctggtgatggtgggagtgatggtggtagttgtggtgatgatggtaggagtggtggtggttgtggcagtgctggtgatggtgggagtgatggtggtagtggtagtggtgggagaagtcgtagtggtggtgatggtaggaatggtagtggtggtggtggtgattgtgggagtggtggtggtgccatgagtagtggtggtagtggtggtggtggtgattgtgggagtggtggtggtgccatgagtagtggtggtggtggtacgagttatggtgatgatggtagtgatggtaggggtggtggtgacagttgtgatggttggggggtggtgatggtagaagtggtggtgttggtggtaggagtattggtggtggtgatggctgaagtggtggtgggggtagtaggagtggtgatgatgatgatgacctgactggtggtggtggtggccgtattggtggtgttggtgatggtgggaatgtttgtgagcactttccattgcacttaaaaagaagtggacaaactcatttctcttgcctggacagttgtgagtgaagatgcctccatccctgagtctgaaccataagcttggaccacatatataagtgattcacttttccatagcatctgtgaagacatacttatagaaacaatgtatagttctgaaaatctgagggtttaatgaaaagtagcaaaaattcagttggtcatttacttccctggcacatttgatgattgacatttggtgaaagaacactttattgtgaatctgtttgaagtggtggggcaatgagtattttggaggtgcccataatcaagctctttcttcagaagcttgtgatcctgagaaattttgtgttttctgtgttgtctataaactgcaacttaaggatgtttgcaaggcctattggcatcacgtgctctctctctctctctctcacgcacacacacacacgcatgcgcacacacacacaagcttgctagagcttgccagtcatatgacacactcaagacctggtgtaagtactttttctattttagtcattcacaacttaactggtttacttggcaaattcttgctggggaagaacacttgcttcagaaccacatatcccactgaaatgtgtcagcaaagcaacaaatatgtatcaagaacaaggcaataggctaagtgctgagtgggcagatatcacagttaaattattgcttgtcttccaaaatttttgtatcatctccaaaggtttgtttttcctttcatatcacaacaaaatgatttcccagtcttctttccacacattgggcaagcttaaaatacaacatttctaaaaatcctctttggtgttccctgaaaagatctatgagctatggaaaccaagcttaccctaagctgtttagcattaagaatttgaggaaaccaaatctccacttttatgcagaaatcaaagagtagcataagtaacttgtaggttattttaataatacaataattttgtaaacaataaacagctcaaattttgtagctggctttgtaagtagagaaattgaaataaagtcactcaaaatcacactgactgattgattgtcaaaactcttagttcttcccaggactttttaaaaaacatacttttaataaaaagtacagataatatttacatattttgtatcctgatttatcttgtaatattaaaatatttcctgttttcctaattgttcattgacttactgcataccattctatgagtggatataccataatttacttaaccattcccctagaattggatctgaggctgtttccattttggagagttttttttgtttgtttctgttggctactgaatacttcctatgggaaagaggtggacgtggtggaaaatgaattgatagaatagtttgcatttgtataaagcatgcaaatttgtatttccagatatgtcacagtgaaaggatagaatctttcctgtagaaagaacacagaggttaagaatataggttttggagtcagacagaccttggctttatttctgaatccacataactgtgtgactaattttgaaaattttctcgaactttctataagcttctttctttcatttccaaagtgggagtaaccactctctcgtgggatcattgtgaggataaaatgattgatatgtagcaatgtgcttggcacatagcagtgctcataaaattgttgctattttctatgtttatcttattcctcatccaaactgtaacactcattcatttattttagtttgatttttttgagtcggggtctggctctgttgcccaggctggagtgcagtgatgtgatatcggctcactgcaacctctgcctcctgggctcaagccatcctcctacctcagcctcctgagtagctgagactacaggcgtgtgtcactgcacttggctacttatttgtattttttgtagaaatggggttttgctatgttgcccagtctgatcttgaatttctgagctcaagtgatgctcctgccttggcctctcaaagtgctaggattatagacatgagcctctgtgcccagccccattcatttatttattcagcattcaccagtggctgttgcatgcctgacactgagctttttactggcaagacagagctgaatgaggctgtgtttctgccttgtggatcccccagtatccagggacccagatcaaacagataggcaagacccctgtgtcacggaggaaactacaggggaaggtactgctttgggagtggctcttgggaaaagtaatagcaaaagcccagaggttagcaccgaaaaagatcaagttcagactctgcctttgctattttcttgctgtgaggccttgggtaaattacttaaagtttctgaatttcactttctgcatttgtattcttactctgcaaagtgttgtgaaagaatatacatatagctctgggcatgtagcagatgctcaaaaatgattattatgcttgaagtcttcagtgccccaatcatggctgattttcatgaaaataaactcaacaaactccagtgttctgaaatgtctgagactggctcagctgtgctcaggttagtctgaaatacattttattacagtctaattctctgcaaagtggtcatccacacatcttgtgtatggtaatgtggaattagaatattcagctctcagtgaatttcatttattggccattatgtaaaaaccaaagaaaaatgacttatgaagacattgattgctttgtagctttaaaagacctccttagaattagaatggctacacagcctctaagaatatggtctacactatatattttgtcatcctgcagacacaacaaggaagtgggaatcgaatctatttagcaggatgtctttatgtgtgaagatttcttgctgcttataaatgcttatttgattatttattcatagatatagaaagcacttttctttcattctgtttcctcattgtcacccaagtaagagtcacttattactgatgtgctaaagaataacagatgagcattgaccttcaattaaacattagcaaatatgttgcagtagcataaaaaataacatccacttgcttcagaatagaatgagatgatgcttaacatttctattgctgaggtcctacaagtaaaacatgtttgtttcataaatagaatttatattatcaattggaacatattcttcaatgcattattttgcctactgcttaggaatatcttcctttgttaattttgagttattttatttctactattcagccatgcttataaaatgcaagtcatatttctaatattattgctgttcttttagtcagatgggttcaaccacatttattcagcacctgctatgtgcagtacacttgtcagcatgctggtcttaaaccttgagctataaactgaaatcacattaacaatgtctagagaaagactgcagagatttaaaataaagaaaagtcagagaaaggtgagttatgttttttttgaccactgggccaaacagtgaaaggagaataggagaggaggttttcttaaaggcacaacaggattcacacaaatgaggagacttagaacagaaaggtcacaaatacagcacttgtagtgctggggatatgtattaatattttaaaaaaacaacatattttgaaataattctgcacttaaaagtcacaagtatgctacaaagagctcacatatctccctcgttctcatgcctgagctggctccttcctgagctggatacatacgcccgttatcagaacgtttttctgaccacttgagggcaagctgcagttttatttttatttttattttttgagtcagggcttgctctgttgccccggctagagtgcagtggcatgacatcggctctctgcaacctctgcctcctgggctcaagccatcctcccacctcagcctcctgagtagctgggactacaggcatgtgccactgcacctggctaattttttgtagagatggggttttgccatgttcccaggctggtcttgaactcttgagctcaagcaatcctcccaccttggcttctccaagtgctgggattacaggtgtgaatcatgatgccctgtcactcctaagtaccccattgtgtatgcccccaaacaaggacactctcccatgcacccaacctgcagcttcctccaagtcatgaagtcagcattaatacaacactcttctccagtctacagactccattccatttcaccaagcatcccagtgatgctcctttctcctttctgactcaggaacacacattgcatgcagttgttgtagctcttaagtttcctcctgcttgagcagttcgttagtactcaatcgttctctgtcttccatgtccttgaaagttttcaggagtgtagaccttacattttgttggatgaccttcagcctgggtctttcggatgtttcctcatgaccgggttcaggtcatgcaaccctaatgagaacctcttgaaaatgatactgtgcgttcacatcaggtgtcacacagtgtggacccctcccgccactgtggatggtaaccttcatcaccccatcattctgtggctgcctggtttctccagcactaagttcccatgtttcctttgatatctaaatagtatgttgtgggcatgatagtctatgattattctgatgcctactccccatcaaacctccatttcatcattcatttccattagtcagcattctattagcatggatgtgttggttcctattctattcaatgggctgtactttattactattattttgattctcaaagtgtctcaggtttggaccagaggaacttcttcaaggtgtctcttgtgttctattgacatgtccccatctgcctttgaatacttcttaatttttttgtacaacaggatgctgtagacacatctgtactgcccctgcctcagccctggcctcagccatttctcccaggagctctggttccttgtatggatggatggagtttagaaatcaggatctgtgtgcttggtgtgctcatggctactgaggtgtcattacttataggccttctcagtaggtaaatatagaaaatgtgtgtaaatgcactcatatgcttccttcgctatttccatattcacctgtgagtttgcatattaaaaactagtagtttatgctaatgtttcctattccagtgcaagtcattagaatcttttctacccttccccctttccatgattatatgaacattctccaataatcaggaatctggccctctttatcttcatatccttaactccttcatttgtttacatatttctccatgtaacctatctcccatttatcccagctgtcttccccatctgttgctgtgttcccctcaatgtgcctcatccccaggccctggcaggctcatcaccacgtgtgctgccagatgcctctgtgtggcagcccctgcatccttggtgctggtgagtggaccacctgtgtgctcctgtacaggctcccaacttaccaatggggccgctcactgccaccttttctgcacaggaagagacgagaaggcccatttatgattttgaccccagtcccaggcactgagttcttcaagggccaggaatgtgacttgttgatctttgtagcccaacagtctagcaatgttgatttacccaaagcattattaattgagtgacagatgaaataataaataaacatggcatttgaaaggtgaaaaagtcttgtcaatcattttcaaacactctcctctcccagaggaaccatttccacatcatctgtatcacatggacaaagattgccaatctgggtaaagcttcctccttcccccattgaggtgtgaaatggactgaatgcttgtgctccctaaattcctgtgttgaaaccctaattctaatgcgatggtattaggaggtggagtgtttgggaggtgattaggtcatgagggtggaggcttcatgaatggaattagtgcccttatacgaagaaaccagagaggtagcttgttccctttctgccatgagaaaaagcagctgcctgaaacgtggcagctctcaccagaacctatgctggcagcttgatcttcgacttccagcctccagaactctgggcaatacatttctgttgtttaatctacccagtctatggtactctgtaatagcatcaccaaccgacatagacaaggaggtctctgatgcacctttttgttgttttgtaacaatcatgatttaaaaacatcttgtggaggtattggtcatatttagcacatgaggctctgggacttaagtggggtaagtgatttgtcaaatagaatatgcacagttagtgtcaaagccgttgcccggatctgggtaccctgatccttagtgttgtgattctgtgttctatccaatgtcatgcctcccccacatgactcaggagcagagtgctggcatggagaagagacagggtggctgagcgagctcacttatggtcctgaataccaggggcagaggtttggactttgttctgcagcgcacatggagtcctccaggcctttgagaagattttgtattacaggttgagtacctcttctatgaaatgcttagaaccagaagtgttttaaatttccaatattttggggggttttagagtatttgcacatatataatgagatatcttcaggataggacccaagcctatacatgaaattcatttatgttttttgtacatcttatacacatagtctgaagttaacgctatgcaaaatttcaaaataattttgcacatgaaacaaaattttggctgcattttgactatgacccgtcacatgaggtcagatgtgcagttttctacttatggcatcatgtcagcactcaaaagctttcagactttggggcatttcaaatttcagatttttggattagagatgctcaacctgtaacatgtatgaaagtgcttggagtaatgccaggttctagcagatgtttttgcattttgtttatcagcttgtgcacatctgagtgttaggaaggtcgctctgttggtgatgtgtggaaaccatggaaggagtgagagagatcagacagagagtaattcatccttgcagaagtgctgacaactcgggctaggctggggacagtagaaatggagaagatgtgaatatgtcaaataccacgagagactcacgaggatgtgaaatgtaggaaggggacaaagcaaaggatctgaagctgaaggcctggatgggatagtgcccttgcaaggcagagtaacatcttgctaggtaggtaggcagtgaacttgatttaagacttgatgagatttatgtcttatattcatttagttgctaatgaaaaggatttaaagtggcttatgaaaaacataaaaaaacaagaaacaaaacaagggaatctaaagttcattgtaatcagactcattttttgaacaatttgctgtgagccaggcagcatcccaggtgcaggtcactatacaatgaccaacactgtgtcatggcgcctcttgtgtggatgcatgaggccttgaacatgtggaggtgagggtgtggtggcttgtcaaggagaacctgcctgtgcttagggagctggtggagcagggagcctacctgcagggttggcaggaccggaggagatactgggatctgatggatgaggtagacttagcctgggctggatgtggaggaagaatgactctggcaatgggaacagcagattctcctgaaccctgctcccagctcctctcacagcaactttgcttttctggttccctatgtccgatctgtcagagaacatctcaaatccgaccacttttcaccacctgcgctttcatttccaggtcctggctacccttaggtcttgcctggggaacatcaatgaccctgtgactggtctctgggctctgctcttttacaatgtgttcccaacagagcagccaggtgatcctgttaaacatgagctagatcatgccagtcctctgctcagaacccttcagtggctctcatctcccttggggtaaacaccaaagtctgctacaatggcctatgtggctctgaagatcaagcccactctcataccctctctccctcatctactactttccccttggttcaccctactaggagtgttccttacatattaaagtgtgtatttgcctcatggcttttgcactctctgggagctccctctgcctggaatatccaattagtgaataggggtgtcattcggggaacccagagagccagcttttgtcagaatggaggtaggatgcacccttatgctaaccagaggtggtgctggcagggcaagtggaagctcccagtagctgagagctgtgagagaggctggagtaggagataatttagaaattggagatcatcactgaagctatgcaattgctggtgaccatcctgatgcccttttctaatcataagataaaaaataactggggctgagcatggtggctcacgcctgcaatcctaacactttgggaggccgaggcaggaagatcacttgaggccaggagtttgtgatcagcctggacaacatgacaaaaccccatctctgaaaaaaaaaatttaaaaagcagagcctggtggcacatacctgtagtccaagttacttaggaggctgaggtgggagtattgcttgagcccaggagtttgacatcagcctgggcaacataacagggctccttagctacaaaaataaaaataaaaaaagctaggtgtggtggcacatgccagtagtcccagctacttgggaggctgaggcaggaggatcgcttgagcccaggagttggaggctgccactgcattccagcataggtgacagagcaacactttgtcttaaaaaagaaaaaaaaagagagaaagtagtagcttttgatgcctacccttttgaaggtgagtggcagcatggaacatagctaatcacagatattttctgcgataacttatatctttctttggaattcactgtgagtgtaatttttggcagccagtttaccatttccagtgtttaattttgttggatcagcctgcgctctctgagaacttttcatctggcagtggatgggtatagtttagaatatggtggatagcagagggaggctagactgggtttttaattttttttggaatttgaagacagtgcttaatgatattactaataatagacaatccttttgaacacatacaaaatgccaggcatgatgctaattgctttgcatagattatctcagttaatcctcagaaggccttatgatgtagatactattattaaccccattttttacagagcaggaaactgaagcttagaaaggttaagtaacttgaaggtcagacagctagaagtggcagaggtggggaagactcaggttttctcctaacccctatggaacagatctggatgcatctttacatacattatgactgtatcaggatgctattttagaacacataattcaagaactttattgcgtttcaccaaagttttgattttgtagaacaagaaataaagtattaagaaagaaatcatataaacatttgaacgcttggtgtcagttaaggaatgggtacatttcagcattatcctagaaggtcttatcaggccagactctactgcgtattttaagtggggtgtctgaattagcttctgcagtaacttctgaagaaggtggcacagactgagagatctgctaattacttgggacttgatcttggacagatgaccttttctagtatccttagaatgaattgttggtaaatgcttagtttccggctataggtttcaatgcatatgacaagtttatgtgaatgtctttcacttaacagcctcagagacacaagttttctgccctttcctctacctttttgggacccaggtgtgtgccttgggaatgggccagtgcaggaaaagagttaggttgagaggcacgctgtgactcatgttactctctgctggtgtatgataaaaggttttgttcaaaatgacaagcttgttgaaaaatgtgttttaatttagacttgagtcttatcgaaatggcctccttagaaaatggcttatagagccataagaacataaaggagtctttgtgatctagggtagagactcttaaaacattttctcattatgacgtaaaaaaggataatgtttgtattgcacactggggttaagtagtaagtgctcatggcccgaggtgatggcttgggatgttgagctgccttgggcccacccactgccccagggtgaagaggatcaaggtcttagtgcactagcctgccaaggaccaccagaagggaagctctgatgaatggtatcttaaaacccagtccttccatttctgcagaaatagttttatgtgaaactttctaattctttgggcattggattttgaagataagttctcattagaatggattccaatttctgcagtttatttaaaattttttaacagtttgataccaacactttattagcaaagaaaatggaatcaacttttttttttggtgccagaaagcatagttttttttccataagaaacaaaggtggaatatatgtaatgggtgagagacaaccaaaacttgccacataagggccagtgagagaatcccaacaaatagcaaaagttcatttttccatgacctattcatcacagaacaattaaaaagctacacaaagatcagtcttacctgcattttagaaatacatttcaattcacccatggatccaacatgggtgatctatctatctattcttttagatagacagagaagtttttattaactaacttaggaaaaaacactacctgttaaactcataaacaattgtttaaagagtatctacagaatccttctctcaatgtttccatgtaaaagaaatgaaaatcaatagtccagagatcctcgttaataatgagccactgataaacacaacatatttataacttgtataacattttatttatgtgcattctttgagcattatgcaatatgtaattaaaatttttttaaataaaaaaatttaatatctttattttttgtcaattgatttttccccacttaaaaaatttgcaaataataattgtgcatatttatggggtacatagcgatgcttcagtacatataatatatagtgatcagatcggggtaattagcatatccttcatctcaaacatttattttcttgtttttttttgagacgtagttttcctctgttgcccaggctggagtgcagtggcaagatctcggctcactgcatccttcatctcctgagttcaagtgattctcctgcctcagcctcccgagtagctgggatcacaggcgcctaccaccatgcctggctaatttttgtatttttagtagagacggggtttcactatgttggtcaagctggtcttgaactcctgacctcaagcaatccacctgcttcggcctcccaaagtgctgggattacaggtatgagccaccgcgctcagcctaaacattcattatctttttgtgttgggaacattcaacatccttctgactatttgaaactctaaaatatactattgtgaactatagtcattctctattgctgtagaacactagaacttattcctcctatctagctgtaattctgtaacttttaacaaatctctccctatccttcacttccccctacccttctcagcctctaatatcctctatcctacctcttacttctatgacatcaaccctgatacagtttggatgtttgtccccttcaaatttcatgttgaaatgtgatccccagtgttggagatgggcctggtgggaggtgtttgggtcatggggtggatccctcgagaatggcttgattctcttcccatggtaatgagtgagttttcactctgttagttcacatgagagctggttaaaagagcctggcatttcttgcttgttccctctcttgccacgtgatgtgcctgctctcccttagccttctgccatgattgtaagctccctgaggccctcactggaagcagatgccggcactaagcttcttgtaaagtctgcagaaccaggagctaaataaatctgttttctttataaattacccagcctcaggtattcctttatagcaatgcaaaacggatttaagcaaactcattttagcttccacatatgagtgaaaacatgtttaactttctgttcctggcttatttcacttaacataatgtctttcagttccattcatgttgccaagaatgacaggatttcattcttaatggctgaatagtatttcattgtggatatataccacatttgctttatccattcatctgttgttggacacttagattgattccatatcttgactaatatgaataatgctgcaagaacatgggggtgtagatgtctctttgagatgctgatttcctttcttttggataaatgtgcagtagtgggattgctggatcatatgattgttctatttgtagtttcttgaggaacttccatactgttctccattgtggctgtactggtttacattcccaccaacaatgtataagagttcctttttcttcacatccttacaagcatttgctgtttattttatttttttgtctttttgataatagctatcctaactggggtgagatgatacctcattgtgattttgatttgcatatacctgatgatcagtgatgttgagcattaaaaatacatttttggccatttttatgccttcttttgagaaatgtctgttcggattatttgcccatttttaaattagattgtatttcttttgttgagatgattcagttccttgtatattctggatagtaatcccctgttggatgggtagtttgcaaacattttctcccattctgtaggttgtcttttcactctgttgattgtttatatcttttatttttttgagaaagggtctcactctattccccagactagaatgcagtggcatgatcacagatcactgcagcctcgacttccccagactcaggtgatcctcccacctcagcctcctgagtagctgggactacagccatacaccaccatgcctggctaatttttgtatttttgtgtagagatggtgtttcaccatgtttcccaggctggtcttaaactcctgagctcaagtgatcctcctcgtttagcctcccaaagtgctgggattacaggtgtgagccattgggcctggccttgttgattgttttctttgctgtgcagaagatttttaatttgataaaatctcacttgtttagttttgctattgttgtctgtgcttttaaaatcttattcataaaatgttttcccaggctcatgtcctctgttttcttctcataattttatcatttttggtcttacatttaggtctttgattgacttagactagattttcatataaggtgagagacaggggtctactttcatccttctgcatatggatatacagttttcccaacaccatttattgaagagaccttcttttccccgattagtgtttggaacctttgtcaaaaatccaaaaatcagttgacagtatatatgtggattaatttctggattctcaatactgttccatcggtccgtgtatctaattttatgccagtaccatgttgttttggttactacagctttgtagtatattttgaggtctggtagtgtgatacctccagtgttgttcttttcgcttatgattactttggttatttggggtcttttgtagttccatacaaactttaggatttttttttctgtttctgtgagaaatgtcattggtattttgatagagattgcactgttattttaacaatattaattcttacaatccatcagtatgggctgtcttttcatttgtttgtatcctcttcaatttctttcatcaatggttcatagtttcccttgcagagatctttcactttcttggttaaatttatttctaggtattttaatatttttgtagttattgtaaatgaaatttccttattaatttatttttcagctagtttattgtccatgtatagaaatgccactggtttttgtatattaattttgtatcctgcaacttcactgaattcatttatcagttctaagagtttttttgctagagtctttaggtttttctagatataagagcatgccatctgcaaatagggacaatttaacttctgccattccaatttgaatgtcttttatttctctctcttatctaattgctctggataggacttccagtgctatgttgaataagactggtgacagttgggcatccttgtgctgttccatttattagaggaaaagctttcagcatttccccattcagtaagatgttcactgtgtgtttgtcagatatgccatttattacattgaggtactttccttctatacctaatttgttgagaatttttttattatgaagggatgttgaattttatcaaaagctttttctgcatctattgagatgatcatatgatttttgcccttcattctattgatgagatgtatgatgtttattgatttgcatattttgaaccaactttgcattcctgggataaatcccacttgatcatggtatattatctttttgatgtgttgtttgattctgtttgctgatattctgttgagaatttttgtgtctctcttcatcagagatgttggcttcttttgttgttgtgtccttgtctcattttggtgtcagagtaatgttaaccttgtagaatgagttaggaggaattccttgccttttaattttttggaatagtttaagaagaattggtattaattattctttaaaggttcaacagagggtgtgggataggggtagggagtgcataccttatgcttctaatctggagcaatgcagctgtgcagattcctggcagctctccaaactggactcagggcttgcaagaactgtgagattctccttttgcaaggactgtaggtgtttgctctgtcaataggagttgatgaggtccttctgtttatcttttccatgcaataagaagtccctcctgactctaggcagattcaatctgagcaggggatacagggctgcagaggctgagtgcctgcatgctgcctttcgggactgccattcatcacagttgcatcttcactcccccaccgtactccagcaccctcccttcaacactccagtcaaatcttactgtttattcattgccttggtccttttttttggtgggggtaggggggcaggcagaatgccaggcatctctaatcaaccatctgctgacagcactcttccaaatgatctggagtttatttttatttttaagaaaaaattgtattacacagattctatatttataccttgtgctagtagacttaaggtcattaaaatataatggaacccctactttacaacagaagtggatatatagtcatctatagcatggtagaattcgaacatcatttctatggctttagtaactgtgactggctctatctggatgtgcaatttctgaggaaatgttaaaatcaaaacttgggcactgattttttttaaaaaaggaattatataaaactcaccaaaattatagtttttttccccaaaatgctcattgtgcttttaaaataattaatattaagtatccctctgtgtgaagaccataattatgaccatcatatcactatgtcaacaatgacctttgagagttaaaagaataaaagataggagagccatcttgttggtgcaaagaagattcttagaagtaccaactgagagatgaaagtgctggcctagaaactagtaaccttgtcatgatgtcattaaggcaaaggttcactacaactggagctaagagacaccggggttatctattttgcatttcatggttcttttttcctctctctcatcattttgggtttccagctgagacgttacaacaaagattagaagacctggaacaagagaaaatcagcctgcactttcaacttccttcaaggcagccagctcttagcagtttcctgggtcacctggcagcacaagtccaggctgccttgcgccgtggggccactcagcagtgagtacttgttattgtcaccattttcccctcatgtttcttcctacctctcagctcccacatagtgaagagctgggaggagcttagctgtagagacccttggctgcctttcctgggaagggccctttgttggttgggaaggtggggattcttccagagtgagagatggcccatgtgggtcatgcatctctagaaagaagacttttctgccacttgacagagagagaaccttctcaagtgcagctgcatcccaggtctgaagcatctcttgggaaactacccttctaagaaggtgggggtaatttcctcttgttgaggcagcttggtgtcctgtgatcatattaagcctgtaagataaacagatggtcccctctgaccgagcgatgaaaatccatcctcctcctaactctgccctccagctcctgagcaacatttgctatttgtgtcacccaaggctcttctgagattagtaatatatagagagcagttgctgcttctaatccagtggcttgtggtcctggctgcatgtttgaataacttagaaagatttaaaacctaccaatgtccaggctcctccccaggcaagctaagtcagaatgtctaggtgtgggaggcaagggtggctcccagatattggcatgcttaaaaaggctgagaaccactgctccatctacctttgtgttccctttctccctaggtaaaggtctgagagggaacaggtgttttatgaatgctatgagcctcatcacaattcaggtctggttctacttagttatcaacatggccacaacaattccactttatttcccacttccaattggtcctgacaatcacaagattttctggcctattcctattaagtgtacatggcattatacatttagtttatcttgatacctccctgaagcctgcctgcacagtgagaccaattactttcagttttgccagtagcatgtagcagagtgaatggcacctggtaggtgcttccttataactatttgcttagtgaactttttcatggttctcctcaatagagagtgaatttcacctcgccattttcattcttttctaatctgacattgagacatcattgcaacattgcgtgttcctctttcacacatgaggccactcccaaattgactttctctgtaattctctcattcttttatctttggggttatttcttgtcgtctactccagtcttgggaaaattttagcatgtaaaaatagggcacaaagattgaggcacaataaatttagctaatttattatgtttctatttatttaacaaacactttcataacagttactaaatgctttagaagttactcatttaatttatttccatttttgaaaattgtggtaaaatacacataacataaaattcaccatcttaagtgatggtgattaagtgattaagccattgttaagtatacagttcagtggtattgaatacatcataatgttgtgcaatcatcaccaccattcatctccagaacttttccctcttccccaactgacactctgtacccattaagcaataaatccccatcccttcccctcttccagtccatgacaaccacattctaatttccatttctctgaatttgactactctgtgtacctcatagaagtgacatcatacaatatttgctcttttgtgactgacatttctcatagtatagtgtcttcaatgttcatccatctgatagcatgtgtcagaatttcctttctttcaaggctgtataatactccattgtatggatagactacagtttgctcattcattcattaaaataactcatttaatgttcatgacaattctagggggtagtattattttatatattgatatctacatgtatagagatctgtatctacatctatacctgaattttaggatgaggaaaccaggcccaatagaagctaagaaacttaggcagatccaccagcaggtgagtgctgagggagacacaaacccaactgtctggcttgcatcttggccctgaatcagtgcactgtgctgccttttaagtctgggctggcaaaggctgctatattattatctctttagccatacctgcaatttggatggagagtgtattagtccattctcacattgctataaagaaataactgagactgagtagtttataaatgaaagaagttaattgtctcatggttctacaggctgtacagggagcatgatggtagcatctgctcagcttctggggaggcctcagtaaacttacaatcatggtggaaggtgaaaggggagcacacacattgcattgccagagtaggaacaagagagagagagaaaagagggaggtgccacacacttttaaacaacgagatctcatgagaactctatcatgagaatagcaccagaggatggtgctaaaccattcatgaagtatccacccccatgatgcagtcatcttccaccaggccctgcctccaacacttgggattacaatttcacttgagatttgggtggggacacagatccaaaccatatcattccacccatgttctccccaaatctcatgtttttctcacattgcaaaatacaatcatcccttcccaatagtcccccaaagacttaactcatttccagcattaactcaaaagtccaaagtctcatctgagacaaggctagtcccttctgcctgtgagagtctgtaaaatataaaaccagtcagttacttccaagatatagacattgggttaatactcctgttccaaaagagagaaactggtcaaaagaaaggggctacaggccccatgcaaattgaaatgcagtagggcagtcattaaatcttaaagctccaaaataatctcattttgactccatgtctcacatccagagcacactggtgcaaggagtgggctcccataaccttgggcagccctgcccttgtggctttgcagggttcagatgctgcagctgctctcacaggctggcattgagtacctgcagcttttccatgctgaggatgcaagctgccagtggatctgctattccagggtctgcaggatggtggcccccttctcacagctccactaggcagtgccccagtggggactctgtgtggggttccaactccacattccccctccatattaccctagtagtggttctccatgaggggtcctgccctgcagcagacttctgcctagacaaccaaacttttccatacatcctctgaaacctaggcagaggctcccaagcctcaactcttgcactctgtgcacctgcaggcttaacaccatgtggaaattgccaaggcttatggcttgcaccctcagaagcagtggtccaagctgtatctggacccctttgagccatgtctggagttggaaaaactgacagcagtgtgaggagcagtgtcctgaggctgtgcagagcactggggccctggggctggcccatgaaaccattccgtcctcctcggcctccaagcctatgaggggaggggctgccatgaaggtctctgaaatgcctctgaggccttttccccattgtcttggctattagcactcagctcctttttacttatgcaaatttttgcagcctgcttgaattcctccctcgaaaatggtcttttcttttctaccacatggccaagctgcaaattttccaaacttttatgctttgtttcctctttaaatataagttcaaactttaggtcatttatttgctcatgcatatgagaataggttgttagaagcagccaggccacatcttgaatgctttgctgcttagaaatgtcttccaccagataccctaaatcattaatctcaagttcaaagatccacagattccctagggcaggggcacaatgcagccaagttctttgctaagcaataacaaaattgacctttactccagttcccaataagtttctcatttccatctgagacctcatgagcctggctttcactgttcatatcactaccagcattttggtcataaccattcaatcagtttctaggcatttccaaacttttcctcatcttcgtgtcttcttctgagccatccacactcttccaacctcagcccattacccgattccaaagccagttccacattttcaggtatctttactttatagcaatgccccacttcttggtaccaattttctgtattagtccattttcacattattataaagaaatacccaagactgggtaatttataaagaaaagaggcttaattggctcactattctgcaggatgtacaggaagcatgatctggcatctgctctggcttctggggaggccttaggaaacttacaatcatggcagaaggtgaagggggagctagcatatcacatggctggagcagcaagagagagagaaatgggaggtattactcacttttaaacatccagaccttgtgagaactctatcacgagaacaccaggggatggtgttaaacattcatgaaagatccatccccatgatttaatcacctcccaccaggacccacctccaacattggggattacaattcaacatgagatttgggtggggacgcagatccaaaccatagcagagagtgatgcttttattctggtcttgcctaagtttttgatagaatatgattaactaaaagccagtgtcatgcattcttaattatattaggttacaccataaggacatctcttacatttgactgtttgaaacctgtcaaacattttatggggtttgacctaataaggcccccagaaaagtataggcctttattaaacatttatcaagccaatattaactgttggcttactttatgagctatcatcctctttccttttccttacaggagttgtctgtatactatgccagtgaatgttccattcactttttgagctcactgtgacctgatttctgtctctatcactcctcaaatatctaccaaagtcagcaatgactttctatttgctaaattaaaagacatttttcaattcttattctgctatgtatcatggcagtgtctagaatttttaataaattcattcttaaaactcccatttcccttggctttcatgatttcacccttcctggtcttcttgctacatttctggccattcttcttaatgtcttatgagctccttctctccctttcttcttaattttttttttttgagacagagtctcactctattggccaggctggtctcaaactcctggggttaagcaattcttccaccttgggctcctgaagtgttgggattacaggcatgagccaccatgcccagcccctttctctttaatattgttgttcttctgtcttctgttctagcttttcctctcttctcactttgtatcttctttctcagtggtctcaaatgctctgagtcttcagatactgtttctatactattttttcccaaatttctatctctagccctgatttatctccagaattccaatccacaattcagttttctattgagcatctcccttatttttacttaatttagtatctttttatccttaacattactatctctttatccttgccttttactaagtttgctatcttttaaactaaatttaccgttgttctccccattcagttccttctcctatatccccagtctgaatgactttcactgccatttacccagttacccaagcaaggactctgaggcatctctggatcctccctttccatccctccccacagtcattggtcaccaagtcctgttgtgtccactgtctgtctctctcatttccactcacttgtctcacctgcacgtcagtgccttagttctggtctttaacactttaaatctggattattgcagtggtctccttgctggtatttctgattccattattaccacctctcattcatcttccacaaggccatgatagaagtattatgttatgctcaatggctccccattgcttgcaggataaaattcatgttgcttagcatgacattaaaagtcttcacgatccagcccctgcccagctgcccagcatttaactagacattaccagcatctgcaatagacacttactgaaatattatatgttacccccatgttctgaatgtttctagattctatgatcttgctcatgccccattgttgttaccaactatttcccattcttccagacttagtgaaacaaatatccttgcaaagctctgctcatcagtcacactgcctctcccttgaatgacagcataccttgaatgacttcacttctgcttaggtcttaaatacatctgttttcttgaatatctgtttactctgacagatgataagctccttgaggacaagactgtattatatttatctttctatttctagagactagaatagtgtctggcccataatctgtattctatattcattgaatgtgtgaatgtgacacatatgcatatgtagctatatataggttcacaaacgtatatgtgaatatcgagagagagagagagagagagagagagagagagagagagagagagagactgacttcactttattgggattcagttgccctagaggaaaatgctccttcagcttttcttctgaaattgccagcaagaaggagttgcacagaatcttgacagaaccttggataggaggtcatttctggtcctttctcttccaccaaccagctgtatatggcttggtgtagttattgaacctctccaaactccactcccttcatcagaaaatgagggggttaaattagatcactggtttctaaactaaagttatttatgtattattaccgtaactagaaaataatgagttgaaattaactttctgaaatttttctaatgcatattaacacaattacctagttgctaaaatttttttaaaacctgcctttcagtgtgccagctacccactgctgttttcacagtgaccaccagggggccatggtggcaggctgaagctcacacagcccagagcctggacagaaaccgctctcacaggctctccgagtcaaggggtatatataatatttttgaatgagtggtgcttttcctgtagaaataaaatagttgtatcaacatgaacaatgttaaatactaattaataactcaaataagtgcctgctaacctttttgggatcaagtatttatttattttgagaagctgatgaaagctatggacttcctctattgcaaaaccacgtgtacacatgtacatatatgaaaactattgagtgtattttcaggagactcacagatctcctgagttattcgttcactccttagagggtctttggttcccaggttaataaaatttagtttaggataagatttaagaagctgtaaacatccagatacaatcagatcctttatgttttattatttttgttgtcactttgttcttaaaaatcacaatttaattcagcttttgagaaacactgtttaatcctctgaactcgtgtagaagagcaccctgggttatgttcagatcctgccagcagccaagagagctttaactatcttcaggggttaactaggttcctctgagggcagatatcggtactcaaatcatttgacccctcttctgtggggtcctctggcccagtggatttgcttgttggaggaataacttttgtgatatcctcttgattcctgtgggaagttcatgtatctttgccacaacaaatggtatatgctcaggccaccctataatagaccctctctgccctgctaggtctctctaattcctttttccctggtttacggggcagcaacacatcagaagtgagttccttagacaatgctgcagcggacagtttgggtgccctgtgtcccaatccctcggcctccctccgattccctccacaactgggaaccgtgctcacagtcggctttccactcaggcacccaacaggtttatcaggcctgtctgcacaggtgttctcggaacctgtgtgaggaggggtgggggtggataatgccccagcctacccagcctcttagggaacagttctgaggcatgctccacactgtttttttttttcttttctaggaggttcctactactaggaaccttttggccaatgggttccatttgtccgtgtggtgaccagctccacagtgaaccttttattgaattttcctctttctcactctagtttcttgggaatcacttcttcaaaatcaacctgtactcaggtccctgtctcaggctctgctgttaagagaacctaaatatagacagatatccacattagagaattagatgtcagtcttccttcttgcagggcacccccaatctttgtgagtgaatctagtggattttttttttcccactgggtttgagtagaagagtttactcccatgtgggccaggtttggataggcaaaccatggggtaggggagaaaaacgcgtctctcatgaagacccatttcctagatggccaacaggtgggtgggtattgagagtggtggttgattgcagaggtgctggttgagcagccccaggggtaggatgccccactccatcattagagattcagaatgcagagcgtttaggtctaagtccttcagctttagtttccaactctggagcaacaggaaatttctacctcaatatcctgtgacaacagggattagaagagaagagagaggcttctgttcctactgtgtcttccatccaatcaaaggaaagttatttttcctttctcctaagagagagagttggcctactatcatcacacatttggaagataattttctcatgaatacattgcatttctctaactatacaccccgtgcatttttccactggatcttcaccaatatgtacgtgaaaactggtcctatttgtaaccgaaattgtgtgtgtgcatgtatgtgtttgtatgcatgcgcacacaaaatgtcctttatcgaagaccttttcccaggggataacctcaaactcaagcaaacaaaatataacaaagtttaaaacaaagatctatcatggttttccagtggggcttatcaaggcagagccccagtttgcctgtccacatatttcccactagcatgcttcctttgagatgggaacagagaagatgaatgttgtgcagcaccatgatgcttttgggcagagtcattcttcctctggccaattgctcctcaattgcctttgaaacatgtgattctttatttccctttggttgtcatgtagggactaacacagcattcagtgtcacagaattcagtgatggtcagctctaagcccttgagattggaatcaagatacactgtatgagaccagaaaataactgttacagacacattttacccaggatataaaatgagatggctatgatacaacatgaaaattatcattactggctggacatggtggctcacacctgtaatcccagctctttgggaggctgaggcaggaggattgcttgagaccaagagtttgagacaagcctgggcaacatagtgagacccccatctctacaaaaaaaaaaaaaaaaaaaaaaaaacaaaactagccaaatgcggtggtgcgtacctgtagtcctatctacttggaaagctaagtcaggaggattgcttgaactcaggaatttggggctgtagcaagctatgattacaccactgcactccagcctggctgacagagcaagatctcattgcaaagaaaaaaatatttgttattaccaacaaaccaggatatatttctgaaccccaaatcagatcaggagatggggaaccacaagagacagtggcatgccctgtgtcccactcccaggagaccaactccagacccttagtcatggcctcaataagagaagggagaaaggacaaggggggaaactaagttgtgagccacagaggacttctcagaacttgttttggaactattggattataaataaaagctcccaaataaaggtcttccatatcatcctctttgttcaaactcaactgaagtctgtcctgaggccagtggcatcaggcataactattatgaacccagataaatattcatgtgcctctcttgccgggtggtcatttctatgagtgggataagactgtataaaacaatgaaagtcagtttcctgaggaatgtgacttctccatgctccttactttttagacccaaatcatatatatatggaagaattataaagttacctgtattgtctctggcaattttacaacattcttttgtgctctcagaatggccttggtaatttagggcatcgtttattgaatgagcaagctatttctaggtgtaaagtgcacttcattggttctatgactggcccaaaccccttctttatcaccatcttcccaaagagggagagatgggacacacaagtcatatgacataaatttgataggatagtcatcagtagggagcagcaattagcaaccttaatcagaagagttatacccaactcttgttgccagaaatagtcatctgaaaggtggcactccctcaggaccgcatttttctaactgtgatgatgagaacatcggccttgttagttttggtcctttctgagactaaaattctgcgattccagcagtggcacaaagcaatttttctggtactctaacaacattaggggtctcagagaaggacctcctgtgttacatgtgaagatgcgtaaaccacagcactgtcactagcagttccggtgcaggattttctcacaggtggatttcctggcttctccttgccctgtcaacattctcacttgcagagccttctgaaggtggcctctcgagcaatgccaagggcactagagggcccagggggcctgagtgctgggctgggaaggaggtggcaatgtaggctcatcacaggcgtgcaccccttaacaagccggcacaggaaccacagacacctagacgatttcttcattcttttttcaatattcctgaaatatgcattcaaacagtcacatggtaaaaattgtcaatggaatgaaaacatttttcctgtgcaatttggagctaattttttctgtgatgtaggatcttattttatgaatggattttatcctcctttaaattattgatggaaaaggccaagatttttcccgtactggggtggtgccgatggagacctgtatgacactgtgggttccgggtgtgatcttggacacctgaatccccattggcactgagtgctgttctaacattctaccctgcatgtttttatttcttgcccctgagggatttgctcattttgcatactgattagtttatagcagcgatgctaatttgcagcgacatcagggattacactttgagaattattaaatataaaggggacaacattgcagaacacagaggaagcctgactcagaggacagaagatttgaaagacggaagcctgtcccttgcttcattactgagctacagtgtgtcctggggccatcactcagccatctgtggatccctttctctacctggagtaagaatatgagagacagacagaagagttttggttaatcacaggggattttcttatggattaagtaatgaatgtgattaatgataatatagagaattcccagttacccacctgtagctactctgtagctaattttcaattctgtgttgatttccttcaatgactctcatcccgacccagatggtatctatttaggaaattcaagaaacttgtactattagcataagcagagcaaattggggaataaattagtgcaaagaaatgatccaatgcaatagaaaagtgaatataaacggcttctggattttctaggacacagactcattgaatcattctataaatatttgccaagcccctactaagtgctgggcactggggccctcaccatgagatgagctcagtcccttccctccaggttcacagtcaggaacgagtaactgcaaacaggctgagactctgcagcccagaggaggaagtgcgtttcgttcccaccgacatcagtacctgatagtagtaggatggttttcttccagacatttcttctgatgcatataaatatcctcagacacattttatctttttttctttctcttttcttttttctttcttctttcctttcttttcttttttcttttctttctttctttccttctttctttttctttctttctttcccttccttccttcttttctttcttttcttttcttttcttttctcttcttttcttttcttttcttttcttttcttttcttttctttattttctttcctttcctttccttttcttttcttttcccttcccttgcctgcctgcctgcctttcctgccttcctgccttccttcctttcctttctttgtggtattgattgccctgttgcctaggctggggtgcagcagcatgatcatagctcactgcagcctctaactcctgggctcaagtgatcctgccacttcagcctcccaagtagctgggactacaagcacgtgccactgtgcctaatttttagttttgttttgttttttttttttttttggtagagataaggtctcactgtcttgcccaggctggactagaactcctgggctcaagtgatcctcctgcctcagcctcccaaagtgctgggatgacaggcttatgctaccatgcctggcccacatctttcatcagaaagtgagtgctttggacatgagtacagtgttcattttcccgatacaaacattgagatcctggggagatcaacagcaagcttcttgaggatggaggcgttctgtctccatccccaaacagccccagtgtgagggtggggtctgtatacaggagatgctcagtggtcatttatcaagtcaatttgttccccatagttcgctccatagggaaaacatttttgttatcatcatcactgtcattcttattttaaaaatgaagaaggtaaggttcagagagattaggtaagtgcggtgacacggtgtgaaaaaggcagaaccaagacccaaagcgtggcctctgcctccacaggctgtgcactctccacgttgcactgccctgctgatagccaccaagaggactcaaacaatgggtgtgtgtggatgtgactggaaggcaggggagatggaagttcctgggcttgctgagaacctaatggtgtgtggtatctgggaaagagcaggaacctgggactcagcctgatccgcattgactcctaaactctccttcctgctgtctgcatggccatgggctagaccctgacccaccgggactcggtttcctcatcagcaaaaccggtataatagcatcttcatcttaggtctgtaatgagaagcgaatgtgatcaaaagataacgtctggtgaacagtacgttcttgattcatagtactttattttattattatcagtttaaacatttttcttgatgctgctatttgcctttaagcatatgaaacaaagaatagatttctaaattatgtcatatgttacaataagtgacctttaagattattcagagaatccgatctttttggtagacttaggaacaaaagcaaggttcctttttctgtaactgctgttttctaacttacaaaaactgaggtgacatattttctagtattgtttctaaatgtatgaagcctgcttcattgcaccatggttggttggttcacttatttcgtaagtgtcaagttaagactcagcaagggggaagagtctcccccagctttaaagtctaccatcattggtccatgccttgcaattgcccatttcttcattgctttgtaatttttctgttgatgcatttgtctctcctgctccatggaagaaccaagaactgtttagcattgtgtccccagagccaagcacaatccatagtacataccaggcaagctcagtaatgttttttctttttctgaatgaatgaatgaaatggggtaggagtaaaaggagacaaacatagcacaacatatattaggatcagttactcattttcttttctaagaaatgcggaggcagtgcgaccctgtgttaatatataaataagaacatgcagcttcacgcttctcctcattgagccctgaaataagtacaaaggtggcagacaagatctagctgtgtgggagatgccacggaaggaggggatagctttaatttggcctttttaggatgctgagtttatgcctccaagtgtcagagccacagatgtctgctcctctgcaggggaagatgaccttgaaggtgatgttattgatgagctttatttaaaacacagacatgttcaagagctacaagactaattggctatgttaaaatatttaaatcaaaagtgcagctacttagtgtgtgttggcaaacattttatgttacagttgtaaaaatgatacatgctcactatggaaactttagaaagatcaatggaaagaaatgattccaccacccagagataagagctgatagcattttggtgaatatttttcttttattttatgtacatgttattaatatttttacaatggttcatttcatactttttgccataaaacctgctttagaaaaaacagcaataaatagtgaacattttccaggccagtgtatcttcttgtgtgacctaattttagtggctgcttagcattgctaagaggtcctgatgctggagcacgattagagtattttcagtcttgggctactaagaaaaaaacctgatttttgtagccgatctttgtgtaaaactatgattatttacatgcaatcaatacttggatgtgggaatgtagaatcccaagttatatttaattttaagtctcattcttattccctactctcaccaaagacttatggtttgggaaaacctttgccaggccagtttgttaggtgaaagatgatgttttatgttgtttggattttcatttaaaactatctttggaggctgggcttggtggctcatggctgtaatcccagtactttgggaggccgaggtgggcggatcatcaggtcaggagatcgagaccatcctggccaacaaggtgaaaccccgtctctactaaaagtacaaaaattagctggctgtggtggcgcgtgcctgtagtcccagctacattggaggctgaggcaggagaatcgcttgaacctgggaggcagaggttgcagtgagccaatatcatgccactgaactccagcctgggcaacagagctagaccttgtccaccctacaaaaaaaaaaaaaaaaaaaaaaaactttggataataatcattttccttaacacttcatttatttctgaggtgggggatcttgctatgttgcccaggttggtctcaaattcctaggctcaagcagttctctgccttggcctcccaggtagctggaactacaggtgtgcatcaccacgctcagctgtattccatctttttggaatttctggttggtgcacttgtcttttccttcataatttgatataactttttataaattagcaatattatccctttttctgccatatgtgtagcaatatatttttccccacttgtcgttcgcctattagttttagttgatggtgttttcttggacatagcttccttgaattgttgacgtagttttgcttagggtaaattgttttcctgcccacctgcattccattagaaatggattgttttaagggtgaatttgtcattctttggtgacaaataccaacaagtctcctttaaagacagctcaccaacataaggcccaaacctgtgccacattttcactagcatcactgtcaccaaatatgtcaactccggcaagaataagagaaggagcctaaaagattttaagtggttttgacagtctttagcaaattttttctgagaagctcgcatccaagcatctgcaaatcagtcacccctccttttaatctccatcaaggtcagctctggaatttggccctgtttggatagtccaaggcagtgctatccaataaaatctagtgtgagccatatacacaattttaaatgatctagtacgcacattaaaaaagattaagggccaggtgcgatgactcacacctgtaatcccagcactttgggaagcggaggagggaggatcacttgaggtcaggaattcgagaccagcctggccaacatggtgaaaccctgtctctgctaaaaatacaaaaattagctgggcatggtggcacacgcctgtagtcccagctattcgggaggctgaggcaggagaattgcttgaacccaggaagctgaggttacagtgagccgagattgcaccattgctttccagcctgggtgtcgcagtaagacattatctaaaaaaaaaaaaaaaaaaacaaaattaagaagaaagttaatcaagtgaaaattctgccaggagctcagttgcattttctagaactcttggcctaattttattaagttcaacactgtgataagtgctgggaataaaaaaaaatgtgatgtgggctctgcaagaagtgtatttctctgtgattaaagctgattgtgggagaaaatcttctgaaaccctcagtgacaaccttctcattgactagtgactcaaacagtagtgcaaagcagaagattctaaatgagcagaggaagggtggatgccgtaaaggcaagggaactggtgaatttcagaaatagtgagtgtgaattcaatactcctaatcatttgaataaagatcttgttatatttccccaaaattaataagaaacaggtgaaattaattttaacaatacatttaattcaatatatccaaaatattatctcaacatataatgaacttaaaaatattacgaatgagttattttgtactctttattttcatactaagtccttaaaatctggtgtgtgttttacacttttagcacattttaatttgggctaaccatgtgtcacatgtggtcaggggctaccttatcagacagtgcaggtcccataatagtattctgactaaggggaaaaagcaaaaatcacaagagattgtaattcaggtagaaacttacattaaggagctcatcctaaaaacaggtaagtaccctcctaagtatgagaacagatgggggccacttgctggagtttcttactcttaaaatacttgtcaacatgaggatttcagcttctgcatacctgtaccaataggtatgtgttgttttaagggccagcggagatgacacccacaccccactgagaatggagccgaggctgttggaacccactgctcaggacagcttgcacgtgtccatcacgagacgagactggcttcttcaggaaaagcagcagctacaggtgagcaggtgaaagatcttcaagaaatatgatggcatttttcttttctttgagacagggtcttgctctgttgcctaggttggagggcagtggtgcaatcatagctcattgcagccttgagctcctgggtgattctcccacctcagcctcccaagtagctggatctacaggtgtgaaccccatgcctggctaaatttttttatttttagtagatatcaggtcttagtatgttgctcaggctgctcttgaactcctgggctcaaagaatcctcctgcctcagtcttccaaagtgctgggattacaggcgtgagccactgtgccaagctggcatttttcttatccatttaagccaaatgcagccaagtgggtctttgctttccttgagtgtagcaattagagccttggctgtcaagccggggagtacaaaaaggagagatggtgggaatgactagaaggcttgtaggaatctcattacaaacgagggttgaaagctcagcagcatttgtgcctgttttcgatattcctcattaccttttgaatatgcttttcttgtctgccaggccaatttgccacttcttatcattttgacctttagaagcttcctttgaaaggcagaaagacatctgtgctgtgaaaatcctttatggaaattcctatcatggtagactgtgagctgcagttgttaagagacagagttgtaagtccggactgagcaaggcaggcacatctggaatgattagccggctgcttggaagcccaaggttggcccagaacagaggatgcaagttggctaagaatcatcgaggggctacaaatctgggtgcagcactgagttaaagccagagctctggaagttgacagggcaccccagaggcagggaatcttgaaacagggtccatgcctgcttgagcctggagctgtgaagcatgagtggggagcgatgaggacacattgattagaagcaacgtgacttcatgggaagagcataggtcttcaacacccaagttcaaatcctggcttaggctggaggcttcccatctctgatctggtcatagagtattcgtgttatctgaatttgccccatccaggtcactgcaagagtaaggtggcaaagggaggaaaaagtcagggcaatttatttgaaattatttcccccattaactgagcacttacacagtcaatgtgcatcattataacgtttggatagctgaaaatgtgaaatctggtttttgatttttaataacatttgccaattaaaggtctacacaattcctgagacagtcagagtgtacagaaaataattttagccctgggcacttagtgtttgaagcactctgggaaaggtacttgaatattcatttattcaacagacatttattaagggttcactaagtgccagatactgtttgaagtgcttgagattcctggggagcttttgatccactaggggtgagaatggggagatagacaataagtgataaacataataaaacctctattagacaactggtatgttatagggtgataagagcttcacagagtagactaagatgatgtggaagaggatggagtctactcattgagaagtgagattggacccaacagttcaaagaggagagggagtaggccttgcagaaatttctgcaagaggatcctgcagggggataccaacatgaaggcttagagtgggagtgggcctcctgtgttcagagggcagctggaggcccatgtggctggagcagtgtaaatgagggggaggatttggggaggaagtcagagataaaggggccttgtaaggatgtggacttttctcagattgcaacagggaaccattggatggtgttaagcagtgaagtgacatgatctgaccacattgtaaataatttgactcctgtattaggaataggatgtaagacaataggaaaacagtatgcagcttccttaaaagattaaaaattgattgactgtgtggtccagccacccccccttctgggtatatatctgtctcaaaaaaattgaatgcacagactcaaagagatgtttacacacccatgttcatagcagcgttatacacaatagccaaaaggtgataacaactcaagtgctcatcaacagataaatgataaacaaaatgagtatgtacatgtaatgagatattagtctcggaaggaaattctgacacacgctacagcatggttgaaccttgaggacattatgctaagtgaaatacactggttgcaaaaggataaatgctgtatgattccacttgtctgagttacctagaggagtcaaattcatagagaaagtagaatggtgattcccagggacttggaggaagagaggataatggggaattagttttcaatgggtacagagtctcagttttgcaagatgaaaagagttctgtggatggatggcagtgatggttgtacaacagtgtgaatgtacttaatgccactgaactctacacttaaaaatgctcaagatggtgaattttatgctatgtgtgttttaccagttaaaaaactaaaagaaaaagaaatgaatagggtagaggaagtcaaaagtggaggtagagaggttagttgagagactttgtcagtaatactgtaatcttggagcaattggggacactgaggcattacttttttcttagaatgtcttggtagtagacattctttagtaactcccaattcttctccccactcccagacttgaagtgggttcaggaggtagaagttgtaggtgatgcacctcggggctgagagaacgtgaataagaggttaggagacacccaggattagagttgcactcttatccccacaaaacatgggagtatgttaattaagagtcttattttttattcagaatcatatgggcagtgagacgatgatctactataacactgtgcacatgacgggcattcattacctgctgcttgtttagttaattgaccttaatattaaatgatattttatggattatttggatttcagtttgctatattcgtagaaattaacatttatttatattttcatgaactttagactgcatggcaccatctgcagcatctcattcttctctcagtcctgtagacttaagaagccattttttacctttctctgtactctgaattcttgtgtttggggacagactaacaaagggtcagttgcatttcttgttttggtctgtggctggggtaacctgcttagctcagacctcaggtgcatgaggctccctgccctgtgtcctaactcatccatccgaaatgaagtaagtcatttatgtatctgctgggttcttccaagtcaaattcttggctcaccccccatagttatttaatgcttgggagggaatgtttttccttatgccaactcattagaaatataaagctggcatgatttttccagttcagcaaacctccaaaaatctgcctgcctcagaaggggagttttgtagttctggtgcatatggccaattctcatttaaacgagtcattatcctcagagcagtttgccatgagcagggttaatttataaaatcttgtctcctcattctctacagaaagaaatcgaagctctccaagcaaggatgtttgtgctggaagccaaagatcaacagctgagaagggaaatagaggagcaagagcagcaactccagtggcagggctgcgacctgaccccactggtgggccagctgtccctgggtcagctgcaggaggtcagcaaggccttgcaggacaccctggcctcagccggtcagattcccttccatgcagagccaccggaaaccataaggaggtactgctgatttcctaactgattttgggtcgctcgcctctttgacctgttcattggaatgattttgtctgctgtctttcatttctaaacatttcccaagcagtctgtatttttcagtggaagcaccattggtgttttgggtgggaaaattcttcacggtgtggggcagtcctgaacattgcaagttatttcacatcctggccacttaacccactaaatgccagcgaccctccccattccaatatgcaccctccccatttccaaacaccctcaggacaccctctctccctttgagaactgttggaattcaattggttagagatcctaatgccaacttttgctgaggacgatcatctggatgaactatgcctctcactaagggaagaggcaagctattgagagagggacacccaatggcaaagttcccatctggaagaatggattgttactggtagctaaaatgtattgggtgcttaccatgtaccaggcagtgttataagctgactctatgagttacttcggttgttgtattcattttacaggtgaggaaaaacgtttaagaaggttataatttgcccaaagtcctaattaataagagagaaaactgatatttacactcctgttcattaattctgaagcccggtcttctccactctactctggagtgagaatcatccctgtattctgtatcatggaatgtctataagatacacatctactttttgccctttatttatttatttatttttaagagacagggtcttgctttgttgcccaggctggagcacagtggcacaatcatggctcactatagcttcaaattcctgggtttaagtgatcctttctgcctcagcatctggagtagctggaatcacaggcttgagcctccacatctggctatttttttttttttttaagttttttgcagaaatggtgtctctctatgttgcccatgctgttcttgaactcctgggctcaagcgatcctccctgcctcagcctctggaggagctgggaccacaggcttgggtcaccacatccggctatttttttgtttttatttttttgtagaggtgaagtatttctatgttgcccaggttgctcttgaattcctgggctcaagcgatcctcctgccttagcgtcctgaagtgctgggattacaggttttagccattgcacccaatctttcttgcccttcaagagctcatactctcatttctgagacatacagaacgcacacttacacatgttggacaaaaaagtgagtggcacccgaatgggtagatgagcagtaaggactgcaagtgctacgtgggtagagccgggagtgggagcaatggtcggcttggccactgtgctgaagaaaagacttcctacaactcaccacctgtagtggttgagaatacggactctgccacctgattgtctggagttgaaggttaactcctcactcaactcattattggccgatttgaggaaaggggtgttgataataatttctaccttatagagctatcacatggatgaaccgaggtagtacatagaaaactctcagcagagcctggatggtactaagcactttattgtgcttccagtcgtcgtctttacaatggaaatttcagtcttctgaaggccttcaaactacccgtccaaatatatcttactgtatttagtgaacgactgttctgtcttccacatgtatcacatcctatccagcctataaaagcaagcggcacatgctattctagggagttacttaagatctcctcccattccccttctatcccgaatatttccaggcagaactctccatggctcatgcccatggagagggggaagagatttggattgttgccggttttcatccaaatgaaacgggtcccttgaatacagcttgttctctcccacctgcactttcagaaccattcttcagctattgccttggtgtggtgggtaggctgttatgttctcattttgcaactgaggaatcaaaaactcagaaagtttccctgatgatgtagctagtaagagaccatcttgtgtacaactatggctgtctttgggcctgaatcaataaaagatggtttccttccagagagccaaggaagctatttctatgactaattctctttcctcatctaaaactaggagctccacagtgtggttggctgatagaactgagcccaggacttggtctggttttctacctgtatcctctggtttgccttttaatcaggatgagtcacagagaaggcatgaatgtgtgacttttactgagtaatctagtgctttgataggtttttatttgtttgtttgtttgtttgtatgttttgagatagagtctctctctgtcacccaggctggagtgcagtggtgcgatctcggctcactgcaacctccgcctcccaggttcaagcgattttcctgcctcagcctcctgagtagctgggactacaggcttgtgccagcacacccagctaattttttgtatttttagtagagacagggtttcaccatgttagccaggatggtcttaatttcctgacctcatgatctgccctccttggcctcccaaagtgctgagattacaggcgtgagccactgcgcccagccaattggataggttcttgaaaggcatttcaaaagttgtggctctctgattttatgatgctgttagggaaattatatatgaggatgatcttgaacatggggcactgctgtctgtggggtcctctgggagaatccaccatccccaatccgaacattcccactgagattacagaggtcaggtcaagaacaggctgccacgtttttgtgctgaggtgactataggaaaacctgggtatctcaaggactttcagaaaccatgaaggcttcatgtaggaggagatcttgggaatgtaaggatgaggctgggaacagtggctcacacctataatcccagcactttgggaggccaaggcgggaagaatgcttgagtccaggaatttgacacctaatctctacaaaaataaaaaaataaaaataaaaaaaattagccaggcatgattgagtgtacttgtagtcctagctactcgggaggctgaggcaggaaggttgctcaagccctggagttcgagattacagtgaactatgattgtgccactgcagtccagccagggcaacagagcaagaccctgactcaaagaaaaaaaaaaggaatgtaaggacgaagagtttcatgggagggtaggaataaaccagagcagttaaaaggtacaaaaaacctggacaatctaatgactagttaataacatgacctttttctcctgcgataaggaagttatacaggtgcctgcagcatggaacactcatggtgagcagaggccatcagcactggctggagttttgtgtggctggagtgtggccaccctgagacatcctcattatctgggtgattctcaagaggtgatccttggcatcagtctaatttactttgaggtgcagggaggtatcgccgcatctggagggcagtgtgggctgtgtaggtgcacactttgattggtcagcttcctttcttccaccgtagggaggtgaggcatcagactgctccctgccgtaaccatcaactagagaaacccgtcatcaatatttgggctttccatttgagaaggaagatatgtcttttccaagctgatgggctgcagcatggatctgcagaagtgaagcatttgaaagaaaccagtgtagaaccagctggctcaacaggtcagagagattgcttggtctagtatttttaactaattggacatgatcctgttaacctaatttttgctatgaagtattggttgtcttttagaaaatgctgacttagggataatatccagtaggtatatttagccaaatgaacccatcttacatacatcaatagctgccttcattgttcatgagaataggctcttcttaggccagatggagagagccagacaaattgtgaaacaggaaaaccttgaagtcttataaagagaagttgcagagactagtattgttacaaaacctagagaaacagatcactcaacatcacgactgttatcatcatctataaggattttcatagaggccagatacaaactcagggtgctgtctgtttgtaatttactcaactttctaacatgcaattgcaatatgctttgtttcgtggacaacgacccaacaacttgaaaagaaacttcctaatagcatgtgacatttctggatgtatcactggtggagacttacgtgcttacatggaggccatcgtttctgactgtggcttgttgggaaccccctggccaatgtcagagatgggctggaagtagagggcatgtgctggggtcaaggacacccagtgattttcggtttgggtgtgataacagaaatcaaagaagccatttgaattttccaatgtctgtgttctggatcaaataacttagcaggaggagatgaagtgttaccatcccagagacatcatagacactttttgagtagagccaacaaaaatggttttcttccagtgaacaaaaggagaaccctttttaagcactggagaatgaggagctccacagtatggaagctgacagagcaggggtgcatgaaaggacaggaaccacttaaaagatatgttcatttgaatttctctcatccctgggatttcaacccccctgctttaaaacaagtgccttgttttctcctgcaagtcttcctttgatgtgagggtcttagaagaaccacttttacccagtgagcttggttcttctcccacacgaaacccatggttcttgttctaccccctgctgactgcattcccattctctgtaatgggattgtgatgtgggggtgggggagggaggagagggaaaagatgggggacacagggaggaggtgacagtgtccaagattttctgtggatcacatccctccatgtgggcatgaggttccatctatttatttggcgtaaattgaggagaatggtgatttgcagctcctctctgtctagcaccattagctcagcctcccctggcttcttgttggttttatgtgtctggaaaaccaggggagggcagcctgtgacctcatgtagcatgcatatggctaacggcaaagtgagggaggaataattatagtaataatcacagtgatgacgtggagggctgcctaggatgctttctaacattctttagaaatgcttgactggcttgtattcatctggggttgagttcttcattcctcagctcctcggatttttcatgcttcatgtgaaatgtgttctggcagctcatggatgaattgtcgcttgttgtgcatttggaagaaacatgtcacatcttttgcaaaccattggcacaaagtgtgaaacaaactcaacccctggttacaggagggctggctgggccttggttggagagtctgggagcctcgcggcagggctgggctggggaggctcgcgggtggagaggagccaggccttctgcagtatccatgcccccagccactagctaaactgcccagggctccctggcggggggcactgattatgcagggtgctgcaggcatcctcatcacacctgtgctcagtccacaacatgcagtccctgctgatgtcagtcctccttgcaagtgagatttgaaataatgtaattcaccagctcacaactgaatgggaaaaaaaaatcaatagttcctcttttccctcagaaacatatcttattagagtgaggagacagtgaaacagcaaggcctgaattggaaaactgatcctgaatctaggatcctggtagagattctcaaagagggttgcgtggaagcagctgtgtgggaggggtcccattaatgctcttgccccgggggtagtgcgaggcagccaccaaactggagtcacgcacagactgcagagaatatccctgcagcatatcagcagtctccaagctgcttttccattcgtgaagattgttgttctttaatctccacttctgggctttgtctttctctctcctctgtctttgccttctacctgactggtttgccaagacaatggctttatgggactgagagcattagacatcatctggagaaagaggagagctttgcatttttctaggaaattctgattttccccctcttttttttggtaaggacttttctctcttttttagagatggggtttccctctgtcccacaggctggagtacagtggtatgatcatggctcattgcagcattttactcctgagctcaagtgatcctcctgcctcagccttccaagtgctaggattataggtgcatgtcaccatgccaggctaattttttaattttttatagagatgggggtcttgctatgttgccaggttagacttgaactcctgtcctcaagtgatctttccacctcagcctcctgggtcactgggattacagggttttcttttccccctactcccctttttaagtaaagtagacatcttgcttttctcaggaattcatgaggcagaaaaatatttgccaagacagcaaatgcagtgagacagaagtgtgtgagcaatgctggccagatgggtgcccggagtgaggcttccctgcataggcgacatctgcagcattcaggacctccctgcaggtgtgccctctccactcaaatttatcctgtctgtccttcttagttagactggtgttgatactcatacactaattttaatatctctgttatttacaaactgtcatacttgcttacaataaccctataaggtttgttgggcaatatattgtttttatgcccattttgcagatgaggctgctgagggtccaagttcacacagccgtaaaagcagtgccctcacgtctttggcgaatttttaatgcagagggaatccagagatggttcacactgatggcctgggagagaccaggccttggacagagtgcttagtccagcaatggaaagaagtccagcccagcagagccgtgggtctgtgggcccaaggaaaagagaatggatgtccctccaggccagggatggttgtctctgtgtgcctagtgttgcctgattggattggagccaaggtgtatctaaaaatagacagcctgggaagaccaaacaggcaactgtctcaagtcagagaggtctgtcagacgttccaaagaaagaaatgtgagaggaagagaaactggaaagcaaggccccgccccagggctagaggaactggtattacttgtctgtgaaaagagggtttgaactgggtggcgtggaaggtcttttctggttctacaatactttgcactttagtattttatgaaataagttaaaataagctgcctaaaaacccctcataattctattcctaggagctctatgttaagttcctccttaaagttccctgggcaggctgtgaccatccacaggccacggtgctccagctggcactttccttagggctggtccctagagcatggacctgagcaaagcctgctttccaagcactgctgctgcagctcagagcttagacctcgcctcaccattgtatatcctcccgttttcttctggaaagtcgtcattacaggcaacgtggaaaagtttaccagactataatctggtaaaggaggaagaggattggaaaagaaggaagagatttgaaaatcccccttatctggctgggtacagatgctgctcaccaggaaatgtaggttcccaataaaacaggacccccatttccagttttcattgcgttgtaaattaaaaataaaatcttaagcccccaaccactgaacggattccctcttggccaaggagaccccagagactccatacaaactgagttcccagccccagtgggccgggaggtcggatttgccttgttataatccctcccttttgtagcttagacataacaacagagcagcatcaatgctaaaacgagatcataagaactgatggaacagactctcggtggcaataagataccaaattataaacaagacctaaggccatgccaggcagggataagtctcacacccctgcacctaaagagtaaactaccttctgactgccacctgggttttctttttctctagcggctaaacgagcactgtcctagagatgagctatcttaaaaccatggcagtccttgcaccgccagatgctgactgactgactctctgttccaccagctgtaactgcagctttcattagaaaagagactgatttcagtaactttcttctgatacaaagaccaactgtggactgcttctggcctatttagagacacttgagtgcctttgtgttctgaaaagaccttttacatagagggcctaattgtaatacatgtaaatgttaagtctccaccctaaggtgaacataagttgtatataacatgtatgcttattcagtatgcatgcattaggaccaccttcatgaatattcatagctcctgctataacctgtggaatatgtatacttggtcaacctattcttgtttttttttttttttttttttttttttttaagatggagtctcgctctgtcgcccaggctgcagtgcggtggtgcatctcagctcactgcaagctccacttcccgggttcacgccattctcctgcctcagccgccggagtagctgggactacaggcaccggccaccacgcccggctaattttttgtatttttagtagagatggggtttcaccgtgttagccaggatggtctcaatctcctgacctcgtgatccacctgcctcggtctcccaaagtgctgggattacaagcgtgagccaccctgccggccttggtcaacctattctgtgtaaattcctgtcttacctgtccctcccttaaagtgcctgctaatggcttctgccggaggctatgcttcccagcctgttagaatggcaaccttgcaggctgtaacccccttttaagaaataaagctctccatattctcactcataggtgggaagtgaacaatgaggtcacatggacacaggaaggggaatatcacactctggggactgttgtgggctggggggaggggcgagggatagcattgggagatatacctaatgctagatgacgagttagtgggtgcagtgcaccagcatggcacatgtataaatatgtaactaacctgcacaatgtgcacatgtaccctaaaacttaaagtataataaaaaaaaaaaaaagaaaaggaaagaatgaaaaaaaaaagaaataaagctctcctttctaaattgataaattgtgtgatttttaagttaacaggctatatttgagaaactcaagggaggaattctgttttacttgcagatgtaaaaaaagactttattaacacatgaaaaaatgctcaccatcactggccatcagagaaatgcaaatcaaaaccacaatgagataccatctcacaccagttagaatggcaatcattaaaaagtcaggaaacaacaggtgctggagaggatgtggagaaataggaacacttttacactgttggtgggactgtaaactagttcaaccattgtggaagtcagtgtggcgattcctcagggatctagaactggaaataccatttgacccagccatcccattactgggtatatacccaaaggactataaatcatgctgctataaagacacatgcacacgtatgtttattgcggcattattcacaatagcaaagacttggaaccaacccagatgtccatcaatgatagactggattaagaaaatgtggcacatatacaccatggaatactatgcagccataaaaaatgatgagttcatgtcctttgtagggacatggatgaaattggaaatcatcattctcagtaaactatcacaagaacaaaaaaccaaacaccgcatattctcactcataggtgggaattgaacaatgagatcacatggacacaggaaggggaatatcacactctggggactgttgtggggtggggggaggggggagggatagcatctggagatatacctaatgctagatgacggttagtgggttcagcgcaccagcatggcacatgtatatatatgtaactaacctgcacaatgtgcacatgtaccctaaaacttaaagtataattaaaaaaaaaaaaaaaagaaatgcagcctcaggaaaaaaaaaaaagactttattttgtgctacagtaacatttacgatgatttttgaataacagaacaaaggagggtgaaaaaatggatatgtattttataatgtattttaatattttacaaattctctgggcttgttttctttaagaagcttacaacatgagtctattattcttagtattattattgtttaggtagctctttggaggcttgtgatttgtagactcatccatttctcttttatataatttaggcattagattaattattttcaactggagccccatggattatcaaagatgagagatagtcattttagaatgatcatagctagttctaatattgcagctgcaagtcaactctcacaaacatgaaagcatgtgacctattttaagtctttctcctagtcgatttcaacttttagtttccattattatcaccttgagaaccgttctgtacttaatttctctgtaatttacatgtcttttgcaatcttctgttccctttatcaaattctcttattctttcctttttgtaagttgtatgttactattccctgctttaacttttcagatgttaatttagtatgtcaggctaaactcttcttcagaccttgagctgaaactctgggagaagagcagaagttttggaatcttccagcagaaggtaatttagctctgctcagatagacccttctccagtgactgctgggaatatccccaggttggaaataccagctcattgcttgctgagaatttaatcagttaagagcacacctttttcgagctgtggaagggtgattaatctaccctgtgctgatagctaggctgctaagatgacccaaagcaaagccagacagaacctctagatttcctgtagctgctttcagtggggggtgctgtgtcttatatagagatcgttgttaaacattcatcttgactttcattgtcattactattttgtacttcctctttggcaaatttcctcagcttcctctttgagtcatgttgatattggtgtttggattctttatgcaatttctcctttagtcaactggatttttttcctactgttaattcattttgttcaccagtggtctatttttgcctttcttatccaataaagtatgtccatctttagtttacccacagaaacattttaggaaatgaaatcgacaatattccccagttttaggaatcaggctgatctgttgctttgtcaacagattttgagagatgcttgagagaaatgtgaggttaaaattgtctctgcagacttgagctgtgcatatgcttattcttagccagacgccagttaattaaagatgattcagaaaagaagaataaacccacagcagaaagtacaacgtttttggcaaactttacagttatggaaaatataaatacttagaaatttaggagaagcaagaatatattttaaaaattagtattaaatataaatgcaaatggctgggcacagtggctcatgtctgtaatcccaggactttgggaggccgaggtgggaggatcacttgaggtcaggcgtttcagagcaacctggccaacatggcaaaaccccgtctctaccaaaaaaaaatacaaaaattagccggctgtggtggcacgtctgtgtagttccagctactcgggaggctgaggtgggagagtcacttgaacatgggaggtggaggttgcagtgagtggagatcgtgtcactctactccagcctgggtgacagagcaagactgtctcaaaaaatataaataaataaataaatgcaagcagttctgggtatttatttaaaaactcagttgttcccatgggagcaactggacattacttattaaataatcagaataaagggagggtagtaattgaaatggaaatatgaagttgcctttggatcacgtaacagaataagcctggatgtagtatcattttccccttagatcaaattgttgtatttaatgggttgaactcacttgagtttttgggggtgggggaaagctctagagcccaagtactgaaatcttgtttggacatttaaatacatcgtggttatgagaatagttgaaaatatggagctgacttgattaccagtctccaaaaataagaggctggtaatttatttccttattattgtaaaaaagaatcattaaagaacttgtgaggaaggggtagaaaggagaatgattatgatctgaacataaggctgttcatatttctgattcctgttctcattttttcattattaaaccaccaaaatctcctacagacaaatgtctgtaagtcaaaatgtttttggcacatgaattacagtttcagtttggaataagattgtttgaggcaatgagaaagatctggttgaattaatacttggagtatttggataagccaaataccaagttcatcagttaaaaataaagctgtgatttttgatgaaactggacttcaatgcatgctgtgatgttgtgatgtcaggcagagcttcccagtaagcctggggacctgcaagagatggcacggggtgagggtgtgaacgcgtgtgtgtgctaccttactcatatgtgccaatttggaaagttcagcaaattcactattagggcttactcctttcctctgctcactgccttcgcatcagcacctcgatcacaggaaatcttttccaagaaaagatcagaaatccttacgttcatttttttctctccctttctccctccattatcccttatgatatgagttacatgggtgcatcttattaaaaagggttcatggtcttataaaatgtgacatcggccgggcacagtggctcacgcctgtaatcccaacactttgggaggccaaggtgggcagatcacgaggtcaggagtttgagaccagcctgtcccaacatggtgaaaccctgtctctactaaaaatgcaaaaattatccgggcatggtggtgcgcacctgtaatcccagctactcaggaggctgaggcagtacaatcgcttgaacccgggaggcagaggttgcagtaaaccgagatcgcaccattgcactccagcctgggtgacagagcgagactccgtctcaaaaaaaaaaaaaaaaagtgacacctcattttcagtttgctgagttatttggagaagttgtactttactaaaaggactcagaatatcagagtgtagcatcctagtggaagacacagtatccttgaaattcagagaaatagggagaatgtgagtcatgatgtgatgtatattcagagaaagagtcaaataactaaacgttcaagtatttccctggcctagttttctttggttcttttacttaatgtccttagccttggccagagagaaaaactctaaatatatttattccacccttgttaaaaatttatgtgcctgattttggtcagattactctaaaatattttcttactataatccaggtatggtatatgtctgcatgatttatgaaatggtctttctatactttattcctcccatgatcctcaaaagatgagctagttggtgagcacgaaggtgagtgtctggcttgagtccagcaaactctctggcttcagccaccaccgaactggaaagagataatgtgctttgccaaaggtggcaaggatggcaagcagaggccctggtgctcctacctctcagctcagatctctctccactgcaccacactggctttcccagaagtaaaggtctcagataaccagcttgagtcatttttatagccatgtagaatggcttgcttctgatgaatttatgtaccctgttgtgcctttacaagatcatctgaagtctagaggaattcatggcgtttgtgcctctgtgctccatgaatgtccgatgggtgccatgtgacattatcagctgtgaggatagaagggtctaaggaaagaaaaaggccaagtagataaacatgaatttcctaaagctgtttccacagtcattttgttatccacttgccctctgacagaaggtgaggggactgagcagtcattggcaaggttgttaatgtcaacagatttgtctgtgtgtgtgtgtgtgtgtgtgtgtgtgtgtgttattttatttatttatttatttatttatttatttatttatttagagacaaagtcttgctttgtcacccaggctggagtgcagtggtgcgatcatagctcactgtagcctcagcctcctgcactcaaggaatactcccacctcagcctctcgagtagctgggaccacagacacatgccaccatacttggctaatttttgtattattattattattattttggtagagacgggttttcaccatattgtccaggctggtcttgaactcccgcgctcaagtgatctgcccatcttggcctcccaaagtgcatgggttacagacgtgatccaccatacctggctgtgtgagtgtgttttcattaaaggcccaagtggcgcctgtgctaagatagctgtcattttgctctttccatcatttgtagtttgctttagtaataatctctcattcttaaactgagttttacaaaacaagaaacaggggatgggatagggaatgtagggagagggtagagagaaggctgtgcagagataaaattgaatttctcatctcctgtagctttttgctaaagtggaacttcagcataaagcctcatatacacaggcaggaagatgagcggacaactgtctgcagggagcaggccgtttgcacagttagcttcctagctgaaaaacacatccctcacttagtgtaaaaaatgacatgtatctcctgtatatttgtctctattttatatttctaatatctaatatatctctattatctattatatttctgtctcatctaatatattctgtatatttctgtctctattactctattatagagtgaaaaacaacatgtgtctcctgtatatttctgtctctattatcaatgttatctggcactttataaaacctttgtgttttttagattctctaaatatctaaaaataaaaaaggctagtaaaagatttttgaatgatagagcagttaaagaatatgggatagtagacctggaaggattttagaaataatttagtccaacttcctcattttgcaactgagaagatggagccccagatgaaagtgacttgttcaaagtcttgcagaaagttagtggggtagtggtgtaattctctaagtgtcgttaatattctttttctgcagaggtccagataagaacaggaacagagtttgttcttcaaaggctcatctttggggcaggtgtgttgttgtcaccttggatgtgcctctaatgctgagagccaaggctttgcctccagtgccaggtgctaaggtggttatggctgggttactggtggtacgaggaactggtaataaactaccaaagattgtatggaatgagaggtggtttctttaggatataatattaaaggggtctatccaggcctgcctgacattgtgtcccagagtaaggggtcaaatggaacttcccaccttgtcttcctacccttgcacctgtcccacaccatggtaggttttatatggggacctagcctgtatgcccaggtaatatctacacccttcaaggtagcctgtgctcgactactgcttgggcctagaggtatgcccactgcactgcgtccacccttgggagcagtgctctgagaagaggccctggcaggatgcagcctcctggctcttcaggaaggcacttctcgttttggctgcctgaaccagctttgggtggcatgtacctttggcgccccatttacccttcccctcaaggataaggaggatcaggacagagccctctaatgtggcagagagcaaggaaggcgttcttttgcctggatctaaagcaagtattggaagtattactaagagagggtgtggatttaagtatgttctaaggctcttgagataggcctccctaagggacaggatctggagaaaccaagtaaatagttgttagttgctgttagattggggcatgggattctctaaccaaatagatgcccaatgcagctgtcttagtccattttgttactacagactgagtaatttataaagagaggagatttatttcttacacttctggaggcttggaagtccaaggttgaggggcttgcatctggcaaggaccttcttactgcatcatcccatggcaggaagcagaagggcaagagagagagagagggaaagagagagtgagagtgagagcatgtgtgctagagagagcgctggagagagagagacagagagagaaagagagagacagagaaggggaggggaccaacctcacccttttgtcaggaacccactcccccagtaatgatatcaattcattcatgagggcagattcctggtaacctaatcatttcttaaaggtcccgcctctcgacacggttgccttgggaattaagtttccgacacgtgaattctggaggacacattgacattatagcaatagctataggtttttgagaaagatgagcaagatgctctaagcacaatcactctgaatttttggagccatcttctgatcttggcagcatgttggcagtgagaactcatctggcacttgctagaggcaggctcccagatgagaccactggtgccctttgcccaggcctacacctcacttccagacttctagctcttgccattctaagaggcaggaatgagtgaccctccacattgtcttccttcccatgccagcctgtattagtttgctgccataataaagtagcaccacagccacctggcttaaatacagaaattcatcatgtccagaagtccaagaatcagccgggcgcagtggctcatgcctgtaatcccagcactctggaaggctgaggtgggtggatcacttgaggtcaggagtttgagaccagtctggataatatggtgaaaccccatttctactaaaaataccaaaaaaattagctgggtgtgctggcgcaggcctgtaattgagctacttgggaggctgaggcaggagaatcgcttgaacccagaaggtggaggttgcagtgagctgagatcacaccactgcactccagcctgggtgacagagtgagactccatctcagaaaaaacaaaaaaagtccaagaatcaaggtgtcagcagggttgcttccttctggggactgttagggaaggatctgtatctctccttggcttgtagatgatcattttctccctgtgtctttttatatcatcttccctctatgcatgtccccgaatccacatttccccttttataaggacaccagtcatgtcggagtatggttgatcttaactcaaagtacatttgcaacaaccctatttccaactgcagtcacattctgaggtatggaggtcaggacatcaacatacgaattttgtggagaacacaattcaacacgtaatatagcccatgaaaggaaaccctctaacagagataaaccagagagtaagacttctgagagatgcaatttaaaaagaagagaggacatctcatgagttgactccaagtttttaacctctttctctgtcatgcaaagcttcaccccagccatacagtcaagagcagctgcagcctttaatccctgatcctgagggggcagtggtgggaaatggggataaagatggagccgctccccatcctgtggtttgagtgtgcctctagcagaggtgaggatgcttccaccccaccccaccccaccccacttatttcttagaagacaagaatgtgagtattgttcattaatcaaatactgattaggtacacactgtgtaccaaacacttcaagtacttctccaaaaatccttgcccacatgaaattctttttctagtagtaaaaggcagaaataaataacaaacataataaattagtaagttacatgtaggttagtagaaaatgaacatgacctggggattggggtggaagacgagtggatgtgaaatttacagctgtcaatggagtagtcaggccagcctcactgagagggtcagaggtgagcaaagacttgcaggtgggagggggttggcccattatgtgtgctggggcagaaggtcaaggcagtggaccctgcaccagccacagcagatttgaggagctgtcaggaggcctctgtggccagggtggagtaagcaaagaagggagctgtaggagaggaggtaagagacgagatgagagatgggccatataggcccttgcagacttggctcgactcaggtgtgatggagtgagtagtggcattttatttgacatgcttgtgaaggctcaccctggctacatctcaagaacagactgtagaggagtgagggtgaacgcagaagaactgttcagagacgattgcagcatttcagaggagaggtgcccgtggcttggagcagagtggtagaagtagagacagttctttggtcagatcagttatctttagaatagtttctcactcccaagtcattgaaccaaaattgcctggctgtctcctcctgtataaatgtctcattctggcccttgaccaagtcttggcttgtattatctagattagtgactgttattgacttaaattccatttgcccttctaattacggctgaaaagcaatctgtcccccaagattgaatttttattactcatatttttgacatcagtggttgcattctgtttgtgatattgcaagagtattgaagggagccctttttcatttctaatgaatcatttttaaataataattttaaaatcataatccgtaatcatttttaaaaatgatttttaaaaatccataataatcatttttaaaaatcaaagacattctcacataacattttcttagagccaccattcataatacttagctcctaaactatagacaagaatggtaatgttctacacacgtttattgtagcagcccacagtctcccagttgggcagattgaaaagaaaccgtcagtctgaagaaaacaaaagagtgccgtgttttctgttggacctgctgctgctgctacatggagtagcaactctttttacctggatcgtgaagcaccagtgtgcggtgccctaataatacctataactcatcataccacaagaaatggaaaattccacatgcatgagtctgctagggctgtcgtaataaagtaccatggacagggtggcttagacaacagaaatttattttctcacaattctgaaggctgaatgtctaagatcaaggtgtcagcagggttggtttcttctgaggctcctctccttggcttgcagacagctgtcttctcttttgtcttcagatgatcttccctccacatgtgtctgtctccgcatctcttcttttcttttcttttcttttttttttcaagatacagtctccctctgtcgcccaggctggagtgcagtggcatgatatcggctcactgcaagctctgcctcccgggttcacgccattctcctgcctcagcctcccaagtagctgggactacaggcgcctgccaccacgctcggctaattttttgtatttttagtagagacggggtttcaccgtgttagccaggatggtctccatctcctgaccttgtgatctgcccacctcggcctcccaaagtgctgggattacaggcgtgagccaccgcgcctggccttccacatctctccttataatgataccagtcatattggattggggttcaccctaatgactttattttaaatttattacctattgaaaagccctatctccaaacatcgtcacattcttaggtactggggattcggactccaacctgtgaattttgggggacacaactcaaccggtgacaggcacctttgcatttaattttcttttgccacagccaccccccaggctcatctgctgctgatgtgcgttctgtgcagcacattcatgtcagtagtctcaatttttgtaaggtttcatttgttgagggtgaaatttggtcatatgattggaacaccagtttgtgagtaaagaataatgtgtatttacaaatcatgatattgattatattgtacattgaacaaaattctgaaatagaattccaaaaatttgtctggatgacagttcattttaaataggtacacagcttctgggagcggtagttagtattgttcaacagttaggagagcagatagttagactgcctgggttactgtttaatctcccataattaccagctggttacccctgggaaagttacttaaacctctcttagtctctttgatgaaattttcttgtcttacaatgtagataattagagtttctacttcatagagttataataagaatttaaaatgctcatctacatagagaatttgggagagtgtctggtacatagtaagtgttcaataaatgttggattctattatttccagggcagccactttgaatgaagatactcatttgggtaaattaggtctggtgtgtttctgaaaatgtgttcgctaatttgtttggacaaattgtgtgtttctagggtaaggcataatttcatctgagaggataattcttgtttgttatgaataatatgcaagttttttaaaaagtggggattggtttcactcattaaagtacacggaactcctgcttgtcagcattgaactggtcttattttctggtttttggtttgtagtcctgctcctggaattacggtttggggaccacagtgtgatggcagcagcaacatgtgtgtatgtttgggggactaatgtgacatctttgtaccctaggccagacaccccacttcaataaaagcagattccctgttatctttattatgttttatagtgctgtgtaaactttggtttgagagaattcttctactataaatagcctgaaacccactagcatagtatatagattcctcatatcgtctgctcccccaacaattgcttttattctgtatataccccagggatacaactaatgttaatagtagcctgagcaaaacgtaattgggaaggcaaatctgttgcaaaaaggaatatagcataaattaattataaatcaaattaaatataaaacaatgcatttaattatctgcatcactgcccccttcctctaacatggatatctgagaggagactgattttctttctgggatagggccagatctcagcccagacaagtgaactgtgtcaacccctggaaagatggtgcttgacctccttttttgtgatatgttgggcattagctaaaggcactgctgttttggtcagctaaaatttcagtatcagtaagaggatctactacctatctgaattgttaatgcatgggctagtctttgtgtgtgattgggaacacctacttataatatactattaaatgctcatataggttcaatgatgtgttgaaccatttattaaaaatgtatttgttgaatggactctaacgagcccagcaagggaaagtgcatttctgcccaaggaaggttttcagtttgggcagcaggcattagccacccaaagctggtgctgctgttagaatcagaggaagaaccagtacgggtccatgttggatgccctctgtccttctcaccaccctaagtggtttcatctgccccaatctccatgtctgtgtgacactctgacttatgttctctcaaaagatccaatccctggccagccagagtctagcattctccaggcaggattccaaacattgttttccactgttcccactgagtacacgaatttttgtcagatggcagctcctgaattctgaagagtctggtgtcacatgccccacctctgtcaaacctcacttcttccatttgggctgatctcagctggactggaaaaccctcctctgttgaaagtaggtctaaagtggtaatgactgattagtacggacctgcaccagttcccaggtatttacaattgaaacgagtttgcttatactcagaagtgacaaattggtggatgtgataacatcaaaatatagttcttacagttgaagaacaaacaaacaaaaatcagcaaattggcagcttagctccgatacttggcaacctctggtatgagatttcctgacaccctgcacactttcccttcccctcccaacacataccccagaaaccttgacctttccttttgcaggtcatcatttaatcaagtaatcactcctcttctagcatctattacattttctggcatttctagcagcagtaagtggttgagggcaggaaccatgtcttcatgctctaagttcctagcttgctgggtgcccagtagatagatatttgaagaatgagtgagttcatgaatgcatgaaagagtggaaaactctagaatggatgttctcactgctgtgagcatccacgtaatccagtcctgttcttccctcccctttctgacctctatcactctgcggggcccatggaccccatgacgggcttacactgctgaagaggcctgtctggttttgtcacattcagactttcttcctccaaatcatctctcatagtgcagcccaattatttttctaaaaaacaaaaacacttctttcatattgctctcttgctagaaaacggaatgatttctcattgcttgagggataaaactcaaaactccttagtctggcatttaatactgactagagcttgcccttgatatttctttaaaaatattttaattgtaaatatttcaaacatatttaaaaatacaacactcatttatccactactaggattaaacaaatagtagaatttaccccatatttactttgattttctttccctgactaaataaatttattttattttattttttggagatggagtcagggtttcaccatgttggccacgctgctcgcgaactcctgacttcaggtgatccaccccccaaggcctcccaaagtgctggaattacaggcgtgagccaccacgcccggcccctaactaactaaataagcaaacattatagatacagataggccccaccccatccttctctctcttttcctaccagaaatagtcatttccctgtttatctttcacaactcacctgtgagaaccatttggtccagttagatttatcttcttgctagaatttcattctctttttttttttccctttgaggcccagttcacttttcacctcttctggtaagctttcccaccaatctcctagttctaaattgctgcacaagttattttctccattgctcaaggcagacaatattctgcctggtgacagcttttatgtagcagttttctcctagtattgattttctattccccttatagtaattttttcctcttgctttatattatagcgaattgcatgcctgtgtttcttctctactaggtaacaaccttcagaataagatctgactcttaattgcattgattcattcattctacaggtattgactgagggcttactaacaagctccaggaattctttaagcactggggatgcaggagtggacacaacagacagtcgctgccttcatggaacttaagttccagtgggagagagagaaattacatgaataaataaatatgtcaagatgaaaagtgctatggagaaaagagaagcaggagaaggctaagggggtgctatctcagacatactttaaatatgtgtctctcctttatgctctatgtcaatgccgaatgcagagggctctgggtacatatttgctgaattgcattgtctacccaaatagatactatttgattccctgctggtgagacactactcagtgtagaccttccttctgggaaggttgttataggaatgcagattttaatcctttgctgccaggagcacaccttggctgttcttcccgttatcagtagatgtacactctgggcatgataaaattgtaatactagctttagtaaggcataataagggaggaaaggtctgtttcctagcaccatatttattccgtagtcaaatggagccaatttggcatcatctttcctcctgcacttcttcgtccatcagaccagtggaaggttcactttttgcagtgttcctaactgtagtggtattgaattgtggttaccaagaagaccaatctcctctttttaattcttccagcctccaggaaagaataaaatccctcaacttgtcacttaaagaaatcactactaaggtaagtacctttatattcccattttccaaagaagcctatgaagttttcgtttgacttgattttacatctagatcttaggatacctggcttctgcaaaaaaagatgtagactttgtcaagccattttgcaggcccaatgatgagttaaaagagccaggagagagtgcttctgtcatagtggaggtcttgacttgtggacaccccagaaatggactggtttggcctttgctacaaaaggagctgtcaatttagggactgaaaaaggactgccactatgcatattgaaagcctttgcttaaagatgcattcggggcttggtgcggtggctcacgcctgtaatcccagcactttgggaggccgaggtgggtggatcacctgaggtcagaagtttgagaccagcctgaccaacattgtgaaaccccgtctctcctgaaaatacaaaaattagctgggtgtggtggagggtgcctgtaatcccagctactcaggaggctgaggtaggagaattgctttaacccaggtggttgaggttgcagtgagccgagattgcaccattgcattccagcctgggagacagagcgagactctatctcaaagaaaaaaaaaatgctttcaggatggtagtaatttgagaaattaattacttttcttcccaggagggcaatgtctggtttcctcacaaatagaacagttggtgactgtttttttgttctttaaagcttttcagttggcttgacaatcattttgcctactttatccatcgtttatactgccatagcaagaccttggtttgtgtacagacagaatacgtcttcactattccctgagagcacagtcaattaaatagtcaatgtcctcattagttaggataaccacagtttacaaaacaaaagcccttctcatatgtatttatacctggccaagttattgacagactgagaaacaggatcaattactctgtgaattatgactaaatgttgtggcagagaactgggtatgaatcattcattatttgcagttggtctgggaacgaggggtggttgtaccatggtcgaaatgtaaaaaggacagcttgctgagcagagagcaacgcagctgagagcctgttggcctggaaggactctcttccggtctctgtgcccagagaaagaaagtcttggaccctagatcaggaaacacgccaagggatcaccgcagctaagccagaatcagggatgtgatgttggcaaaaatgtctggagttactctgtgcattttctccatttctgtatctaatttatttctgaaaacaacacccagtgattttgctaaaggtcacagagccgcaggtttgatgggtaattataacttgtgtacaaagagagcttcctgttaggaacaagtggtgcccgtaagacagcatcggagccagggacccagaaatgcttgacttctgctctgctcacccaaaggttctttcacccaggctggagtgtaatggcatgatcatagctcactcagccttgatctcccgggctcaggtaatcctcctgcctcagcctcccaagtagctgggactacagacacatgctaccttgcccagctaaatttgtttgattttcagtagagaccaggtcttgctaggttgcccaggctggtcttgaacttctgagctcaagcgatcctcctgcctcagtgtgtcaaaattgggattgcaggcaagagccaccgcacctggcctctcttacattttctgctgtttatcaggtgcgtcctgatttctatgtagaattaaaagatgggaggactgtcttgggtctgagtttattgactatcttcagaacatactgtatgggtaatatgaatgcatctgtacaccaacgttgaggatacaggtgtgcctattatataatgcagatggtgcttatatgggtgtgtgtatgacactgtatattgtggatacacacatatactgatacgcacccaatattaggatttgctgaaaatttccaactactattaaagatcttagattttcccaaatatcaaaaatgggttgttttgtttgcatacagtcctactccatttgagttgctataacaaagtactgtagacgaggtggcttataaacaacagaaatttaattcccatcactttgaagcctggaagtctgggattagggttccagcatggctaagttctggtgagaggccctcttacaggctgcagacggttgacttcttgttgtattcctgcatggaagaaagagggcaaggcggggtctctggggcttctttataagggtactaatccccttcatgggggccccaccttcatgacctgatcacctcccaaaggccccacctcctaatgccatcaccttgggagttagactttcaacctatgaattctggggggaaaaaaacatctccaaccattgcacataccttctctaatacatttataaaactttataattacttcgcttttccataaaattaaggaactcacatctttgattttaaaatgtaaacataaaagccccatttgataatgagttccttggtgtccaattttatttgtaaataaaaaggatacacaggttttccggggatgtgtatgtgtgtgtgcagaggtggataggtgtgtgtgcacagacacgaatgtgtgtgtgtgttgtgggcaggggcctatattagtccacagagaattaaaacaaaactgtccagtcacacaaaacacctttctctgtactttaaactagattgaccagtgaccatgagctgagaccaaggtctcagcttgacatagctttctttctctagtgtgttagacacaccacacacacacacacacacacatacacacacacccctacctgatattctttagactcctgtctcagaaagaaatgaaaccttccttgcactcattacatttcttaaactcttatgggttacccaaaccaaaagtaattaagggataaatgagatggaagaaacagttggaaataaatgggatatctggagattggtagatattagattacatccagcagagcctgaaagaatgaacattgcaatttaataagaagattcagaaaggttgtttagtattaatattgacagcttgaaagatagatttgctcaacaaaggagaaactgactcaattatgattaacagatactgttcactaaaatcagaaatattggacatagactggatgagacataagaattatggctggtattacagagttaatggaaatgccctcagacctcagatgacattattaaatagataggatgggatgtgccagtcatctggtttgagccaaactaagacttggtgctgggggaagggatatcttccttttaactctgaaaagtctcaagactgttgaaagggtctcggaattaattactttggacatgcggatgcccaacatcaccagtgcatgctttggtttagaggctaccaaactgtcgtcttcatccagagaatgatttttataaaaaagatgttggaaaattacagccactgaatagaacatatggaagtccatgagagaaagagtggagtattttaggcaggatggatgaggtctgctttgactccaaaaataatctcaattcaaactgatataagaaaacatttaaagttgatggtgtcccacaaaccttagattcttctgggaaattaaatttttctaggtagctcattaatctgagtctagataacattggcatatgaaaagtcttgagactgttgaaagggttcaaaaaaggaaaaaaatgtgatgttttagcctccaacaaaataaaagataacctaaagacatttggaaaaagaaattaagatttaagatttttgctatttttgaaaaaccaaagaaaaaaactgtaggatgtgtttggaatgtcatgggaaaatacatgataaaactctcaaacctcactgagagttacttgtctatttttcaaatctgacttgccagcaagtatctatctgccttcaaagtttccctaaacagtgtctattaatactagtaattaaatttagcttgcagctttgaccttcatctcaatggccatatctttattcactgcgtactatgtgccaggcacagagcaaaatccctgctcacatagagctcgtatcccggcagagagacaatgtggaaacaaaggaatagaattacatggtgtatcatctgagaagaagctgtggaagaacatcaagcggagggagggccaagggaggttgctattttgggtgggtaatcagtaaggcctttcctcccaggcagcgactgagcagtctggaagggtatgagtgagccaaaccacctgggactacaaaagtgatgcagacaggaggattcggcagaggcctggagaaggcaggaggcaggagtgatgcaattatgttaccttgtggttgctctctgttagctcctatcagttttgtaattggatgtggggtgatgatgggcatatgcttggaatatgccattgctcactttatttttgttcttcttcctacatttccctccctctttcccttctctttccttcccttcctttcccttcccttccccttccccttccctccttcccttcctcccttcctcccttcttcccttcctccctttctccctctcttctttgtctttctttttccattgcttgctttttgaacattgtcttccaattacttttcatcaccccttttaattacttacaaacccagaaatctctgacctggctgttccactgccttctgatttttagctgaataaatgatgattcctggtcatttctctccccctaggtgtgtatgagtgagaaattctgcagcaccctgaggaagaaagttaacgatattgaaacccaactaccagccttgcttgaagccaaaatgcatgccatatcaggtaactggcagtgtaggagacgttgaagctatccaactaaaataatgacagctaccagcacatcgtgttttgtcctcgatgaacattccactgttattatgtcattctcagacatctgaaagcttttctgggaagtttcactaatatgcacataatatagcacaaggtgtttaagtgagtaccgtatttgggatttctagttcctgtttcttgaagagtttagagactaaatgtactctgtgtgccatatgattctagcagatgtgtatgtagcattgtgtatctgtaaatactgaatgaggattattgggtgtgatcagaccctttctatagagggaaggatggtaacatgagacaaattaatgtcctcagtttgaaaatctagtcatagaaacttggcgaatcctcaaaggattatgctccctcttactctggcagttattaaggaattgatttttccattaattagtagcttgaagtcaagagtctctgaaaacagtatcttatcctatcattagtgctttcaaacactcctctggatttgttattttattttcacaattgctatattaggtagatgggagagagaccgtgatccgaatggactgatggggaggttgaggctgcgggatgtgtcctttggttaaggtcactcaattagctaagcagtggcagatctggggctaaagtccaggccctctctcggtgtattatatatggatttttaacccagttatgtaatgacagcttcaaaaagttctctgagttttataggttgaatggacccagagctcatcagcttcaaaagaatcataaaaatttaacaaaaagttataataattacatgtatgtgttttaaatggtattttaagaaggaatacaataattggcagcttctgctcctcagatcattaaacaactaatgttggcagctaaagagcaatttggtccctggtgcagtcacgcgtattatttacaaccagtagaacatggcagcgcaaatgtggggcttggctcttaacagcttcacatgccatttacttgaaattatgggcccatttgctgagagaaaaaaaaagaagattgaacacagtcttgcttctgagttagggtttttgcctttcttaatcaatatatctttcccaggaataccggtggatgagtctgtgttaagcccaagggaagaaatgtataattcaggcagcagagatagcctcactccaatgtgaacaatctctcttcagactccagggaaaatgcccaagatggtatattgcgtatggcgcaggtttcctgttttagctctttggtcagctgagaaaattggtgaggcctgatctgctgaaggatgacctgaattaaccctcctggtaactgaccttttgacctcactattttctggttgctctgtatgctctttggtggaccggcctttcagtcaatgggctgtccccataggtgatactagtgttaaggtggtcaacatttgtttgcaactcctgcatataatgctatttctgttttctcctgtttcaataccttcagaatgggcaaaccctcactccattaggtaagattcttaaaagccctattgaatggaaaggatcttggtttttttttttttttgagtcagagtctcgttcagtcgcccaggctagagtgcaatggcgcgatctcggctcactgcaagctctgcctcctgggttcataccattttcctgcctcagcctcccaagtagctgggactacaggcacccgccaccacgcccggctaattttttgtatttttagtagagacggggtttcactgtgttagccaggatggtctcaatctcctgaccttgtgatctgccctgaatggagaggatcttaagggaggtccttaagattcttagggggtcttttgtctctcttcaagtttctatgattcgcttagatttatagactgattgatatggtttggctctgtgtccctacccaaatctcatctcaaattgtaatccccacattcaagggagggacctggtgggaggtgtttggatcatgagggcggtttcccccatgctgttttcgtgatagtgagtgagttatcatgagatctgatggtttaacaagtgtttggtagttcttcctgcattcattcttcttgccaccttgtgaagaaggtgccttgctttccccttgccttccgccatgattgtaagtttcttgaggccttcctagccatggggaactgtgagtcaattaaatctattttctttaaaaattacccagtctcaggtggtatctttatagtagtgtgagaaaggactaatacatccatcctgactttgatctatggcctgaggtcctttcttaatttgagaactatttgccatttgcagagagtaggaataacaaattagtttcattttcaaacctggccagatctagctcctttacatgtaacagtttattctttaattcatctctcttctcttgcatttcatcacaggcattgacaggaggtcagtgggcacttctgtattctgcctggaaatctccttggatgtctaactacaattattagggactcttttttatattgcctcagttggtaactgagtgacttttttgccactacaaggatctttttttttctagtttctgatatatttttcttacttttttctatccttcacggaggcccatgaggcttctgcctactaccaagtcccaaaaccaatgcctcgattttaggattttgttacaatagtatcccatatccagataccaaaatctattttcattatctactgctgcctaataaaaaataagtcactttaagacgtagtgacttaaaatattaccagttatttattttgcccatggaaatacaatttgggcagggcttggcagggacagctagggtggctcaactggggtgggagaatccacacccaagacggtgcatacatatggtctggctgttgagtgctctctatgtgggcctctctataggcagcttggcttccttacaacatggctgctgggttccaagagcaagtgtcccaagagaaaggaaatggaaatttcttattcttaaggtctgaattcagacactggtatgcatctctattaatgtgtaataagtggctcctcaaacttcatagcttgaaacaacaaacctttatctctcacagtctctgagagacaggagtccagagtgacttagctgggtggctctgactcagggtctcataaggtcgctgttaagacacactttggagctgcagtcgtctaaaggcttggctggggctgaaagatctgcttccaaactcatgcatgtgttgttaacagacggcttcagttccttgccacaagagctgtgccctaggacctcttgcaacatagcagctgatttcctcagagccagtgatctgagggagagagaccaaataagacagaagccacagatgccaggcgcggtggctcacgcctataatcccagcactttgggaggccgaggtgggtggatcacgaggtcaggagatcaagaccatcctggctaacatggtgaaaccccgtctctactaaaaatacaaaaaattagctaggcgtcatggcgggcgcctgtagtcccatctactcgggaggcggaggcaggagaatggcatgaaccgggaggcggagcttgcggtgagcccatatcgcgccactacactccagcctgggtgacagagcaagactccgtctcaaaaaaaaaaaaaaaaaaaaaaaaaaaagccacagtgtcttgttaggacttagccttcaaagtcacacagcatcacttctgcttagtgactagaagcaagtcactaagttcagttcagcccacaggcaaggggaaggaaacaggttttaccccaaaggaggagtattaaagaatttggtgggcatatttttataaccactgcagtgttacttccattgtgttttattggtccagcattcctggtgtgctgattcaagggaagggccactataacaagtctcaatgagaggtgtgtcaaagagtttcagtgccatgctctaaaaagtgccacagtatttattgagacagaaggattttgagaactgaagacctggacagagagcttttcagtcagaggaaatggcctgttccatttatttactgaaaagaaaaatatcatggagcatctaggaagtgtcagatctggttctaggtactgaggatgcagtagagaacaggacaagatcccccttcctttttttttttttttgaggcaaggtgttgctctgttacccaggctggagtgcagaggtaagatcatagttgactgcagccttgaattcctaggctcaagccatccttagccttagcctctggagtcacgggattataggcatgagctcctggctcaatgtctcttcttttttgatagctgtgttctcccattggccacagtggaatggagctagatatgcctgggctgaatatggaggaaaagctcagctatttcttcaggaggaagagtagtctagattggcccaggtataggattagtattgtggtagctcttcagagttggttaggaccaggatacacagagctctgaattgtaggctaaggataagagcattttagggagccattaccatgttttagcggagtagtgacatgattaaaaccagactttaggaagcgtaatccggccatgttgtacacagagggatggatgtggcaacaccttgggagtcatggaattaactaggatgttagaaatgaaaaaaggaagaaaagtaggctagtataggaaatctactacagataggtaattattgatttttggaagaaccatgatgatattaatatatccagttgagtgtggtgtaatacacattaagaaaagtaatttagagagcccattgtttctctcccttttcactgtacacaccttaagtgtgttttagtcacctctttttttccattctctatttctgtttcaattttcctgttcagcagaggatagtttggtttaaagccgatagcaaaagtggattctgttgttgctgtccttcctagacatatcagtccgttctcacactgctgtaaagaaatacctgaggctgggtaatttataaagaaaggaggtttaattggcttatgattctgcaaattgtacaggaagcatggcagcatctgcttggcttctggagaggcctcaggaaacttacaatcatggcggaaggtgaaggggaaaccagcacttcacacggccagagcaggaggaggggggtgggggaggtgccacgcacttttaagtagccggaccttgtgataactcactatggcaacagcatcaccaagagggatggtgctaaatcattaatgagaaactgcccctatgatccaatcaccttccactggtgctaaatcattaatgagaaactgcccctatgatccaatcaccttccactggtgctaaatcattaatgagaaactgcccctatgatccaatcaccttccaccgggccccacctccagcactgaggattacattgcaacatgagacttggatggggacacagatccaaaccatatcactagtgaaggaaagtacagccctgagctctatgcatactgacgtagaggaagaaactcaccagggcagagagatttctcagtttttgagtgcaggggccatacagttagtagagagaaacttgggctttggaatctgagaagtgtgtgttcaagtgcagctttatcatgacttggctggatgatcttgggcaagttgtttaaccttttcaggtcttgtttcttcatctgtaaaatggagctaataataatattgcccatcacattaggttgttgtaagaattaagtgaggtagtaaattagaataatgtggtacatcagtatattaatacatgagataataaatatatgaacgtgacgtggctttccacagagtgaatacatgcttagctagtgattgttagccatgcatctgagttgggggagacccagccagtgggtgactctctgatcaggtgtcactcagtgctacaggttcccggccagtgtacctgtagtaaaagggcagcggtggcatccatacctcttaatccaaggcagctttggcctcagtgcctgtgcagtctcctgactggccaactaggctgggccacttgtcaatggggtggattcgtttttttgtttggttttcagctccgagttcaaaagcctgggagacctgctggcttctctcccagagctgggccacatattgttaacctgggccctggaggtctgatcagtgtcctggctggatccagtgcagtaagagaagccaaggagaagacagtcttccaaggcctgaagcaggtctgacctaactgcccaatgtaggaggtttgccctgggacaaactgaggtcctgcagggttcgagggtgaaaggcctcttctcccaggaggcagccccagacccaccttgctgaactggctgcctggaaaggaagtgagagcgaagatctcaaaaaagagcagctctttaacctctgtgctgctctcactgaacgctcccgccctctgcccaggacttgatggctttggccctggccctggggcagagcgaagggaaagcgtcagtgccctctagggccgagagtgtggcactacagcaagtgtgtggtgggtgcggctgacttgtgctctgtggctactacccatccccatcagaaacctgggcctgtttccttctctggaatggtgctgggactttccaaaccacggacctgtagtgatgagagttggtgtcttgagtccgcgtccaccgtctgcatgccatgcctgccttcccactgctgggggccctcaaccctcctccagttcccgtgtctaagacttagcaacaagcatccttcctgtgtgttggactgcgggtctgcaccattgtgagacacggtcctatattgggccctacctctatcggtgctcgttgacctgactggtatcacagtcctcatctggaacggggccagccaagctctggccactcccttgtcctgggacgaaggctcagcccctgaggcccggcgagtagtcaaggctggctctctgatgcctggctgctctgatgctggcatcctgcatgcacttccagctccagccttgtcctgctcaaattacccctcattattgatctggtccatctgttgagtcaccctccaatttttttcttccactttgtttaatgcctggcactcaaaagacagccagtagaagtatttgtttttcaaaaaatggaccctcattcattggttgctgatccctagaatctgttgttttcatacctccttcacttgttaagattttcatctcctgccctgacttcagtgggtacgtctggttttaagccccggtcctccttctcataggatccatcctctgtcaggtgattggagctggctgatgttccagcttctggatgctggaagccagcagcagcagctgcctgggtgacagcctcactgtgtgttggcaggcttctcactcaccttttttaatcaattggacctgaaaatcttgaagctaaacaaacacagccctgctattttggcacaagatgaaggccagttttaagtggtctataaaagctgtgaaaaaaacttttaaaagagaattatatccagggcacccaagctgcttccagatgccagaggcagccctgcatttaataatatgcttgctggaatccctttaaaatggtagatgttggccatctttttcttttttctttgcattcccaatggaaggacttccaatgtatggccctggtattattccctgtatgttttagagaagctctaaattcctgtagccgtcaaacttgggtttttcactcagactgcatgttaggatcatctggggaaatttaaaaatactggtacccaggcagcaccaaattccttcggtcagaatctctagggatttttcctgaatataggaaccttcataaacagtttccaggtggttttaatgtcctgtgagggctggaaccaccgtaccagtgagaggagagctggcctgccctgctcactgctggcagatggactcttgagtcacatctttgcacacccagaagctccaggcccaccgtgtaccatggctaagtagctgcaactgcaacctggccttggcctggaagcacatcttgtaggatcactgatgttactctcccctggccttccccttgtcctggaagatgtgcctaaggctgagagacttcattgtttaattcatctagctgtctgttctgagagcccccagctaaactgaggttccagtcccagaacagctagcaaaccgccctaaggggaaatgaaggggagatggacatggtatttactgcgtgcctttcataggatgctgcttttacctggtagatggcccaatgcctagttgtctgacctgtgaccaggggctcctcgcctgggaaacttgtttatatgtctttgtggctcttgcctttcctgtgttcagtttatgcccccctgcccattgctctggcgctgggagccaaaccttgtgctctcttgggcatcccaggtaaaacctggcctggggcatcccctggtcttcagatggaaggtgcaaaggcaatatacaccacagtaggaaacaaattcaaaaagtgtaatttacggatcctgggcagggagggcacagagagtcgggagggcagtcctttgtccctgggccactccagataggaatgaagcatcatgcagagagagagaaacaaggcacatggcaactggcagtgtgtatagggagtagggtctgggccactgtaagttttcaggcaaatgtgtgaatggcctgtttaaaggaagcaatggggaagcagggagcccagtctgctagtcaggggagatgtctctcagtttttatctctcgccactggcttgagccattgggtgtggtttccttctaatgcctgggcagctgtgtcattcccgttacagtcaccacatgattctaattgccttagaatttataggaaccagttggcagcccaacaagtgggctcctctggccagtgagggagcctggtgagggaatcttcccaggacaggcagagacatctgcctggttcttagattggccatctttggtctcacctcagtttggatatattttttttttatctcacctgcccccttgttatctgcttccaagaagcaaacagctcaaataattatttattttaaaataacataagaggatgtctctgatactccaaaagccttttttttttgaaaaaaaaaaaaaaaggatggttctatccaatttggcatttcatttatagagaataaaaactatctatacccaaaatgtattcattgcacacaattgtattttaatggcagctggaaatcttctctcctaactgatgcttttggggaaaataagaacatttggacaataaactagattttctagataaacacaaaatgggtgaatttcacacatccaataatgaaagtagttttttccttaaattagaaacaaggatttaaatttccgtcttcttctgaatgaatatttggcacccaaccaagaaatatatatcattttaaattttcttgaggaaatatccttttcttgcataattaatttaaaggaaattaaattgaccatgtcaattgtcaaacaaggaaaagtaagaattttgcttatttgttattaattttatttacaatagtatttaaaggtaactttgtaaaataacccctaatgatataatctaaaataaaatagattgtatttaaatcacttttttattatataccacgaaaaactactgaatgattaaaacattcttaagtgggttcttaacattgtatggaactggaaagagcagttcagatcacagaggcatgggcactgtgttctaagtggtcactgcactgattcagaacagcaggggctggctctgtactgggtgggtgggtgctcaaggccagacctacacagtgctcctgtgtctgcagctgcagggaagcaggagaaacagtgacgatggtccaggagaggccacctgacatatggcagaaaaacaaaattcagggtacagacagtggctgggagcatcaactcaatcgctttctttctttactattttccctctttctaaaaaagtctgtgatttagatcagtggctgcaggagggagacaagagacccagtaaagatgttttcaaagatgatgcctatctggtgtgaaaagaaatgaagacctacccaaagagaataagcacaagctattatgcagagcttgctacagcaagggagtcagacaccatcatttgcaatttggcagagactcaaaggtgggcagacgagtgggagagctttatagtggaaaaaggcgaaggcttcaggtgtgccctgattggaggttatcaatgcggggaagctggaggcggctcactagaagcgaacatcctgtgtgcttggtcaggggaccatatttggctttctctggtgggtcctaagttggaatcggggacaaaaattagggaagccatcagttattaatccagtcctgaacattttgagtcaattgttacagaagttattatttagcttcctggatagttactagagagcaatttggcttccagcaggtctgatttagagcaggccagcttccggggttgctttttgtgggtaagggtattgttttctgggaaagttgctgcaccttgtggatcagagttccatcttttgctatggcctggctgttgtccgattgtatatttagtcagtcaccaggcaccacattttctcaagctgttggaaagttctgtgtgtccagtgttatcttccccttgtattctatgaaatatatggctgtctgaaatgtttgatgtgcaaagccagccatggggctctgctcacagcaaatgcttttggctatctcaggaagtcatggtgactggaagaaatgcacaaccctgacagaaaatggcaaattctagctgaagggacctccagggagaagggggttggggaggggagcagttggctggggcttcttggccgcctccattgccctttggttcagccagcccaggctcacagcagatatccttgacctcgccaagggaaggctctgaaagacaaaggtagatcttgatcttgcttaactgtgcacttgagcttgcagttccttgaaagatctggtgtcacgcaagacaaattatacagcagtctactatttctggagtttgatcattctcagcactggctttatttctttttttccactaaagaagtatagttcaaatgtggataggtgaaagtagataatcttaggaggatcgttacaaactgaaatgtccatgagttcaggaaagtgccatgtgtgtgaggggtaccccaggtgagaggcctgaggattgggcaggtgaaagggaccatgtgggcacaggattcatccccctcaccatgtgctttctcactttagtgatgatttctacccaggtgttgactaagtcacttattgacatgagatcagaagaaacaagttcaagttcagcaatctcgcatgggatcttagaactctgaaactctgtgaccttcacaaggttacctctgggtctagattttttaactggaaatcaaggacaatcattaagagactccccttcctggattgttgggaagattaaatgatacaatccaagtaatgagagcattgtagtctctaaaattggattctcaaataatgacctcaggtgaaggaagaactgttcaaaatgccccaggtgctatgaagtacagagggcaggtattttttttaagggatggggtcttgctctgttgctcaggctacagtgcagtggtatgatcatgactcactgccacctcaagctcctgggctcaagtgatcttctcacctcagcctcccaagtagctaggactaaaggtgcataccaccatgcctggctaattttttaatttttttgtagagatgggggtcttgctatgttgcccaggctggtctccaactcctcaagcaaccttcccatcttggcttgccaaagtgctgggcttacaggtatgagccaccacacctggctgatatttgaattcagagatgctgaggataagtaatgttggaacagagtgggtaaagtcagcttcagcataaattgtgtttatttaatttaaacacaatttaaattttgtgttttggctatctcaggaagtcatggtgactggaagaaatgcacaaccctgacagaaaatggcaaattctagctgaattttaaattgtgttatttgcggaataaaatatgtggacaagtgctttaaaaaacctatgagaatacccaggtttcattcccttgttgaagagcaggcgggcacaggcatattgggatgccataggtggcatattcttctccacacacatcctccttggcatttagaaagggcctgtgaaagttatgaagattcaggtcacagatctctgatgggactttcttcaggtatcagcattgactttccaaattttcatcactgagctcctgaccaatttcagcagtcagagaggtctgcatcaagcaaggtcttggtctaagcctcatgggattagatctgaggtcaaaatgcctataaagtattgatcaagggcattgtagcattttctcatggcttgggtcttgatatgaatggtctctttgggaattcacactcactctgctcttttaaaatgttcacttttatcactgtgcattttcccaagccttatccctcaggatcaaagaaagggcctaggctttaattaatgatctctcctatgttttaccaagggcactggtctctcgagcttgcagtgggttgcaagggattagagggtgtatttgcagcaaaacttctgtaccggcactcgctctgtatatatagtctcttccaatctgcttttagagatcttttctttctgactgtttgcaggaggacatggcacgctgtggcatattctgcctgatgtctctggaggcatagttggtgcccatcccactttttattaactctcttggttgaaaacacagcccagaagacatgttgggacttcataagcacagcctaaggaggaacattggaaggtacaacattgtacatgtggccaccctgccccaacgcagtcacacctctgtgctggtcctcctgcgagctccccagagcatggggtcccttgaggttctttgtggcatgcggtagggggctcgatcctcagcttccttgacttggccattgttcaggatggaaattaccgatccgggaaaagttttatttgaggttactgtttacagcttgaagctcatggaagtgcagtctgctctcctgtggactttgtgggtttttcctaaatgggtccaacccatcagcttggcatttggggcactattgttttgaagcaacttccttgtgagtttagtctcacctcctaccccttgcccattgctctctaacctgggttcctgtttcttcttttgggactcttatattcttccctcctgaaatctgcctcagtctctccttctggaataatctctcttctcctctgacctctcctagtatttgttttttctttggaaggcaccttatcccctttattttatggtaacttcctggagagcaggagcagtacttgttctcttttgtgcttgtcatgttgcttaaaacacatgagtgttttaagcagtgagagacaaacacatgagtctcaatagggtctttatccaatcatggcattggaaactatggacttcagtgacagatgttatgtgctaggtttcagaatgcctttaaggtgggaaaacattttgtatcattttcaacatttgtatcagtttgaaatctgcctgctaagtaacaataaaaaagttagcaacataatttatgtttaaaaggaagtgttctggggtgatgttcgagttggaaacttgccctatgctttactgcattgtgatcttcagcaagatattttagttctccagatttccgctttcccagctgtaaaaggagacaacaatatgaatttcagtgaacataaaaagcacccattattttatacattgcaaagaaagaaaaattgctgtcaattaagcagtaacagtgctttctatggtttagaatttttatcttatacttaactgatatagctcttttagatgtatttaggcttttgaaaaatcacatatcactcattaaaaaggaaaataaattggttaaggttttccttggcatcttcttcttcattctgagtcttccgaaacacatttggactcaatgttgtcgaggtttgtgtttccccacacgtcatcatcctgtgaaccattgaagttgatgggaggcaactttttctcccccaagaataaagagttttctgtaggattgtctgccaaactgctaacacctttcttcaagttttgaatgctggtgctttcccagtcttacaaatccacatcaacacaagattttcaggcaacagccagtacgcagatggtcctaaaatagtttgtacattgaaacaccagggggttgcagatggtcagccaggccagggaaaataatccagttataaccactgcatcctgaccacttcctggctgatggtgattgtaggacacatccctgtttcagagatgttaaaatgtaaaataataataataataataataataataataataataataatataagatataacctgtttcctaaagttgtgatgacatttaaaggtgagaaagtttgtagctattattgtgattatggttactataaattctgagaaaacacagtggggttttctaattaacactaactaatttatgggacactcattaatgttatatatttatttattgttcaatgttcatgcttaaaaatttcttaatttttcctctttttaattgaggtatggtttatattcagtggaatgcacaggtcttaagtgtttacagttcttgaattttgacacctgtgttacgaacagccctaccaagagatagaacagtctcatcactcaagaaacaacaccctatcttgtcccagtcatcatcgtccctcctctgttctgatatcttctaccatggattaattttggctgttctagaacttaatggaatcatgtggcactactcttttgcatcttttctaaagacatgtacatgttggctgggcgcggtggctcacgcctgtaatcccagcactttggaaggctgaggtgggtggatcacgaggtcaggagtttgagaacagtctggccaacatggtgaaaccccggctctactaaaaaatacaaaaattaactgggcgtggtggtgggcacttgtaatcctagctacttgggaggctgaggcaggagaatagtttcaaactggaaggtggaggttgcagtgagctgagatcgtaccactgcactccagcctgggcaacaagaacaaaactctgtctcaaaaaaaaaaaaaagaaagaaagaaaaagacacatacatgttgctgcatgtatgacgagtttgtttctttttattgctgagtagtctttcattgcatagctatagtattatttttgcatcttcctgctgatggatatttaggttgcttccagtatggggctggctctcaggcataagacactgtgaccatttatttgagtgcaaatatttttgatgacatgttttcatttcttttgtgtaaatgcctggaagtagaattgttggattaaagggtaggtatacattgaactttatgagaaactggcagaacttttcttgaagggaccattttacatgcgtgagagttccagttgctccatacccttgtgaatgttgacatttttagttgagttaatttgaaacatttgagtgggcttgtagtggaatatttatggttttaatttttcttttcttaatgattaatgatgtcaaatgttttttcatatgcttattagccatttgtgcgtctactttgtaaaatgcctgttaagtcatttgaccatttttcaatggcaccatttgtgttttaattgtcaagttgtagtattcaattccttgtcagttaaacataatgcaatgattttctccaagtctgtgacttgtcgtctcattttttagttgtgtcttttgatgagaagtttttaattttgataaagcccatttatcctttttaaaatagtgttttctgtatcttatctgaagttcttgcctactccaaagtcaatcaaatattcattttttttttgtagaagctttatagttttaacttttacatgtaggcctgtgatccacctttaattaaatttttgtgtggtttgaggtatgaatcaaggttaatattttttccatgtagatagctagttgttccagcaccatttattgaaaatactttcttttcctcattgacttgctttggcactttggttggctgtatatgtgttaatccactcctggactgtttattctattccatcgatctgtttgtctatgtatattcaatgccatattaccttgatttatagtggctttagcattagtcttgaaaccaagactcactttttaaggattgctttggtcatcctcctcctcctcttccttcttcttctttcttcctattcttcttcattcttcttcgtcttctccttccttccctccctccctctttcacctcctcctcctccttcttcttctttctccctctccttctttgaggtggggacttgctatgttgcccagacaagagtgtagtgcctgttcaaaagtgcaatcatagtgcactacagcctcaaactcatgggctcattcttctagattttatttgtttagatatgaagtctcactctgttgctcaagctggagtgcagtggcaggatctcagctcgctgcaacctacacctcctgggtttaagtgattctcctgcctcagcctcttgagtagctgggattacaggtgcttaccactgtgccagattaatttttatatttttagtagagatagggtttcgccatgttggccaggctggtcttgaacacctgacctcaagggacccaccacctcggcctcccaaaatgctgggattacaggtgtgagccaccatgcccagctcactcttagacgttttagaataagcacataaatttctacaaaagaacctcataacattttgattggaattatattaaatcaatgaatcaatgtggggaaaattgaaatcttaccaatattgaacttccagtgtatgtatacagtgtatcttttaaattatttgtctttaatttgtctcagcagtttttgaaaaatagttttcactgtagagctcttgcacatatttaaagaaagtatccctcagtattatgagatttaaaatattttgtaaattgtattttttaaaaaatcatcttctagttttttgttatttagacataagattgatgtttgtacattgaccttgtatcctgcaatcttgctacatttaatttatcatatatttttctcagtgctttacagaatttcttgtttattcttgtaactacctatgagtcaggtgttattgcaattatattttataagaaatttgccccagatgtgctaattttcttgctccaggtcacacagtttgagtaacaggggaggaatttaatttaggtcaactctgattatatggcccagtccccttccacactgctataatatgtcatctctttgacatttgtatattattggaatggagtgacacattgtggtttctctggattccactgggacatttttgggtcatcatactttacgggagatggcttcaccaatggaactcctaaaatggacataacaagaataaggtgataaaaaagacggtgatttttccagtttaaggagagcaatatttttccaggttctttccccagaggactgctaaggaaattgtacataatctcaaagtgcagtttctttgcccatgctgtgaatgtacattagctgctgctagatcttccatgtgtgtggatgctgtaaagcttgttttcccttcttctctcccacaacgtgctgtaggaaaccatttctggacggctaaagacctcaccgaggagattagatcattaacatcagagagagaagggctggagggactcctcagcaagctgttggtgttgagttccaggaatgtcaaaaagctgggaagtgttaaagaagattacaacagactgagaagagaagtggagcaccaggagactgcctatggtaggtagtgcacaactgttccccggcaagatattgatgatatttgttgtgttttgtggccaggcagaatgttctgtgccttatgtgaacgtcactgtgaagagcgtcatggagcacatggcccttttccttggggacatttttggcaggtgccttggcaaaccgaatggaattacttgtcagcttttgttttcatattcacatgtgacatcttttctttttctgttccctgtctcaacctgtgtttgctaatagccttgttattatttagagcagtctctcccttggcaaggtcttgagcattttctttgtgtgacaattatcttccttttgtcccttggctccgtctccttgtctgcacaatcatagactaaatcaggctgatctgagaaaattgagggaagaccataaagtggctcttctatgtgtggacttttctgagagcttctttccccaggaaggatgatgtagaagatgtgaatgccgccttagccaaagtgtctgcttgtcagaggagttcaagtgttcctgtgttctttccttttccagtcgttaggagtaactgagcttactctgagatttgcacccagagggatggtctcagaagacgctggttcagtgcaaattgagatggtaaaaacttatttcttaaaaaatggttcctaaataaatgtcatttactaaaacaaatgaaagaaatatatatatgaataaatggctatattttacaaagtatgcttatttaagaagaaataagaaaaaacggggctgataggaaccagatttgaaatagggcttttgtaaatactccgtaaattgaagtaaatgaagagtagtatttataagctagattgagaaatataaaatcagctagctgaagttaagtgcatcaatttgggatgaataaattttcttttaaaatgtctatcaatttgtgtagagagaggtttcttcatagagatattagaatcaaagagtatgttcagtagttattctgtcttctttgtaatgaatacccttaagttggcttaacagctcagtacttatctttttttcttttcattcttaaaggtcaagccttgtttttgccgtatatgtaactagagacagtacgtttgaggctaaataactgtagtactagggaatgacaacacgctcacccaagacaccgcagcctggtttactctgtcatgataggaatgaggattgtacatttgaaataggtttctgctattgattttttaaatgtataaacgatggaaactacggaatttctcatgttttcaccacatagttttttgtcataaaatgaagaatatattatatccaagaatgaagaggaagtgaacaaatttgagcaaatttagtccagcaatattttcatttgaatagttgagcccctgaaagccattaatatctctttttaaaaaaagaaccatgcagtatttttgaatctcatcatcgtcacttcactaagtattttcacaatgatgaataaaacataaacaaatggaatgagagattgttaccatggatgattctaaattgcagatggctcattactgttgtgaagcctctctttatgtttttacacttggattttgctggatcagccaccctttccctatacattgatttacacgtgcttaattttttttaaccaatttgaggtgagttggctttaggtgaaccaaattaataatctagggttgagagtgtgggaaacaaataaataatgaattcctgaatacattgaagcttttatttattaaaatgtgataaaactggggcaaagtccatattcagctttttttgtgttttgagggttaaaaattcagagggagctctgtgttcaagtttaaatgtagagaaagtacaaaggagagtgtacttatgcacatacacatatgcatgcatgtaccatgactctttttagccttagagaatgaaaccatttaagaaatgagcaatatgtagtattcttaaaaaaagattttgatttccaacaatagttgtggaatgcagcgttcaggggaaaaaggcaactcatggatgatcaagccaccctgcttgtcaggaacccagactcttctatcttgttcttctctgccccacaacatgtgccattcagtccaacctggctgacccagccacatgcatgtccaagtccagtcagaaaaaaaacaaagaaggaagagagctcatctatcccctttaagtacgcttttagaaatctgcacacatctctgctacggccacatccctgtgaccttaaccttgttatatggtaacagctacttgcaagaggggctgggagctgtccttaccctgggcagcaatgtgccccactaaagtgatgaattctgtttccatagcaaaaggggagatttgcagtcttagggaacaattagcagtgtctctcgtacagagaccttttaatgatgtgaagtgtatctctaatgatgcacctgagatgaatttgctgcatgcatcacttaaaatatcattgtatcttgtgtctctggctagattgtgagtccaccgaggtcagaacattgttcttaggtttcactgtactgctttggtgtccagcatgatgtcttttaaaatagtaaatatactataccatcaatatttgttcatttactggggccagatgttaaaatgacacatgaatgagtcctctcttcctgcattttagattgcagatctggaccttgaatcttctgcttctttattcattttccaaattaatgagggtagtgataagtttgtctttcttggaaggtgcttgagttgtctgagttggatattcagtttggagtgtcagtaatagaacaatacggtgatagaaaaggaactgaaatatgccaaggtactcaagggcaaagggagacagacctcatcaccgaatccattggcttttgttgccaagacacaatctctataaagagatgataaagaagtgtgctttaactcctgtcagctgttcttgagacttcaggataacacatttgaattcggagcaatgttaagtgcagtgaaatagaaggaaaagctaaatctatcttccaagccttgaatatttatggaaattaactataaacatttaattattgtggattccaatgtgtgtgtttatttaaagaagggtggaatgaaaaaaatcagcaacttttacagtttgctacatctgcttttttttttttttttgagacagagttttgctcttgtcacccaggctggagtgtaatggtgcgatctctgctcactgcaacctccgtctcccaggttcaagcgattctcctgcctcagcctcccaggtagctgggattataggccagctaatttttatatttttttagtagtgacggggtttcaccatgttggccaggctggtctcaaactcctgacctcaggtgatccacctgcttaggcctcccaaagtgctggaattacaagcgtgagccaccgcgcccggcctacaactgcttttcaagttaaaaggacagccctcagatttacgcagcagtttttcaccatcccttgtgtataaattggtaatctgtattgtactttattaatattgttgatttcgcactgtaactcagctataaaggaaaccgacgtcaaggggagagatttaatcacagaataatcaggactagaattttaaataggacatcattagcatgttaatgaattttcccaccttatgccagctgcctgagtagaaaagatactgcagatgtagctcaaaaatctggctggttccctggcccagtgagctgtcaggaatctgtgtagggtgatccataagctaagtgaagggattctaagtgagaataccaagcagcaagattttgtttttctgagaacgatggctaactgtgcccagcctaaactcatttgtctttcggtgagtaagaggggaatgggaggcagagaaggggcagttgaagggcaatgaggttggagtagaggcacctttccaattatggtttgggattaggaccttttgctttagatagaaaagttgtaagttcgcaatgacaagatcctgccctaattcttggcacagtctcacaatttttgagcttgaaatagctaatgaaaggaagcatgagtgtcttagtccatctgcgttgctatagaggaatacctgaggctgggtcatttataaagaaaagggaattctttggctcacagttttgcaggctgtctaagaagcatagtgccaacatctgcttctggtgagggcctcaggctgcttccactcatggcagaagatgaaggggagctggcctaggcagatcactggtgagagaggaagcaaaaagagagagaagggacatgacacactctttttaacaaccagctctcccagaaactaatagagtgagaactcactcattgatgaccaagctattcttgagggatctgcccccagacccagacacctcccattaggctctaccttcaacattgggggtcaaatttcaacataaggtttggaggtcaaagaaaagaaactatagcagtgacagattatactgagatatcggtttaactctgaagttcccagatgcagctacttgcagaatttcacttcacacctattaagaaaagtcttttagtttagaaatcctgtgagttacaagttctgcatatataggcagtaattcttttttccatatatgtcagatatatgtagaagaaactgatgaaaaagtagaacaaaagaataaaatctatgggtctcttttattggcagggagagggaggaaatggagagccgggacaatacatacaacaaagataaaaacaataaaattagcaaacaacaataaaatttaaaaacaaagacagaagaaaatgccaatgtcaagtgttaattatttggttgagaatatgatgtgataatgaacttcctagaagtcacagcaaagaagacagttgaagcatcatccttcttcctcaaaagcaccttgaaaagcacagagctttttgggaattcagagtgatgctaaattcttcaagacacttctcttgaaagcatagtggaaagtcctcctgaacagatttataacacatgcagaaagctcttttacttgtattattattttttacaactttttattttaggttcaggaatacatgtgcaggttctttatataggtaaattgcatgtcatcggggtttggtgtccagaatattttatcacccaggtgataagcatgttatccgatggttgtgaccaactacctctaggaaaaaaacatgttggggatcccttcaaagcaggagggactgtgcacaggagagactgaaaccacatacacattttagatatgtaggtatggacagtttttcccacaaaaagatccagtttttcagcagatttttaaaggggtcattctaagagtcctcaaattttaagaaacattaagatattaaactgtcgaggtgaattagggcttgggctagtgaagtttaaatacggcatcttccaatttctgacattatttcaagatgtaacttagcacctaaaaagtggctggagaacatatcctgtacactcaccaaatgtcacttctttcctctgagctttggctacgacctatgtataagaaaacttagctctccgggccagaacggtgatagtgctcttgataacagagggccaagccgtctgctttggaaccagatgagtgttgcggtgctatgtggcaagaaatgtagatgtttatatgggaaatagatatgtgtctgcctttccaaattcgaaatctttggtcatttagatttaaaaaaaaatgtcaaataggatcttttggaagaaataaaaaaaattcaaaatcttttccctcaggtttttctgataggctgaggttttaaatctctaatcatttatctttgatttgccttattgattacattatcactttatcaggaccctgactaaatctgtttgtgtttttaatttctctccattttttcctttccagttacatccttgcatcactattagtgtgattatttcccttcagccatttttgcctgtgaatttctaagcttgaaatttgcagctaactttctccctcctttattaagtcgctgtgataattcttttgggaggcatcgccatacagtggaaaaagcctggattaggatgtagggggtgtcagtttaatctcagtcctgcccttccctaatcatctgcatagcacctgatatatatagcatcagaatgtgaggctcaatgagtgaatagtcttcagcaactcactgaattttatctgagtctcagttgcttcatatgtaatactgtagaaccagatcttgaagttgcatttctatccatccatccatccatccatccatccatccatccacccatccatccatcctttccacaagcatttattgagtacttacgatatgctaggcgctgtggcaggccctcatggtccagagatgaattagatagtccctgtggctactgaggtcccttttaactctagccccttgtatgtgaatttccacaattcaatttatactttgttcatttattttcttgctctcagctacttttgatctccttttcccacttttatccatatatatttttaaaagtccctactattgttacatcttttttcacttgtctctctacttgtttctatactcccttaaaatacgtaagtcacttaccccctgggtggaaatacaatgatgttttcaattatatttcataccaaattgcaacaggcagttttttttaacatgctaattcatttagagcagaaacttgactctatgtaatagggtgggtacatgtgtctcttaaatatctgccagattttctgctttgtagattattcttactaatcagtgcgtattagagtctctttgtacatttcatatggactctgggctttgataatgttggtagacacatgtactcacacacgctcatataatgtcctgaagattaagctaagagtgatggtaagtggaacttcatgatcttaaagaagagcctgtttgccatgttttgggtgaggggattattttttgcatggggaccctatcatgtattgcagaacatttcacatccctggtcattgcctgcatccaactcttcttcccacccccatttttcaatgcccccaggtaggagtggtaccaccctcagttaactgcataacttcagcctaatgctttatgttttagtatcattcctatgatttgaaaatatgttgtcatgatcattagaacttgcctggaattaataaaaccaaattctattattcacattatctttcatatagatccttagaggtttaaatataagttctttgcagatttcttcctccattgctctttactgtgtatattcagtatgtatttcttgggcacagatttctgtatgcacagatgagctacagctattgtggatttaatgttttctttgaagaaaaggaatgatcccttgtacgaagtcaccctctttttagtaacaaagacttatctctttctttttgtttctcaagcacttgagaactaagcactgtgctaagacttgacatactttagtcatctcatcctctggaccactgtggtactggctccactggaatctctgttttaccgatgaagaagctgaggtttaggaaggatcctgtcacagagccttcagggggcaaggccaccatctgaatccctcttacctgccattttacctttgacgttgctttatgttctttatgaatttgattcctaacgtcagtctctctatccatcccgcaacctcatcaccctacctaaataccacactcatagattcataattacaaaacaaaagtctacaaaaagtcatctattgatgatgttgaacatcctgaaaagacgggtatttttttttcatttttgaagttttcaagcaccctccctatattttgtgcctgagatgatgatccactatctgccatgttgctatagcagatggataatttaactcagtagtaggaggttctataggttatcaccctatgaaactcatatcttttgtattaaaacctcaattctttattttcgaagggtgactaggagaaacttggcatactaccataaatcagagagaagttgaactatagcaatttaattggatacatattcattgacttcaaaccagttttttgtatattttcaaataactgttgctagttctgcttcctgttctaaaatgctgaaatggcaatcttttcctttgtattcctataagtaaatgcattggttattagattgatacattactcagttcctggtaaagaagtagttagaagccctgtttgcttttaagaatttactaatttgtcatccttttgattacaaggaattcaatttgcaacatgtgatgtcaatctttctaggcttcttttgagtgagttttgaactttagttaaataaaacaactttttttttttgagacagagtctcagtctattgccaaggctggagtgcagtggcacgatcttggctcactgcaacctccacctcccgggttcaggcaattctttgcctcagcctcccgaatagctgggattacaggcgcccgccaccatgcctggctaatttttgtatttttagtagagacggggtttcaccatcctggccaggctggtcttgaactcctgacctcatgatccacctgcctcggccccctaaagggctgggattacaggcatgagccactgcgcccagcataaataaaaaaaatttaaactattatataatttaggattttcaggaatgaaggttgattctttataaaccaaatttgattttgtgaggttttgcagaatttaattttacttggctaaatatgactcattatatggtcacagttttttcagtgattaatttggtaagaaatattttgttttccttcttggcattatcatgagttatggcttcctgtacacattcctgccattttagctgtgtactttttctttgaagctgccatatgttaacaacccaagagagatggtatttactggagagatcatcacttgttgctaatttgatttttcagattctctgagctcttataaaagagagagagcatgctgatgaatgttttcctgccctgactgctttgaggagctagtgtttgctgccctgggcccttagtcctgttatgttttggacttggggatggccactgtgtctaatccactttccattgaggcttttctgtaactggggagggctggatcactgtaggggtggacaaccctgagtgaatgcactgcaatttaatatttatggccggtacagaaaccatcaagaattgcttaaggatttactgacttggcatcatcaaaaatttatgaggcaggtatgctcatattccacaagcctgcatagtcatagaatgtcgttttatttttcttttgacattatgctttctggagtcttaataactcattaacaactgatgacacactttgctttttggtttaattataaccaaattttatttgccaaaatgatgtttaatttttggtgtattaacccattatgaatatctctcttttcctatgtttactatgactgctttacatttccagtctccccaaatctgatattttccttccccaagaaagaaccaaatacaattatattacgattgcatggtctgtctggtttggacagggtgactgaaatcttgggcttctttttaactctgaattggatccagcattctactttgcttcgtatgtaccaatcttgttgggtgagacatatacattggaatttggcaagtgtttgtatacccttcacccaactccactgttttcagagactgagtaacagtagtggctggatgccaacttcatgtctgccctgagaaggtcaccaaacacggacattcccccgtggtctggaatcctgcaaatctgtcagcctttggaagtgaagtgattctcccctaaagattatagagaaattctctgaagtcaaagctgatgaaatgtctggagttactctaggataaaattttaatttatttcaaaaaatacctttagcataaaataaaatttttgaaatgaatgaaaaatgtttatcatgcaacttctataacagagcccctggttgcatttttttgcattatcttctgtgcttgcagtacaggaaagctttttacacacttgtactgagaaagaggatgcaattttacatcctctgtctgtccttaaccttataggacaatagtgagagcatggcaccagttaatcctcgtaactgtagtttgagacaaatttatcattcccaatctaccaatgtgtgaaactgaggctcagtgaattaacagagtggctcaagtttctgtatatgggaagtaaagaactggaatgagactcatctgtctcacttcagagccatactcttcattatacacttctgtgtccctggttgcattataatattccaatttattattttttatgtacataacagtccaaggatcaatatactgtgacttacttaactacctccttattctgaggtgtttaatgattatttcatgaattttttctgaagataaagtgccaagattgggattaatgacccaaagaatgcagtcatttttatggcttcatacatactgcagattgatttgcaggttatagcaatttaattgatactaccaatccatcctttctaataataagcattattttttaaaagattattagtataataacagagtggtatttaattcatgccttgtttttgcatatctttgactaatacattcaacttgtttttttgtgcctttataagttttttttgagacagggtctcactctgtcacctaggctggagtgcagtggcattatctcggctcactgcaacctccacctcctgggctcaagcaatcctcccatctcagtcttccgagtagctggaagtacaggcatgtgccaccatggcctggcttggtagagacgggattatgccatgttgcctagagtggtctcgaactcctgggctcaagtgatccacctgccgtggcttccaaaagtgttgggattacagagtgagccaccatgcccggcctactttttttcattgtgtgtttcactcacgtccgtgtgaagagacaccaaacaggctttgtgtgagcaacgaggctgtttatttcacctgggtgcaggcaggctgagtctgaaaagagagtcagcgaagggagataggggtggggctgttttataagatttgggtaggtaaaggaaaattacagtcaaaggggggttgttctctggcgggcaggagtgggggggtcacaaggtgctcagtgggggagcttttgagccaggatgagccaggagaaggaatttcaccagataatgtcatctgttaaggcaggaacaggccattttcacttcttttgtggtggaatgtcatcagttaaggcaggaactggccatctggatgtgtacgtgcaggtcacaggggatatgatagtttagcttgggctcagaggcctgacattcctgtcttcttatattaataagaaaaataaaatgaaatggtggtaaagtgttgggatggtgaaaagttttggaggtggtatggagagataatgggcgatgtttctcagggctgcttcgagcgggattagggcggcatgggaacctagagtgggagatattaagctgaaggaagattttgtggtaaagggtgatattgtgcggttgttagaagaaacatttgtcatttagaattattggtgatggcctggatatggttttgtatgaattgaaaaactaaatggaataagagaaggagaaaaacaggtattaaaggtctaagaattgggaggacctaggacatctaattagagagtgcctaaggagattcagcatagtcctgccagcaaagattatttatttacttcaaaagttaagagcggcagtttggggatagcaccaggagatatcagctgtgatggcttggaaaaacagtgtaaaccggcagcgtaaacaagagcagggcatgtatgagtagttgagaatggtgaataggagtataactagacagaagatagtagggatgacaagttttttggggcacagtccaagttggtctggtgtctggaatgagactggggcctaataaaaaggagcgtccatacaggagctcaaatgggctgtaccctgtagcattccgaggacaggcctgaattctgagaagggaaagtggtaaaagtattgtccagtcctttttaagttggtggctgagcttggtgaggtgtgtttttaaaagaccattagtctgttctacctttcctgaagacggaggaccgtaagggatataaaggtttcactgaatactaagagcctgaaaaactgcttggctgatttgactaataaaggctcgtctgttatcagactgtattgaggtgggaaggctaaactgaggaattatgtctgacagaagggaagaaatgactgcagtggccttctcggaccctgtaggaaaggcctctacccatccagtgaaatggctacccaggctaagaggtattttaattttctgacttggggcatgtgagtaaagtcaatttgccagtcctgggcaggggcaaatccccgggcttgatgtgtagggaagggaggaggcctgaacaatccccaaggggtagtagaatagcagatggaacactgagaagtgatttcctggaggatagatttccaggatggaaaggaaatgagagattctaagagacagggtagcagcttgtaacctatgtggaagaggttatgaaatgacaacagaatagaatgggactgtgaggctgcaaggagatattttccttggtctaagaaccatttgccttgtgtgggaagagactgataggtggaagtttcagcaggggagtaggtgggaatgactgatgtgaaggagaaaaactggccgtgagggacagaagttggagagctagctgcttgtctagccaccttatcagcataagcgttgcctagagcaatgggatctgacgccttttgatggcctttgcagtgaatgactccagcttcctttggaagtaaagccgccttgagcagagtttttattagaggcattaatgatggaggacccttgcgtagtgaggaaacctctttcagcccatgtgaccacatggtggtgtatgatatggaaggcatatttagagtcagtataaatattgacgtgtcgtccttttgcaagagtgagggcttgagttaaggcaactagttcggctttctgagaggtagtgggggggggtgggcagagtggtagccttgatgatagatgtggaagctactatagcatagcctgcctttgctggtgagtggcgattaggcctggtggaactgccatcaataaaccaaatgtgatcagggtgaggaacaggaaagaaggaaatatggggaaatggggtgaatgttaggtggatcagagagatacagtcatgagggtcaggtgtggtatccagaataatgtgggaggctggattgaagtccatgccaggaacagtggtaattgtgggacactcaacaaagagtgagcacagctgaaggagccagggagcagaaagtatatgcctcaggtgtgaggaagaaaatagattttggaagttatgagaactgtagagagtgagttgagcatagtttgtgattttgagggcctctaaaagtattaaagcagcggcagccgctgcacgcagacatgagggctaggctaaaacagtaaggtcaagttgtttggacagaaaggctacagggtgcggtcctggctcttgtgtaagaattctgacctcactaaccatgcctaggaaggaaaggagttgttgttttgtaagggattgaggtttgggagattagtcagacatgatcagcagggagagcatgtgtgtttttatgagaattatgccgagataggtaacagatgaggatgaaatttggacttgactgaagtaatgggggctgtctgtgaagccctgtggcagtacagcccaggtaatttgctgagcctaatgggtgtcagggtcagtctaagtgaaagcgaagagaggctgggatgaagggtgcaaagggatagtaaagaaagcatgtttgagatccagaacagaataatgggctgtggagggaggtattgaggataggagagtatatgggtttggcaccacagggtggataggcaaaacaatttcattgataaggcgcagatcctgaactaacctgtaaggcttgtctggttctaggacaggtaaaatgggggaattataaggagagtttataggctttaaaaggccatgctgtaacaggtgagtgataacaggctttaatcattttaaagagtgctgtgggatgggatattggcattgagcaggctaagggtgattaggttttaatgagatggtaaggggtgcatgatcggtcaccaaggagggagtagaggtatcttatacttgtgggttaaggtggggggatacaagaggaggacgcaaaggaggctttggattgggaagaagggcggcaatgagattcagctgtagtccaggaatagtcagggaagcagataatttagttaaagtgtctcagcctaataagggaactggacaggtggggacaattaaaaaggagtgcttaaaagagtattgtcaaagttggcaccagagttggggagttttaagaggtttagaagcctggccgtcaatatctacaacagttatggaggcaagggaaacaggcccttgaaaagaaggtaatgtggagtgggtagcctccatattgattaagaaggggaaggacttaccctccactgtgagagttacccaaatctcggcatccatgatggtctagggggcttccaaggcgatttggcagtgtcagtcttcagctgctaagctgagaagatctgggaaggagtcagtcagagagccttgggccagagttccaggggctctgggagtggctgccaggtgagttgaacagtccgattttcagtggagtcccgcacagatgagacgcggcttaggaggaatcctgggctgcgggcattccttggcctggtggccagatttctggcacttgtagcaagctcctgggggaggcaggcctggaggaactcctggtcactgcggtttaggcatttggaagttcttgtgtgctggagatgtggctggggtttgtctcacagtggaggcaaggaattgcaactcagaaatatgttgctacttggctgcctctactctattattgtacaccttgaaggcacggttaatgaagtcctgttgtggggtttgagggctggaatttaatttttggagttttatttaatgtccagagcagattgggtaataaaatgtattttgagaataagacggccttttgaccttttagggtccagggctgtaaagtgtctcagggttgctgccaaacgagccatgaactgggctgggtttttatagttgctgaaaaagagcctaaacactatctgatttgggataaagaaaaaggagcattaaccttgactatgcctttagctccagccacctttttaagaggaaattgctgggcaggtgggggagggctagtcatggaacgaaactgtaagccagaccgggtgtaaggaggagaggtgataaaaggattatagggtggaggagtggaggccgaggaagaatttggacctagcttggcctggtgaggaggggagaggtcagatgggtctgtagaaaaggaagattagaaagacttagcgacgcttggggttgggactgaggggacaggcaggagggaaagaaggaagatttgggatgagttgtactgggaacagagactagggagggaccgatgtgtaaaagaatgtctggacgtcaggcatctcagaccgtttgcccattttatgacaagaattatttagatcttgtaggatggaaaaattgaacgtgccgttttctggctatttggaaccactgtcgagtttgcattggggtcaagcggcatcgcagaagaaaataaggcatttaggttttaggtcaggtgtaagttgaataggttttaggtttttaagaacacaggctaagggagaagaagggggcaatggggggcagaagcttgcccatagtgaaggaggcaagcccagagaaaagagagagtagagacacggagagaagggatggggggttcttgccccctagaaaagctgtacttgccactaacggtgaaggaccaaggcaggcgtccctgcatggtcagacacctctgaaacgtgggtgaataatcatgcagacatccccgtgtgattaaacaccaagcgaagactgtcttcccgagtccgtgaacagcgccagagttttgggtccacagataaaacgcgtctccttcatctctaccagaaaaggaaaggaactgaaattaagagaagggagagattgaagtgtggcgccaagattgaaaggaaaaagaggttgagggatagtgagagaggttggagaagagagtaaaaagaggccgcttaccagatttaaaattggtgagatgttcctttctgaggaccagaggtcataggtggatctttctcacagaacaaagagcaggaggacaggggattgatctcccaaggggggtcccccgatctgagtcacggcaccaaatttcactcgcgtccgtgtgaagagaccgctaaacaggctttgtgtgagcaacaaggctgtttatttcacctgggtgcaagcgggctgagtccgaaaagagagtcagcgaagggagataggggtggcgctgttttataagatttgggtagataaaggaaaattacagtcaaaggggggttgttctttggcgggcaggagtgggggtcacaaggtgctcagtaggggagcttttgagccaggatgagccaggagaaggaatttcaccagataatgtcatcagttaaggcaggaacaggccattttcacttcttttgtggtggaatgtcatcagttaaggcaggaactggccatctggatgtgtacttgcaggtcacaggggatatgatggcttagcttgggctcagaggcctgacagtgtgttgtctatgtcctttgtctattggttttattttgaaggaggcacctagtggttttcttctctttatataatatggttatcaatcattttttcctatttgtatgtgtgtgtgtatttgtgaaattaacccctcatcatgattaatagtggaccaagaacaaagtataaaaaataatatcatgttgagataacggcgtagaatggattttaaacttcttaaggcattagggtgcaaatgatgtactaaacaacatagaccgatctttctgagtaaaaacattctatttccactctgtgtattaatatgcattggacctggcctttcaaacaaatgtgatattcagggaactgccgcaattgagaccttaataaaccagtttcacttttttcagtaaatctgtgaaagctattattttacattttctccactgttaaaatacagaaactgagagaagtactcagtatcctacccaagaccttatttttcttcaggatgcatctcatgttttgtacttcactgtctttggctgtttttttctttattatttcccattttcttaagttgatccattataaaaacaactcctgcttacaggactatttttttccttccagaggcgtgctgttatttcataggtgaacggatagtgtcctgatggtgtctgtggcccttgaagctgcagtttaaggagggggttcaaagaaatggtagagaaaggtcgtccctgtcctggaaggatgccagataggatgacctttgaaactgtcatcaggggcatgaaaaatgggtttgcaatagttccctgcccttctctgcataaacgatatacagcatttgatattaaggcaaatatccttttccattgatgatgtgtaatcataaatcataagaaatttctatgatttcggattcaggaaggcacctttctacctgttgcagctgttgggagtcattctccctccagcccgactatgtcttcccactgcttttggctgctcccattggctttcacattctcctctccacctttggactatcccctacccttatctatccacacattcacttcctacattccttccatgcctaatcaggacagctcctctgaccaccttaacttcccaggatgtccgcgtgctctggataaagtgcgctaagccccgccccgcccctccccgtgtgatctgagtcgggccccactgactttcctgtacacacatcctctgtcctccttggacccatatttgtagtcagtctctctgttcccactctgcatggcacttgacctgatgctgggaaggtaatcattatccttcccttaatgtttcaggattattcattctattttgccacatctaatgtaatatttaatagcatctatgaagaagactgaagactataaataaatgctctctttagtattgagaaggttttatacaatataaaatttagagtacatttttttctcaggaggtctgacattaatattaatcgtgagccccaaagtagaagcttttatattttagcataatgaagtttaccatatgtttcaaagtcacttgcaattgtgcttctcataatgacaattagaaaggtcatcctgtcttgccaggctccattaggtatataaaccagctcgtggctgtcaccgtgaaatgtgttaaggcacttgtatgcaaattcaaggggagacatgatattttttatcccaaagtgtagctgtttacaactaattaccaaacacggaacaagcctattgtttgcctttacatgatatatgggcatagaagtttctaaagcaaattctcctctgtcttttcattgattaaattatatgaagtcaccctcttccatgaagcattacatgatgaggtccccaaagcactgaataaagaaatttgtcattaagacactggtgtatttctgatctatatattttttttctgcttcactcgaaaaacttattatctgcatgaaagtatggaaactgttattgtatttaactctctacatgtgaaccttgaagaaatgatggggcgaaggtgaaatgaaagggaggaggtgaggaggtggcacaagtaatgttttcttatggaacttgtagagggataaacctacattagaatcaaagcaggtaattcctgggaaactgaagcatgccattgtggatgtgggtcatgggaactgatgctggaaaaaaatagttcctaatctggtttgggaatggcaaatttcgttgccacaattgaaatattaatatacttctttgtatgaatgccctttcatttatgccttttttttctttaagaggcattttttctctaagacaggctaaacttctgtattcaagcctttagagtttgcaaacatttatcaatagataagaaagatagataaaaagtcctggattcattaatgcacagccatggtctctacagttttgaaatttctcacttgggagaatactggagaagaacatgtggcccgtgaagcatttgactgttaatatttcgtactattcagatgctttaatttaaaaaattaatctatgaatttaacatatattcatgggatgcagacactgcagagagctgcctcttcctggactttgtgtttgaactgtgttccggttgcttaagcacccatctatctggaatggtttgtgttcaggttttccagaggaaaggatgactccatgttcctcaaggaactttccaggcaaatgaatttgttattttttaatattaaaaatgatcctctttattgagagtgatagactaagggaggaataaacagttgtaatagccctgtgacacataagcggagaaaaaaaccccctccatgcttatgtaaataggaaggagaacaactgtagcaagaatatagaaaaggactcctttcttctctcttccactctcgttttttgccctttggcctaaagctgttaacattgggtgaatgtccctgtcgtcatcttgcattatatctcacgttctgtttgttattgatagggatggatatctgagagtctatatcaagagttacatgtaagtttgtgtaaggcccttcttttgcttctacatttttgctcgttgtatggatgcattttgtaggactgtgagcttatatttaaaacacctgtgcggcagtggacccttgtcaccgcctggcctctcattcagaagccttagctgttcctcaactcctacaacacatgttgagcgcctctcaggtgccagctactgtaataggcactggggagtggaagggtgacaagatgtgattcctgacctcgagagctttgcagtcactggtggaggaggcacatgtacacagcaaatagggcacgatgtgttgaaggatgaaatgaagcaataaacaataggtatcaagacagcagagataaggacaaccaaggatgccttcgaggagccagggagagcctcacagaggaggcagggcatgcaccgtggcttggggatggaaggggcagaaatagtgttgttttagggaagaggagagcacagactcagaggtcggaggaacgtggcacatctaggagacagggatccaatcccagccctaccacttagcagctgtgtaactttgggaaagtttgctaatcactctgagtatgtttcctcccacgtaaagtagaaatactactactactaatattgacctcttaggaccatgagtagataaagtaataatacatgtaacaatcttggcttagtgccctagcgtatcataaggactcaataaatattagtttatgtgattattgatctggctggactgaagggtgtaagagtgagaatgataggagatgaggctggagaagtggacagagccaaaccatgagataccttttcaccaggctggggttttcaagatttctgaaaggtcgtggctagccattaaaataacttggtttctgtatcctccactgctcctgaacattcttgtattacagttgtgttagtctgtttgcattgctatcaaggaatacctgagacagggtaatttgtaaagaaaataagtttatttggcttatagttctgcaggctgtacatgaaatatagtgctgccatctgcttctggcaaggcctcaggaagcttacaatcatggtggaaagggcaggggagccagccagcgtgtcacatggcgagagagggagcaagaggcgaggtggaggtgccaggctctttttaaacaaccggatcttgtgtgaactcattaccacagaaagggcaccaagccatgcataagggactcgcccccatgacaccagcacctcccacaaggccccacctccaacattgaggaatcacatttcaacatgagatttggagggggcaaacatctaaactatatcaaaagtcgatgattaaacacagtctcatataaatgttctacctcaaaaaaacccaaacattgcacacctattatagctaagataaaaaatactgacaacaccaagtgctgccaagaaggtggagcaggccaattgctcatacatctctggtgggatgtgtcagtatcgcaggtggggttgtaagagtagagccatctggaagacagtgccagtggcttatggagttaaacattacacttgccatgtgacctggcaattccactcctgggtacttatttatcctctagaaataaaaatttatatcccacaaaactgtaatgaatatttatagcagctttattcataattgcctaaaactgaaaacaagtcaaatgtccttcactgagtggatagaataacaaactatggtaccttcacatagtggaacacgactcagtaataaggaacaaactatcaatatacacaacaacctggctgcatctcaaatgcattgtactgagtgaaagaaaccggtctcaaaaggctacagctgtagcatcctattcatacgatgctctggaaaagagacctctaagggcagggtttaggaattgggagaggggctggggaagggtctgattagaaattaacagcccaagggaattcttggttttgtttttatttgtttttttttttttgttgttgttgttttttgagacggagtctcgctctgtcacccaggctggagtgcagtggcgcgatctcagctcactgcaagctccgcctcagcccaagggaattctttaggggccttgggattgttctgtatcctgtttgtggtgatggttacaagttacagcccatagaactgtacaacaagtgaattttactgcgtgtaaatttttaaaaaattttaaaaacttgaagacatatagctcatttaccacgtgctttacctgtcttatctcattaatcctctctatgaggttgatgttattattacaggtcgaacattcctaatctgaaaatccaaaatctggaatgctccaaaatctgaaactaattttgaggcctgacatgatgctcaaaggaaaagctcatagaagcatttcggattttgcattttcaaattagagattctccacaggtaattatgcagatcttccaaaagctgaaaaaaatctgaaatccacaacacttttggtcctaagcatttcagataagggatactcaacctggatctctattagtgcattattatctactactattattattattgactaagcagaggctttgtgaggtttggcccaaacctacataccttccagggggcaaagcctggcacaagccaggtgtgcacactccccagaccctgaatttccacagcattgcgagtcacccctcaaggagtcacagagtgggtggtagatctttctcaggctcttgtacaggctgtacctcagtttatccagctcattggtcagtgtggttgctctgtgcatcatgctggtgcaacttttacaaatgtgcaaatgttctaaatgcttagtatcagagagggagttgcctactgaatattttctgacctcttaacttgtactgttggctaagttaaatttggcttcttttcaaggaactattaccttcctagagggtttgtgtggtgacacccataaaaatcttattcagtttttgttcaattcacagctttaacttagtctccttaaaaatcactcaggttgtaaccaaaggctgctctatggctgagtagaattgttaattattaataactgttaatcaactacatagtcaacctcagacaagagggcaagagataattccttagacacacacacaaaaagagcaaaatgttaaggttagaaaacagtaaaaacgatgaagatatttaattctttttgcatactttgtaattatttgtttaaatatctatttccctagcctctgtgccagtttttctcgttgttttttatttttatttttttaagagagggtcttgtgttgttgcccaggctggagtccagtggtgcaaccatgactcactgcatcctcacactactgggctttgatcctcccatgtcaggctcccaagcatctgggactacaggcatgcaccaccatgcctggctaatttttaaaacatttatttttgtagagataggatctctctctgttgcccaggctggccttgaacttctggtctcaagcaatcctctcaccagtcctctcacctcagtctcccaaagtgctgggattacgtgagccattgcgcccagccttttcgaggctattagttttctgcccccagttcctgtaatccttgcatagaataggggcttagtaaaggtttgctgagttagaactccatttcaatggatattgaacttgagaactgagtgagacttgtggagtatggtatttcaaagtattatttatccaacagtctgatatttcagaatcattcatttgccaactcctatgtaggtactttggagatattatttgagttattccttatgacatcccatgcagcaaacatattccttcctaaatgaggaagctgaggctaggagtgatgagctgatttgcttgttatgcacactggatggagtgtgagctgtgtcagacacgtgaattcagatcctcttgtgccacatcagagggacatgagatggggtggctgtttcacttctgtgagcctcactttccccctcattcatttatgtaaccaatggtgggtccaggttttgtgaggactggagcttacataatttgagtatgggtcttatttaaagaaaaaaatatataggattatgattcaaatttggatacagggtttgaaggagacttgtgacagcgggagaccctgaggcttatgtgccattagattcatggtaaatctgtccttgctttcaactaatttttatagtgagccactacgtgccgggctgtgtctgcagctggttacatgtgctgagcctaacagtaccatcctggtcctctagaatgctagagcccactgggagacaccaccagaaaacagacaacggcagtacagtcagataagtgtatgatgggggggtatgaaaagccaggaaagatttctggaagagatgacatctaagctgctactcaggatatgtaaaagttaaaatggagaaaagtatggaaagcccatttgaggcagaagaaagaacaagcatgagaggtgatagagaaaagcttccttatggggaccgcaagaagtttagtggggctgcgttgttcagtggggtggaggagtggggagagcttcagctccagaattaggcaggggccaggtgggcttgtgtaccacttaaggagcttggaaatggtcctaagagcaataggaggcacttggacagatttaagcagggagtgatctgataagatttgcattggggggatgaaaaaaaggccacactgggtccagctgcagaatggagagagaatccactccacactgggcacgagggccatccccctgcagggaggccagtggcaggagagaggaaggtgacaggagagcagctgtgagaacgaagaggaggggatacgttacaaagttgttcagcagtgagaaccaacaggacttgagagtggatgcttagaggcaggaggtgcaggtggtcatgagggaggagataagatagaagcttagctttctggcctggaccaccggtggggggtattcattagaatcaaggccataagaacagcagatttggaagagattgtatgtctcctttgagccagttgagtttgcagtttctgagtgacatccaggtggagatgttcagtggagagctggttgcaaagctgaggaaggagagctgactgacggcagggctttggctgttctcagcatatggattataattagagccgtgcagatagatgagagtgcatgggaaaagccagtgcagagacaagatggtgcaaaggagtatcctaaagaacattaatctttaggggacaggcagaggacaaggagccacaaagaagcatgagagactgtggtcagggatgagggaggaccggcaggagtgtgtgaggagcacggattgtttttgtttgtggggttcccctgcctttagggacacactaactctttggatatcaactggttgtcccggaattccatttagctctgtttgacttgtgtgcagagcccatggttttcacagcccctcttcctttcagatttgataatttgctagagtggctcacagaactcaggaaaacactttacttatgtgtacaggtttattgtagaggatacaactcaggaacagccacatggaagagatgcataggacaaggtatgggggtggcgcagagcttccatgccccctccaggtgcgccacctcccagcactgccatggcctcaccagtccagaagctctctgaaccctgtggtttagggttttcatggagcttggttttatacattttagggagacatgagacatcaatcaaatacatttaagaaatacattggtttggtccagaaaggcaggacaacttgaagcaagttgggcgggcagggaggcttccaggttataggaaggtttaaaaattttctgattggcaattggtcaaaagagttattatcaatacccagcattttgggaggccgacgggggtggatcacgaggtcaggagatcaagaccatcctggctaacacgtgaaaccccgtctctactaaaaatacaaaaaaaattagccggacattgtggtgggcgcctgtagtcccagctactcgggaggctgaggcaggagaatggcatgaacctgggaggcggagcttgcagtgagctgaaactgtgccattgcactccagcctgggcgacagagcaagactctgactcaaaaaaaaaaaaaaaagaaaaaaaagaaaaagaagagttattatcaataagaaggaatgtctgggtttcgataaggggttgtgcagaccaaagttttctcatgcagctgaagcctccaggtggcaggcttcagagagactagattgtaaatatttattatcagatgtaaggtctgtgttgatgttaataccggaggttatattgaggcatgttcaaccctacttcccgtcatagcctgagccagtctttctggttaaattttagagttctctggcctggcaggaagtccatttacatggttgaggaacttagaattttgtttttggtttacaaagagaaagcagagaagggtgatgaatgactgagccacatcctggtgccagctctggaccgggtttggctgtggtctccacaaacatttttgaatgaatacatgagtgagcaagtgagtgagtgaatgggaggaaggccggcggggggaggtttgaggagagtggggaaaggtctgctgtggtgccttgaaggtggggagacctggcacggggaacacagaattgccaggtggatttggtgcccatttggaaggtggcccagtgacttctggggacagtgtccctgagatcatgcattttccccagctgtgctcagcagcctgcttcagggagagggaatggcaacagcgagcacgccaggctgggggtttccaggcaggtagaggagaaaggcaactgggttcgggagccgaacggagtggcaggaaggtgagtgaagtgatggatggctatgtctacactggaggctggtaaaggagggaaagtgataggttgaggggtgggtggctggatgtggtcgtggaacaggggcagaaataactagggtgtgggaaaagtagtggcaaggtggctgggagcttggtggctcagtttgggtgcagatgaagccaaggtgaagtgaggtgggatggtgagaccaaggacccagaagcccaggtgtcggcctggtgatgcaaggatggcggcagggctcggggtggggaggaggtggaggtccaggatcaaggcccccagagaagggggatgtcccagatgagagcaatgagaaggacagcttggggtgaagctgaatgccctgagccttcaagcagcaacagattatacctgaaggtagagccacgttagggacgtgggaggacactgactgcttgggtaggggtggggagtgagcatccacactcgagtggctgcagatgccatgagaccctccagggatgaccaggtcactgttagggcagagaggaggaggcagctttcagaaggagctgggcatgcagagcttgctaaccagtgatggaggatccaggcagtagggcaaagcccatggaggggcccacaggacgggacaactgcgaagacactgtctgccttggaatgcgggcagcatcttcatagccatccagggatttggaccctggccttcctaagtgagagctatactttttctataatttctgccactacataactgccaaagttaaaggtggaaaaagagtctctgtcctagcttttcagcgaccttttcaaaaggaacagaaaatcgtcgcattctcttgcttctacaatgccagtgattgtctgatgcatcctgggtttgacaaaggcaggtagacactcacggtgcagggacacgccagttccaaatcgcatctttactttttaaatgttgaaaaacaaaaaagacagtgcatcttagaatttaaggcaacatagtaactaaagtggcggaggcttttaactgaaaccagaagaaacagcatgagttttgttgttttatgtttcttagctgaaagaaactcaggcaggataacgtgataaggaaactcaggtatttgcctgtgattagttcaggaactgttgaaaaaagactctgggattgggcagaactagagtttagatcactagcttgaaggacttttaagtccagtttaagatttggagagagttataaaggctaagccagcatttataagaacatgtaccaattagagaagtacaggagaatttgctgttttctctgttattataaagatcattgttcttaaatttacattagtttgctaggcctactgccacagaaaaccacagactgagtggcttggacaacagatgtttattttctcccaactctgaaggctggaagttcgagatcaaggtgtcagcacaaatggtctctccctaagcctctctgcttggcttgcggatggctgtcttctccctgcgtcctccctcacatggccttccctttgtgtgtgtctgtgtcctcatctcctctcctcatagggatgccgatcagactggtttagggctcaacctcttgacctcgtttcaatgttatcacctctctaaagaccgtgtctccaaaaagtcacattctgaagtaccagggattagggattcaacatataagttttgggggaggacacagttcagtccatagcagcctattccatacctcattttataagaagagaatttaaaatcagatccttgcctagcactcttctggtcacacacagaaaataatccagaaagttaccttggctgctcagcctcatgtggctggaccctctgttgctgtaagacctcaccggacccttctccgtcctcgctccttcacatgctggaaccttccttacatgtctagatgcaccgagcttgttcacaccttcagggtttgttccctcatcctaaaatggtttctccttagttttatatggctctacccttgtgccgttttggcctcaagttaaatcctgttcctccaagaagcctccctgaccatccaatggaaagcagccacccagcaatgccagatctcattaccctgtctcagtgctcagtgtggtacttattgctggctgatagggcctcatttatcttgtcaccaatgtccccagcactgaagcaatccccagcagatagtgtcactcaaatatttgccaagtgaatgaataaaaccagaagagttaggaagaaatgaagagagctgctgggaagaatctggatgttcctgaagtccaagactttatttttttaggtgggattcatcaaaaggaggcagtttgcctgaggtcccttccctgggcaaagagagaaacagggctggtctgactccaggcctgtctgtgactttgattgttttaacaagcagatggtatttttgtacacagtgacttggagaacttggagacgcgtatcaatgccgcctttcttccctgtcccttgcataacaggtttggtgtcttggatcaagtttggggcagcatcaaactgcgccactagactgcatcccatgatgcagggattatactgtattcataccctgcagtgtttgcacatagaagccttactataaatgtttgctgagtgaaaaacagtgaataaatgagcgaatatttaaatgacaagaactgacagtcttttgaagttcctctagggtggtgcttctcaaacttcaacatccatctgaaccatctggaaagctagttaaaatgccaggtgtgattcaggaggtcctgggtagcctgagaatctgcatttctaatgagtttccaggtgatggtattgctgctgttctcacctctcctttgtgtagcaaggctctaggatgtgtccaggatggtctatctacttggtcaaagataatcttgtgagttgactagtcatcaaaagaccccgatcacttctcagtttgtccaaaaaataacacatgcaactttcagccagttacttaacctcagtgatcttcagtttttgtgcataggtgaattggagatgggtgttaatgccccctttcttccctgtcccttgcatattatgcctgatgtcttggatcaagttttgggcagtatcaaactatgccattagatcatatcccatgatgcagggattacattatattcatactctgcagtgtcttgcacatagaggccttactgtaaatgtttgcagtgaaacaacgaataaatgagtgaatatttaaaagacaagaactgacagtcttttgaagttcactttggaggaacctagtccaattatcaggagatattatttgttgaacactttgtacctgctgagcttcttgatatgtgttacgtttacttatcacaacaatcctgcaaggtggaattattatcctcatttttttttttttttgagacagaattttgctcttgttgcctaggctggagtgcaatggcctgatctcggctcactgcaacctctgcctcccaggttcaagtgattctcctgccttggactcccgagtagctgggatcatagggatgcgccaccacgctcggctaattgtgtatttttagtagagacggggtttctccctgttggtcaggctggtcttgaactccctacctcaggtgatccatccacctcggcctcccaaagtgctgggattacaggcgtgagccaccgtgcctggcctatcctcattttacagaagaggaaactagtgctcagagaggttagacaacatgcccaaggtccccaagtcaataaccaggagccaatagcagggctctgatgatgatgatggtggtgtgtgtgtgtggtgtagtgtatgtttgtgttttcatgtgtatatgtgtgtgtatgtgtgcgtgtgtttccctcataccattttgcttatcaaatgatcctttaggttctttccaagtgtatgattttaggtcaaagacaaagcatgacacagttcactgtgaattgtgagttattatacaagctcgtgaaagagtcggataccttttctgatcctcaatataacctttgtcttctcaccaccataacacgttggtttctcttcaggaactgattttatattctcatgagcacatatgcagtttctcctatagactgtaagatttttaagagcaaggactgggtcttatttttcattttgttccctagtgtcttcgatccttattgcattcagtggtttgatttgaacagaactttggaagcatgagacagactctgtacattaaaatgcttccatagcactcaccatggtgtccaaagtgtcactaaatctctgcttgcttgagatttctctatcttaattaatcttgatttgtgctgtttttttgtgaaagagtaaatgcctgtgggagggctgggccccacctacggagaactctggtagtgtgccaagagcaggactgcataattggtgcatgtgacaaggaacagaggagggtggaggggtccctggatgatgggggtgccttccccttggctgtctttattgtgtccccattggcattactgttactgatcactaaatcagggaacagacggaggcgtgaggtaggatcctacgacagacagcctttagtgagttaccaatgacgccagagttgaccatcaagtaaatctgttgccttttgaaagacatgtctgtctgtacaatgctagggcctgagttgcttaaccttctagtaaaccatgaatttctctcacatgtccttggggtgatgaaaatcttcccgagttgtgcggagcacaaagtagaacctgacagggaatgttctgaaataattgtgtggatggtgacaggtaaaaaaatacactgtagagatatcgatcctgtacgaatgcaacctaagttgttgctctgttaagaaatgtttttggagttctctatacactaaaaaaagaagaaaaagaaaaagaagaaaagtggaaaagaagagatttctcttgtcttgtatgaagcagcaaaataatattctatcagaattgttttaaatatggtaaagtggggcacttctatttaaagggtacgtctttggttcattttttatttttaattgccctgatatctcttgcttttgggaaagtccgacgctctggctctctgctatctgttcaaccagccttcagtagaagtgggaacacaaatggctcattgttttaattttggatttccttaggctgttgctgtcacctagtcatctcagacactcttacagggaaaccagcactggcagggtgtgaaggtcatagcaagggcagcagtgcttcccaggatcatgtgcctggagaggtcggtatgccctgacttctaaagggggcctgccttccacaggacgagcacttactgagcgcttctcctgcacttactgtacattctcatccatatgttagttgttgttaccataaatgtcctcacacatctgtggcatgtggatgacaagagcttctgagtatattttttaactgtgtttttcgaaggcaactttaagtgctaacaatccagtggttatatgggtggctattgactttcagattcacggcactgttctgagtgggtaattcgccgcctttctctaactcttatgtccttgaggaacctgaatgtacagatgattacctgtacctactgagcatcctctcgcagagttggggcgataaatattctgcaaaacctgctataaaacagtcagccctgagcagcaggaaactgaatatagtcactcacatagctaaacccaggctcaggcctacattcacccctgtccgaagggggtggaaatgagatcatttcacacctcacgtggaagtgcagacctgaaagaagacttggggctcccccagcacaggtgtccccattttacaggtgccaaaggctgtggcggagacagagatggggagaggagttggccgtgatgcgtcgtcatggagcatctcacatgtaagggccatgagaagtgaggacagaagggcagccaggctggctggtcttcatgtgggccagttgagtgggaatgcggcactcatgggccggcggaagaatgagcatttgaggaggcagagttccccactcttctgaacctctgttttacaaaaggcttagaatataaaattgggaccttgtctttggaaaggagggagcaaagtgttaacctggacagctttggtgcacaaggtgagaggtttaccaagggctcacacactctccttcatgggagagggtgcttaggaggaggtctggctgctgtgaggtatgggacctgtccaggaccttctactgctacagcaggaacagcactgtgagggagggatgttgctcttcctatttttcagtcgtggaatggaggctttgaaaggtttggtaacttgctcgagatcacacagccagcgaggggtagcatgggattaaacaccagtcggctcgtctccaaaggcctgactgacaaaaacaaatttcaatcgattttacttttcctctgtaatagatgaggaatatctcatttcagaatatctgaaggtgacatttggagtcgctcattgattcaattattctgaatctttatttcagagcttatttatctcaggttttagatgtggaatgctccaaaactattcattggtttcacttccctttgcattgctaagtaaaggtgatataggagaactactcacaggggtgagcacagcaagggtacgaaggatgtaaaatcagacgcagcttttctatgcatctttcttaaacacattactcttaacccaagagacagtcaggatcattaactttcctaatattttgtaaagcattcatatagcaacatttggggcacatgcgcacctggtcttaagtcagggcaggagtggctaattaattctctgattcctcaggcttcacagtttcaaccagccataattattttgcaattgaggttccctctctgcctaaactggcattgttatcaagggctaaatgcatcagctttgtaataatgaagatgggtgccaattccatcttccatatgggatgaagttggggagagagatggtggagaagcactggtcagactgtaaatgatattgagtagaaagaagtcagggatttttgtttttaacatgaaaaagaaatagaattgtaactaagttgaagtttctttactgtttcatcttgtttctttacaaatttcatcttgtcatttacaagggtttatgccacatagctatggatttgggcccaatatttgaccttatagcagctctaaaatattagactgcagttaggaaatgccctctggatctgatttttaaacagaaccaaatgcattctactcttttctgcttttaagagtaacactgcttgaggatgcattgaagccctgctagacatctttgctgatatttgcctttagcattctttctggaggtatttagaccttaataaactaccattatcctttaaaaaaggactgaagagaataatatttgtagaaacaaaataatcaaatacagtcctacatcatttaatgttggggatacgttatgagaaatgcatcgttaggcaattttgccattgtgtgaacgtcatagagtgtacttacacaaacctagatggtgcaccctactactccacacccaggctacgtggtgcagcctattgctcctaggctacaaagctgtgcagatgtgactgtcctgaatactgtaggcaattgtcacactgtaagtatttgtgtatctgaacatatctaaacagaaaaggtacagtaaaaatgtggcattataatcttaggggaccaccatcacatacatggtctattgctgattaaaatgtggttaagtggtacatgactgtactagcattaaagttcttccgaactgagagggtggaatgggaacaggtactggcttcttgttttaagggctgactggtgaggaaggtactggcttcttgttttaagggctgactggtgaggaacttgccacacagagcagtaggctcaactaggctgaagacaggctacagataggctccagggttgacccagggccaagatacagcagctgccttgtcctggctcgctgcagacttggcatctccagtgcctgtatccctgaatttgtggttgaacttaagtttggggagtcccagtggccctaatgagactggaaataccaccctaaacaacctcctttagcagctaaactgacaataataatctgttgtttactgcacaggattgagatgagtgtagcaatctcctagacgaagggccaagctgctcacccacctgggtggggtggtcagtctagggcggcccaaggggattcttgggtcacgatgaccctggggctgataaccaactcacaaatacataaatcaacaacatgaggctagcttctgctggggctttgttgtctggtgggcctgagttttaatcccggctgacctcttactaagcgattgtgggtaagtcattctaatttttttctctctgagcctcaaattcctcatctataaagtgagcatgaaatcctattctagtcattctgagggacaactgaagtgatatatgtgataatatgaaatatgatagatgtgatatatagaactgtaaaacttttaaaaagtaaaatgcctgttaaattgttaaatatgtgtaatatgtgaaaaactcctggcaaatatacctactaatatccatttccatatagttcttttatataaaaggtagagcagtatatgtaaaccttctaataaaacacttagctataactatgaattctcatgtgctcttcctgctgtcagcctctcctcctgctgttaaatctttgcaaggcctcctctagttgttatgttctgaaaagattcaagtttgtatcctagacttcaaagttatgtctctcctatgtggtgtgtagtgtatcaccctgtacttggaggtatgtgatacaagaaactaaggaactaaaataattctttttttttttttttttgagatggagtctcactctgtcgcccagtctggagtgcagtggtacgatcttgctcactgcaacctctgcctcccgagttcaagcaattctcctgcctcagcctccggagtagctgggattacaggtgcccgccaccatgcctggctaattttgtattcttagtagagatggggttttaccatgttgaccagactggtctaaaactcctgacctcaagtgatccaccttcctcagcctcccaaagtgctgggattacagacatgagccactgtgcctggcccctacaataattctttaatgtgaggaaatatcaaatgaaaaataagaaattctaggtaaggaaagaaatttatttagtggaagtataaaagaagtttaaaaagttgaagaaaattttggttttcaatgttgatgcagcgtcctttgtgaatctttaaatttgtgaatgacacttttgaaaacttacaccttattctaagtgtctttgttcactttacactgtactgtaaagaatcacaagagtgtctcataaataactttctatataacatatgtatatttcaaaataacatgttatatatataatacatacaatttttgtcaattaaacagtacattaaaaagacaaaaataactttccataaatgagctcaagttatttcatccacagtgatagaatgttccccatttgatgcaagcagttaaacttaatttccattaagacaaacacacaaataacaatcctccttcccaaacttgaaatcccacttctctttttgaactctcttcatgaagtagtcccttttcttgcatttacaacataaagcctaaaccacaaatattggagggcaggggttgaggatgagcaattgagatgttcctcatgcccttaaaccaagagtccttatgttctctccttcctaaggatggcttattttcctttccatgaaatgactttaagggtgattatatccaaagggaagtgacgtttccccttctttctcttcagttggtccattttcacagcccacaaaagcaagtctctattttgggacttgattcaaagtctgaaaaaaccctaggcacatctttcttccttgaatatgttcacgtgcatattccaaaaaaggttctcattgctagttaggccattaattttatgtgatatccaaatcaaacagctgctgtactgtttaagctcagatcctaaaatggcactttgaacatgaaagtcgtggctgccactctctgggtgactatctggcgatgacacttctacctttgtttttggcttgacgtgcatatgccgaaggcttcagcaaacataaactcatacaaatgtttgtcaagctgtgtgagcctaaacagctcattagttgcaagaatgatcagatgggagatgggttgcccaatttggatggtgacctttatgagctctcggttggattgcagatttatgtgtcacagtatattggaacataatgaatcctgtggcatgtggatggtaaaatgcagggcagaaactgtgcaatgaagaggcaggaagaaggtggcaggtccaggagcataagagcagttaatcatctcctttcccccttcccctattctgcttcccctctgttctcggggtgcccattaaaatgcagttggtaatgaaatgctgtttgaaatcaaagttgctgaaacgatattgtggatattgtggaaatctgtcatatttattttaaggatgagcttacttccaggtgctttcagtgactgcagccccgttagttcccatgcattgattctctctcatgtccctgctccagggtaattctcttgtgctcaggcacatgaccttctagttgaaaacctgcccctgccatttctttatgtggctgcggcccagggagcttgcctgccctgcgtgagcaggttgcaatgataatggttcccagtgctgctcagtggggaactgaagtcatggtattttccttcatatcctcttgaagcttcattttgtgaccttgtttcatcctgctgaacctatcattggaattaccccaaaggtggtggcaggggagtccccagaaaggagtgccccaaactttcccaagatggggcctactgagattccaaagaaagaagcagtaaaagccggagtgatcaggccaaagcattcattaggggaacttaggcagagtgggctgcagcaatcctgcggcggcagcatgaaaaaaggggtcttccacctagaaacatccacagcgaggtgctcaggggatggagtttacatgagggtttaaggaatttggctcagggccagggccagtttctttcagtgttttgggcaaaagcttagatacctttatcaatgtgtgggaatgttcaaggccctggtttgggttcaagcctgctgggaaaaacctgcagctgtctgggtcacagagcggtcaaggcactctgtgagttttggtcacaacagaaaggcaagggctctaggggaccctccagaactacaggctccaatgtcagccccactttcccaagtacaaatttatttcagaaattaaagtactactcttactttttcacacatttcgaagtcatgtgttgacatggatagcctgtctagcacaggaaaactctgatctatttaactgatttaaaatgtttataattgattatctggataagtgtcattgatggcactccaggtgtgtgaagacgccactggcctgggaatcacgacatgtggggtttgtcccagatccctgccatgccatttaaacaccatgggcctcagtttcctcacagcaaaaattagggttatactagatattctccatggaccttgtcaagtattttttgtgatgctgggattttataactaacacaggcaaacaataacctagaatgattagaatcttgaaaaaattgtttgtttgtttgttacagagaatcttgaagttagctgaagtctgtttgactttcttccggaatcagcacagtccaaatgcagccatgacttctcactgtcagaactagacgggccacactgagaaagaatgtggaccggctacccggaatctggccgcttagtgcattcaagaatgctgcttggtttctatggctgtgatttccaagcattttctaaaaggtttatagaggagaaacctaagtgcctgccaccaccccaccttgaagtcaaatttccctgaaggcactggagcatagggagaaaaacatcagctaactcttgccgctataggcccgggttaaattgcagctgggaaattcattccagtggcttgccgggctaaacacaattgtggatggagcctggagaagcatccaagcaaacaaccacttgtgtcagttcagttgtttcaccaaaaaagcaaaataaaacagattttactaactcaattaataattcagttgccccataaaatatagcctcactgaatttcttgtcttgtcttccaagacattaagtcagggcttatttatagccatggctctcttccctggctaagcatccgggccacctgccccagcaggtgagcccttggtccaaggtggtcctggactcagcatgtttttcagagttctccaggtaattccaatggttagtcaatttatcattcatttggggctgattatcagtcttccatgtggataaaaccagttaatttcctgtggatcttttttgaaagccttattaagaggagggagccctgttttgaacttgtgtcatgagctctgcctgtctctggaggtggcaagggtgctcgtcttcattctggtctgtgtcgcatgctgtcttcctctgtctccctactctttctcaaccttcttatttactattccacacactcgctgggttagacaaccttttctgttgttctgctgaaggttacaatttggagtcgattcctgtagtcttcctttttctattcactttcaaaaaagatgttgcaaagtccaagtttcttcctcatgttccgtttctctggggagtgctgtagaagtttccataccttcctgcttctggccttcttagtattttaataactcctgccttgttcccaggtttctccaagtatctggggaaagtagaaacctcagagatggggtctggaaatgtaaaacgttgatgaatttcagggtcacccttgtgaaagctattgattggtcagaggtattctggaaaagtctgcaaaatccagaagctaatttctattttaaaatacaccctataactcttgagtgcccactcatcctcagctggctctggggaatggtgtctgagctcacggtctcatctgtgggagctgcatctcatcaacctattttggaaaccagcatacttctggttctgagaattcagcttaaaaacaatacctagtttcctgtataatccccccaccaaaaaaaagtttgcaatgtgccccatgtctatctgacacattttcacttgtttcttcattgatgatctaggtctgtccaccctgctctatttcttttctatttatctccattatcttagtctatttgggctgctgtaacaaagtaccatagactgagtggctcataaataacagaaatgtatctctcacagttctggaggctgggaagtccagaatcgaggcagtggtagattcggtgtctggtgagggcctgtttccaggttcatagatggtgccttttcaatatgtcctcccattctggaaggggcgagggagctctctggggcctcttttgtaaggggactaattccattcacggagattctaacctcatgacctaatcacctcccaaaacccctacatcttaataccatcactttggggttaggattttaacaaatgaattttgggagggcacaaacatcagtctgtaacatccatttgatgcctacttcaaaattttttgttgctattacatcaaaatctcttctggctttttctgaaagtggatttctatgtaaccactttttcttttcttttgcattatgactgagtttttcttcttaagaaaacaagaagcacgggtagatgatggagagcatgagtgccagagagtaaggaagcctgggctcttctgccagattttgtgttagtcgaaaactgaggacttggagggttgatgagctgagcttttcgttggggctcctctttcccatccatgtattgttccagcagtacaaagtttgtcaatcagagagaatgtgggttcccatggcaaggtggcttccagttttcatactctgtaaaaggcttagtgatggaaggagattaaatgtaatgctttaatgtggatttaggtagagatcttgtaccacagaaaattggaaatcaagggtgaaaaactgagaaatggagtgatgtctttttcttccatacaatttcatatttgcaaagtaaattaactctaaatagtttagggttttattccagaaatctataaaactttaaagaatcttggcatctgattgatttttctagtttatgaaaaactgacaatattctaagccaaagacacattcaacataataatgcaggttgagtatcccttatctaaaatgcttgggacaagaactgttttagatctaggatttttttttgaatatttgcttatttacctgttgagcatctctaatcccaaaatccaaaatccaaaatccaaaatgctcctgtcagcatttcctttgagcatcatgttggtgctcagaaaatagtgaattttgaagtatttcaaatttcagatttttggattagaaacctacatatcaaaaaatattttgtgtaatgtcttacgatgtgtgtgtttggagccttctcagggttaagcagatatgaaaagcccacgttttcaggaaagaatttaagaaaataaagggccaacattttttccctcctggctccatgtcataatatttttgccggctgcagagtctattcttttctatcttcagacttatcactatcaagcattttccattccccacaattctcattttttgttatctttttgcacttgcaaaaactgaaatttggcttgaatgagacacatttccctaccctttatctctcactgattcattccttgaattgggacaaatctttctaatctaagccagtttcatcctcttgcaatacagtaacctgtgtcagcagagtcctgtgtgggatgagtttggcagaattgcagactgcatttttggacctagcagttgaagatgattctgtccctcatcaatttttttccgcaagggcacttttgccttcaaatggtattgtgtttttctatttcctttatctaaagaaagtcttcaacttagttttgttctgttcctgtgaatgtcggcacaggaaagtccttgaagaacatgagaaacaggagcttcatctgggctctgtgatggtgccgttgtatgaattgaaatccatcagggtgggactggttatctgtctaacaaagatgcaatgagacatgattgagagtgtgcttcgggtggagtgtttcttacattttgaggttaaaagccttttctgtcattgtttttattatttatttatttatttttattagagatggggtcttgctacattgaccaggctagtcttgaactcctagcctcaagcaatcctcccccaccttggcctcccaacgtgctgagattacaggagtgagccaccgcacccagactctgtcattgattttgacaagtttttttttttttttttttttttttggttgttgtggttttggaagttttttctcagaaaggacatacttgactgaatttttctcagagcctaattattatttagttatttgtatttatttctttttgatttcactacagtagccactatatcttgagtaaatttagcttttaacttgctttatgttttggtagatcttagtgattatgcaatttgatgatgccattcaatatcaaggtcagaccacagcatgtacaagaggttgctgtcacgtgatagttttccattttcatctcttagtatgctctcttatttcttgctttgctcattcaatgaatgttttaagagtctgattgagatattacctcatagtagattactttcccattaatgtttgctcactcatctgtaggcctaggaagctttatatctatttacaacagcgactaggaccaatagtgtgctgttaaatgcttaggaaccagttctccaggaaaaaaaagtcctgattggcaacgtctggccatttccatagtgtaaatctcccactgtggctgatttccagctatttctggtttaacaagtggctcacaaaattcttgaaagtttatcaactggctttcaccaggaggatggctccagcacccaccacacctgacaacagttatattactttacggatgataaaaagctttatcatcgttttatttcatttctgatttacaaaacaaccctgtagagtaggtggtgttatacatgaggcatctgagactcagaggcctaagtggctggaagtagctccagcagctattattaggcagagcagagacggaatgctgcctctgagctgagcattcttggtgttctcatcactcatggcaactgtttcccattgtgaggattcccttagtggtgtcatatttggactccaaagaagtatactcaagaaatgactacttcgatggttttagcagataatttttagttatttgaggataagaataatagtctgcttatgaaagaacttggtaaatttttttcttctggttttcagacaccatttctgtcttaaaatgaagtatctggtgtatgattgctttgtaatattttgttacttaagggtggtatccagacaacccttagttaatgttatagaatcagaactagaaagacctcttcattcttcacttagagaggatttaagtgtctctggccatttattgggttaataatggtgacttcaggtgacccaggaaacatctggatatctttgctgcacttggaaagacatttagtttggtaatattttcaaatcatgtgatcatttcaaaatgacttgcaccatggagtgatgatgtgatgctggcagcagctgctgtttgctggagatttcgtgtgtcaggcacagttgggaatactttatgaatattagtttatttctcaccttgaaggtagacattgttctcagtttgttgaaatcgaggctcatgtaagtaaaaaatgcccacattcatagcaaataagtgacagagttagtctctgactctgcaaaggacttatatggcttttgtataataaagtcggtcaccccattttggtaataaaagggataataaaagaccaacatgtcttcattagcttgcagataagctccaagtttcagcacgtgcatagtttgtaatgccttcacagggtctgcaggctcattgccatgaaaatatagaactaacaagaatttagttttgattgacatagtttataaaatgttcttttttttttttttttttttttttttttttttgagacagggtctctgtcgcccgggatggagtgctgtggtgcgatcttggctcactgcggtctctgcctcccaggtttaagcaattttcccacctcagcctccacagttgctgggactgcaggtacccgccaccatgcctggctaatttttgtacttttagtagagaaggggtttcaccatgttggccaggctggtcttgaactcctggcctcaagcaatttccttgccttggcctcccaaagtgctgggattacatgcgtgagccaccgcgcctggcctaaaacattcttttaacaaaagcatagctttatgttgaattcaggtagattcgaaccattgcttttaataacacaatatgggttctttcagactcttggggtagattgaacttctcataaaactgtcatgggacagattgcaacttctttccaatatcagaatctctaggtgagttcatccatttctttgccctgggctttttaagaaagtagggcaatgagtccctggtttctcatctccgtaagaatgaaaggatgctggagccaaggtctagtctactgccagtttctccaggaacaatgtggaggaaaaggaccagtgtagatcagaagtccatgagcggaaactcagaatcaaagcggttattcaattcttcttttcttggaggaaatcactgtgtcgcctgaagagaaaaatgccctttgctcttgcactggggagttctgtttagtgcctttccacacaaaagggctgtactggctggtactatagactttttttatgaagatctttttcataaatccttttgcagagtatagccaggtagcagtatatgaacattggaagtgaaacagttttcgtcacacaaaattaacaataaaaacacatgcaactcatcatttataaaacgatacttgcaatttttcattattctcatttctgagggcaaattttcagatgaaaacagaaaggaattttagaagttttcatttttttttcctacatcaggcttccctatatgtcgtctgtataacaagcaccttgatctttcattcagttataagaggctcataagcgttgcctctgttttctatatattggtagtgtcattagaaaacctgagggggtgggccgggcgcagtggctcacgcctgtaatcccagcactttgggaggccgaggtgggcagatcacgaggtcaggagattgagaccatcctggctaacacagtgaaaccccgtctctattaaaaatacaaaaaattagctgggtgtggtggtgggcgcctgtaatcccagctacgcaggacgctgaggcaggagaatgacatgaacccgggaggcagagcttgcagtgagccaagatcgcaccactgcactccagcctgggtgacagggagactcgtctcaaaaacaaaataaaacaacaacaacaacaaaaaaatctgaggaggtggacaccattttttggtactgaacgacgtgatgaccgtttaaaaagaaaggtagatgttaagaaggcttccttatacaaaaattcttgtatattgttccttccaattttatttttgttttgatatataaaatcctatgttaaatctctcatcctgaatatcagattactccagaaggagaaagtagcagtctaattattttatttggttgcatggtagtcttagtacaattaaatctcctttgaaagtttctcagttatgtagttatgaatagcactccacaccacggctgtggtagacctggagcggtattgggcccaggtcacagaatgatcacggaaggactttagagatcacctattgagtccctgttttagagaggagcaagcaaaggcccagagaactcactccatttgccgaggtccgacaagtgtgtaggtgagctggagccagaaccctgtctttcggtcttcactctagagctctttccaaacaaacatgctgacctgggagtctgacaattgctgtatgctgatgtttcctatttgaagtccacatgtctttgcaggggaagagaactgtctagtatcattcgttaatgcccactcttcatgtttgccaaagaccaccaatacattgggcagtgattgtgttacactggtttgtttatatgtcggctttcagtgatgccagaaaatcaaaaggaggttaagaaatcccagaggttcacccaggatgggaggctaaacctttgttgtattatgagtcttaattcaagataacataatctttagcctaataaacattaggttgaattttgaaaatgtaaacactaactgatctgtagactgacgtctatttccttggcaatattggccacacccattaaataataacttctcattctcccttcccccagcctctggcaacctctgttatactttctgtctctgaatttgactactttaggtgcctcatataagtggaatcacatgacatttgtccttttgtgatacgatattttctttaaaaagaaaatgaacagaaacagtacatgagagaagtcaggtctgcagccagtgtgcctcttagttccatattcatacaaggccctcttggaagctcagctttggagttggatattatctcagaagatatggcagcttaggaatctggtactctaaaaacagatttacaagcattagacttttttgtatatgtgtgtggtcattttagtcttcctgaaagtggttaagaccaatatttctaggcagactatagttcacatcatccagttctgtaagtcttggattatcgatctcattgtcatggcaaaaaaggacagggattactcaactcattactcctgtgtccagacctctgagcctggtgtgtgtgtgtggctcagtgcctttcccatagtggcccacatccccccagggccctgcatgaagttcctgcaagtctaccccctgcacgtgcagagcaaaaagagcttcctatgggagctgttctccagaaaagacagaaacccgggccctagagaaccagaagatttaaattccagtttcagctctgccattgactaattaggcaactgtaggcagttcactttgctcctttggaccagagatttctcatctacaaaatgaaaaacttggatttaattatctctgaagtcacttcaagcagaaatgtaacaattctttgatttattaaaaaaaataacatcaatgataatgaccaacagctaacacttattacaaggctccaggcactttcgaagtccttaatatttaatgttcactactaccttaggatataagtactgtgatgcagagtcctggacacagagaagtcagcaacttgcctgaggacagcctgcaggacacagcactgtgatttgaacccagagagtctgacttcagcccccagcgctcaactactattaattgaaagggcattgaagagacatgatgtcacaataaaaagaaaaaaagaaaagaaagcatgtcttcgactttctagtcgtcgtgctttgtatggattcttctttcaaccttgacgaaagggtataattaatctgtaattaaagtcatgatagtttcttaatacgaggaaatgttgtcattgcggttctctgaacacaagagggcgctgtggcaaagcttaccgtctgctcatcctctgccgcaggagggtacaacttcgtgggacaaacttcaaaagaaagatgccatttctaagacctcatttatcacctcgggatagccagtcttcatgcctttaaaatacagaataaaaataacacataaatcaacacaattacattccttcttgatagcatcaccagtcttcttgaggcctggcgtcaaacctctgcttcagtgttacgttatggggtgcgcatgcctccttaatacccaccttgctggtattctccagatggctgtcttcttagcagtagatggaattaagcaccataatgagggatattttatttaaatctgctttaaaaaaatcttccatgacttattcacatgtctggttagctatcacactgccattccatgtgatcggaaatgagaaagatttaaagaaagtaattgtttagcatgcaacggatgtacctcttttgcttcagatgagaataggcagcatagaagaactggggctgattcgtggaaggtttcccgtagctagaccctggaggctgtaaaaagtggtttggaactgtgtttaaaaaataataataatagagggaatagtgaggtacccaggaggagcagagagcgcctgttccagccatgggagcatccccatcagatacatagatgctcttaattaattaggctgctagtaaaccctcctgagtgtgcaagacaggaggaattcattttcaagctttaaaaaaaaagatgagtcttgggcacacagccctgtggcccatttgttattgtcgtgttacccataagtcagcagttttcctctcctgttgttgatagatcttcccatacagttagatttgtttagaaagaaaagttttaagtccttgcaaatgattacaaactgaagaactggaaaccctggttaggaaacagttggagcactatgtatggcttaatgaaaggacacagtctttttaatccaccaagcctgaatcatcagcttggttaagactatctgtagaaggtgagcagagcacttcaaacctggctattcgcaagggagagaaaaaagctgccactgtccccttagcattcctctgctaatcttccatttgtattgcaataagtgtgctttggcattgagtgtgttttaaagctcctgcttagctggctgggggacaagaaaaaacaaaacctcaaggacactcccactcagaggcataaaaaaataaacagttgtttctgcttttgagaagaagtcacatgcatgtggctggcattctcaggtgggcaaaggagagtgctcggtagcacctgccccaaaccctccgttctcccctttttaggtcagaagagattctgctttcctatcttgtctccagttttcactcttagtccgtattttccctccccttttccactctgtccttaactcctctttccctctttcatcgttttcctttatttttcatctctttctttattccttctcaagggtttgccggtggttttgctaggcaatactgagcttcaaggccattgtcatggaagtttccagtcctgggatgtgcttagaccagcctttaactatgcatcctcaaaacagctggcagcctaatcaaaccttattttttttctctttgtgagagaggtgaagaagatgataacatgaaatgaaatctagggcctttagaaagagtcagcaaaccaggcgcagtggctcacatccataatcccaacactttgggaggctgacgtggggatcacttgaggctaggagttcaagaccaggctgggcaacatagcaagaccccatctcttatatatatatatatatatatatatatatatatatatatatatttagctgggcatgatggtaagcaaccatagtcctggtcactcaggaggctaaggcaggcggattgctcaagctcaggagtttgaggcttcagtgtgctgtcattgtgccactgcactccagcttgtgtgacagagtaagaccctgtctctaaaaaggaaaaaaaaaaaaaaagagttaccggttatacatttttgagaggagcaatggggaaatgcaatcatggggtaataataatcaggacaagagcaggggtatgtactgtcccccaccctgctgccctaaatgcgaggaagaagccacgttgctggacccataatgagaagcttcagaaggttgatagagagtgctgatcttgaagacatttagtctgggacacagactacatatcagcatgtcaaacatatcagcttgcttgaatgaatgagtgaatgaatgaatgagtgaatgaaaagtggtgaaggaagaagatggttgggtttgggagaagaatatgcctgaaagatatacaaccaccggggagtctgtcttgtctttacagacacttaactcaggaacagacttgctggttgtcttgagtgtgaagtgcaaaaagtaaaaatggccctcaacaaaaatataagtcaggcctgtgttagagatgtcaaagatgaagacctttatgatgatccatttccacttaatgaatagcgtcttagtccattttgtgttgctataatggaataattgtggctggggaatttataaagaaaagaagttcatttggttcatggttctgcaggctgtacaagaagcatgacactggcatctgcatctggtgagggcctctctctcttgcttcctctctggccatgtgatctctggccagagaggaagcaagaaagaggaggaggtgccaggctctctaggggaactaatagagtgagaattcattcacccccgagggagagtattaatctgttcaagggtgacctgcacctatgcctcaaacactgcccattgggctccaccttaaacattggggagcaaatcttaactttagctttggaggggccaagtatctaaaccatagcagagtctctttgtgatgatcttgagatgaaattgtttgggccttggttcctgagaggggtcttgccttattcagcagtcatatttttttctagataaaattcttactatcctttagacctggggagagcccgtgtatgaaaaacagaacacaaagctgaactttaaataggaaaaagctagcaaagggactacctctgggggcactacttgagcctgtaagcagtggttataggagatctgagagtacgggggtcctagagacaggtcagttcttagggtggggcaggatataagaaagggagagtgaggaaggctcagaagatgaggtttgaacacttggccaccattttgcctgggatcatcaggggaagctcctgatatgggaacatcactcccaggcaggacaccaagaggcaaaggccagtgtgctctatttagtaagaaggtaagataagggccagtgtgataagtgcatggagccccatgggcatccaattaataaaacagtgatggaccactacaatttaaagtcctttcttagatgagctaggcaaaaggttctaattagagatacctgtcttcccctcttttccctataatgcatccaaagatctcaggtgtaatagatttgcagcttgtgctaagtagttggccagagtgtgctggcttctgtgacctacgtctctgagcggcccgagctgtctgttggtgttatcctgagctcgttgtgggaaggcggctggagcagggtgtgctgcccatctgtggctccatcagtttccataaggcttcagcttgatcagtgcagcacgaactgtggcatgtttggggcttgggagattgacagccccccaaaaagccctcaaaacatggtcaagaggacaaacgagaatgatagacatatcttcactattgccagcctattaacctattacaaactctgtttttagagattagccattagtttttccttccaaaattattatgctgttgtcatttcctccacaaactgagatatgcctggtgaaatgacttaatttaaaaagtaatttagattatattttaaagaataaatattttcttcaaacttatctgattttcaaacgatcatgactttaattttgctctagtaatgcactttctgagaataaatgggaatctcagttttgtaatatgtacccgcatttttcttcttacagctattgggggtgcactgttggggatgtggtgggggctggacctgtgtggaatagggcagatactgtgtgcgggtctccacttctcaaaggagcttcaaagctgactgcactcattgatgcagcgagcccagggccttgtgatgttccctttcttgttggttggtgctggaaagtctagagtcacagactccttttggactaggctacaaatgcatatatactttgcagagcattgcctattagtcgccattctctcctcagagtgtaggcaggttccgagggcagcacctaatgtgggaagagagaatagaaggttctggggaaggggacccactctgggcagagggagaaggatagggaggtggtttggggaagaatcacaattatccttactttaattaatacatttccttggatggcaccaacagtgaacacagccacacgctcttctgcccatagtgctaagtgggttctctctttgactagaattgtgtcgttgatgtggaagtgcagttgaattcaaacgatctgtagctggtggcttttattcctgaagttcaaatatagacaaccctcttagcaggttctaccatagttagccaaccatgtgttaatctccgcctctttgtcttctgtcttaaaggaagtcaggccaccctaaatcgcggcctctcatgtgtgcctgactgtggagagtgtgattttccagcagtctctgggtctctataatgtttgaatttgcctttggggtgcatgcgtgtggtatgtgtggcatgtggggtcaggggtggtgagaccagctctgaattgtttccagggcagcttggtggcaccctggccagatcctgcatgtgacatttcccaacaactctttctggtctgtgtctcacctgtcataattcaaaggagagccatggactgtgtgcgctagattcaccttcagagactggagagtcccatctctgtaccacagtggacacctggacttcctctggctttctcttggggactctgccaggcagaggccctcagccgtgcactgggacagttctgtgggcagtggcaggaagccttcagtcggtgaacagcgtgaagacagcctgatgcatgcaaggcgtggggatgtgattgtgtgagcagatcaatacctatatctcaaaaatgagaactttcactttagtacttgatcacttaccctattgaattatgtaagtacttcagtctcttcaacattggctttctttcattctttgtttttttaatctcccatcttagtaatgaaaaaaaatgtatgtgtcatgtggttcttttgtaaggttatttctctccatttgcttgaaaactttcttattactcctaactccttttattgatttaatgaaaccaaatgctcctgaaagtttgaccctgatctagtgatacctgcactctctaggtccctaaaagaccccacagtctgatgctttggctgcagatggagtcacagagatgattcagctttaaagtgtctacatcatagattggaaaatgcctttctagggctgaagttgtagctgtttcctagacttctgtttattttccatttctttacccactcctgtttgaaattctgctgttgtttgcatactgtttgcctttgtgtccctctttccatctgttcttaaaaaaaaaaaatcaactgagttttattttaaaccaatccagatgacaggcacacagtcttcacaatgctttggaaaccagttgaagaaaattctcgttaaattactcatccacccaactcattttccatacatgatctattgttggggtgcctgcctgttttcaagataagtgcatatttcagaggcaggttccttttaccccagataagcaattatttaagaaattggctttgacatacattttcagttttaatttttccttttacattctgaaataatctctggttgcatgtagcttcccactactgcatataaagatgtattccaggcatccgtcaggagtttgtctattccatgaagaagacattgctgtggaagaagatggtgggaggcctgatgtcctttctgtgcagccctgcaagggccttcacctccagggcctaatctcacaagcaccgactctgcactcagatttccgacaagaacttccgctgtaaagtggccatgttaaggagaccttgggaagaacaaacccattatctgatattgagtcactatgaaagatttgggagtctttcccattagtgccccggtaagagaggcacaagaggatcatgcaaggctactaatctccatttcaaagccaagaagaggtcatcgatttatagtagggctgcagatcagaagcaaagcggacttcagcctgatagcagtcaaaagacttgtgtttcagcccctttctatgatgtcctttgactagcgccccttcctgtgtccctcccctgcattccatagcattgggtcttctacaagggaccaattagtgatttgaaaaccaaagtgaatagagtacttataaaatctggtggtgcactcacaggaggaaatgagttgggagggtttaggaattgtaaggacaagtttactaatgagaaggcaggttaggaatatccttaaggaaaatgcagatgacaaatttccagctatcaaggaaaacgggttcaacctcataattagatcattcccgcagagccaaggaactgcaaaacacatggatcttcactgccatgggagactgactctaagaagagaaagttgtcagaggggttctgtgtggaaaatgtaattggaatccccaaattatgacttctatcattggaagagggcatcttaaataatttttttttttgagacagagtctttctctattgcccaggctgaatgctgaagtgcagtggtgtgatcttggctcactgcaacctctgcctcccaggttcaagcaactctcctgcctcggcctctgagtagctgggactacaggcatgcaccaccacgcccagctattttttttatttttggtagagatggagtttcactatgttggccaggcttgtcttgaactcctaaccttaggtgatccacctgcctgtcggcctcccaaagtgctgggattacaggtgtgagctaccgtgtctggccttaaataatcttttattttttatttttgagaaaaagtctcactctgtcaccaggctggagtgcagtggcacgttcttggctcactgaaacctctgcctcctgggttcaagcgattctcctgcctcagtcccccgagtagctgggactacaggcgcacaccaccacgcccaactattttatttttattttttagtaaagatggggtttcaccatgttggccagggtggtctcgatctcttgacctcctgatctgcccacctcggcctctcaaaatgctgagattataggtgtgagccaccgcgcccagccaatattcttaaatagtgatgttcaaacacatggcctggccaaagacttcctaactcactctcattgatttttgtcccttccattcaaaaacacttgttttcatagtcctgtgcttcaaaacgataaaacaatttaaccctgtattaaccatctgaattattaagaataccaggctgggcgtggtggctcacgcctgtaatcccagcactttgggaggccgaggtgggcggatcacgaggtcaggagatcgagaccatcctggttaacatggtgaaaccccgtatctaccaaaaatacaaaaaattagccaggcgtggtggcgggtgcctgtaatcccagctactcgggaggctgaggcaggagaatggcgtgaactggagaggcggagcttgcagtgagccgagatcgcgccactacactccagctctagcctgggtgacagagtgagactccgtctcaaaaaaaaaaaaaaaaaaagaataccatagcattaagtgagataccaatgaagacatgaaaaaaatctccatcagaaactagggtggagacatcgtaagccctcaggatccaggcagagcatcatcaggatgtggccatctggttctctgccaaccttatgctgtcctctcacttgtccaacccatggatatcaacattgatagaaaaatcaatctctatctttgtaaaatctgccctgtctgtggcaagctgaatttttattaagctgtaatgtttggagtagatgatccaggatgctatggtcagtgatggggagaggagaagactctgcagctgaacctgtggggatgtgagccaccagcccccagccctggtgagtgaaggccacctgtcagctgagtaaggggacatgagccaccagcccccggccctggtgagtgaaggccacttgtcagccgagtgttagacagccaggggctcctcatcctgctgcctgggcgctctggcaatgtctgctgctgtaaggatggccagctgcctaccaaggtttgaaatcatgagcagtattgaccaccccttgggtacttgggcaattcacttccctggccttgacctcacttccctggccttgacctcactgttttctcatctgtaaatgagaaaaagagttgagatgatctataaatttccctctaacacatctaggtctgtcattacttaaacatattagtttgaaggaactaaactgtactccctctgaattaccaagtacaaggctccatggtcattttcactctcctgtctggtgacagcaggtgtggcttggctttgctcttctccttccttccaactttctcctaaggtctataatggacaacttagatcattcacaattcccccacacagtttggcttgggaattggggccacaaataaagaggacaaaagagggaaaaagcataaagcaaactgtaccagaaggtatgtgaatggcgaggacacgttggctaatggggccatggtgtagcccattaactcagcttcctaggaatgctcttgaactagtttctatttggaaatgactgataaatgtgggctgaaaaggcccgagagccccaaaatacagcaaggacactctgaaggaccccctctgatatacttgggatgtgtgtccctgcccaaatctcatgttgaaatgtaatcttcagtgttggaggtagggacctggggggaagtggttggatcatgagggtggatccttcatgaatagtttagcactatcccccgcattgtactgtcctcacgatagtgagttctcatgagatctggtcatttaacagtgtgtggcacctccccgcttgctctcttgctcctgcttttgtcatgtgatgtgcctggcctctttttgccttctgccatgaatgtaagcttcctgaggcctccccagaaaccaaggagatgccagcctcatgctgactgtaaagcctgcagaacaataaaccaattaaacctctctttttaatacattacccagcctcatgtatttctttatagcaatgcaaggatggcttaatacaccttcccatctcatatgtcttttaacatcttaagttcatagcaagatgtagcaacagtggctgcagcctacagagcaagcttgccttttgtagaaatggtgtaattgccactgacccgtgatagttgcaatctgggttttaaagctttgtgtctgaacactccacacgttttagcattcctgaatcctagaattcagtgctgcaaatgagtttcaagagggtttatggttttcaggggccatagcaggctgtctctgaagccacaacagaggtgggcaggctacagctggagtaatcatgactgattcttttctgctgctgcctcagtgacaggtgtgtggatttactttgtccctcccacctttctttgcttcccaccctcattgttaatggtaaagcaaaggcttagggtaatgggcagggtgtgtacagggtgcccactgctaaatggttccacctgggtggagacaggcctgatgggagccacagaagagtactgatatcatagccatccaaagggccactctggagggagagcggagagcagactggggttacatgatcagtgggtcaagtcagcagtcacaaggaaatctccccaacaagtaaagaactaaaaattagaaggggaagatttagtttaatataaggacccatttcttatcaagagctctgtgacacctaccattctcaggggtggctcacatgagcccaccttcttactgcagagaccaaccttgtacagccaggaaaatttactggatgttcacagacatctttgttgtatattcctgacataggttattgacaagcactgttacggttttgagcaccaagagaaaaaggtcatgttgtactagtgaatcaacataagtgtaatggctaaattcaatgtacaatgggactataacttacctcctttactgtttaaagaaaagtcaagatacacgcattcagaattttgtatttttatcttgttggcagagggaaaaaataagtattgatgtaatcccaccaagtaaaggttaaggtactcaagttaggctgcttgcattcaagtcctatttctggttcttactaaccagctttcaacgcaatacttgaataccatgaatttctggttcctcttcccattggtgttcagtggatgttaggtattaatgttattttaggtatactctatgagactgtatggtacagctgcccactatcagcctattggtggaacatatatgttcagatcagtttttttcctccatgtaactttgcatggttgcatttacatttaaaacaaaaaacacaactgaagaataggaatgcatgactgatgtgcatggaatcagcttatttcgactgaacagatgaaaataaaaaatgacttaatagctaaattcaaagatataaaatgagtgcatggtaaatggaaattgagtggtaagtatctcaattcatattcactgagggcaagatgaagacaaatgaactcaaattgcagcagaaaggaattaaacattaagaatcaaacttcttaacttcagaaaatagtcttttgaaatgtctgatgcaaagaaatgacactgataccttaaaacagttcattcttttattaatatttattaagcatattctatcaggcaggtgctgggctcggtgctgcagatacagtgatgaacaagacagataggcactctgactgcatggcccatagtctagttggagaggaaaacttaaaacaaagacacagacaggcaattataaattgtgacaagtgacatgaaggaaggtacaagagtgtgaaatggggcacctgaccaggctaggggcagggcacagagagaccgccctgaggaagtgacaagttcagatctgagggacaatagtaacagatggggagaaaagagcaccagaccctctgaacaagcaacctgtgcaaagactgcaaggccgcagggggttgctggcttcaagggatggagaaaagaccagtgaggtcagcagagatggggagctgagaggggtagccctgggttcaagacacaggcaggaaccagactaagcaggttcttgaagcaagtgaaggactttcatctttatataaagggcaatggaaagttgtcaatgtcattgtcattttttttaaatgccttttttactggagtgtaatatgcatgcaagaaagcacccataggggtgagctgggtgaatttcattgtgaacacatcaagcaaccagtaccaaatcaacaagcagaacattattaaccccccaaaagtcacccttagcccttccaagtcctcagcatgttccaaaggcaaccctatcctgactggtaacaacactaggttaattttgaccatttagaaactttatataaagggagtcataccatatttattctctgtgcctggcttctttcactcaacaacatgagactcatccagattgctacttatagccataaatcactgtgtgaatagaccatgatttaattatcagttttgctgttgtggacatttgggtagtgtctggctgtttggaacagtactacaggttgagtatcccttatctgaaatgcttgggatcaggagtgtttccgatttttttttttttcagattttgaaatatttatatgatacttacttgttcagcattcctaatctgaaaatctgaaacgtgaaatgctccaattagcatttcctttgagcgtcatgtcagtgttcaaaaagtttcaaattttggaacgtttcagattttgagattagggatgctcagactgtactatgagctacttgttgattctatttcacctgatcttcggtgtttttctaagagtggaatttctgggacttaaggtatacatatgttcagcactaatggatcctgccaagcagttttacaaagtggccatttcaatttgtactcccaccagcaatgcatgagagctcccttaaaaggtttgctatagccagtgatgtgatcagatctgtgcttttcaaagaccatatggcctcctgtgagcagaatgggctaaagaggcaggagtgcctggaaggacctgtgggggctgtgatggtaggtcaggcaagcagaagacagggaaaggccaggatggaaagaagtacatggacccaggagagatgggtggggaaatggctgatgggctcagggaatgttttgggtggggggcatggtgggcaggagaggggagaggtgacaagaatgaccctgttcaagacataggcaggaaacagatgggtggtctatgattggttagatggtagtgtttgaactctggagatggatcagagatggggaggtgtggatcataattctagtgcattaagagtacataatttgaggtgcattagctatgtccaagtgggaatcaaggaagcaattagatacagaggtctggactcagagtgaagtcccaatggacatgcaaactggagagtcattatagcaatatgggtgagtactatggtttgaatgtttgtgccttccaaaactcatgttgaaatttaatccccaatgtggcagtattgagagctggggcctataagaggtggttggatcatgagtgctttgcttttatgagtggattaatctattcatgaaataatgagctaatgggttatcctgtaagcgggactggtggttttataataggaagagagacctgtatgtagtatattagcatgctcagccctctcaccatgtgctaccttgagactctacagtgtccccaacagcaagaaggctctcaccagatgctgcccctcgaccttggacttttcaatctctgtaactgtaggaaataaattccttttctttataaattactcagtttcaggtattctgttatagcaataaaaaccagactaaggcaatgaggaaatgtcacctgggttgagagtagtgagaagaatgtctcattttgttatttcttagtttgagagcagttattcaaattaaagtttgaatatttaaaaaatttttgctcttgattttatttcaagttaaagcttcttaggcatgtgtttatttgtagggcagagatgataacactgttataggaccgacaggttcgtatgcccactgcacagtaatagacccattacatggagacagcaggatttgcagcagaaaaagagttaaaggtcagtgggcaccaagtagagaggggaggagaccctccaatccatcttcccaagttgttctgggttggagtttttaaggggatcatggagagcaaggcaaggtggggggggggtgaaatcatcgggatgtggaaactgtattcttgggtgggtcagctcctaagggatccctcagaccagctgagtcagccattccctcagaatgcaggacctgaaggaatatctcataggaaaaacttaacgtttcatgatgttcaaggtgttatcaatagagcagttaaggggaactgtgatctagggtctatgtgattccaagacagtaggcagcaaacaactctgaggaaggggttggagcccaggctggcctcatgattagcgccgagtgtgctgcaagctgggtttattttcctttctcccctctctttcttccctgacgaattttatttaaaaaaagtttatagggatggtttcaacaccatgctgtgggcaagagaagagagataccttgttctctgctgcagggcaccacagtcaccaagagtggactttccctggcaacagggcctctgcattggtctgcttgagctgccataaccaaagaccacaaaccgggtggcttaaagaacagacacgcattgttgcactgtcgtggaggttagaagtctgagaccaaggcgatggcaagattgcagagctctagggaaggatctgttccaggcttctctccttggcttgtaggtggccgtcttctccctctgtctcttctcatggtcttccctctgtatctgtattagtgtccaaatttctcctttttattagaacccagtcatattggattagggcccactctaatgatcctgtttgaactctgattacctctgtaaagaccctatccccaaataagttcccattctgaggtcccaggggttaaggtctccaacagaccttttatggaggggggcacataattcaacccatccacactcccagccagggtcccgcttcaagacgagcacttcctcttccttcaccatgcactggggcaggtgactctaactagtgcccatttcgtgtctcattctggaaactatgccgatcagtcaaagtttccagagccagtgaaatgttctcatcacacgaggcttctgaacacccagaaattttgaagcaagaacatgacaataatcatcactctcattcactgtgtgccatgtccttgcaagttcacagctagcccttacaacagcctgcacaggtctgattttccactctgcttcattgatgagcatctgagacgcgggagcataagggaacttgcttgaggtcacatgcttggtcggtagagactcagggtctgcgcccaggcttgccagcccatctccactcctccctgctgtgggcccagggcacagcggccacaggaggacttggctcttctttcaggctttgcgggcacagattagctagacagagtaaatatgggcatagcgattgttaacggctaacttttgggtgaatgcactattcttgccatctcaggctgatttatttgcagcactggtagtcagggtccctatagcaagggaagagataatctctaaatattccctggagccatgagaaacggccaggtaaagaagcatccttctttcagctggaacacggcgatgttttctgtcacctcaaatgaggtgatctccagaatcccctcaatcctgtctcattcattcaggcacaaaaaaggagaaacgtagttgacaggaattagatttggaaatctttcagagaaaatgtgtttgatttttctgtcccacacctcagcagtttatttcacactgcgcttattggtctctttgtctcacttctccttacataaatcctctgggtgtcgtttgaataatcaaaacgtattagatatgaaacataatgtgtatcatgttgcatcatgctactgtacctgcatgttgtaatttgtaccttatttgctgtctatgcaatgaaaaggctttcaagtttttaggtcagaaaggtttaatcactgtccctagtcaggtttgcagcacagagccaaatggatggtgtgcatttggacctgcccttggaagtcaggctgtgattagctagggcaaaagtttccttatctaggtgtgaacgtggaatggcctgaaatgtgtggacagtctattatgtgttcatcactgtgcataatgatgggcctgtagggatgtctatgacacgcaggatacagggctgggcacctggttgttggtatcagatatgtaggagaagaaggaatgggtgggggttggagagaacacaagcacaagcataagacggttctgctcccaagaggctcattcgtcactcagtgctgtagacacacgggagctgccttagcacagagcgacagatgccatgagacaggcgcccactgctgatgtgggcagaaagagatgtgcaagcgaagggcaacagatttggttgctgagattaataagaggaatcaagaaatgctcccaagagaaaaatgatacttaatctgggtcttgaataaccattcattcattcattcattcttctttctctctataatattaattagatttctgctaataagtacctagaaatgttctaggtgtaggaaattcagcaatgaacaaaaaaaacccaaccctccccccagcaaccccttgccgtcgggagctcacattcctataggtaagtggtagtggtgtggagaggggctggcctagtgaaaggaacagtgccagctgttgcacagggacttgtgatgaggttggtgtgagtgggtggccatgtggggcaggtcagagaggaggcatcgagttgccaggtgctgggtcccacatgaggagtctgtgtgccaagccaaagagctttctcctgaaggcctcaaggggccactattggtctcagcaaggtgggaggtcatattgtcagacttgtgctgggcaggtgggtgcaggttagaggcaggtgaggccacaggcagggagcctgaggcctggtgagagctggtgagagaaattcaaggtaaggatgaggatttcaggatgagtctcacttgctggcttctctaactacatggggatggtgcaattgactgagggtaggatagaggaaaaggatcaggtgcaggagaaacatgatgcgttcagggttagccaccttgcttgtgaattctagaattctgccagcctcacctgagagcccctcagaagtaagaggggcttcttactgtggaggaagtgtcagaatgccaaccaaagatggccagctaaataaatgacatgcctggtggtctgacatgacaaatcctgagggtcccttgcccagggtgggtggcaggggaatggcagttcacattcaagatctaagttttgagagataagaatcatctcctttggggtggggcagctgcattccacactcgggacccggccactcagtgcggggtgatggtgcagttcccagggactgatgtacctgctctgcagtggcaacctccgagtgtgagagaacccagctcttttttggacaattttgcatctgactcattgttggtatttgataacatttgttatgctaatataacaaatacaataacaggaaaatcagaaaatccatgtgtactcaatgtttagctcccacttataagcgagaacgtgcagtatttggttttctgttactgcattaatttgcttaggataatggcttccagctccatccatgctgctacaaaggaaatgatttccttctttttttgtggctgtgtagtataccatggtgtatttgtaccacattttctttatccagtctgcccatggatgggcatttaggttgatttcatgtctttgttattgtgaatagtgctgcaatgaacatacgcgtgcatgtgactttatgatgaaacaatttatcttccttcgggtgtataagaacaatagacgctggggcctacttgagattggagggtgggaggagggtaaggattgaaaaaccacccattgggcactatacttgttacctgggtgatgaaataacctctacaccaaaccttcatgacatgaaatttacccatacaacaaatcatacatggacctccgaacctaaaataaaagatggaaaaaaaaaaaaagaaaaccagaatataatctgagttctgtctttgtgtgatccggttttacaggaggaattaacctgggatgggggaaagaatgcatttagtgctggaatatataaggagaggctttcctttgtcccctcctctcttctctctcccccaggattttttgtccttccgtgttccacctgatccatggccaactggctttagaagttgttattaatagttcaccatgctcattttcatgaaacagagggaaaggcagcctcttcatttgatgtctagttagcaaaccagaggtttgacaccagctggtcataacagacatttttaataaaacttacctccaagttaaaataaaaatgctgccttagagattttgtcctgaaacatacgaaaaaatgacaggtgggatctctcatgcaaaacttctgctagggctaaaggaaaatatctgataaggttgtttgcccatgggggaaggattaattggattttctgtcttgcccctagtgtctgaccctctgttattatgacaggaccacagaagttttgaacgctgaatcatagtttatagttttagcatttttgagctatggagataagagtgagggaaggagtgtgataggggagtgagttcccacaatactccggtgatagttattgtctgcctgaaaatgtgaatacatatattcaagcccgatgtagcctgttcgaagcaacccagccgggcaatgaaagcctcagctgaagatcataggattaaatccaggctcccctctttcaagtaccttctcatgctactgcatttatgtgggtagtctgggattatgtctgtggtttgtttcactattttctggatcacttctgaaccagtccgtgtgtcagtcctgtggatatgcatttcatgaatgagactctgaggaattttctaatagtgtctatggtggcagtgggaaaaggggaggaggcagagaaagaggtggtagcaagagaaagaactgtgtggagctcgagctaaaagcaggagctaagactgcaagatcaaatgattcccactcagttgtcctagtctctgtctttgttttctgtggccataacagaacaccatagatttggtaatttataaagagcagaaacatatttcctcatcattctggagactgggaagttcaagatcaagggctggctcaacagaaggcctcttgctgcatcatgccatggcagaaggtggaaaggcagagagggcaagagaaagcaagcaagggggggccaagttgtccttttctaatgaaccccctcccacagtaacggcgttaatccattctagagggtggtgcccccatgacccaaacacctcccattaggccccacctcctaacactgccacattggggatcagatttccaacacattaactttggggacacattgaaaccatagcactcccctatagcaatgagtaatatccgcatagtgattttacttattaaataatattgacatggcttcctctttatggtttgctagtggtaaatcttcaaaactcttgggtagtttctgtggatataaattccaggtgaggttagttgttaaggaaagaatttccatgaaagaaaaacgtgtctgagttttgaataaggaaagacatacattgtgggtaactgaaatgccattttttcactcctccttcccccaggctctaaataccattgctgaattgaatccgtccagtttgcacgtgtgtctacagacatgtgtcttatggaatacaaacaatatgtgcttcttcttaaggttttgttactttgtcccttggtggtaagttttacaaaatgatttgaaatactcagcgctcaactccaaaagccaataaaacaggaggaagacattgggtatgatttggaaataagcaaaacagaacagacctgactttctttctctttcttcctttcttccttccttcctttctttctttctttctttctttctttctttctttctttctttctttctttctttctttctttctttccttctttcttttctttctttcttttctttcctttttttttttttgaaatggagtcttgctctatcacctaggctggagtgcagtggcgccatctcagctcactgtaagctctacctcccgagttcaagcgattctcctgcctcagcctcccgagtacctgggactataggtgtgcatcaccatgcacagctaatttttgtatttttagtagatgcagaatttcaccatgttggccaggatggtctcgatctcttgacctcgtgatccacccacctcagcctcccaaagtgctgggattacaggcgtgagccaccgtgcctggcccagacctgactacttgctagtataattttattttaagtgcacgttattattctgctttctccaatatcagacagctggttccttcttgaagcaactcttaaagacagatggaccagccatgtcttcaaagtgttggcttcttctgtggctcccattttcccgtttatcctagtgccttgacttcttcacttgccaacctctgttctccctggcaccaaggagttatgtagcgttgacttgttcttttccatttgaaggtggaaggatgaacataaggtgccagactgggctatttcatcatagacagatggatttgctccattcatgtaaagagttccttttgtagaatatctgaatcctggtatatcctttagatggatgtataatccctatccccacacttttctcccaaacttgtgagttataccattaagtctacaggactgggcagttacgacctggacattagtccagttttctgtattctactacaaaaacttgtttctgttgtttaaaacatgggattatttgtaagttatcactccagaaatgaacgtaactgaatttgctgaggatttaaactaattacctgacatcaaattggttcaaggaagtggggaaaataaagcaaggattgacaactactatttgcttcattctaattttaggccctttaactttaaaggtgaattattccttgttttattcttggagtatttattttatgaatacgtgaattacaataacaaaacactataccgttgtgaaagtgagtgagctacagctacacacaccaacatggataaatctcctataaatctcatcatgtcagcgagcaaagcaagttccagaagaatgcccacagtatgagaccaattatttggtttgaaaatatgcaaagcagcattccatatgtcttaaggacacatacatatgaagggaaaggataaatccatgatgggagtgataatcccctaaaaatagttacttgtgggggaagcagtggagcagaatgcaatctgggaagggtactgagggagttcttatgcgatagtggacagggatggatttatgagtcaagaagagcaactgcacggcgtctggaaggcgtgctggccctgggctctcaggtggaggctgtttgcatgatcagcagataggaaggtaccatggatgcaggagtggtctgaataccagaggaggcaagccaagtgagcaaggggagggagacgagaggtgccaggaaaaccctgtctcatgagaagatgatgatcctagaaggcttagaagagtgtccaccccaggccaggtgcggtggctcacacctgtaatcccagcactttgggaggccgaggggggtggatcatgaggtcaggagctcctggctaacacggtgaaaccccatctctactaaaaatacaaaaacaaaattagccgggcatggtggcgggcacctgtagtcccagctactcggtggctgaggtgggagaatggcatgaagccgggaggtggagcttgcagtgagccaagatcgtgccactgcagtccagcctgggcgacagagtgaggctctgtctccaaaaaaaaaaaaaaaaaaaaaaagagtgtccaccccagtgcccccatccacagggctgctgctgctgagcacccatgcccttgggtgacccactctcctgctcaggggcccagaggatgcttctttcttcttcctgggcactttcccctgagttttggtttaaaagatagaattcccgatgaagccagcttgctgcagtcctgtagccccacatctccatatgcgatcctttgagggaggctgagcatccctgcatctctgaatgggagacatgtttccaccttggctcctgggggcactggggccattttcctggtgttctgagcactctttctgcagaagaagacactctcagatggtagattttgtggatttcagtaagtagagtccttcaggagtctctaggcagaaagcacctgagttgacagcaagaactgagagttctgggtctgcctctagatggctgtttgcctattccagcctcactgtccccatatccagatggaatgacatgagcatatgggcaggacaagccacctttgtccataaggcggggtacacgccaccaagactaagactgaagcctaggcaagacttagagtatttgtggcttcagagaaacatcaaattgagaggaagatcgagtgataggaaaactgaagccagagaagtaagtattttgtatgtagagtatcattttccaatcatcgctggtgtgataaaacagcaaacgtcactgcttattgaaagcaactagacagtatgtttggtgattggattacaagttttccttttagataaggagagatttaatctgtttctggactctttaggagcacccttcccagttttacagttgagggactggagaccctgagaggtgaaggggcctggctgcactcacagtggaagtgatcccactggatctcagatgttgagtttctgacactcaatctagagctcttcttaccacacatgcttcagtctacacagttctccccctctttttttttcatcttctcatctactgcccttattctctctctctctctctctgtaaaacatgtaggggctgtcatagacgacactgtgtgagcggaagttgatgacttcaattgtgacaaatatcaggagctcctctcaaaaattaccttcacaaaagccttttagaaaagccatgttgctgtcacatgtagctataatttgggttgtgctcaaaagacttaaattggttaataccgaagggaggtgtggtttattctgtatttgttgttctagtacttcttgggtttgggggctgctgttatgagaaatggcttctcctagactccagaatgtgcttagagagtgtttttctctttgaattgctaaaaatgttatttcctctctcctggatttttcaaggtgtgcaataagttgtgtagttgctgctctgctgcacagagctttaatataaactgcgtattttcactctgtatgtgtgagtaatctgtgccttctatggctctgatcgtgggtaagcaaaaaacacagtgactgctgaaatatagggatggaaatttctttgtcagtaaagatcctatgaaatcatatactgagcatacgtaaaatatggaattgtctctgcagtattgatcgagcacaaatccactttcgtaaagcaaaaataaaggtcaaacagaaaaggcacatcactggtttttaaaattttttgaataataagtacttgaaattccttaaaaatgacactatatatgatttaacttagatagctagaatagtcaaatacatagaaacagaaagtagaatgcttattgccaggggctccagccctggtgagggaggaatggggagttaagtgtaataggtgctgggttttagagatgttgacaaagttctgaagatagatggtggtgatggttgcacagtagtgtgtgggtatacttgattccaataaactgtacacttaaaatggttaaagtggtaaattttatgtaatgtatatttaattgcaataaaaaggataaataaatgataccatatcctcgtgatttcagatacatctcttacttactgatcattcctcctgagaatgtgttctgtctccatgctgattatgtttcctgttctcttcctttgggtgctgtgttcagcatttttaatttttttaatagttaatgctatgctcacattttcctcccgtgacaaagagtgggaataagcgtggcacatgtgtctctcaccatctgtttgatagctgcacagaagccttaggaaactgagggatagacacgctcccactgagttactcaacactgctgcttccttgtcctctggctgaaggctaaaccctgccttctactgtttacaacaggagtgtccaatctttcggcttccctgggccacacttgaaggaggagaattgtcttgggccacacataaaatacactaacactaacaatagctgatgagctaaaaaagaaaattgcaaaaaaaaaatctcataatattttaagaaagtttacgaatttgtattgggccacattcaaagccatcctgggctgcatacagcctctgggttggacaagcttggtctaacatatagtgttaataggatatttgaggtggagtaagaccaatagatcaggaggtaattgccactgaaaacatagttaattactcacagatcctgtgtcatgccacgtgaggaagcaccagggtgggtcaggagacagacggagcagggaaataaggtcaagagactttattgtggtttctgcaggaaggaatgctgagagagagcaagcccattgaggactgggtagtttgaataattccagtgggctctgggatgtaggggctgcccctagttgtttggtacctggtctggggtgattagggcaggggatagtggtcctgagtgtaagatcccaataagaggagttgttgggtggtgtgggcgtcacctgctgttccattggcctttagccagggcctgagagctgggtcaagataacataatttaaaaaacgatattatacccaccagactatttcttcctggtagaaagcaccacagatccatatggacttggatggatctgtggacagattccatgtttgagaagtagccataacctgcggccttcccctttctacaatgcagactccatcctcagttcaacagaagtttcacaggtatctttacaggtgtttcctgagtcagccgatcaggtgaattgtttgaccccattgccaagaatatttctggctggaggttccgaatacctcaaagagaaagtccctgcttgaaagagtctcctgcccccccaccaccttagaacagttgctggcattgattgctctaccaagagaaaaatgctgatacttgacaaactattgacacacctgggaatggccacataatgttggataatgttaatgccacaggtggaggacgtgggtgtcaggaagaagaagcagtatatgattccacaacagtaatttcattcttcaagaagattgattctattgggaatgatttttcccattaagcatgaaacacagagaattcttggtggatccattcatttagcaagcagttttgtggaagacagcatgtatgactgccaagggtgtggggtgtggtgtccaagtagtgccttcatggctccctcacttataaactgtgtccttgagtaaactgctgaaccctgtggtgccttggtgtcctcaccaataaaatagaggtaaagatacaagctcactggtttcctgtgaggactaaagaacacctgtgaaaagattattaactatgttagagaggagggtgatgatactaattggaaacaaataaggtactaaaacaagtgagacgtgatcttcactctgatggaatccagtgggggaaatgaatataaaaacaggcaattataaaaaaaaaaatggtaagtctgctgatatggagtacactttctaggggcatcaggatagacaaacatagaagaaaaataatatttgatttgagccttaatggataaataaataaataaataacggggagaaagatgattcctagagtagggagtatctcatgcaaatgactgagaagcttgattaagttaatgtttaagaatgtggcaggccaggtatagtggctcatacctgtaatccaagcactttgggaggccgactcaggtggatcacctgtggtcaggagttcaagactagcctggccaaaatggcgaaaccctatctctactaaaaaatacaaaaattagccaggcatggtggctggtgcctgtagttccagctactcaggaggttgaggtaggagaattgcttaggctcaggtggttgaagctgcagttagctgtgttcatgtcactgcactcctgcctgggtgacacagcaagaccaccatctcaaaaacaaaaacaaaaaacaatgtggcatattaaaaattaaaatcacaataaaatcagagggtacattgtgagaagttaatagcaggagataaggttgaaaaaaaaacttgaacccaggtttaaaagaccttggatcatgtgtatatgtgcggttaataaaattcgatagtctagaaagctttaaatgcaatataaagaggactatcttagttgagcaggcacttattaaaattctcagcataaaataacagagtataggacagaaaattatcttggaaatttaataaaaggttatatattagtcagctcctgctttgataatgtagtataacaaacagcccccaaatctcagttgtttatatgaaagcatgtatttcttgctcatggacttgtgcgtcatcttgtggctttgccatgtgtttgtgggttgggtcagatctactttgagtaattctgggactgggctacctgcggggcatgctattctcatggtagactggaggaaggaagatagataggaagacaatcatacaagggcatataaagcctctgggtgggcgtggcatacgttaggaccactggtgttccattggcaaatgcaagtcacctggtcacatccagaggcatggggagatgacgcttactctgcctgtactgaagagaaaggagagaaggaggacttatgaacaatgaatacaatctaccactgatggtcattaatggcctcaaaaatatcacaaagacatgaaagccctgcagtggcttccaaggatcttcttattagaaatgatatgagggatagagcagtttaccacataccacaaacgtgtataccagcatgtgtatactagtttgggaattcgtagttttgagtatggaagtatgggaagtgaaaagaagggagaagtagaagtgttgggtgtggtattgcatacactggctggagacaggaagaaacaacgatcttctaatataaactttaaaatggtcccagagtaaagatataatagtgatcagagatttcactccatgaacatatgaatgtctaatttgtctcttgatatttaacttttaacatgaaatatttcagaaatatagaaaggtacagaaaaaaacatagtggacacccatgtatagtagatgctaacattttaccacatttgattcaagtgaacctatgagtttatttttagtgttgttctttttaattaagaaaatattacagatacacaaatgttgtatattctactattctactctggattgtctttttttccctcagtgtttttgtcatacaaaaaattttaaaacttttaaataagatgactttatcttttccttcttttttttctatttcttgtttaataagttctttcctactcccaggatcatgtgaatgttttctgtattttcttctaatttaaaaagttttgttctgtagctttactttttaaaaacattttttaaagtttccatctggaatttttgatgtgtttggtgtcagagatataattttgtctttttacatatcaatcagtatctaatagcatcatgacctctacagatttgtgtgtcatctgttgtgtgtgtgtgtgtgtgtgtgtgtgtgtgtgtgcatctttttataagtccccatactcttccattgatctgtttgtttctttctgaattaataccacactataattgctatagcattagaggacattctgatatgtggtatgacagctctccctctccccatcttatcacatgcacacacacacacccacacacacacacacggactgagcatcctttgtcttcaaagttgtcatgactactaatgggaatatttaatcaatttatatttaatataatgatgaataggtttgactttaaatctactatcttgctagtggttttctatttgtcccatctgttttttttttcttttttgctcttttctgccttcctttagaactgagtattttttagtattctattttatctcttttttgaattattgttatcccactttgttttattttcctcagtggttgctttagggttcattctaggcatctttaacttgtcatagtttcctttcaaacaatattattccactttatgtgtaagtatatctaatgaataatatatttaaaatttttataatataaagcctcataaatattttatatgtatactgtcactcacacactatatatcactacatatttgtatatttcccccatatatgtatctccctcatatatatgtgtgtgtgtatatatatatatatgagagagataaataattttacaacagcatacttacatttttccctcctgtcatttgtgttacagttgtcatacttttacttccacatatgttgtaaattcagatcaataaggttttaaaaattaaattattgttttctttcctgggataaactctattattctttaccactgtgctagatttttttttttactaattcttaaagcatttttgcatccatattaataaatggaattaactgtaattttgtttcttcatactatctttccccgttttggaatcaaggctgtaccagtctcataaaataactgggaagttttccatcttcttattctccctgggcatcatcctcacctacctgaactactgcagttgcctcagaaccggttttcgccctctcatccttgtcttacagtacacctttgccccaaattcatttcctacaatgcagtgattcttctaaaatacgcatcccttcatgctgctccctggtctagaacacgtcacagttcatggcttattgtcgtagctcagtaattccttgttttctggggtttctctagcatcatacaggctgaaatggtgtcatgtgattccagaggccaaaaggctggtgtggagtcacggcaactttcctaccactacattcccccttcctatgagtttcatcgtaccaatgatggctgttcatagccgactgcatcattttccatgaaattactggtgtggtgtgatctggggtcgcattcaatgggaaagatttagggaatggtcatgagagtggcctcctggatgcagtaacctgacataccctacattttccctgaacttggggtccggggcaggcttttgcaacaggggtatggtatttaaataatcagcataaaacagctgcccattttaaagctgtctcttcatagaggtcaggacagaactctaggtatggcttctactgacagctactccttcaggagatacaaattatatgccgattctgacacttttgaaaaagaaacatatcaaatgcaacttttttttcccatttcagtttatgcttttcactgttctcccttcttctaaaaattgtggtgtttcatctttctgatgctcttctagaaagtgggctaaagcactaaaatataatcattccagtttcatagagccagacttgaggcttatagatgttgtataagtggctcagggacccaaaatatcttctgtggtgacccttgctttaaatctcagggctatgttttcttagactgtctgagcaccagtttcctcatagaatgtggcagattgaggaagattgtatctaaagcaccaacatagtgtctggcaccatgtcgttcagtaaatattatacctgtgaaatacatcaaatagaggcgccactgctgggaagtgaagtcctgcctataattgagtttaacaattccatagaagatttttttgaatactgatgtgcaaggtgactggcctcatgtcagattctgtgagtaaaacaaaggtaattgagcaatggttcccacccttctggcatcactgactgttacgggatctagaaacatacatgatcagacacgtgaacccacggggccaacaccacagcaggagtgaagaaggagctggaaggggaaggtggggattcagtttgcctggcagggcatggtaagacttcagaaaggaagtgacattggaggtacatcttgaaggatgagtgggatttcaacagacagaggctgtagggctgagcacttgaaacagagggaggacatattgcagccaaacctcggtgaactgacttgggtacgcttctgccagaataagaagaggtcttgaattaatagtcaaacctgatatagcttctttgtcactgtgtcttttccttgaaatgaatggaactttttgattttttatccttacttttctcctctctgggttacagtggcaggttgttttatcttcccctgatgaattgaccggtccagagagcatttcagttatcatttatggaacaaggccaaaatatttctttcacacatacagcgcagactaaatcgtctttttctttatcttttttttttcctttaacaacctccagcctatttgtggcatctgaaaaactttgcggcagcattttacagccatttattgtagagtctgttgctttgggctgctgtcagtgctctgacacaaagtttcccatctgattttgagctggtcaccgtgtgctgttgtagtaacagggcctcctggtgcattgaaacacctggtttacttcacacttgatcactggcatgttcaagaacagatagagctttgtctgtcatttgctctttttgggttagcttaagctagggatgttttattttaggtttttttcttaaaatcagagaaaatctagcttcaaaactcaagtgctaaaacaccaaaatagtgttatagaggcacattcctggggcaggggagaggtggccctcgattaaatatatacgagcaagtatttattgaatctcattttaaggacatttgcatttcatgagaagtgagaggtgtggttgctgccctcaagagacttataacagaactagggagaaagatacagccacattagacaaaacagcaagatgtaatcaagtgaaccaaagcagtatcacatgtacacaggagtgtgagcagagagttaaatctatgtatattcactttaggtcatttataaccttgacctccagcaaatgtttaacaatggttataataatatttcttacaattacacataataacccatccgtgcaaatgtctgtgtacttgggccctaaaaatataacatactgattctaggaagaatgcatattcttgtcactgttttcatgttcagaaggaactcaggggtgggccagttctggagtccatgcagctggacagactctggaaggtgaaggacccttgcccaggaagggctgaaatctgtgctgggcagagtgagtgaagggaaggatgttttgattgaccgaagattttctttaattgaacttgaatgggatgacaagaggagaaaaacctggacatttacttcttgaccatggaaagttttgaagtaaacagaacaccataaaacaaaatataatttgggaattgaggttgaaaatcaacaagtaaggttggaaagagttgttgaatgttctacattcacaaataactaggccgggtgtggtggctcatgcctgtaatcccagcactttgggaggctgaggcaggtggaacacttgaggccaggagctagagaccagcctggccaacatggtaaagtcccatctctactaaaaatacaaaaaattacctgggcgtggtggcgtatggctgtaatcccagctactcaggaggctgaggtatgagaatcgcttgaacccagcaggcagaggttgcagtgagccgagatgatgcaagtgcactccagcctgggtgacagagcaagactgtctaaagacaaaacaaaacaaaaaatgaaacaaaacaaaaaaaaccccacaaaaaacaaaaaaaaaagaaaccaaaccaaaagaaaaccaaatagttagatgagatggaaagagtggaggacagatcttaccaggaaatcctcagaacactgactactcagtgtggatttcccaaccagctagctacagtctgcttgtctctcctgagggttttcagactagtttccaaaaagtcaggaaaagggtagaattccaccttccgttccctatttggagctctgggacagtagctgtcacatgatagtatgcgtcagagtcacctggtgggcttgttggaaacacaggctgctgggtccctgagcttctgattcagtagtctcaggtaaggcctgagaatgtgcatttctaagacattcccaggagacacagatgctgctggtctggggaccacattctgagaaccactgcctttgggtgggtgtgggccatgacctccttccaggttctatacattggccccttggctcatcaaaagggtcttggtctatgctttgatctatgcctttccttcaatgtggctgtaagttttacctaattaaaatgctgcatctggtttggatgatctgattaatatggaggatgaaatttatagcaataagtactcagagctttgctcccttcccgatcaaagcctgaagtttgatttctttatacatatattttaataataatcatcattagtgctgaaattagagataaaatgtcccaatcatcccagtgtatttttagtttctaagcatgagcatttgattattgttttctggcgagtctgtcgtgtaatccagtaattgcaataaaagagaaaagtactttttgctaatatggtacaaatagttttgtccacagtgatcaaatacagagattttccattctatgttattttatacccctctctactcccctgcttcccaaaatagacacataatatatacaactagatttattggagcttctgatttatatgagactatataagtgatttcagtgtatctaggatagagattttgttcccagagtaaaagcttctgaagctgtgcaatgcaaataaaattattttatttattatagcattaacttaagatgcagtcttctgaatagtctagcctgtgtttgggatgtaaatttctcttgcaccctgctgcctttcacgtttgctttcagcagggactatagtttaatctttttcttcctccttgccccgcaacaggagatcgctattatcagctgacactgaagctcctaaaaaaatcttagctcagattctctttgtccatctgcaagctagcgcacattccccgttcatggaaaaataccactgcggtggagctttaagtcactgctgttctgcaagaaataaattttatcacaagaagcagtgggtgcagttagaaaaagtcatctagatctaagggaaggcttcaaataattctcctaattggcctacgtggttcactagtagaaaaacctttcgtttttgtccttcaaaaacaaaactgagagagttccctggagacattagtttgctaaatgacttgcccaaggtcacctggtaatatgaccaagtgaagaattatgggtttgtgaaccgttatattcggtataaggccatattcttttgttactttcttaggaagagaatcaatggcttgtttcagaaagctttgcaaggaatcacttactcttctgccatgggaaaaattgtgtatgccagatgcattttgttcatttttaaagagctcctcaatgctattgctgatctcctagggtaagagaaggacagggaggttagacaaggcaactggtagtcttctaatgcctacagtaattaatgataatattcagactagtttcattcatcaaagcgtatacattacagggtttgaaaaataggcgtattttcttagatgcaacaaaatctactctagctgctgcctcagggtgctcattaccacctttctagtctgccatgggtttggttgtggggcagaagtcagccacatacagaacctcaatggcaaggcaatatgggaagtgcagtcctttgctttaagtctctagtgtagagaaaacatgttttgggggtggaatggtagactggagtgggtttgaataaaccaattggcagtaactgctgcagatagttctcaaatgttattgcttccaatggggtgaactaagtcatgcagtgagccaattctcttgcaaatggctggcctgtcttcaaacggctatcagagaaacgtcaatttcagaatatggttagatacagtgtatggggataattagtagatgtcataatgctatgcaatggtattattcttatttagaaacgaaaattgagtaaatttaattttagaacattctaatttcaccaattttgcttgtccataatttccttaaaatttgctcttagatgacagcatggtgcatggcaaatgcaggctttgcaaacaattagaaccaagttcatatctcagggttgcttcttcttagctacctaattggggtatgtcactaaatctcattaattctcactggagaggatgctgatattgaagcgtactgggagcattcggcccatgaagctctgtgcacctttaatgaaatcaggcttgtatggcacactgcagacactgacaaattgtgtggtagttattctgcttctgacccaaatgcaatgatgccatcatcttctgatcccattttttcttgttcactggcacatttactttccaaactgtactttattattatatatttaatcatgcagtgtattgctttaatattgttcataggttatctcctgtttattttgtttctcagagtgactagaatctctctctgtcagaagagagggtgtgacttcttgctgcctttgccttgtatcttatcctgaattctgattctaaccttagtgcttgcatcagttgttatggacctggatcagggccactgtgtatggttgttcaggttgaacagtgaacaagagccccacgtttcatgggataccagtcactttgaatatttattatatttatcatatacataattattaaacatattatatttattatacatattaggtcttgtcctcataaaatcagtacattatggcaactttttaacaaaaagaagtgaagtgtcttaaagaggggcactgcctcttgactggcacagatgcaacgtatgggcaccacagaggctagcagtggccctggataagttgatgatcatagtgggtgatagcataggtttttaaagttgttcacttattcatatgacaattatttgtttttgtgcatattatgtaacaggcagtattctaggtagttggatatacaattattattataaaaataataatcactatatgggaggccgaggtgggcagatcatctgatgtcaggagttcaagatcagtctggccaacatgatgaaaccccatctctactaaaaatacaaaaattagccaggtgtggtggtgcatgcttgtagttccagctactcgggaggctgaggcaggagaatcacttgaacccagcagaatgaggttgcagtgagccaaattcacgccactgcactccagcttgggtgacagagtgagattctgtctcaaaaaaaaaacaaaactcattatcttagtgtcctggggctgccatgataaattactacaaagagaaatgtattttctcacagttttagagggcagaagtctgaaatcgaggtgttgttagagcggtcctagctccaggttgtaggctttgatttgtcagtccttgatatgtacacgttgagtatccctgtctaaatctctgcgatctgaaatgctccaaagtctgaaactttttgaacaccaacatgatgctcaaataaagtgctcattggagcattttaaatctgaaacactttggttgcaagcattttgaatgaaggatgctgagcctgtatacatatcatagcaatctctgtctccattgtgtgttccctccctgtgtatcactgtgtctttgtatctcttcttcttctccttctcctctctcttcctcctcctcctcttcctcctccaccttttctctctctcatttttttttttttgacagagtcatactctcttgcctaggctggagtgcagtggtgcaatcatagctcactgtagcctcaaactcttgggctcaagtgatcctcccacctcagcctcctaaagtgctggggttacagacatgagccatgatgcctggcccatttttcttcttaggtggataccagtcatcagatttagagcctcccctaatccagtatgacctcctcttaatgaatgttatgtgtgaagacctcccttatttccagataaggtcaccctcacacgtactaggagttaggacttcaacaatttaacccattataatcaaagagcctataagactgtagatttacatggagtaaggcaggcaaataaatgagcaataaatatataatatgtgaaaaaatataccatgtgtgagtgattacaagtgctaggaagaaacatcaacctcaagccataggattgcctgtgtggggaggctctattctaggctgtgtggtcagagaaggcctttctgagaaggtgccattggagcagaggcctccgcaaaagcagcagacgaacagtgtggatttatggggagggatgttccaggcagagggagcctcaaggcctttaggccacagtgtgttgggcatctgtgaggagctgctaggaggctcaagtgaaacttggagagaggagggctgggaaagtgagaacagcaaagaaaagggcaacagatcatgtggggttgagtataataggaggttagaaacttggtgattaagatgggaaaccatttcagagtttttaacagaagagtcacgtgatctgactcgtgtttagaagattgttctgttgctgcagtcaagagactatagagagtcaggctgggcacagaagaaactgcaaaagcagttactgctttggagcaggaaaggggtggtgtggttcaggcagcatgggagccaggacactggccctgcagggagtgaatttcaaggttaagtagcctggaagctcagtcctctctcttcaatgatccatgcttctgtcctggttcttcatctatcttcatctatggcacctttgaggtaggtattgagttggaggctttgctcatcctgtgggtgcaggtttggcagcccagaaattatttttaggattaacagctacatgttgttccaggccagacttcctctaacattacatttccctcaaaaattcattggagctcttctctctcttccttttttctgtcccctcccttctcttccttttctttcttgaacaatttagcaatttagtcctaaatatattaggtcaggtagagcaaagtaagcactgctggagaggggagctgcaatgtcacttctgtggggtcccttctgaaagtttggtgtctcactagtgaagaagagaggtacaaataaggaaagggacaaaactagaaggaaccctgtgttatgagagtaaaactggagtattgctgtgaacctaaatacacagatagatacaggaatatcaaggcagtgtgtatatgtacacatgcatttatatgtctactcccagctttgcccactgagagagtctgggagcaatgacatcccaagaacaatgaacacccctagaatccaattcttggcttctaaatactcatttccactaagaggaaccagagttctttggagcaattgctgaatacaagcagggaaattgcagactgagctagagccctgttgatgctggaaaacaaggaaaagatcaaaaaatgggggacatgcaaaaggatcccaaagtcagccagattgggttcccactgccaaacagggcataaatcgagcataaaaataaataataataatattagtggatgataacccagtgaataagatagaaaaatcttgagtccacacatatgcaaataaacacactgaaggtttgatgaggaatggaatttttacatagttccaattacatcccctcagaatacttattaaatacaaagatgaaaagaataactttacagtggagaagcctgacagagagcaccttaatcaaattatcaacgttatcatcagcaatgggacaagtcgaagtccttagccaccacagtaagctacaagagcaccatgacatcagatctgggatttcccaacaaaaatgcaaacctgagtttgatcatgagtaaataccagggaaactcagatggaggtgcattccacaaaataactggcctatggtcttcagaagtgccaaggtcaagacagtcaaggaatggccaggctgtacgcagtggctcatgcctattatcctagcactttgggaggccatggtgggaggattgcttgagcctgagagttcgaaaccagcctgggcaagatagtgagacccccgtctccacaaatttttttttttaattagcctggcgtggtgatgcacacctgtagttcctgctgcttgggaggctgaggtgggaggattgcttgagcctgggatgatgaagttgcagtgagccatgattgtgtcactgcattccagcctgagcaacagagccagatcctgtcttaatttaaaaaataataaaggaatgactaaagaactgttcccaactcaaagcgactaagagacatgggaactgagtgcaatgtgtgattctgaggggcatgggggacccttttgttgttataaaaggcattatcaggacatttggcaacatttcaatgaagcctgaggattcgatggtagtagtctatcattattgatttcctgattttgatggttatattgtggttatttaggaaaataccctcgtttttaggaaatatccactaagatattcaagggtggaggaacagcgggtcaacaactctcaaatggtccaggaaatagaaagttctttgtactatatttgtaacttctctgttgctctgtgattgtttaaaatgaattcgctgaggtcatggctttttttttttttttttttttgagatggagtctcactctgttgcccaggctggagtgtaatggcacgatctcagctcaccgcaacctctgcctcccaggttcaagtgattctcctgcctcagcctcccaagcagctgggaccacagacatgcgttaccacgccgggctaattttgtatttttagtagagatggggtctctccatgttggtcaggctagtcttgaactcctgacctcaggtgatctgcctgccttgacctcccaaagtgctgggattacaggcatgagtcaccatgcccggtcaagggcatggattttgatccatgaatcagtgagagagacgtaagtacaatgcctgatatttttgcctgttcttgaggatagcatccctactattactgaattcactcacatgtgtgagtttggggatcttaaaatatcccagggggtgctttatgcacgagggatatggattaagtccagggtgaatataggtgtttcccagtgataaggcagctgtgtgtgaaagcaaggacaaaaacaaaactaaactaaactaaaaagaaaagtcaggccagtgtgctagatatttacagctgggtgattgtgatggcactgtccttcctaaagctttgtaacagattaagtagattctctaaagccgttcaggttagagggtgccttgcccttattgctccaccgtgctttttggcagacattgtgaccatgtttagtaattatatttggctccctgaaggtaaaacaggagctgcgtacgtattaggggagaaaaaaggaagcacaagaagaaaggaaaaaaaaaattaaggggcttatgttttatatcctcccagtcaatcccatgagtgaataactacattacaaattttgtctcgatgtggtttttaagcctaaagaatctttctttttctttctttgctttctttctttctctttcttttctttcttttctttctttctttctctctctctttctttttcttctctctctctctttctttccctccctcctccctcccttccttccttccctccctcccttccttccttccctccctccttccttctctccttccttccttccttccttcctctttctttctttctttctttctttctttctttctttctctttcttttattatactttaagttctagggtacatgtgcacaacgtgcaggtttgttacatatgtatacatgtgccatgttggtgtgctgcacccattaactcatcatttacattagatatatctcctaatgctatccctccccactccccccaccccacaacaggccccagtgtgtgatgttccccatcctgtgtccaagtgttctcattgttcaattcccacctacgagtgagaacatgcagtgtttggttttctgtccttgtgatagtttgctgagaatgatggtttccagcttcatccatttccctacaaaggacatgtgggctcctgaacttatccttttttatggctgcatagtattccatagtgtatatgtgccacatttgcttaatccagtctatcattgatggatatttgggttggttccaagtctgctattgtgaatagtgccacaataaacatacgtgtgcatgtgtctttatagtagcatgatttataatcctttgggtatatacccagtaatgggatggctgggtcaaatggtatttctagttctagatccttgaggaatcgctacactgtcttccacaatgcttgaactagtttacagtcccaccaacagtgtaaaagtgttcctatttctccacatcctctccagtacctgttgtttcctgactttttaatgatcgccattctaactggtatgagatggtatctcattgcggttttgatttgcatttctctgatggtcagtgatgatgaacattttttcatgtgtctgttggctgcataaatgtcttcttttgagaagtgtctgttcatatcctttgctcactttttgatggggttgtttgactttttcttgtaaatttgtttaagttctttgtagattctggatattagccttttgtcagatgggtagatttttaaacttttctcccattctgtacgttgcctgttcactctgatggtagtttcttttgctgtgtagaagctctttagtttaattagatcccatttgtcaactttggcttttgttgccgttgcttttggtgttttagtcatgaagtccttgcccatgcctgtgtcctgaatggtattgcctaggttttcttctagggtttttatggttttaggtctaacatttaagtctttaattcatcttgaattaatttttgtataaggtgtaaggaaaggatccagtttcagctttctacatatggctagccagtttttccagcaccatttattaaatagggaatcgtttccccatttcttgtttttgtcaggtttgtcaaagatcagatggttgtagatgtgtggtattatttctgagggctctgttctgttccattggtctatatctctgttttgatatcagtaccatgctgttactgtagccttgtagtatcatttgaagtcaagtagtgtgttgcctccagctttgttcttttggcttaggattgtcttggcaatgtgggctcctttttggttccatatgcactttaaagtagttttttccagttctgtgaagaaagtcattggtagcttgatgcggatggcattgaatctataaattaccttgggcagtatggccattttcgcgatattgattcttcctatccatgagcatggaatgttcttccatttgtttgtgtcctcttttattttgttgagcagtgatttgtagttctccttgaagaggtccttcacatcccttgtaagttgtattcctaggtattttattctctttgtagcaattgtgaatgggagttcactcatgatctggctctctgtttgtctgttattggtgtatgggaatgcttgtgatttttgcacattcattttgtatcctaagactttggttaagttgcttatcagcttaagcagattttgggctgagatgatggggttttctaaatataaaatcatgtgatctgtaaaaagggacaatttgacttcctcttttcctagttgaataccctatatttctttctcctgcctgattgccctggccagaaattccaacactatgctgaataggagtggtgagagagggcatccctgccttgtgccagttttcaaagggaatgcttccagtttttgcccattcagtatgatattggctgtgggtttgtcataaatagctctcattattttgagatacatcccatcaatatcaagtttattgagagtttttagcatgaagggctgttgaattttgtcgaaggccttttctgcatctattgagataatcacgtggtttttctctttggttctgtttatatgctggattatgtttattgatttgcacatgttgaaccagctttgcatcccagggatgaagcccacttgatcctagtggacaagctttttgatgtgctgctggattctgtttgccagtattttattgaggatatttttgtcgatgttcatcagggatattagtctaaaattctcttttttttgtcgtgtctctgccaggctttggtatcaggatgatgctggcctcataaaatgagttagggaggattccttctttttctattgattggaatagtttcagaaggaatggtaggtaccagctcctctttgtacgtctcgtagaatttggctgtgaatccgtctggtcctggactttttttgattggtaggctattaattattgcctcaatttcagagcctgttattggtctattcagggattcaacttcttcctggtttagtcttgggaggatgtatgtgtccaggaatgtatccatttcttctagattttctagtttatttgcatagaggtgtttatagtattctctgatggtagtctgtatttctgtgggatcggtggtgatatcccctttatcattttttattgcacctatttgattcttctctcttttcttctttattagtcttgctagcggtctatcaattttgttgatcttttcaaaaaaccagctcttggattcattgattttttgaagggttttttgtctctatctccttcagttcttctctgatcttagttatctcttgccttctgctagcttttgaatgtgtttgctcttgcttctctagttcttttaattgtgatgttacggtgtcaattttagagctttcctgctttctcttgtgggcatgtagtgctataaatttccctctacacactgctttaaatgtgtcccagagattctggtatgttgtgtctttgttgtcattggtttcaaagaacatctttatttctgccttcatttcgttatgtactcagtagtcattcaggggcaggttgttcagtttctatgtagttgagcggttttgagtgagtttcttaatcctgagctctagtttgattgcactgtggtctgagagacagtttgttatgatttctgttcttttacattttctgaggagtgctttacttccaactatgtggtcaattttggaataagtgcgatgtggtgctgagaagaatgtatattctgttgatttggggtggagagttctgtagatgtgtattaggtctgcttggtgtggagctgagtttaagtcctggatatccttgttaactttctgtctggttgatctgtctaatattgacagtggggtgttaaagtctcccattattattgtgtgggaatctaagtctttttgtaggtctctaaggacttgctttatgaatctgggtgctcttgtattgtgtgcatatatatttaggatagttagctcttcttgttgaattgatccctttaccattaagtaatggccttctttgtctcttttgatctttgttggtttaaagtctgttttatcagagactaggattgcaacccctgctttcttttgttttccatttgcttggtagattttcctccatccctttattttgagcctatgtgtgtctctgcatgtgagatgggtctcctgaatatagcacactgatgggtcttgattctttatccaatttgccagtctgtgtcttttaattggagcatttagcccatttacatttaaggttaatattgttatatgtcaatttgatcctgtcattacgatgtttgctggttattttgctcgttggttgatgcagtttcttcctagcctcgatggtctttacaatttggcatgtttttgcagtggctggtactggttgttcctttccatgtttagtgcttccttcaggagctcttttagggcaggcctggtggtgacaaaatctctcagcatttgcttgtctataaagaattttatttctcctttgcttatgtagcttagtttggctggatatgaaattctgggttgaaaattcttttctttaaggatgttgaatattggcccccactcttctggcttgtagagtttctgtcgaacgatccactgttagtctgagaggcttccctttgtgggtaacctgacctttctctctggctgctcttaacatattttccttcatttcagctttggtgaatctgacaattatgtgtcttggagttgctcttcttgaggattatctttgtggcgttctctgtatttcctgcatttgaatgttggcctgccttgctaggttggggatgttctcctggataatatcctgcagagtgttttccaacttggttcctttctccccattactttcaggtacaccaatcagacgtagacttggtcttttcacatagtgccatatttcttggaggtttgtttgtttctttttactccttttcctctaaacttctcttatcgcttcatttcattcattgatcttcaatcactgataccctttcttccacttgatcgaatcagctactgacacttgtgcatgtgtcacatagttctcgtaccatgtttttcatctccataaagtcatttaaggtcttttctatgctgtttattctagttagccattcgtctaatgttttttcaaggtttttagcttctttgcaatgggttcgaacatcctcctttagctcggagaagtttattattaccgatcgtttgaagccttcttctctcaacttgtcaaagtcattctccgtccagctttgttccattgctggcaaggagctgtgttcctttggaggagaagagacactctgatttttagaattttcagcttttctgctctggtttctccccatctttgtggttttatctacttttggtctttgatgatggtgatgtacagatggggttttggtgtggatgtcctttctgtttgtttgttttccttctaacagtcaggaccctcagctgcaggtctgttggagtttgctggaggtccactccagaccctgtttacctgggtatcaccagcagaggctgcagaacagcaaatattgcagaacagcaaatattgctgcctggttcttcctctggaagcttcgtctcagaggggcacctggccatatggggtgtcagtctgcccctactgggaggtgcctcccagttaggctacttggcagtcagggacccacttgaggaggcagtctgtctgttctcagatcttaaactgcatgctgggagaaccaccactctcttcaaagctgtcagacagggacatttaagtctgcagcttgctcttctttctttctttctttctttctttctttctttctttctttctttctttctttctttctttctttttctttccttgcttccttcctttcttcttctttttcttcttctcctcctcctccttctttccttccttccttccttccacccccgtttctgtctttcttcctttctttctgttcttcttttgataggatcttgctgtcacccaagcttgagtgcagtggttccatcttggcttactgcagcctccacctcccaggctcaagtgatcctccctcctcagccacccaagtagctgggaccacaggcacacaccactatgcccagctaattttttacagtattttttatagagatggggtttcactgtgttgcccaggctagtctcaaactcctgagctcaagagatccaccagcctcagcttcccaaaaaagcatcttgtttttactcgcatgctttgtccctctgccaggacagtagctgtaatcccatctttgccatctagcttgcccccagtggctggtggggcacaagttttatttcttgataaggctgtcatattatggctaatgcttataaatgcttcatgttggggtagagtggaaattgacactttcagccatgaagacctcaaattttgggactagctagcctgtattgcaggctttggtgaagccaagggcgtctcctggaagggctgtattcctttccttctgtacatttccttctttccctgattgtagcaggataagggtgaggcagccgaggtgcctgggctgcaccatttaagcaggcgtcactctcagggttgtggaaatgcgggacaggcacctgagggtgatagcctctttacaagtgtcttggggacctctcgtgccttgccctggttctgggcctgcagcctggccagcagtcatctgagtttacttctcattttcatgtcaagaaggaactgcagatgccaatgctctgactctttcctctgatttccccagtatcacagcttcctttgacaagtagtatttccgggttgcagtaggtgaccgtttcctcacatttttgccaacatgcaagaaaccagatttccagccagcaacattattgtccacatgccgagtgtccatctcctctcctcagccatttttccatcttggttctgttacttgtgacattctgtttccgtgtgagagatcctgcatcagttaggaatttcatacagctctaagtgacagaagcccccacccagtggcttcaacaatttgaggatttatttttctcttgcaacaaggaaggagttctggagggagacagcccagggctggcatggcgagtccctgagaccctgggtcctttgatctttccactccgcccatccctagcacagtttgccgccttctgtttacaatacggctgccccacctgtgacattacatctgtgttccaggcagagtgaattcacgaggaaaaggtgcataggagatgaatgtatttctctagtaagtagctttctcagaagctcaaccagtagctcacacttacatcccattggatactgttgtgtcccctggaaaccctgtctgcaagcggagctgaaatctagttgtttttgttttatttttatttatttaattttatctaagtacacggggtaggataaaagcttggctatgatggagagaatatctattgggtggttaactggtcatccttgtcacagctctgctcggcttcttttgcaaaccggtcacaccatgtgggaaggaaaatagccattgaggtagcttttggtggcctccaacttttatcctttgccttattgtccccaccacaggaagaaatatttttcttttagttcctgtctgtcagtctcaggggagactctgactggctcagcttgggtggcacatccgtccccgtgattatacagggacacgagcaaatgcttgtcatctccaacttatcatgtggagggaaagggatggcttcccccagggtgggacagtaatggagaaaaacagcttgcactgtaagtccacgacatttctgtcctacattttgcaagatgtgatcaatatgtattggcaaagacaaaatgagaggctacttttggtggagagcaaatgatagtcaagacaggaatgtgagaattgtgtatactgtcctgtaccagtcaggagcctagcttggctgcagaagagggctaataaagaatgaaataggtgaggcaacaaggagttggtagttgaaaagcatttatgttctttctagtgaggttgttttgtttagattcttctgcctaaagaggaaatgtgttacgctgaccaactatgcgcctccagccatctcttctacttgtttttgacgttatagttgttagcattgggttacttttgacagaaaaccccaaataatatttgtttaaaatatacctgagtttttttttctctcatatgaaagcacagataggcagtccaggattggcttggtgcctcatggtttcaggggattgtgcagcttctatctttctacttcctgtgtggcttcaacttcatggcttatggaaacatcacactctaggtggcaggatgaaagcagggacaaagcataagggacatctgaatgttgtcccttaaggaaggtccccagaagctgcccaatgacactcccactcatgggtctttggccagggccacggctagctgcagaggaggccaacagatgtattctgggaagctcggctcaaaacactatcttgtgggccaggcgcagtggctcacgcctataatcccagcactttgggaggctgaggtgggcggatcacctgaggtttgagaccagcctggccaacatggtgaaaccccgtctctactaaaagtacaaaaaaatagccaggcgtggtggcaggcacctgtaatcgcagctacttgggaggctgaggcaggagaatcgcttgaacctgggaggtggatgttgcagtgagctgagatcacactgttgcactccagcctggacaacaagagtgaaactccatcttagaaaacaaaaacaaaaataaaaacaaaaccccactattatgtggatgcagaagagaagggatattggagggtgtcagcaggctctggccaggctcagtcctctgctcagccttcacagccagagatcatggggataacaaggtcgtggatctcagtggacctgactgacatgaatctgtatttacaggagcccaagggtatttcttgcagggggtggattatacagctgaaagcagctgcttttacaggattacgaagagtagctcttttccacgaaacaatttagaatatacacagtgaaggaagtctcactaacttcctttaagaaagttgaggtgagaaagagtagacagtttgtaaatggaagttacctaactgacttctgcaacttggggggctttgtagcatcaacgggtcagaatttcttagaggcattcttactttttaggtctcaaatctctgtcatttttcccttacggtatttgcacagtgcagcctgccttctgtgacctacagaagcacattgcatttaacctgaggcttatttgatattacactttttttctttccttcaaatatccacaatctaaattgtgttcagaaagttccgaagagctgtaatcccagcactttgggaggccgaggcgggcggatcacgaggtcaggagatcgagaccatcccggctaaagcggtgaaaccccgtctctactaaaaatacaaaaaaattagccgggcgtagtggcgggcgcctgtagtcccagctactcgggaggctgaggcaggagaatggcgtgaacccgggaggcggagcttgcagtgagccgagatcccgccactgcactccagcctgggcgacagagcgagactccgtctcaaaaaaaaaaaaaaaaaaagaaagttccgaagagttgccagggctctatttaatccattgctcttcttagcaatgcaaattctgtgcacaatgagtttattctccttgagaagataaattcagtttggctccagtggctaaactttgttggacaagtttattcctgtgtgacacctacctggccaagcactgtcatttggatgggtctttgggcaggtaagtttctgtgcacctagaaaccttccctgctgggatcttgtcccttgagctcctcctctccagttataaactctgttggcatctttataggatttactttttatgtggattccattgtctccaaaatgccctttcttagtaactagttgggccatctacaccctctttcggggtacgacatagaaatggaaatggaaaatttgtaaggaattatcataatgttaaaatacagataccttgtcattaaaccattaaaaacaaaattttaaaaaccttaagtgttggaaatattaggattccaatgctccttccacttcctgaaggactcatttctctaacatacttgctgaacaatcctcctaccttttccctgcggcattactaaataattcagattgtttgaaaggcattctcttctatcgagatgcaatcaaatttgaggagtgtatttcattctcgccgaatgcattatatcttactttccataaagagaatccaggtatgtgctattgccttatgccttgaataatatgaagcgaagcgatcactcttatatttggggagtgttttagagtcttcagatgctttatgtagtatcaatggctccaaatataataatgtacaattaagaatcacataggcaggcatttttataatgaggaaaagtgaagttaggagagctatgttcactttccaaggctacatagaacgagtgagcgccaggactgacagttccttcctgctctggatcttgtgggctgtctcctccagcacactgcatgctagaggaagccagcgatggggcaggagtggggcgagcatggcagctgaccccaagatgggcagagagcctgcccctcactgttcttccccgccttaaaggacataatctaacctctggacatggactataatctagcctcttctttattacttagggtaaatagtacgtctactgattattaattaaataacattcttcacctgtacatgttatcacactttagttcttagactgctttcatattcaacgtggagatctggaaaggcaacttgactatcttcattttatgattgaagaagctgaggttgagggaggctaaatggcttgtagttacactacaaacactaaaacagcaaaaattagcattggaactagaacatctgatgcatgttcatgactttgtcctcacagtcatatcctcagggtctgtggagtggtgtgtggctgtggccccttagttagaaggccgcagggactgtggtgttttaggttgtgccggctcttcgtcattttcccttttctctcctgtcccttccttccaggctatgagctatatttgtgccagcaagcagtgtttctctctgtttcagctttttcctactttaatttgtttgttttcatctgttaggatcttagagatgagagaaccaagatgatttggaatttcatgtgagattacagaggatagactcagagctgaatggtagcctatcaataacgttcatacatacactctctgacctctttgttggtctccaagaagcctttcagatttagttcctggttgtgggttgtgtttgaggtaggaggaccttgttgatggttgcattccaattaaatgagatggcagccctcacggtgcctgtgtgcatacaacatgtctgtgttagatgtgcatttgcaagtaactggttatagaacattattttggaatacaacacttgtgaaatctgaagatggttttaaactttgtatttcacctgcaattatcttgtagtactctcagttaggagaaaaatatctcttttgctctgaggattcctcagtatttttagtgactctttgcatgcttgcaagggctgtgagaacaagcccagcaggaaggaagagacatggggggaaggaaggaacgtgagggaagtgagaagaaaacaaaggcaaagtttcatacctggaaggcttcgtgcaccagcgcccacctccggaaaccacagttgcaaatattcttttgtgacctaattttggttctaggtttcctttcctctgtatgtgtttgctacattccattccctatgcaatagagtttttctgtaaagaagtttccaagtataaccttccatgaagctcctcaattttaagaatttataattttccagtcctagtgcttaactcatccatcatttgttttccctcatattttacctgtttggttgcactcaaagtcttcgttgatggatgaaacctgatggtttttaagaagcagaaaataccagttttgtcctcactgattgatttttcttgacagatcacgagtataatttataccagctcagtggcactaatcttgttttgttgttgttcttaaaacagctttgacaataagctgctactctgactttgattcttaagtcagcattaatgaaacaccattcatttgtgttatcagctgtaatagtcaaaaacaggtttaagactttaccatgcttgctttcaagttttgggtggatttctttttcctccaatgcttaaaaggtttagtaagtgaatgtcaggtcaaatgggcgtagctgagtttctcccaggcacgctggctttcctgtggatgtttttgtcacagcagtctaagtgtatgtctttctggcccggtcagggaactttgctgtggggatttggtttaaattcatgtttaatctcctaattttatttttgtggctatagtgcctcaccgagtatactgaggctgagactggacagaaagaccttttttctgataagctcttaatctgtatcaggctcagccaaaatgagttctgttagggctgctatttttagctatgtgtaatagctattgtggtctcacaatacacttgtttatacacagaatatggttttatgcacagaattttgtgtattaaaataataggcaaaatgattacaaataatttcaggtgtgccttgatacaaatcagatctgtatacttgtgttttcctgttgacatctgtcatttgcagtcagtggcttaaattaaaattttttggtcttagggaaaatcatgtgagggtttccctgttgcctctctcgggcagagcttctcagccttgacactattgacgttttagaccagacacctctttgctgtagggggtgtcccgcgcatcgtaagaggtttagcagcacccagggctctacgtactagatgctagtgcctcccctcccccactctcagttgtgacaatcaaaaatgtctccagaccttgccagatgtcccttggggatatagggctgccagagttaacaaatgaaaacacagcgtgcctagttaaatttgaatttcagataaaccatacaaattgtttttattgtggtaaaatgcacagaatataagacttaccattttagccatttttaagtggctaaaatttgtaagtggtcagtgtcattaaggacattcacattgttgtgcaaacatcatcaccattcatctccagaactttcttatctttgctagctgaaactctgtccccattaaacactaagtccccatcctcccctctcccagcccctggcaacctccgttctcctttctggctctatgcatttgactcttctgggtatctcatgtaagtagaatcatacagtatttgcccttttgtgtctgtcttatttcacttagtgtaacgtccttaaggtttgtctgtgttgcagcgcatgtcagaacttccttccttcttaaggctgaataacattccactgtgtgaatgaaccacatctccttcatccgttcatccatagatggacatttgggttgcttcccgcatttggctattgtgaacgatgctgctatgaccactggtgcataggaataattttttagtgtaaatatgtcccatgtaattttagtacatacttttatactaaaaaattattcattgtttatctgatgttcaagttaactgggcaagttgtgttttattttattgggtaatgctacctggttgagaaccactgccctaggactgaactgctatttttttttttttttttttgagatggagtcttactctgtcacccaggctggagtgcagtggcgtgatctcggctcactgcagcctccacctcctgggttcaagcgattcccctgcctcagcctcccaagtagctgggactacaggcatgcgccaccaagcctggctaatttttttttttttttgtattttagtagagacggggtttcaccatgtgggccagggtagtctcaatctcctgacctcgtgatctgcccgcctcggcctcccaaagtgctgggattataggcgtgagccactgcacccggccagctggactgctttctttgcttgggcactggctcagggctccaaggaccgtggtcataccttcccatgcaacttacatatgacttccaagacttccagcacagtgaagagcacatggaaaaatgtgggcaccataacatctacataccctaaatgtgtcagtcagcttcaatttgttctcagtttgggaagggtgatagagatactgacccttccttttgacctgcctcttttccattttattctggtaaattcacgcttattacagcacttacagagcaaatcatattatgattataattttatgatatcatacttttatattatgaatatatttataatatgtgaaatgattggattcttcaaaaagtcaacttaaagaaacaagttgggatcctcactggctacttgtgtcctatttcaaattatttctatttgccctttctgctcctctccccagctttattgaggtatgctggacaaataaaaattttaagtatttacagtataaaatgtgatgatttgatgtatgcatatgttgtgtaatgattaccacaatccagctaactaacatgtctatcacctcactagttacctttttttttttttttttttggagtgtgatgagaacccttgagatctactctttgggcagttttcaagtatatgacacattgttattacatagagtcaccatgctctgcaatagatctccagaatttattcatcttgcataactgaaactttttaccctttgatcaccatctgcctttttgctttttctatcactcagagaaagccagttagtggataatttctacatgattgaggttaatagaatgcggccacctaaaactgtccctgaggcttgggaatttgggtgaatcctgattctgccatgcactttgggcaagtcacataccttcagtctcagtttcctcatccgtgactaaagaatagcatgaatgccttctccatggaatcaccatgagaggtaaaggaaatattgcacttacagctcctgctatgaaatatctagcatctgacagacatgcaccttgtcttagatgtcgagctgcagagcagttttcatggcagaaagagagcaatagttgctaacacgtatgaactgcttactgtgttccaggtactagtctccagtttttacttgtattgttgcatttaatcctcacactgagcccacgaagcttgtcctaacattattccctttcacagatgaagaatgtgaggcacagataaggtacattcatccaagggggtatgtctggcaagcagcagagcagggcttggcattgggacatcctgattccagtgggaatagttgtcctaaccacttggctagatggctgcctgtggggtggtgcttctggatggaacacagggtgcacagtgatgcatttgggaatcactgaggcaggagagtgaccccacccagccctccaggccagccaggataaccctggagggctggagggggacacgtcctggtgagtgcaccctccctctgggttccaggtgagatacccttccagctggccttttgagcccagctcccccaggaaggaaggatgctctccagcaggcgctcctcagagcaccttctgtgttttcacctcacttagaaaattcaacctctaaacatcattgagtggcactgagaactaaagccacaaactcccaacttgagaaaatgcaatatttatacatttaaaacagccaaaatggttttaacatacctatactgattcaaaaaagaagtctgagccaggcgtggtggctcacgcctgcaatcccagcactgtgggaggccgaggcgggcagatcatgaggtcaggacattaagaccatcctggctaacatggtgaaaccccgtctctactaaaaacacacacacaaaaaaaaagttagctgggtgtggtggcatgcgtctgtaatcccagctactctggaggctgaggcaggagaatcgtttgaactggggaggcagaggttgcagtgagtcaagatcacgccactgcactccagcctacgcaacagagcaagactctgtctcaaaaacaaaacaaaaaaaaccaaaaaaaaaaacaaaaagaaaaagaaatccgttttcgctgagccctcctggcaggtagtaaaatcttggacttgagctccctggggccctgtccagttgccctcagtgccctcccttccagcttacccatttaccaggtgttcgaggctgctgccagtttaaattttgtttcaaaaactggccttctctttctttttattctgcaacattttgccttggggcctagaaaggaatccttagtcactgattgccgtaggcggatattacctcacccagtatgggcttccacgctaaagacctctcacaaacacttgcacactttcagactcaccctgcacaaggcacgctgacgctgtttcctagggcttattcccaagaacacgtctctgctgagacttggtttttgttaggatgtgtgtgattccatgtgttccatcatgcagaggagatgctgaaagttagcgctgtggctttcaagatcctcctgtggcgcctttgtatatggcatagaatggtgccaactggagtagcctgagaaccagaaacaagaattctgcttctgaacagcttcgtttttgggccagagaattgtttccaccagaggaggcgtgatgggagcttaggggctatgtgctcggtttctgaagttgtacaacggagcattcatttgcattcatttgacaagtcctgctgtgttcctttacttgttctcttctgtattatgggcttaagggtcgcttatttcaacatcgctatattttccctgaaagctcagtaatgattgaatcacagaatttaaagagcttgtcgtataaatacatctaaagtgaaaaagatgaactttttccccagtgaatacaggcaaccatcaatcaaaggaatgcatcctggaaaagaaaggcacagggtaaagcctgcctcctttgcgggtcccatctgttcttgggaggacagtccccatgtattgctcctgggccctttgtggccgagcacgaacagctgtacatacaaaggaggctgtgtgtttgttcatgatgttggaaccggaagtggaaggggctttcttgtcaaagaaggcacatatgagtgccatagttacgggacaaaaatatttctcctggaaaatctattgaagcctttgtgcgatgttcagcagagtgtgttgggggtgggtgtgaatgtatctgtgtgggtgaaatgaggataatgtggtcatgtgaacaattcaaggcaagaaaagatccggtgatactgagaatcctgattctcctttagagataggagttctctggctattctgaagagtgaagttactgcccatgatgttgcaacatatggacatttgcaacaattttttttttttttttgagatggagtctcactctgtcgcccaggctggagtgcagtggcatgatctctgctcactgcaacctccacctcccgggttcacgccattctcctgcctcagcctcctgagtagctgggattacaggcgcctgccaccatgcctggctaattttttgtatttttagtagagacggggtttcaccatgttagccaggctggtctcgatctcctgacctcgtgatctgcctgccttggcctcccaaagtgctgggattacaggcgtgagtcactgcgcccggcctttttttttttttttttaaatgattcttttgttccagttattgacctagcaccagcatttgtcttatggataaggtgagggcaaaattgcactgggtgtggcaaggtgattctgcacttagaggttcgtgtttatggtgctactgagggactgaagaattcatctggaaacttttggctgctcccctatcctcttcttgctaaatcagaatggcatacagttaaaaatacgggccccaacccgaacacaggatttcaaggcttgctcactgcttgcatgatcttagaaagtcacatctgactccaaaccttcgtttcttcatctgaaaaacggtgacgatgatgacagaactaccacctgcatggtgatgctgtgcactcaggaaggttttagaaccactagagtggactggggctggaatggcttctgtcacagaattcagagttcccttgtactgcagagagtcaagtgcagctgaatggctgctaccagggatattacccagatgttcacaggtcatgaaagtgtgggacaagatgacctcagagcttcccctccactggtgacaacacatgtaacatgcctcctattaagcctgtaatggggtaaattgtatccccaccaatgataagtccaaatcctaacccctgatacctgtgaatgtgaccattcttggattataagatctttgcagatttatggatcttgagataagatcatccttgatttaggatgggccctgaaatccaatgattagtattttttatgagaaaggagtgggagatatgagacacagacacacagaggggaatgacatatgaagatggaggcggagacagtagtgaggcatctcaagccaaggcatgctacgggttgctgacaaccaccagaagctgggagagaggcacggaaaagattctccctcagagcctcagaaggaacccactggactaacaccttgactttggatttctggctgctgggacaatgacggagtaagtttctgtagttttaagccacccagttttcgatactttgtcatagcagcattaggcaactaattccatatctcacccataataaacactcagtgctagcaattggctgccactgtggtgattatcagcatggaactatgtgttcctgaagagaattagttattccatttctatgaatattttcaacccaagattgttgctctcagttaacgtactgaaaatgaacttggtgaaaaagtaaaagctcttctacatatcccatttgtggatagacttaagagaacagaaagcttattatggaataagcaaattaatacctttgggatcaatagagtgtctacagttcaaaaccctatcaaaagtgggtttacgtaaaaaaaactagtgtgttatccatgtgtaatattttgtataatatacaaaattctggaaaggaagactcacaatgccagatcaagtattgcaacgtcagttatggcaggaacaaaggctcttcccagaagcctgtcttcattgtgcattctttcaccagaacaacttgctgtacttaaggcacaggacatttggatctagagatgtagctgttgtgactcaccatttctataaacttggtgactggtcccaggctgtcatatctgtacctgatccactacagggatgaattgacattttcagtggctactttgagtccagatattaatagatcagcctgaaagggaggatgaggcagtaatagcacttttacaagcaatagaagacaagctcagggaacaaaagagaagggaagctccttgaatttaaaggcaagggcagagatttattcctccactaaatctaaggactaaaagcttaggtggagagagggctcaaatgtgaattcacttctcatgctccctttgtgacctcctttaaggaaggccagatttgaggaatgagattttcccaaggggaggaagcattgtcttcatcacttcatgtatttttctcagaaggctattggcctcctggaatttttacttagaattccagatagtccaatgatgctttctaggtaccgtggctttcactgtggtgagagttttggctgcagtgcttttctgtaacccactcacaaagcctttggggatggtgatagtatagggattgtccttccaactagttgctctccaactagaaaggcgaatccctttttctttgagtcacagaggatggagaggctcagcattctcattccctgtttgggtttccacccctgtggccatgcaatttccatttatagccatgtattggcttacaagcctaacaccaatcctgacccaaagcatgaggcactctcacacatttctaggttaagaacactaaaaagcaggatcatcaggttattttttgttatttgtagatttctgcatagcctgatgcttcaacttcccaagacctcatttcttcccagagtagctggagtagctggacctttatcatgttctggtatcaaattctgccttggtaatgtgtacaatgcttcaagaattgcggactccatattactctcctgtgtcatgtagttctctctttgtgaaagccatccttccctgaaacccctcatccacctgcctaccttgccaacattccctgctcctgggttgcccttcattgtgttctatttatgggtaaccatgctttgccaggaaccatgccaagtacttggaatacgaaggaagtcattgatggagaggacacacaagtaagtaaagggccctgataaaatgcgacgagagctaggcacgtggatttttatgcaacaaaggagataggcaaggagcccacgaactagtttccaaatgctggacccccttgatcaggaccttttcggagcaggctcaagtgtctcccaacacctatacctggctggaccccttgccattgtctcaccacaagttgatgctatagttccagagggctgccccgccaggccactctgtcttttgcaattccagggtgggatgtgcattgtctacttggacctaggaactcccttcagcagttctcctgtggacctgtatccaagccaccctacaaagtgagcatgcactctgatttaggaaacttttcttgagagatttattctatagtctccctaacatcccattctggtgtttattccattaatttagagcaaacacccattcagtgaagggccctgaaaggcatagaaaaggacaaagtatcagacttgatttctgctctcagctattaaaactggagaggcaaggcacatagaaagatagggatgacatgatatttaaacaaatgttctacacagtgtttctcaatataatgttcttgtcctgttggggggcttttcagtaagcagtgtcagtgaaagggcagtgacagcaggagatgatttttggttattttctgcataggcatcctcagtatggaaggctgtgcccacctacgtttgaaatgtgcaagcttgattcttctctcttttcttctttattagtctagctagcggtctatctattttgttaatcttttcaaaaaaccagctcctggactcattgatttttttgaagggtttttcgtgtctcttatctccttcagttctgctctgatcttatttacttctcgtcctctgctaacttttgaatttatttgctcttgcttctctagtcctttttattttgatgttagggtgtcgatttgagatctttcccactttgtcctgtgggcatttagtgctataaatttccttctaaacactgcttcagctgtgtcccagggattctacaatgttgtgactttgttctcattggtttcaaagaacttatttattcctgccttaattttgttatttacccagtaatcatacaggaccaggttgttcagtttccatgtagttgtgcggttttgagtgagtttcttaatcctgagttctaatttgattgcactgtggtctgagagacagtttgttatgatttccgttcttttgcatttgctgaggagtgttttacttccaattatgtggtcaattttagaataagtgcgatgtggtgctgagaagaatgtatattctgtggatttggggtggagagttctgtagatgtctattgcgtctgcttggtccagagctgagttgaagtcctgaatatccttgttaattttctgtcttgttgatctgtctaatattgacggtgggatgttaaaatctcccactattgttgtgtgggagcctaagtctctttgtaggtctctaagaacttgctttatgaatctgggtgctcctgtattgggtgcagatatatttaggatagttagctcttcatggaggcaacctcaagtagcattattgttcttttcatccttcctctcctactcacctcccccactcctgacaaaatatggaaatcctgtccctgagtagtcttgttcacaatacacaatctcacagccaaaggaatgtatttttaaccctgagacgccgaaaacggcttgaatcattcatgctgtcgtctattagtctgccgctgtgctgttatttttcccaggcttaatgagccctcatggcactgcgctgcctcagagcacggccgcagcttcaagcacagacaagcctgctctttaaatatgcaagtcatcagcctttcttggaaaatgctatggggaaggtgctctggacaagcaaacagatgatttttgcagactgttctgtagttaatttgggtcatgaagcccactgtggctcaaggagtttaggggacagaaaaggggcttttgaaggattaacaaccagataggaagaaagaaacaaatgaagggtaatcctggttttcttttgagactgacaagaaaaagttgtttgaaaggaggacactccaccattgttgttgttgaaggagagctgtacttctgatgtcccccaggctttgtagtagttaaggggtgagggattgcaggagctgctcaaacctggctgctaatatgacactaggagcctgggtgacacaggggccatggtgccagctgcaggtagcgtgggattgaccctgttagccctggacatcaagcctggagtgagagatggcccgaaaacccctgggacctgatacaagcaagggctttgggatcaaccaggctctcactaacaggcatcactcaaaaagaaaagcatttatttaatagagagtttcagaaaatgttccccttattatttttctttgatttttgaattggaaggcgcttagagatcctgtcctttaacccttgcgttgtgcagatgagagatgtgaggtcagggaagtttagtgggctcagggacgcacacggagttcacagggtagcccgatgcagtgggagggtcgatgggcttctgcctcagagttcttttcaccctaacatttcctgtgcatttgtttacgtgtgccagggatcgctctaagtgctttgtacacattaaacttatctaatctttaccctaactgtgagaggtggctgccatcatttttatttaatttcactttgcattaaatgatgttggcacagtgatgttaagtctgttgcccacagtctcctgcctggaagtgacagtcaggattcaagccaggcagtgggagcctctggagcagccctccaaacctctaccccatatggctcacatagcctctaaaaagggttttgtgactgatctgagatcctaaccattatctatgacttcatattcctgagcagggggcagctggggaagcatttcaagatactcaaggacaaagcacccatcaacatagatgggtgcaaactcaatgctatctagttcgctgcagctgtttggctaaaggaaaacagaatacctgcagagctgatggcttggagactagatgtacaagattacaagccagatgcaagtctagtttctggggtccatctgtgagcagaggtgtgggtggaggcccagaagccaggctaccatttgggattgactcttagctccttacatgcacttaagcagccattcatgaccttttctgcagctcaactgatacactcaaggctgggcatgtccttctgttctagtcattctgcttttggtccacttctatatgtttgttaccaaactagacattgtcttcaagacccactctggccaagtcctggggtcatgtttagtcctggaaggacctccaggagatggaggtagttcttagttgaccagaggggagcaggaccacctgtgctttaaggtcatcctgatttcctggtggtcgttaatacgaaagaagctacaccaggcttgacttctctgagtcagtggtgttcagagcccttgactcaccctggacctgggcttggcaggaatttctctaaccttaggtggatgtatgggtgagtctgcacagcaacttactgttctggtgcatttgtgttggcatgcccatgtaccgggagatgccctagggacttacttatgacatactgacctagagagggtttgcgtataaaattttaacttgactcactggttcctggcagggtcaataaatatttgtggactcactgagtgaatgagtgagtgaatgaatgaaccagtctgctaagaagtgtgagcacagaatatgtgtgctgttctgacacatgatgaccctatcatcctttgaggacctaccatatagctcagttttgacactgcttgtgatttttttttttttttgtctttgagacagggtctcactctatcacccaggctggagtggagtggcatagtcataactcactgcagccttaaactctttgactcaagtggtcctcctgcctcagcttcctgagtacttgggaccagaggcgtgtgctaccatgccagataattaaaaaaaattttttttttatagacaaggtctcactgtgttgcccagggtggtcttgaactcctgacctcaagcaatcctcctgtcttggcctctgaaagtgctggttttacaagtgtgagccaccaacccctggtcccacctgtgatatttttcaccagctcatggccgtggggagcccgactatttgcatacgtgttttttgctcagtactgacacctggtggcagtgtcccaaattgcaagcacagtctctagcattaactcagtgtcaggactttggtgtggcggaccataagaatgggagcctctggagttatgcagagagaagtagccctaccagggaagatgtaatcttgcaaaataacctacctaatgacaatgagtctcttgtcttttcttagggctgcataatcagcaccacctgatttagtggtaagaggaaaggtgctagtgggaaatgccattatttaaaaatacctggagactactgatctagtttaagaccaatatttgtcccacatttgatattagaaccaatagaatgatagaagtaatagctatggtactattaatagctcctattgaggacctgtgaacacactgctggaccctgtatatttagtatttctaatcctcatgataacctaacacatagggattattatttccattttacaaattaggaaatggagctttggtttagtaacatgcctgagttcatggtaggggctggatttgaatctcaatttgcttgacctcaaaggctatgctctttcactctgcttcaagggtactgtgttactccaggggtgccattcacattctagtcagtgtacatggcatttctggggttgtgtgacttgtagccatcctctttccatcattcccactgcctctccaggagccaatcatttatgtctcttctaagctcagtgggtgactgaggctgtgaagacatttaaaattgaataatgtatcctgtagtagatgcattgactatttctctagagttggcatcctaaaattcaaaatttttggtctggcagcctgtgaaaaaaatcgtattttgcctctaattctcaaccctggagagcgccaattagttgtgaagttactttcaaactgaagcccaatttatactcctaataaaaacatccaatgtaatcatatgtgaaatggaggctaatggccctataccacccgagcatcaccatagtgatggtgttgaggataaaaatgatgacaatgacaacagcagcgatgataatagttaaccattgtcgagtacccattatttccttgactccttatagacactctgtgagggaagtaatagtattgtcccaattttcagaggaggaaatgaggtgtagagatgctagttatcttacccaagctcagttgactgtgctggtaagttgtgaagttaacctaggttagcctgagttcacaggctatgctagattaacttcagctagccttgcttttaaccacctacactgacagcctttcaattgtaagaggagctttgggtttttgctttgaaagtagaaacaagtatctagtgataataatatgcctaataattatcctgcacaaaataaataatccagccacttatcaccacctctaaggcaccttctcatcctgtcgttttaaggttttgttcttttggttctgtccccagccaggttttcttttccctccacatgcctggtgtggtgtgagtaaagcatctgccattttgattatggcacacttgctacctcttcttaaaacattcgttctcatccctagtattgtgacattctatcatatctctgatttttaaaaggacattggtgaatttccagaaaaaaaattttccaacctccttttaacctggcatcttttaaaattaaatatgtctctccaaacatattggtggggaagagttggtgtcatgagttaagtaggggtataattacaacccgaaagtttgggggcaaaatggagcagttccctcattggtcagagttctgtcattggcccagcctcctgtcctccacaaatctgagtcagggtgagacaccaagagcctcccttgagaccggcactacttggaaacatgactggctatttctgctgcaggattcctggtccgggctccagcaaaattattgttgtgaacataggccttgaagaggaggctgaagccgagaagccaatattcactcaccttttcaaccttcgacaaggctgaaatttctcccatgatatcactggaaggaagggcagagggagtcaacaatgaagaaagtgggaagagggagggagaatccaggcaggaaaaggggggtgaccgtgcagcaatagctgtgagggcttcccagcagcgttagacacaccctttgcttcagggtcgccatctctgtctgcatctccttcctctccttttgacccagcgtccacttctactttggttagttaaatattgaaataaactcctttacaatgtgtggaagtctcagctcctcagcattgtatcttatgggaatagttactcttcagcctccgtgtgtatccagggcgtggaggccatgctgtggggactgtgtgttaatgtccttgtagctcaagaacagctgctcttaaatggtccgtgctgcggtttagtggacacaacatagcacagataagtgaagaaacaagtcatttttaagtgacgcaaatcacagtagaaatatgcaacttatggaagtgaaatccttttagtattaatttttcttaaatagcaatcctttgtagttgtaagatatgtaacgtatatattgttacctagttggatgaaaaacagctaaatcaatgattgttaggtttcttaggtgaatgatctgtttaataaaattattggaaatgcatattctttattttgatcaggtggacaaattgttccatttcatagttacaccctgacttccatttctgaagagataacccaaaggttactggtggtgatggagggcatcccagtcctcccctcatttgaggggcagagcttaattctggattgtgcttcctccactggagttgatttagagagcggtttggtgaaaaccgaagaaagggtttatctcagagcctcctgctcttgtttgaacacgctgcagggattaacttgtggttgggactgagccagagagataaatgacatagcccttgtgtgctcacattcagagggagctcttgccaaatgggacaacgaaggattgtgttaaagagatttctgaacatcttaatacagcaggatgatacagtctgctctctggactttctggggagcactgagccaagcctcagaggactgtggcatgtcgtgagtgtttaatatgcaagtgacatctaacaggggaaaacaagacatttggaaacaccattgggtggggtgggtcccccgcatttttcagcacggattttttccaacaatctcatatatacaaaggaagtaaattcacaaaagattgggtgttgggggcgtggggcgtggtgaggaaggaggaaagcactaaattttattagagtgcttctcaaaattgagagactgaaatttccaaagcaggaaagattacactgaaagttgggaatgtcttacaaatgtacatattgtcctctacgcgtgatataaaattgttcagcaatagatatttcaggaaacgtaatcagtgtaaaccctttgatgaggaaagccatgcaaacatttttaaacaattgcatccttatgtaatcctggtgttaaggggtgggcttaaaaagccaattcggtgagtagagggactcgtaccttatacagggtttggcgtctaaagctaacacctaaggcataaattaagcacatttacaattgcccttgaaaattccttaagttgcccataaagtacacaaggttttgtgtgtttgggtcttttcagtaatataaatgtggacgtgcacaatggacaacacttaatagaacacacatgcaaaatagaattttccagaatttttattgcacaaaacattcacatagagtgcacctggttgaacgtatggaagttacatgcgcatgtggtatgactgcagttgtggatagacctttttactgcgtcctcctctctagcacctaagtggttcctgtattcttcccaacgtaattaaactgcaggaggaaactggccattccaggtttagacagctgcccctttgaaagagaggaaagtgtgtccatgctggggagaggcagctgcaggaaacgtcctgagcttgtgaaagtctgaccccaggaaccaccctgagctgacctcccaacattctggttcctactcctcaggctcagaccctggttggttcacggccctggggtcatctacacctctttggccttgaccttggacattcggcccagccaccttccactccacttttttttctcatagattcaccgttttcatcctgcccatattctcccagcatccagccaatacctagttttcccagcatctagccccagttcctagttctgctgcccctgctctaggctggtcagttatatagcgttgcttccctgcaggtgaggtggcatgtgacagggtagggtaggtctgacacccctgcactctctctacccacaccgtgaaaaatagtgagacaacagcattgcattcttcattttgaaagaggtttgaatgtttttatttgtggaaggtagatgtcatgaagagttgcataattaaactagtacagaggaaaaactggcagagaagtgagttgcagggctaggtttacatacaggatgacaaacctgtctctaaagatcccgggaactgttgtcacatggtgacagcttatgatggtggctgaggacactgctctttacctttgtgtctcttgtctcgtctctgaatctcaatacctgtaattaagaaggagcccataggagttttatccatcttattttcagatatttaagcagaataaatatattttcacaatcctttgaaactctcttccttcttaaaccttaatgagattccaaggtagcaacaaaggcctttctgatattacctgtacaatcaaaaactgagcttgaacttcctataatttcttctttttggtcaaaggagtcaccctccaactgacatatttataacatgtccctgtgataacgagaaactactcacttatctctgagtctcatgacagaaagagtatcagaaaaaaagatactacaatccttcaaacacacagcagtgtgagacaaaaattacactctaatctgcagttgaacaggttctgtcttccattaaacctgtatccccattttattatatagttgagccagttatatatgactcactgggaaagtgcattttatgatcttgttagaagaaatggtttctctgggatccttttaagccaataattttatacctagctcctcctaaatatctaggacattaacttattagccattccggtgtcaagcctaatgctttttaaatacttaaaacaaataccaggaaactgtgggggttggagaatagattttccataggcatttagtgctttcagagatcagggggatcgatctcaggggatttagtaggaaaagacttgattctctgcttcctctgcacacagagctttcattttatgcaacaggagggacagcatgcatgtgggataaagccaggctgattatttcaaaaccaggctcacatctatttgactgacaaggtcctggcccccggccagggttacagcaccggaacatggtctcttctctgcggctctgctggctgggcctgtgtggaggcgggtggatggggggatgtccaggctgcagctgacagggcacttggagatctcagaaggcagtggaggcttggggactgaaggggacccggcttcgatggggtggggaagcacagcagcttggtaggaaatgacctgaaagagggattcctaaacatgcatgtatgtttgcgtgtgtgcatgaaagcagctgtagatcacaactaatcttttttttttttaattgcaaatcccactcaattcttcaagctttcttggcagcatctgatccagttgatggctttggaaacacttcattcttcattggcttctatagacatctcctcccgggggttcttggcttccctgaccactccatctctgttctctttactggtccttccttctcatccgtcattttaaggttttgttcttttggttctgtccccagctgggttttcttttccctccacatgcctgatgtggtaggcagaataatgaccctcccaaggatgtccatgcccttatccccagaacccggtgaagatggatgttacaaggctaaagggattttgcagaagtgaggaaggttatggatcccaaggtggggagagcatcctggtttatccagatgggcccaatctaatcacatgagcccgtacaagtggaagactttctcaggctgggcacaggaggagccatgaggcagagggagtgacagtgaggtttgaagtgtgagaagtgctggatgacatgccactgcaggctttggaggtgaaggggggcccatgaggcagggcaagtgggcagcctctgaagctgacagcacacaagaagaggaggacctcagcctagggccacaggaaactggattctgccaacaacctgaccaagttcagaaatgtcttgaatcccagagcctccagaagagaacatagccctgcgttcaccatggtttcaacctgtgagactgagcagagagccagccgagtccttctgtgttggacctctgacctacagaaccatgaaaccaatcattcgtgttgttttaatctgctaaatgtgtggcaatgcagtaccacagcgataaaaccaatccacccaccaccaatggtcttatgattctaatagcttcaattaatcctataaactgacagccggtccctattccccattctgctgaatcccaaagcctcacgactttcttctgttaggcaactctgcttggctgtgttaaaaacacatctaaccctacatgttagaaactctgccagctgacaaaaccagaaaccttgagtcagtctggattctttcctctctgttttattcattgtgtaatctcaactccatcaggtcttggttgaaatgtcagcatctcaaatgggtcgcctccgaccacccaatttaagatgacagcccccctcctacacgtaatcgtcctttatgcttatcagcatcagtgtattgtatgtttacttaccactttgcttattattagctgtcttccctcttggaatgtcagcacatatgtgttttattgtattaattctatttaataagtagtaatagagtggtttccaagtgtcaggaactgctctaagcatttttacaaatatccacttatttaatgctgttttttcaccgtcatagctctagctgttagaatagtgcctggcatgtagtagttgctcaataagtagttgttgagcgaataaatgaataaatgaatgactccagcctaacacgaagtcctgtcaattctatttcctaaatacctttcctaacaacccacctttctccatctgtaatgaactaagaaagtggccgtatcattctcctttgtagggcccttgaatggcttccctttgtttgtgggagaaaatccacactcctgcagggattcacacacgtttcttgtcatgccctcagttttctgcctccatctttatttctcaaaactcccctctcatcctctcttgccttccttacatgctaatcttcaatcagcccggacattcagtttccctaatatgtcccatcctccctcttcttccaagcccctgagtatgagctccttaccattggcattccttcctactacctgccctgttctctgcttattctatttttttttttgagatggagtcttgctctgtcacctaggctggagtgcagtggcgcgatctcagctcaccgcaacctccgcctcccaggttcaagcaattctcctgcctcagcctcccaagtagctggaatcacaggcacgtgccactgtgcctggctaatttttgtatttttagtagagatggggtttcaccatgttggccaggctggtctcaaactcatgacctcaggtgatccacccacttcagccttccaatgtgctgggattacaggtatgggccaccatgcctgggtttctccgcttattcttaatattttaactcagctgtttccctcctccatgacaggacacctccctcacctccacctccactgagaaagtgcctttcctggtgctcactgctattctatcttgtcctctgtccttcccctctctctgtgtgtggtaaagactgctttccctgtgcaattctctcaccagtttgcacggttcttgaggccatgagctggagagtacgatccacttgggcatcaagtgcaccagctggcacctagcagggactcagcacatacctgccgagtgagtgaattggtttcccatggcttggtgagccccggccaggcactctcccgagccaagggggacacttgttctgccagtgttgatttgcaggcgcaattatatgtgtaaatgcattttcactgttctgccttaggacccaatttttgaaaattagattttaaatgtcgtattctcctcacttacatcttatgcttcccgacattcagcacataatttaaaatgaaaggcatagaaaattggactcatttagtttactccacttgaaatccatgtgagtctatttttaaattctcctagaccctgtgctcttttagggtgtatttgagaggaaggaagggggtgggggagaaaggaaagaagacactcattgtaagctgttagagatgctgcgtgttattgcactttgttttaatacatgcagttttcacattagcattaccccaaatacaatttctaaagccaaagactgaaattgagactctgccttcaacatttgctgctccatgacgttcttgattgcaatgagaggctggctgtggccagcttgcgggtgcatcacagcagggagctttaacattctttcctctgctttccagctgacatgttcctccggctctgccacacccatcagatggcaggtggggagcagattaagacatgggcattcgcagggtgcacagaacgctggcttacaaacacctcctctcataggcatgcaggtgaacagatgggtgggtgggaggttccccttcacccacagtcctgaatctgagttcttgggttaggatttggtatggcagccttcacattgcacagatatccctacattgggctgctctgcatgcctgcaatgagagtttttgggcttcttgcaggcaggaggtgctgagtattcacaccaaagcttggacatataagacaccagtgacccctattctgctaattcataacactcagccggctcccacattcccactgcctgtgcccatccccgagaggctttcctaccttcctttttctgctttataaagcaacagtgagattccaggagcaaggccagcaaagcgaggtaggagagctcatagataatccaacaaacgtgtatggagagcccaccatggggtgctctccccatgaaacttcaattttttttttttttttagatggagtcttgctctgtcaccaggctggagtgcagtggcgcaatctcagctcactgcaacctctgcctcctgggttcaagcgattctcctgcctcagcctcccgagtagctgggactacaggtgcacgccaccatgcccagctaatttttgtatttttagtagagatggggtttcaccatgttggccaggatggtctcgatctcttgacctcgtgatctgcctgcctcagcctcccaaagtgctgggattacaggcgtgagccaccgcgcctggtctgaaactcgaattctaatgggaaagacaaaaataaaaaacaaaaaatatataattgttaaaaggactgcagaggcaccagtaaaggatcagagagcaaattatcagggtggcagccagccaagaactgtaggggaaagtgccctcaaaggcgggaacatttaagacaaggcttaaaggataagcaagctgcccccacagggagggagcagagcgtgccagacagaggatggggaaagagctcctgcagtggagatgagaaaaccagacgtgtgatctgggggccgggggacgaggtaaccaggggatgagggaaagagggaaaaaacaagcaaggctgaccatgccccacctgaaggttttgggaaggatttggattttattcaagatccagggaaagccagtgcaggtatcaagcaggtttttctgggatctacttccaggaagaaggacaagtataaaaaggtgcaggggaggtgtaggctcctcccctaagagggaacctgctgggtggccctgtagaagacagcagtcatagtttccatcagagctgtgcattcaaccttctctatttcccagctgtgctgcatggaaacagaaaatggcatggccaggccaggggcagtggctcccgcctgtaatcccagcactttgggaggccaaggcgggcagatttacttgaggtcaagagtttgagaccaccgtggctaatatggtgaaaccccgtctctactaaaaatacaaaaattagcctggcgtggtgtcacatgcctgtagtcccagctactcagaaggcccatgcaggagaattgcttggacctgggaggcagaggttgcagcgagccgagattgcaccactgcactccagcctgggcaacagagtgagactgtctcaaaaataaaataaagtaaaataaataaaaaagaaaaaaaagaaaacgccatggccagaggcgccacattttagcttcaggaaactgtgttgaatactctgagttgtaccagcagggacactcccagccgcccataggtccttgtgctgagcaccgggcattgcctccttcctgcttctaccaggaaaattgctgtctgggagccttataacacagactgctggggtttgtgggagatcaaggattatgttccctgtggactagtttgaaaaggttgaaattcagccccttcataatgtcatgctcgtagctaagaaggcttgcctttttctcttcctttgtccattttagaattaactgtggatatcggtaaaagcagcctcaccactaatacatgtggggctggggccgtgatacagcccaaggcccctatactatatatctgcatattttcagagataaatcagtctagtaaatggctaaatatagcatgttctattgtcctaccttgatgctttaaaatgtattattcaggcatgatgaggccaacagatcaggagacagctgccattgaaaagagtttgttactcacagttcccaagggctgggacatgccatgctatgccaggccacatggcgatgcaccagcattggtgaggaggtagagagagtgagggggaagatgtgggcaagagtattgcggtttctgtgagaaggaatgggaaaggcaagacaaccaggcttgggattagctagtttgaataacttccgtggactctggagtatagggggtgtccctagttatctgatgccaggggcaaaggatagtagcccagcatgtgagaccctagtacaggaggtggttgggggtgtgggttctggatttcttggttatcctagcaagggtgtgctcacaggttagttgtttactctttctaggaatgaccctgggaggagcagtccctctagagtcagcaaggccccaagatgtcaaagcatcataaaatactgaaaatgaaaaagaacatgattgttgacgaagaacatgacttgacaatatgactttttttttttttttttgagacagggtctcgctgtgtcacagaagctggaatgcagtgatgcaatcttggctcactgcaacctccacgtcctgggttcaagctattctcctacctcagcttcctgagtagctgggactatggacatgtgccactacagctggataatttttgtatttttagtagagatggggtttcagcatgttgaccaggctggtgttgaactcctgacctcaggtaatccacctgcttcggcctcccaaagtcctgggattataggcatgaaccaccacacccagccacaaatatgacttcttaatgattaatgattaggagaccaggtatgagtttagaatgcttgaattccttggactctatatcagaaagcagaggtgccagagcactggtccactgaacccagcctccagctctatttttctgcacatctctgttttccctcaccatgactgtcttgcagatttgtgcacagacactccagcatgtgtgtctgagttttgtctacctgctctgtggtgggggcttggatgcactgcccagatccttcttctggaaggaagggtcaggtttctgcagctactcccagctgtcagcctttcaggtttgtgggggctaaagaaaatggccttgtccaaagttgcatctggatctggtatccaatcaatgcagaggcataaaagccttgtcccttgtgctaactcggaagaagggctccttggcttcagagctccctgtgggttggctggggcatttgtcagatatgctttgcagctccgtgcaatcctgcctcttctctgcactttcacaggtggtaagcagaagagactccctaataagcatccagggtgttaaactctacctcagggtcagcttcctggggaagcccacccaccacagtccctttccactcactttccttccacatacccttaacgcagcaagccccctgcaccctcaggcctaggaaagcacagactggccatactgtgcacactcagaagcctaagctggcaaagaggcttgcttgagtttaagaagcggaagttacaatcctaatctctgataaaatagactttaaaccaacaaagatcaaaagagacaaagaagggcattacataatggtaaagggatcaatgcagcaagaagagctaactctcctaaatatatctgcacccaatacaggagcacccagattcaaaaagcaagttcttagagacctacaaagcggcttagactcccacacaataatagtgggagattttaacaccccactgtcaatattagaaagatcaacgagacagaaaattaacaaggatattcaggacttgaactcagctctggactaagcggacctaatagacatctacagagctctccaccccatcacaaacttaatccatcacataaataccaaatacattcttctcagcacctcatcacacttattctaaaattgaccacataattggaagttaaacactcctcagcaaatgcaaaagaatggaaatcataacaaatagtctctcagaccacagtgcaatcaaattagaactcaagattaaggaactcactcaaaacctcacaactacatggaaactgaacagcctgctcctgaatgactcctgggtacataatgaaatgaaggcagaaataaagatgttctttgaaaccaatgagaacaaagacacaacaaagcagaatctctgggacacatttaaaacagtgtgtagagggaaatttatagcactaaatgtccacaggagaaagcaggagagatctaaaattgacaccctagcatcaaaattaaaagaactagagaagcaagaggaaacaaattcaaaatctagccgaagacaagaaataactaagatgagagcagaactgaaggagatagagacaagaaaaacctttcaaaaaatcaatgaatccaggagctggttttttgaaaagatcaacaagacagatagaccactagccagactaataaagaagaatagagagaagaatcaaagagacgcaataaaaaatgataaaggggatatcaccacctatcccacagaaatacaaactaccatcagagaatactataaacacctctatgcaaatgaactagaaaatctggaagaaatggataaattcttggacacgtataccctcccgtctaaaccaggaaggagtggaatccctgaataaactaataacaagttatgaaattgaggcagcaattaatagcctaccaaccaaaaaaagtccaggaccagatggattcacagccgaattctaccagaggtacaaagaggagctggtaccattccttctgaaatgattccaaacaagagaaaaagagggaatcctccctaactcattttatgaggccagcatcatcctgataccaaaacctggcagagacaccacaaaaaaagaaaatttcaggccaatatttctgatgaacatagatgtgaaaatcctcaataaaatactggcaaaccgaatccagcagcacatcaaaaagcttatccaccacaatcaagtcagcttcatccctgggttgcaaggctgtttcaacatatgcaaatcaataaacataatccatcacataatcagaaccaatgacaaaaaacacattattatctcaataggtgcagaaaaggccttcgacaaaattcaacaccccttcatgctaaaaactctcaataaactaggtatcgatggaatgtatctcaaaataataagagctttttatgacaaacccacagccaatattatactgaatgggcaaaaactgaaagcattccctttgaaaaccaacacaagacaaggatgccctctctcaccactcctattcaacatagtattggaagttctggccagggcaatcaggcaagagaaagaaataaaatgtattcaagtaggaagagaggaagtcaaattgtctctgtttgcagatgacatgattgtatattaaaaaaactccgtagtctgagcccaaaatcttcttaagctgaaaagcaacttcagcaaagttgctgttatcaatgtgcaaaaatcacaagcattcccatacaccaataacagacaaacagagacccaaatcctgaatgaactccctttcataattgctacaaagagaataaaatacctaggaatacaacttacaagggttgtgaaggacctcttcaaggaaaactacaaaccactgctcaaggaaataagagaggaccaaacaaatggaaaaacattccatgctcatggataggaagaagcaatatcatcaaagtcaccatactgcccaaagtaatttatagattcaatgctatccccatcaagctaccactgactttcttcacagaattggaaaaaactattttaaagttcatacagaaccaaagaaagagcccacatagccaagacaatcctgggcaagaagaatgaagctggaagcatcacgctacttgacttcaaactatactacaagcttacagtaaccaaaacagcatggtactggtaccaaaacagatatatagaccaatggagcagaacaggggcctcagaaataacaccacccatctacaaccatctgatctttgacaaacctgacacaaacaagcaatggggaaaagattccctatttaacaaatggtgttgggaaaactaaccagccatattcagaaaactgaaactggaccccttccttacaccttgtacagaaatcaactcaagatggatcagagacttaaacgtaagacctaggaccataaaaatcctagaagaaaacctgggcaataccattcaggacataggcgtgggcaaagacttcatgtctaaaacaccaaaagcaatggcaacaaaagccaaaattgacaaatgggatctaattaaactaaagagcttctgcacagcaaaagaaattatcatcagcatgaacaggcaacctacagaatgggggaaaatttttgcaatctatccatctgacaaagggctaatattcagaatctacaaagaacttaaacaaatttaccagtaaaaaacaaatagccccatcaaaaagtgggcaaaggatatgaacagacacttctcaaaagaagacatttatgtaacaaacagacgtataaaaaaatgctcatcatcactggtcactagaaaaatgcaaatcaaaaccacaatgagataccatctcatgccagttagaattgcgatcattaaaaagtcaggaaacaacagatgctggagagggagtggggaaatagggatgcttttacactgttggtgggagtgtaaattaattcaaccattgtggaagacagtgtagcgattcctcaaggatctagaactagaaataccatttgacccagcaatcccatatacccaaaggatcattgtactataatcattctatgataaagacacatgcacacttatgtttattgtggcactattcacaatagcaaagacttggaaccaacccaaatgtccattaataatagactggataaagaaaatgtggcacatatacaccatggaatactatgcagccataaaaaaggataagttcatgtcctttgcagggagatggatgaagctcgaaaccaccattctccgcaaactatcacaagaacagaaaaccaaacaccacatgttctcactcataagtgggagctgaataatgggaacacatggacatagggaggggaacatcacacaccggggcctgtcgggtggtggggggctcgggaagggatagcttttggagaaatatctgatgtaggtgaccggttgttgggtgcagcaaaacaccgtggcacgtgtatatctatgtaacaaaactgcatgttctgcacatgtaccctagaacttaaagtataattaaaaaaaaaagaagtgctcccagggacatttgggcaggaaattcccgtgttctgggtaccaagaacacaaaccagaattgaaggggcaaatcttttggccatgtgactgcctcaccatgcagaggggcattctggaggaatctggaagtgagggttctctatcatgatgcccagggcaggggcccctcttgggcaggtccaagggctattctagattgaagcagatattcaatcatttgtatttttcctgtgttaatttttttttttttttttttttttttgagacagagtctcaactctgttgcccaggctggagtgcagtggtgcgatctcggctcactgcaacctccgcctcctgggttcaagcgattctcctgcctcagccttctgagtagctggcattacaggtgcgtgccaccacgcccagctaatttttgtatttttagtagagacggggtttcaccatgttagccatgatggtctcgatcttctgacctcgtgttccacctgcctcggcctcacaaagtgctgggattacaggcgtgagccaccgtgcccggccttcctgtgttaattttttacacatacttgttcatcacccatgtatctatcacctgcatattaaaggagaggagacctctgtgtgtgtcttactagccggggcacgttgtcttagcatctttggcctctaatgagactatgtttataagcaaaaggtgcccagtcaggcatagtgaggccacttgggggtgagggccggtgcctggatgtggacagcatgtgtgtgttttgaggtgggagtaaggcctggagtgatgaatggaactttatagccaacaaaaggctttcctcaaatgggaagttgttctgacctcactttgtgcctctgcttggccaatttggcctttttcctgccaaatccatttggcataaggagcaggcagtatttgggctggctgcttgggaactttgtaagacagttgaggagggcacctccagcctggaaaacactgcgtagtccggatctggctgccttacgggggcgccatccttggccactgagcacaaactacgtcttcctgctagtgtcccagcagctctcccaacagcaacagctcagaccacccatcagacccacaagctcgtgtgtcttttaacagttgcatatgaaaagcctctgctttagagggaaaagtaaattctttccttttgcgaaattccctcagtgaaattcataaattatgtttcatcacaactgggcaacaatattgtgtatttgtaaaaacagagcctttggcataaaacccaagtgggtctgaattttttatttactccttgaaataacttctcccagccccagtctccttatcctcaaaagggatttagagagttattgtgagaattaaacaaaataatgatgcacatggagcatcctgccagcactcagtgtgagctcacttttcctagcttaagaagatcaagtgtggattggggcttgttttaaacacaagttgtttaagaagatagtattccacttttaaaacaaagtcccaaattttcttccttcttctgtctataaaaaagaatgaaaaaaaattctgtgtgtgtgtgtgtgtgtgtgtgtgtgtgtgtgtgtgtgtgtgatgcccatatttcagggaaataacaggagaaagaatcaaagaatgagactgcatattctcagttgcatactactaggtaagacagtaaataggcattctaagcaatttaaaaaatactgtaaattaatctccaggtcacaactctttcagttatatgcgccagaaagcaagaaagcaaacccaaatatgcctgctgctgtcagcagggattgctgagaaaagttagcctgtctttccatttagaaaaagcatttgacaaaggcacgtgagtgggaggctgtgaagtgaacctgcttggaactgatagtgttagctttttactgacctgctttccataccgcaagcatttggtttttcttttgtcattgacttgtgcggatttattagactgtgtgcttcaagggtgtcaggagattgcatgtaggagcaggtctgaactgggaaagtgagaattaccatattagatgatacccaattaactagtgacaggcacagcaggtaaaatcggtacctgggctttatctactataagcactcgccacatagcaaacatttttaaacctggtggagcttccttactcatttctcctttccttcatcctgcccactctcccttcctgtctaagcaaggagctgtgtctacctatgtaagtatcaagacagacaggtctttgagatgtgctccagggttcaagggcaatggggcagaactctctgagggtggtgactcactgggaagcaacagccacacaccaccccacaggacaaaacagtggaagtggaggcatcaacatagttcagaataattgattttacttttttcatggacaactgagtcacaggatgatgtcccagttctgaaaacactttctcactctctgttctttgggtacctttgccagcatgaagttacttactataactcagcattctcactcgcgttactcaaaggtgaaaagctggaagaatgagttaatcttacttccacattcaaatgtggctattagggcagtgatggaagctcagagcctgtgcttttggacaggctgatttcctgtagctactctgtcttcctttggtgtgggatcatgtagtccatctagtgatcataatgtaagcttggagggaaatgatttggaaataattgggaaggtaaaatgatatgacatgaaatgcctgctattctaaaacacaaatcttattttatgtgcaactgctgttttggattatccatgcctcctcctgcatcagctagcatggaaaatcctttaagatctctaactcagtcacttcctcagggagtcatgtttaaaccccctcaagctgggccaaatctccctccatgacgctctgagaatgactgctgcttttatcattgaatctaacttgttgcttttattagtcatctattgatgtgactctttctgttaccaagcctgattttcttaagcccacgggcagcgtgatattcacctctgtggccccagtgcctaacatggaatctggcatgtgttaggcatttaaggaacattagaggcagggtgtggtggctcacacctgtaatctcagtactttgggaagtggaagtgagaggactgcttgaggccaggagttcgagatcagcctgggcaacaaagtgaaccctcatctctacaaaaaaattaaaaaaaaaattagctgggcacagtggctcacacctgactcagctattcaagtctcagctgttcaagagattgaggtgggaagatcacttgagcccaggagttcaaggctgtagtgagctataatcacgccactgcactttagcctgggtgacagagcaagaccttgtctcaatgtctcaaaaaaaaaaaaaaaaaaaaaaagaaaagaaaagaaagaaaaagaaaaaacattagatgaatgtatgaatgacagtcaggtataactccaaagagaaaccctttccctgttaccacttattaaatcagtaggactcatatctgttaattttccttctctctgtaaacatcttatcaacatcagagaaaaagagacagagaattaacaacaacatactgaacgatgccctgacaatgagaaatgaatcccttgctagtttggtaatgatgcatttatttggctctggatctagagaatttatattgagtagtcaaaagcctgggaaggactgaactctgttgaactcagaaatcctctcttgagagttctagctaaactagaactaccacagataggtgatagcagaatgctatatttgaaatgctgcaaataaataaatttatacatgcaaatcaaaatcttttagtaaaatcacacaattaacctctcactggggagaaagagccaatgtagatatatgctttagaaagttgttaatcattttctcattgcaacctactttgaaatcaccttggcctattgtctctcatatatatatgtttatatatatatatatatatatatgtatatatatatataaacttattttacacaatgtagatttattttatgctgatatactagtaatgttcttccctacattttacaaacaatgagattttcatttattgatggctggtctttgcaatggtcagtactctttgtaactctgactttttacagcacagcctttggcctcccctgaatttagtgtctttgaacacctgcttcattttgttggtgctgagatttcacatctcatgtgcactaactgctgtcatgttatatgaagttgaagtcatgtgttttgtaacaatgagctctggaactatcccttactggcagtgtgactttgaaccttactgctgctccatttaaccaactgtaaaatggtgataatggtagagctgcttcactgatactattatttagttcatgtttagaataaggtgtttaaaacaggtcttgcaaaagaaagtgctcagtaaaatggtagctaaattgatatcacgcgtgtccccacagtgggtctccaatgtgacagtcaatatcatttgcgtatgagcagacaaaactctctaggttttaggggttcatggttgatatttcctttttgcccttcagtgtgaaatttcactcacgtaggacttcatggaaagaatctaccatctttcaaggacttacacaatcttatgccatcctacctgccaagacattccagctgcaagtccattttcctgtattgctgactttcctgggtcttttcaggattatgttacctcttcatcctcatgcatgatttaattatccgaacaaaggttctttcattcactcgaagacagcataataccttagatatttgaaaatttctcagagttctaaaatttctcctttgtctttcacatcttccttctgaaagattttactaagtgggagttaattttatctaaattgattcttgaatattgatctttgaaatctgatgacttattttcataaatgtaggccaggtctcacttggtacttaatgttaatttagaaaattccaataacgtggctaatgtttgacaaactttgccattaatgctaaactggggaggtccctttggcttccattttcaaaggtgagatcgtatcgtactgatcccgtctgaggatgccttccttattaattgagggtggtggggctggagtttcttcaaatcactggtccattcaatctgatagcaagtctgcctgggtgttactcatttcccatgctattttcatgtcaaatactgctgtccccttatgcaagggatacaattcattgaaacctcgttggacttcctcccacttaacattctcctaccttccgtgttgacattcttctaccttccaacttccgtgtttacactgaaaaagctgttgctgggtagcttggttattttttttcccatcagactcatagggatatttttggattattggttatgatgaatatttatggaaaatgttgctgtgcactccagtgagaaatcagtgttaaagaggctggtcccacagctatctctgtgtccctggaagccgtgtcctttttctatttaattagtgccaagtatatggtcttgcatgaaggaggacccagattgaactcctgtagcatgatgtgtgtttcatgcactctactgttttcactgatttatggcatttaattatcaatctgttaatccaagtttttgtttaattggattcatcaaactaattgtatgaattccactctgttatcatggcctgaacctgaatatcagattcccttaataaacagaaacagtgaatgtgttcctgcttcttcctgatcaggaagggggtctgcagtcctctggtcttgtttgtttcgtagcatttcattttgtattcagcatttctttgagcgccctctttgcttcttggaaccttgccttactaccattgccattctcataggtccctgctctactggacctaaaatggtagggaagaaaggaagaggaggggagtaagaagagaggaaaaggtgagctctattctccgtcagcgtgtctgtggctgtattgattcggaagtttaaaatctaaactccctgtctacagtcttggcctgtcttgaaggttgtgagtcaacttagtgaatacatgcaaggtacttttaggctgtctggctgggagataagttgggtccaaagaaagtaatagttcaacatagcattctccttagatggcagcctccaggcaagaagaaaagcccatggctttgcctggaagttcttgtgcaggtccagcgtgtcaccaactggtgcttcagacactgagatgaggcaaatacttagcattcagtttcttaacaaagtgattaacttgatagacatctagattctttttaaacataagaatatggctttcttttcttcaggtggtgtgtccatttttgtccatcggatagaaggtgctatcacaatataaatggcgcgttgcgtacgatcagcaagggcttcttaagttactcccacttccttagtgatactgtccagtctcattgcttcaaacgccaatcccaaatgtctctgttccagaactttcccaaactccagactcagatatccaacctcacacttgacatctccactgaatttctaaaaacttctgaaatttaacaaatccaaagccaaattcttgtctccccaaacccatttcacttctcattcttcccatctcagttgatggcaatttctgttttttttctggtggcttagggtaaaaatctgtagtcattcttgacttttttttttttttttttggaaacagagttttgctttttgttgcccaggctggagtgcagtggcacgatctcggctcactgcaacctccgcctcccacgttcaagggattcttctgtctcagcctcccaactaggtgggattacaggcacccaccaccacgcccagccaatttttctatttttagtagagacggggtttcaccatgttggccaggctggtcttgaactccagacctcaggtgatccgcccacctcagcctcccaaagtgctgggattacaggtgtgagctaccgcacccagccaacttttctttttcttacacctggcatttgacccaccaggaaagctattgtctctacctttaaaatatatctaaaatctgcccacttatcactaactcctgctaatgcactagtttaagtccctattgggacatagaatatgatactacaaagtacagcaccttggaaactgagaaaacagcagaagcagaaagggtactctgaccatctgctgtctttctgtgtgagctgatcttaaaggactctcttgcttaccttccctgaaaggtgagcataagacccttatgtgacagttgccctgccccccaatgtggagggaaggaatgctacacagaggccaaggagattctgaacaaacagccatgctgagctccagtttatcaccattagatcatgtttttgtccaatcacgtttctacacagctgtccatttttcatatgcttcattgaacctaagcacaaaaatagacagttttctctgggtcttggggtcttcatctctgaaggctcctatatcacataaaactttggtcacataaatttgtcatgcctttctcttgttaacctgtcttttgttataggggtgtcagccatgaccctggtgatgggtgagaaaaaatattctacttttcctcctccacatctccatcatcttttcctggacgattgcactaacctcctccatgtctttctgcttccaccccatcctcccttatggtgtattctcaacatagttgctgtaggtcaatctcatttttttcttctacagtgtttttccattccactcagaacactcaaaatcctgatagtggctttctagttactacagctgtgcaacaaaacatccccaaacttaatgacttaacatcaccattaattatgtgcaaaattctgtgagtcaggaattcagacagggtatagtgggggggacttgtctccatcctgcgatgtctgggtccctcagactcagaggcactaaccacctgaaagctcattcactcaggtatctggaggttggtgctggatgtattctaagaccatcacccaggttttcagcttaaacacctacacagggcctctccatgtggcttgggcttcctcacaaaatggtggctgggttcccaggacagagagccacgcagaaactggactctagcctcagaagagttatagaatcccttccatcatactcagtgggttggagcagtcacagcctgtccacattcaagaagagggaaatagtctttacctcttgatagggagtggcaaggttctggaagagtatgtggtaccagaaatattgctgtggccattttgagactatagtctaccacagtggcccgcttgattggacctgggttgtttctctgccttcatcttttctccctatggaacatttggctccggccacacttgctagagcatgcagatgtgcttctgccctggggccttcagactgcctactccattcacaagatgcctgttgatgccctcttccctccctccgcaaacttttatggagtgtcatttccccagtgatgccttccctgaccactctacagccccttttcacttgctgtctttttttttccccttaaatccttattgtcattaccaccttctaatgttttatggaatttttatgtttattctttagcatcccttgctgccagaatggaagcactacagaagcaggaatctttacctcttctgtccactgagctatctcacatgcctagaacagtgtgtggcacataaatatgtatgggatgtgaaacagagagggcgtttctggtttgggcatttggaatttgcaataattgctccagctagcacaactaagtttgtgtgttagaccagcataagtcaataaaaaaagataccaacttaaacagttgttttcattgtgtaaatgtctctttttatcttttatggactaatgaaccagaatgtttaattgttttaatttccatctaaatatttagtttctttatgaggaatggcagggtactttttccagttccacagtccctcaccttacacatacacgttgactgaaatagaagtctaagaatccccagacactccagtagcatgaatctgggcagcctggcttctgtgtttccacattctccaactttagagtacatgagcccttcagcataaataacccttcagaataaaggttgtaataaaaacaatgataaaacccacaataacaataataatactagtaatagacatggcagcaccattgataattccagccaccatttgctagaaaccccttggctgcaggtgttcttccaggcacttggtgtgcacgtctgttagttattatcttgagaggaaggcaccttcattcctcggatgagcaaaaggggagaacttgttcaaagtatatcaaatctcttttatccgtaaagtgcctctttgatctgctgaaccatagcaatcattcattccttcaacatttggtaattaggaagcgctgagtttatctcaaagagtagctgatctcatcactcattggatgttgagcagttgatttctagttactggcagaatcccatgtttatgtttatttctcaatgaaaggttccacaatcctcctctacccctagtgtggctcttctggttcatctgtaggatgggaatcattacactgtcaggttctctttccctgtattggacaaccgagccaatggctatcagttgggaaaggtaagagatagagcacatgtcatccagtgatagactatcagtgagctgctttttaaagactacagcaaggctggctcagcgctattgctccagcctctgactttctcctgcagagacactcagagccccggaatcctccccaactcatgacttcaggcagccactccagtggatttgagtgttttccaaacatagaatgttacgattactagtggcttgacctcaatcctttggctttgagcttttagttgaatggtttttattcaattgtgactgcagttgcattaactttggatttcctttttttcccccagaaacaagtgtgaaggaaaatactatgaagtacatggaaacacttaagaataaactgtgcaggtaaggataataatttctgtatttcagactccgtagcacatctagattctttattataacagaatttagttaaatcgatgaaaaaggctggccagtttctctaataaattaacaaagaaagacacaaaaaagccttttgaaccccatcagtgaaagagcaggtgccagccctcatcttgtcttttaaaaagtaagtagattaagatgtttaaaagtgtcttggagagatcacaggagagttttcatgttccataagagaagaatcttctttactatattaaattcagaattcattccttagtctgtttttcccttttttaaactttagtgtttgaggggggtcttaaggattttaaggcggtttatacatggtttggaataatatagaaatgcacgtcttaggttctgttttgaatcctgaatcaaaaccagcatgaaaatttctggttgctttttttaaaacccaattaggaattcatctttgagaaatgtcagggatgctgcagcatgacaagatgtttctggcttttgtggatgtgtgcttaggaaaataaatcccagtcatcagaatctgttcctaattaaggcaacaagtactagagtggaatatgcgctttcttccctggaagtcatttagttttatggagaccattttgctattattttggaatttgttaagtaactctttagataggctatgccttaagcctttgtctgctaggacgctgtcttgagctaaaatgctgcttttccattaagagatgttgagagggcctgccttgcagagccctactcttcagggtaagcaaggcactacaggatttattctcatccttggaaagccgtcctataacctcctgattgatctttccctgcttcccatgctcctagacttgggaagaatctatgacacaggtggttagcagaggtgagcagaatgaggagagcttgcacctgggtcagatgctccgctctgagtctggccttcctcctctcgcatgtgttcggggcagatttctggcacatccttcttgatctcaactgcagctgccattgttttagccatcagcacagcacacaccgtaagttacattaggttatcaggcagccagtggtagccataagcacgtgggtattggtaaagagacacccatttaaattgcagctctcccacataaaaactgcctaaccttaggtaagtgctcactttctgttcagtttgttttgttttgtttttttagctatataactggaggaaaggtacctacctccccagctggtgtgtgaatttaaataccatatgtaaagcattcatgtgattggtagtaggcacgcattagtttcctttaccttcttcttatttatttatttatttatttattttgagatgaagtttcgctcattgcccatgctgtagtgcaatggcgctatcttagctcactgcaacctctgcctcccaggtactagcgattctcctgtctcagcttcccaagtagcttggattacaggcagtgccaccatgcccggctcatttttttatatttagtggagatggagtttcaccatgttagtcaggctggtcacaaactcctgacctccggtgatccacggccttggcctcccaaagtgctgggattacgggtgtgcaccatcgtgcccggccacctccttctttatatcattagaactgattggacacttatgagtggaaaaacacaaactgcttttcctctgttctcacacaatgcaataatcaacacagaaggcttctgtgatgaaatacatgagggtttctccccaccaaaaagtaagcaatcagttctgctgcagataccagctgggtgtcctctaactcatttccgacaccagctacctggagacagcatcagatgccacaggttgagggctcagtcccacaagactgccccacttacaatgccaatggcaagcccaggttgttttgcctgggcttctgaccaaccaactagaaattgggattcccatgatcccctccttgggtttgattaatttgttagactggttcatgaaattcagaaaaacacttatatttactggtttattataaaggaggttacataggatacagaagaagagatgcatagggtgaggtatgggaaaggggcaccaagctttcatgccctccttgggcacaccatcttcaaggaacctccacatattcagctatctggaagctctctgtaccctctcctcttgggccttttgtggggacatcactggataggtgtgactgaagtatggacaaccatgtagcaatgtgattggacagaaactgtgacctaccacaaatagactgagtgggaaaaccccacagggcctgtgtgttcagattccccagcttctctgtgcagcgtttcttccttcaggtgtgggacaggactccttctgaaatgagggtctatgacctacagtcagataaggtaggtcagagaatttctttccttctttctttttcataagttcaacttttattttagattcagggggtatatgtataggtttgttatatggatgtatcgtgcgatgctgaggtttggggtatgattgatcctgtcactcaagtagtaagcacagtacccctggaattagttttcaacccttgcttcccttcctcctctgtcagtctccagtatctattgttcccatctttatatccatgagtacccagtgtttagctcccacttgcaagtgagaacatgcagtgtttggtttttcgttcctacattaatttgcttagaataatggcctccagctacatccatgtttctgcaaaggacatgattttattctttatatgactgcatagtactctgtggtataaatgtaccatgttttctttatccatttcaccattgatgggcatctaggttggttccatgactttgctattgtgaatagtgctgtgatgaacatacgagagcatgtgtctttttggtagaatggtttattctcttttggatatatacccagtaatgggattgctgggttgaatggcagttctgttttaagttctttgggaaatctgccaactgcttctacagtgactgaactaatttacattaccaccaacaatgtataagtgttcccttttcttcacaacctctccagcatctgttgttttttggctttttaataatagccattctgactaatgtgagatggtatctcattatggttttgatttacatttctctgatgattaatgatgttaaactttttttgtatttctgttggctgcttgtatgtcttcttttgagaagtacctgttcatatcttttgcccactttttaatagagttatttgttttttctttgtcaaattgtttaagttccttatggattctggatattagacctttgttagatgcatagtttgggaatattttctcccattctgtaggttgtctgtttactctgttgataatttctttttctgtgcagaaatggtttagtttgattaggtcccacttgtcaatttgtgtttttgttgcaactgcttttgaggatgtgggcatatattctttcccaaggctgatggaaccataaaagagcctgaatatccaaaacaatcctgagcaaaaagaacaaagctggaggcaacacgttacccaacttcgggctaaactgtaaggctacagtaacaaaaacatcaaggtgccggtataaaagctgagaatttctttatggtcagctccaagacagaaaggcagggggagattagggtatatttttagtttttatgaactgcctaggggagaaataacagtggatgtgggagttatgagccaggaattgtagatgaaaacctatatacataatttcaacagtcatgcttggaactcatcctctctccctccatgtcagctcctcctcctgttcatccagcccaatgcacggcccaccacccacctggctgcctgggtgggcgggcaccatgggctcatgctggctcaccagcctcctttctcactcagttcccagtcttgtcagtcttgcctctttagcatctcacaagcctgcctacctttatcacactgacattgtgggcccctcttctgatcataatagcaacttttcctgctttgctgttccactgttgccctcttaccaccttctccaggtggttttgagtaagcatcttaaaataccaatctggtcattgcacaaggctgcccgatgtcctaagcataaagcctaaatgccttaccatggttcatggcattcttccatgtggcctctgcatgtacttgattatcacccatcactaccccatcatcaccctattgtagctgatacttgttcccttatgcttcccaatgccattgcatttttttttttctggaaagaaactaatttagaaggggacatggttaaggagtatcatccagagaaatattcgattgaatcatgtgaaattgctgatatttcaccatttttgacctagaaaaatgacaatttcatatagttaacccaatatttagggatttgaagtcatcttcagaagggtcagatgctgtggctggtctccttgcccttgacacacaggcatagtttgctcccccacccctctgcatgtgccctggtgggcctctcccagcccacccctcaccctcaccaggccttcccattcagccttcaaggtttagatttctcctctgtgcaaccttccctgactaaccaagactgagtaatgtgcttgcaatgatttattctcactttccaagtgctccttgacaggtattataatgtagcaggacaagccgcagacaaaactcctcagacgctgagttaaagaaggaaaagctttattcggctaggagcttcggcaagactcacgtctccaacaactgagctccccgagtgagcaattcctgtcccttttaagggctcacaactttaagggggtctgcgtgagagggtcatgatccatgtgagcaagcagagggtacgtgactgggggctgcatgcactggtaattagaatggaacagaacaggacagggattttcacagtgcttttctatacaatgtctgaaatctatagataacataaccgattaggtcaggggtcgatctttaactgccaggcccagggtgtggcaccgggctgtctgcttgtggatttcatttctgccttttagtttttacttcttctttctttggtggcagaaattgggcataagacaacatgagaggtggtctcctcccttaataagattaagtctgccttgttcaggactgtattctcagtgatttgccagtgctggatggtacaggggttcccaataaacatgtattgaatgaatgattgaataaatatgtgaattaatgcaaataatttgtaaacatcctaggctcatcttttgtcatctccagcctctttctctgcctctccagccatgctgacgttgcaaagccaccttgcccctggcctcttgactcacataccttagctacctaggatcctgccacagtctcaaatgaccgtgatagtagctggcccttcctgtagcctggagggctcttttcatgctatttcaagctattatgaggcctgggtcagatttgtgtgaactggtatggtgtaaacaatggaacagggaggagaaaaagcagctaggagtctgcaacccctctggcagtaggcaattctattcccacttcagaacaggaggccagagtggatgctgcaaaagcctggtgaccatcaggctctctgtgacagagcccagtagttatggttgttttcagacactctgtgggctccactgataatctgtcaccttcacagttcctttccaagaggatcaacattttacaataggaaaacagttttaacacctgacacagtggtgcatcttagtgactgcttgattaacatctcaagttatccattaaagaagatgaaaatctttacctgttcccactgttcagcgtttcttggtcttacaggattttagagaggaagcagctgagaacagaatggattgctccttttccattagaatgctggagagcatatacttaaagatcagagctaagaaacctaaatctggctagtcaccattttggggacaaattcaaatgatagcactatctttgttactctttcaaagtatacagagacctgaagacccagatattgaggcaaactcaggtggactcaagatctattctctatcaagtatatcaagtgagttagaaagtgaggaagtatctgatgctggcagtggttcaaaagcacatcttgcataactcaaagcaaaaagtggacaaatattacatgaggacttttagtgtatgtttaaataagggtttgccttgtaatcatctgagttataagttagatgcatgtattcctgttcctccttctacaaaggagagattccatttgtggattttttttccaatttccacctgttttaggaatatccctccttctccttatccatagccaaatatccaggagggtgactgcggtcagtacctggtcattccatagaggctttcctctgcattcccatcactactacctgcaacttggcccattaaataatctcctaacagcctctcttccctcctggctcacaacagtttagtctgctcttctcccacagccagaatgactttattaccatcctgttaggctcctttcgcatccagcataaattccacagtccttgttatgtcctcctcctctctgccccttgggctcttcatgaggtggttccaggctactggtccaacctcatctctgtaatcctcatctgcaccaggtgactgatgctccattctagcccatttccctacattaactgtgctccacagcagcacctttgcagagccctccccgacctagcccctgtgctcacactcatccttatgactcagcacaattgtccttctctataaagcctttctggcctcctgctatttcctgcatggtacccatagtggtagccatgtacacgaatgtgttgcattcactcagtctgtctaattctattagactgtgagctccttgtagatggagcctttacttttcttatattccaagtggtattctaaggtttccatgccttactttccttgtattctaagtggttgtcgtgctgtttggcatgtagtcaacagacaaattgtatttaatgcaagaatgaatggataaatcgcaattttatcttgttcctccttggaaatttatttgcaacttgattatcttgacactatattctcaactattacattcagaaagtcaaggcaacctgagaagatacagtgcccaaatccatttgtcttgccatcaaatcttagtggtatataatagaatagaaaaaaacgactcttaaagagcttgacccacttactctcactccttcagggttattgtttccagaggtgagtagttaagtgtgattttctcctagcttgttttctcttatggccaaaccttgctttagtttttattttctcaattattacatattatctgttatcttttttaaagggtcaaatgaaatattcagaaggctttactttatagatgggttcctgttgaaatggatcttatatctctaaccattgtacaggaggaattcattattgcctgatggcaaactcatgtctctggaccaagtgggccaagccctccccctgaggatgggtgtagtggttccatttaactgatggaatctcttgtatgcatgactaatgtgaacatttctcttctttctagaatctcttcattctaactctactttttttcctgaagttcaacataaaacctttaccacgaactcttgagcacttggtagattataagtgatgcttgcgtccttgtcctctagtgaacctttaccacaaactctcgagcacttagtggattatagctgatgtttgtgtccttgtcctctagtgtgttcctcaagtgtttgcttccagtgtgcatcacacttttatcatcagaaaagacaaacaaaaaatcttttaactctgttttgaacatagaagaaagtagttggatacctccttggaggcccttcttagctttagaaggaactgtcaagaactcaatggtcttatctgaaatttgctttgaaatttagagattgatgtatttttacaaagatgatccagttctctttttaaaaactaatgtttactttttaattattaaaatttcaataggacagcctgatgtggaatgatagccaggtgaactcaccctgctaatgaatgtccagaggtggccagcagggtgagcagcacctgaaagaccaaggtctgttccctcacccaaggctcccagagcctgtgtgtcatggccaaaagagactgtaaactgccaactgctctcaatctaatgaagacgggaaccggggtcaaaaattggagcaattgtttccaaaacaggtgcacacaaaacagtttactggagtgtgggaagaacatattataatttctattttatgcttcaatttcatccatttatattttatcttcaaaaatgttttatatgtatgtagtaatacatgtataccacttgtgaaacaaatattggagtttcataattttcttactgatgagtgcacacagtttaaattagagagtgttgaaaaaatattcatttacttatgttctaatattaaaccatagcagtctccataaaatattcactgtcggatttatacacttttgaactttgaagctatttgactattttgaccaattttacaagagaaacaatgatttacattaccttatcagtttgaaatgcaaactgtaagaaaatataaagcgcttaattctacaaagttaagtgaaatcattaatttgataatcattgatttttgcatttaacttttaccttatctcatcagaaaaatcttaagtatgcttaaaataataatgcataagctaaacatacgcagagaaaaactgcataaaatataacaaaatggcgtgaattaaaaagagtaaaaaaggaaggaaaacaaaggtaggaaaaacattagtaacactttgatggggattagactcaacaccatagtagttcactattctagacactcaattaagtattaactgatatgtatataaaactaagtaacactttacaatgtaaaatgtaaatgatataatttataattaacttttctgcattggcctattggtcattataatactttccattctataagaacccatgtaatgaattttaactcagaaataactttcccaggaaatgttcacttgtgatggaatttagtttccagagtactcagatcaattcaaaaaatctgcatgcagttcttcggagagagaaagcagagttccatcttctagaattccagctccctgtgttaggaaagcctttccttcaaaacaagaaaagaagccgccaaccccgaattggaactggcctcttcacatctggaccacaacatgggaggcggaggcaactcttcccttctcggtaccctgagcagacgtaccagtcacagtaattaatataaaaggtgtccttcctcttgaggcatcacttttaaagcaaaaacaagggaggctgaggcaggagaatggcgtgaacccgggaggcggagcttgcagtgagccgagatcccgccactgcactccagcctgggcgacagagcgagactccgtctcaaaaaaaaaaaaaaaaaagcaaaaacaaaaacaaaaagccctctcacacacacacaaaaacctgtttctgtttccctatgtaatgaagacccctgaggttggcagcagtgttgtccaccgccaactccctgtggtcgatctggaagcttcatctagggaggggctcatgaaggggcctcatgagtaacccctctcctgaggattgttattttcaagtaccagctctggaatcagatagtctagattcaagggtggccatttctgagctgtgggcctttaccaggttaatgcagctcttaaagacttgattttctgtcataacaatgggataacagtagaataagataaaaactatttcttatatataataagggattaatcaatgatcactagtatgtactagtaggaattatggcaccatggaaattatgaaaccaagaaactctacattaaaaaacaagttttattttataaaaacagaagagaataccttatcttcctggtgaaattgtggggagcagagagatgtttgcttgtagctaataccctccattaaggatctgccaaacctcctgattatccaaacactcagcggtttttttttttttttttttttttttgctgtctgccctttggccagctgttagcacctgctgcagagggtgggaagaaaaagaaatgaggtaattgagttcttgctaatggctgtgacagaagatttagagccttcagtggttcctacagtcaagtggcagggatcctagagcacactgctgctctcagaatggatctggcttgttctggagtttttgagattgacagagctcaaagttcccaggctccagagctcaggtgctggggaggatttcccatcacttctgagatttggggagctccaaggcccctcctcttgaccctcagtggagctcaaaagaaaatcgtcttgacctcagagacagcattttggggtgaagatactccaatatacttgtttctttctttctttcttttttttttttttattatactttaagttctagggtacgtgtgcacaacttgcaggtttgttacatatgtataccagtgccatgttggtgtgctgcacccattaactcgtcatttacattaggtatatctcctaatgctatccctcccctctccccccaccccatgacaggccccggtgtgtgatgttccccttcctgtgtccaagtgttctcattgttcaattcccacccatgagtgagaacataccatgtttggtttttcgtctttgcgatagtttgctgagaatgatggtttccagcttcatccatgtgcctacaaaggacatgagctcatccttttttatggctgcatagtattccatagtgtatatgtgccacattttcttaatccagtctaccattgatggacgtttgggttggtcccaagtctttgctattgtgaatagtgctgcaataaacatacgtgtgcatgtgtctttatagcagcaagatttataatcctttgggtatatacccagtaatgagatggctgggtcaaatggtatttctagttctagatccctgaggaattgccacactgtcttccacaatggttgaactagtttacagtcccaccaacagtgtaaaagtgttcctatttctccataccttctccagcacctgttgtttcctgactttttaatgatcgccattctaactggtatgagatggtatctcattgtggttttgatttgcatttctctgatggccactgatcatgatgacttgtttctttctttctggtgttttccttggagaaagcaaaaatattgccctagagaatggcatggacagaaaagaagaagaaggaagattagaagtgaagccctgtgggcttgtctggtgagggccctcttcccaggacttagctgcatgggaagaatctgggtagcactacaaggatggaatccatggggtcttagttgcctgtcatcattttctttgccattgataattcaacacttaatcaaatgcggtggtgtttttgaaagatagcctgaagaaaaagatgaccatgactttcactaatagaatagaaaatagacaggagattgattatcacttaaaagaaaagggttcattaaacaggttccactgtggtttgcgcaattcaacccaaagtttctctgctgtaaagcaatgataagatcactctgcccactgagatcaattgagaaatcaaccaatatttaagaaggtctgagacttggcaaggtgcccagtgactgagaaaagagggttcctctttctttaagttggcagaaagacattcagctgtgatttttacgtggcctgagctaaggaagagactgagctaaggaaggcagtgtcttgtgtaggttatatccatggtgctctatgagcctgtgcacccattatccccagatatgtcaatgtgcttcacagtgtgcacatttcatggcacctgttacagttttaaaaagacagtttaattaattaatggtgaatgttcaatcttagatggtttttttaaactcaagataaaccattatagcttgagtgtttttgatgcatttggctaggagttaggaaatcttaaagaaaaattaattgctgtatcaatggaaaccccagtaggtagataacaaatttgtcattaattaaatgagaagactttttaatgattcgaggaagcgtcgtcaagattgtatgatagtttaatttgctaacatggtatgaacctgttagcttcagacacatgattaataaaagtaaaacccctcatggaatttctcagatgctcagttctagcatgcagtcatttttgagacttgctcaatgtccttatctcactttactgacagttctgcagcagccgtggcaaatgtaggttagacaaaatattacatgcaacattttctatgattcattttgctcctttttccctcttccccttcctctccagccaccctgagtcatacaacagctgtgagaacattagttacaagggagcgagctgcagacactttaggtgctgggctgtatcccattatatctggatgctgacaccaggatgggttctaaatcccttatcacaactgcctcaggcggaaaacatttttttttccttctgttgctgctgatgtaaagatgaagtttctcgagctgagagaattttatgtgatcgtattgtatgctaagcctcagaagataattctgtagctcccattcatcacaaaagcagggactttcagttgtgcttctttaagaaaaaaattccccagtagaaatcatacaatgatttagcggactaagaaaaagaagagaatcaccaacaggatgacttgtgacaattacacgaagtcatttgactgagttatttcctttattattctgtaaatgggaaaataacgaacatgctggttatcagctgtggttttgcaatggaggcgttccaccagtgcagagcggtaattacgctgcccggtctcactttgcaaatgaactgaagcctgtctcttttaattgactgtctgtctctagaattttcttgatttattgctgctcagtagaatttacttttgttttgttgatggcatttgtcaactctagtagttaccaaatggaaattgttctagagggttgctttggcataagtgtgctcatttgtgtggcttattggagcatgaaggagagatgtgcttctgtaagatgtcttgcccttctgcctgccagaggggcaggggctcacctggagtatgtttcatctggttccttagatctgtaagtgactatgaagcctttcaactggaagagtttagtcaagtgtaactgtatgatttcggaatgaatatggccccatctttgtttcccaggtggtgagtgtttagggcattatatagcagctgtgatgatgagagcttggcttctcatctccagctatgttagtctacctaggaagaaggattcttctctaagaagaattcagtgggacaatatccagagatacaaatctcaagagacagactcctgaaaacaaattgactgctcatttggcttcagattccatgcataaatctccagctctgactgttaatcacagccactagacctcacgacataggattttagaggcagataaagaaaatggcagttgatgtcttagcctgttgctatgccagtctatgatgaactagcactacctgaactcaagagtaagaaacaaatgaaactggttataaggtttaggaaaaagcaaataagctgttactgagagtggtgaccatatggtacctatagggtacacgggccctctgtgaaacccgtaatattgggctgcgtctcctctttcctctacaacctgaccccatcgcctcatttgatggattccagtcaattgtaattttgagaattgtaagactagtatactagtatgccaatgtcttgttcttttttttttttttttgagatggagtctcactctgttatccaggctggagtgcagtggtgtaatctcagctcactgcaacctctgcctcctgggttcaagcaattctcctgcctcagactcctgagtagctgggactacaggcgtgcgccaccacgcctggctaatctttgtatttttaagtagagatggggtttcaccatattggccaggctggtctcgaactcctgacctcgtgatcagcctgccttggcctcccaaagtgctgggattataggcgtgagccaccgcacccggccgccaatgtcttgttctttaaaggaagaataaatacacatttagaagatggctacaacctcatgcaaaggtctcgaagcagcaagttgagggacagggcttactggtccaagagtaacttcaggcagggaagccttttcccatgtccttcaaattgaccacatcacttggatcatgaatgctgttgatctggtttcaaaacataagaacaagcatcttcgacaatgcggttgataaacatcatcaacaacaacccaccgaagcatcagttgttttgtcttgtccgtggcagtcctgcattcagatctgtattcactgtgtgatcacatgcatttttcctaggcctataataggtttccctccgcatgcccaaacagtggagctagcatttttttaaatttacaaacaaagattgtaaaattctaattgggagtgggacatcctttttaaataaatagtcgtattaatagtaatagcaaggagaccatctagatagtactctattttcagggctcaaaagtgaaaagctagaaaagccagctgtttaagcattggcaggcaagttatgatacggagtaccctcctcacacccactccaaactaatgtgtcatctttctctgaagagctgtggctgtttttgtggttctgagttgacggggatttttttttttcttaaagaggtacatgtttatgtttgcaacatggtatttcttgcagccatactcctcaagtcaccatctccaaagctcacacactaaacagggtcaagtgagtcaaagcagcaggagagaaccatgattggggtgtctgggactagaatggggagggcagcatgtgcgggtgcggggctggccttcccgtggtctcaagagtgggaggtgtgagtctctccctggatgtttagctcagcatcacctcagagtttgagcttgactgaaaacaacaacaataacaaatatgccgaaatatgatatcatttaaaataaacccaacaacctttaaaaattaacattattccttaatcggaccattttactggtctttttttcttaagtcaggaaaataaatgcaagcctatgtgcaggtcctcaaaaaatgtgaaaataagcctagtaaaaatctggcaagagatgtggtttcatcagccacctcataagcagatcttttcacatagattttaagagagaaagttggtttatctgcactttagtggatgcttccgtttgcttaacttccgtaataagaatccctagaaaaacctggagataattagcagaagggcggaagcatgaatttaaattcttatacccacacttaattttgtcaactttatgatttgctaaccagtgctttttctgccagtttctttaagtctagcttagtattataataaaaggaatcaataataccaatctatatagtaattttcttcaagaaattcaaaatcctatggaggaatttaagaaatattttaggccgggtgtggtggctcacgcctgtaatcccagcactttgggaggctgaggctggcagatcacgaggtcaagagatcaagaccatcctggctaacatggtgaaaccccgtctccactaaaaatacaaaaaattagctgggcgtggtggcacatgcctgtagtcccagctactcgggaggctgagacaggaggattgcttgaacctgggaggcaaggttgcagtgagctgagatcgcaccactgcactccaggcctagcaacagagagagactcaatttcaaaagacaacaaaacaacaataacaacaacaacaacaacaaaaaacaaagaaatgttttaatatgagaaaacattttgcaaaagaagacatacacatggacaacaggtatatgaaaaagggctcaacttactaatcatcagagaaatgcacattaaaagcacaatgagatatcattgtaccccagtcagaatggctattatctaaaagacaaaaaataacagattttgatgaagatgtattgtagagaaaagaagacccttatacactgccggtggaaatttaaattagtacaacctctgtggaaaacagtatggggatttctcaaagaactaaaaatagaacctccatttgatccagcaatcccactactggaaatctacccaaaggaaaagaaatcaatgtatcaaaagcctgcacttgtatgtttatcacagcactatttccaacagcaaagatatggaatcagtgggtgattggataaagagaatttggcatatatacacaatgaaatacataaaatgcataaaatacaaatgcataaaaagaatgaaagaatgagatcttgtcatttgcagcaacatggatagaattggaagtcattatccgaagtgaaacaagccagacacagaacatcaaatatcgtgtgttctcactcctaagtgggtgttaaaaaatgtgttcacatggacgtagagagaggaatgatagacgatggagatttggaaaggtgagagagtgagagggggtggatgatgaaaaattacttatgggtacaatgtacataatttgggtgatgggtaccctagagccctgacttaaccactaggcaatctatgcatgtaacaaaattgcccttgtaccgcatacatttattcaaatagaaaatgaaatattttagcctatgctcattagtttacttatgaagcatctacatggctcactgcactttcttcctaagttctctgacttctgaggcaggaggtgaggccaggcagcgggcaactgtgcttccctttctcaccaggacaggtgccaggagcaagggacctaactattttggtgcaaagtgttcattaatgggtctgtgaatcccatctccagctccagcatgactccagggcctaacattaacttgagaacagtatacaggaaaagggatgtttttgaaaatgctgattatttcatagcctctgggtgtcagccgtgaatctcactttaagccccgctagggatcctgttgccaccactgagcagaaaatgcagtgcttctgtgaaggaatggggcacagaacagtcaccgctaatgtgttcctgaaaaagccggggtgacctttcttgtggctgcttcagaggatgatgtcactttgggacagaaaaaggaagatctctttccagttcacataggataataagagttctgattcttagttgcatgttgaagaacaaacaaggagcatagacactcatcctggggaaaaagactatggaatctttggaagctgagaggccagccacttactttcctgggatagagcttttataacccccgtattgggcttaactccatacctttgacgctaaagttgttcactctctggggaaaccatcaaaacaatcaaacagaaacaagagcttttaaaaatggaatatttaatacttatggcacaaataccttttagaggattgcaatgatcatggtagagagactgattcagacatttttcaaatgtttgttggaaatgtctgaaaatgacaggcagtgatagtatcaataactaaccttcatcaagatatctgtgcatcggataatctgctatatactttaaagagattatgtgctgtatgcctcccaacaacccaatgaggggtgagcgctattattaacaagaatatgtaaggcaactgggactcagagagattcagcaccttgccaaaggtccaacaggagttcctttagcaagacttaaaaaaatgattactgtggaaatttccaaacatatacaaaactagagaaattataatgagccccatgtatgcaccttataaattcaacaaatttcaatacgtggccaaactagtttcagctatcctcccacctgctttcctaccttattagttttcttttgctgctgtaacaaattgtcacaaacttcatgacttgaaacaacacaaagctattctcttctagttgtggaaaccagaaatccataatcaagtccactggataaagtcaaggtgtccacagggctggttctctctgaaggctccaggggagaatccatttgcttgcctttttcatctcctagtggccacctgcactccttggcttgtggccccttcctccatcttcaaagtgcctcactccagtctctgcttcttcattgcatcactgtctcctttgacactgattctcactcttctgcttccctcttataagaaccttgtgattacatcagatccaccagataatccaggatcatctccatctcaagatctttaatttactcacatctgcaaaatactctttggcatgtaagataaaattcataggttcccgggattagaacatggacatatctggggagccatgaatctaccacccgcaccctagcaccaacaggaatattttgaaacaaatctcagacattttatttaatccataaatatgtcagtgcatgcttcccagaagataaagacttaaaaaataactgcaataccattatcatacctaaaaacattaataagaattctttaatatcatcagctatttagtcagtgtttcagttttacctaattgtctcataattttattttaacaaatggtttattcaaattgagaaatgtttcttaggtctcttttaatctgtcagttccctttccttttcctttacttccttctttgtaatgttgagagaactagattgtttgtcctacggaatttggcaaatgtgttcctgtggtcattcacatgtggttttttttttcctgtattttccataaactggtagttaggtcttgagacttggtcatatttatggccgagaatttgattttatggccaagaatgcttcatcaatagtatgactttctattgtaccacacggggaagccaatgtctgttgtctccctcctgtgatgttaaggttggttgctgagttgaggtgttgagcactccatccttcttgtatcaaattccccatcagctttttacctacattggtgattatggcttagattcattacttcatgagggggggttacgacatagtcataatctaattctgtcattcagttttcatttttagccagaattcttatataaaaaaataaactttcctctatttgattatcttgaggtacaatttatgaaagaaagacaggagaaatgtctaattctttctcctaatgcatccatttgcagaatagtaactggttttctagcatcctacaaggatgaaaaagtgtttgtgtatgaatgtatgtgcattagtaggaagtcacggtttcgtatattcgtgatatttcagttctttgcagaagttattctttttcaaattgccccatctttagccaatgagagccccttcagattggctacagactcttttgacattaacctagaaacttcattgttttctgatgtaacaaaatgttttagactcatcttgtatatttactgtcccaaatatcaaatcagtgtttactccaaaaaagcccgtgtttcttttagtaggaaacggtatttagagaccatactctaaatcctgtagatcaggggtctccaacccccgggccatggaccagtaccagtccgcggcctgttgagaaccgggctgcacagcaggaggtgaacagcaggtgagtaagcgaagctttttctgtatttgcagccaatccccattgctcatattactgcctgagctccgcctcctgtcagatcagtggtggcattatattctcctaggagcctgaaccctattgtaactgcacatgccggtgatccaggttgtttgctccttatgagaatctaatgcctgacgatctgtcactgtctcccatcacccccagatgggaccatctagttgcaggaaaacaagttcagggctcccactgattctacattatggtgagttgtataattgttttattaaatattataatataatagaaataaaatgcacaataaatgtaatgcgtttgaatcaccccgaaaccatccctaacccccttgatctgtggaaaaataatcttttatgaaaccagtccccagtgccaaaaaggttggggaccactgctgtagatgctcagagctacttggccttatcattagtcgaatctagaaaattgtgtaccaaaagaactgatatcaccggtttatactgttattttcaactcaaagttaatattacatggttttacttaatttatgttatattatatttgtatttattttctcttatgctgaaaattttggttccaaatatcctcagtataatgacatatatatatatataaaagcatatgtatgtacatatttctctctatatatgaaataatatatcctctctatatgtaatagtatactgatatatatataaaatatactaatgcaaatacacatgtaatagtatattaggagtataatatatagatacatatagttttataagaataatagtgatgttattaatactataaaaacaacttaagatttaactacagtttcttttgttcttaagataaattctactagagatgtacagttaaatcgcagagttttaaaaccactgtaattaattccccttagtgtaattaaaccaccaactcgatgcatagttagctcaatgcaccaataagcttatgggcttattagttttattttgctttctacttagtgattgctatttttaagcatatgtaacactaaccttatggttcaaagtcaagtgtataaaacaagatgcattcagagaagtatgactcttattctgtctccaacctgacttttattctctcttctaccttccatctatagagaaccattttaattcaatttttatccttctactgctttttaataatttagccatagatattcataatccccggttaggcaaaaggtgtgggactaaacacactgttctgcatctggcgccacaacttttgaaagtaaaactccctaggcttgggaaatagaccattatttactttcttactgtagacctatcagtcgttctatcagaaaagggtcagtcctgcaaaaatgggaaaccagaatcctctttagattttagatttttcaggggtgtgtttgtgactcacactgtgatgatattaatgttttactcattataattccagtataccaccccaaaatcccccaaaacaaacaacagtcataattaattgtatgcactgattttggacatactgctttattttcagccagatggtttgccaaaaaaatatttttctatgttactatgtgataattataaacatggttttaatagtaattgatgttccagtaagtgcacatactataacttgcttaccatatttcattgaagggcatttggttggtttctaactttttgttatatgatagtttaaaacactaatttctctttgctcaagagccctgaagctttgaactgatggggactgacaccatttagctaaaagatatcttgttcttactttcctggggtcctttttttgaggggtggaagtgtgaggaaacatggagatcttctcaacccaaagaaagccttttactcaacaatttcaagagaagaaaaatatgcaaagtacaaaagatgaagatagaagggaaaatcgccttttatattaggttaaatttttgagatagggtcttgctctgtcacccaggtgggagtgcagtggcacgatcacagctcactgcagcctcaacctcctgggctcgattaattatcttaccttagcctccaagtctaggtccacaagcacacaccaccatgtctggctaattttaaaattttttgtagagatggggtcttgcaatgttgttgagactggccttgaactcctgagctaaagtgatccttctgcctaggcctcccaaagtgttgggattacaggtatgagccactgtgcctggcctgaggctgtttaaaagaacaaaaatctttataaaaagtttcattgctgtttcttttctaaaataagttgtctataccagtggcatgcaaaatgtatcatatcagatttaatatgtgaataatgagggaaactgtgactttggacagaagactgtcgtagactgctctagtagaacagaatcttagctaatcaccatgcagatcccctgggttttagagttcatactgatcctcaactgggggctaacaatggtttctcatttatcattttcccctgcttgttcagcattctacttgaagccacagagaatgatggagcttacatatttcctttgataagatgaagaaatgaaaacatacattaatgaaaaataattccagaggttttttggactataggagttaagaggagctgaccagtgagcctgtggttgtccatgaaggttctgtggggtagagtttgaactgtccctatccagaatagtaggagggacagagatgcttatgagtgaaggccgcagaacaaggatggcatgacttgttcatggggctggggagaaccagtctgaccagacagaaagggttattgtggtttgacggtcggtgaaggttgttaggtggcctttggccacataagagagtgttagaaggtagacagattattttaagtgtgatgaaacaagtagcagttggtatgtagagaagtggggagaggtttgatgaaaagcagtgtgttagaaagacggttggcaacactattaagagtagattaaaaaggaaaaggaatgtgaaattagagagaccagaaaggaacctattatgataataaagtcatgattccatggaaagctgaccagaatgatgatactattagagaagagagaggagaagtaaggccaattttttctttaattaatgagtcttagtgactgaatgtgggaaataacagagtagtgattccaggattttaagcgtcggtgattgtgagaatgggcaatgcaaagtattgattaaatcagcagcattagggttgggaactagattagaatgggagggggatgatatagtgaattcctccctccctccctctctgccttcctccttacctttctccttccctctttcttttcttccttcccttcctctcttccttccttctcccctttatttcttcttatctttacatcattatttacttttacatttagcaactattctattccctggagctctggtaccaataaacaaaagaaacaccatcttcgctctcttgaagcttacatttcattaagcagagtggcagataaaagaacaagataactcagatagtcatggtgctacaaagaaaataaaataggcttatgtgctagctggtgattgaggctgcagggactggacagtactactttagactggagcttctggaaagcgacttaggggaaacactcaggtgtcagccatgtgaagatctggtgacatttcaggtcattgcaggcagaaaaaaacaaatacgcaaagataagaacagccttggagcgtgtgaagaacaaaatgaaagctgagggcatgggagggtacatcgtaagagcaaggaggtgaggccaaagtgtgtttcagtcaaggacagatcaggacacagaaaccacctagtaatttaaacagggatggtctaatacaaataattattaattttaacagagaactggagtcatgagggattcaccagtaggaagtaaagacaagtctaaaaaatgtaagagcagcagatatatgaagcagctaccacccataggactgagatacagggtttcctgcccccaagataagaccagacctggttagagacagcatgaccatggcaaactggatagccaagaagtcagtgtcatgctgcagcagtgtaacctgctggaagtgccttctggaagaactttctgaaagttcttcatcaccctccatgatgccaaggaaagctgttcatagggagttgtttcaccagtggcaatctgctacgaaaagggcttctgggaaatgctggcatccagatgctggggaagctgcccagacagtagaagctgaattctggggaagctgtatgcttgcaggagtcagggggtgcagaagctgagtgctggagaagccactgtatcacaggagctgggtgcagaggaaacaacgcgtgcttcagaggctgggtgctagaggcctgtgcatgttgtaggagcagggtctagagaagccatctgcgtttcaggagctggctgagaacacactgatatgggcccgggggtggggtgggggtgggggtggggggaggtagaggagtggcagggagggaggtagtgggtggggcagtaggggagagggggttggggggttggggggaagggttgggggttggggggagggggtggaggtggggtgggaccctttcctcctgcaaggcctttctagctccctctactgacaaaacttaatactgtgtcagctagcaaagaaaatacctaaagggatcagctaaatttttgctgagcagtcaatgtacatttggagctgagaggcaataaactggcatagagaagtagcaatggctctgatctctgagggcctcgcagtcgtgttgatgattgtattcatttagatgctggttgtggttagtgcttgacattttaactaattttttcttccaattccaatgaaatagataattttattattcccactttacagacaagaaaactgaggctgagggaggttaaatcgcttgtccacaatcacatgctagtgcggcatggggccaggattcaaacatgagcagtcagtctaactccaaatctgcccccagactccataaagtcattactgatctttgaacagacagataaatgtatcaggaaagataaatacttaaaataactttgcagtcagtccttctgtaattaaatcagccacctctattttacagattttatgaatttagaaattttagttacttttaaagtaaagtacaagtttaccaggaagtacaaattaaaataaaacgtggagaatacaacacctttcaaaggaaagtagagaaattggatgattgaggtaattagaaaataatattttgagtatagtttgatggatttcaggcttcttcagcttgacaaaaacagaaaagagggggaaagttcaaaaatattattatggatttgaagagatgtcactcagaagatcttgataaaatgttcctcatccctagtggatggggggcgtgggtggcagggtacaaaacgaaacatggaaccagtgatgggcttttaggaaattctatatggagcttttaccatgcaccagacctcaggctggggaccaggggtacaaggatgagtaaggcatctggccttcccttaaggagcttgttgtctcatggctgggacatgataaggacattactacagacatacatgaagagctaaaggaataatatatggaggaagaactagttctgcccaagggggaagtcaagggaaggtaccaagaaagtatagcatttgagttggatgaagaactggccaagacagctgaggaaagggagatgtagattaggggccgacacaagtaaggggttggcaaggacatgaactgaacggggtttgaggaaaaagcttagcacggggtcaagaacgtagattttggggttagagtacctaattctggcctcctcaatattttctagtttcatgatcttggtcaaattatttaatctctctaaatttcaatttctctgtctatacaagaaaataaagtaggccggccatggtggctcacgcttgtaatcccagcactttgggaggccaaggtgggtgggtcacctgaggtcagaagttcgagaccagcctggccaacatggtgaaaccccatctctactaaaaatacaaaaaaaatattagccaggcatggtggtgcatgcctgtaatcccagctacttgggaggctgaggtaggagaattgcttgaacctggaaggcggaggttgtagtgagctgagattgtgccattgcactccaacctgggcaacaagagtgaaactccatccccccacccaccaaaaaaaaaaaaaaaaaaagaaagaaaataaagtagtacccccttcagtgtgttattatgcagactgaatggtatgaaaagcatgttttcaccattactggcacatgctcagttaacgttttcctgccattcgtagcagcaggatgaggagtattgaaaaggctaaagattagagttctgggaagaataataaggattagatgtcagaaagtgtgtcatgacatatttgtgtgtatgcgtgtgggtacacagaattttgaaatagttttcaattgaagcctttgtttgtaaccagtagcacagggagatgttcagttttgcacataataatgaatatctgttatgtctgttagccatttgtaactactattgttgtttctgtcatctccagtaaagtgtttctgtattttcacaaaaatgagcaagacctaccacaaaaaattatcaggtaagggtgactttatgggtcccctgctgtgtatttgacaccaaaatgactcatcacatgagttcctaagtgctatgtttggtttcagtgtagaataggagattgtgtttcccaggtagaaaaggagactttgaaatctttctcataaaatgcgtgaaaatctggtgccatcagatcatgagtcttgtcatcaggatgggactcactcttggatccatcaaccagctagctaggtaactctgagcaaataccagatgctcttcagcctgtgtcctgatctgtaaagttgagatcataatagtaatacttcacagtgaaattgtacactagaatgaaagaatgcataaaaagtgcctcgtaccctgccacgcacagagagggtgatacacatcggtcccctcatccagcatcttctaattcacccctccctgtccccagcttctcctgtctggggatctcaagttgggaatggtcatctttggatgaagctgccatgtttatgtggacttagaggcagatattctattgggaattcaatggatgcataaggcagtgtaggggaactgatttttagggagcaatatgaagtcagagaaattgcactcttatctctacttacgttcctcatggtcccttttattcttttatgacaaatgttacttcaagctacagtagtgttttcaccaggggctgaaggaattgatggagttgttttctttagaatgagccgtaacatccctttgttcacagagattgtaaatcctgcatctttcagattagatcatgtatgttcaatatcagtaggaaacaaacagagaagtaaatatttgctacaaaatgtctttctgctgccttgaaaatgagtttcctaaatctgacatgtcagttaccctgcttcatctgtttcacatttagagtcaaaggaaaacaatcccccacagcaaggagcctgtcacttcacttggtgtatccagtgaagataatgtgaccacagtgcagtgggattgaatactgataaggatgtggcgggccatgcttaaagtttggtgtctcattcaaatgactggctgagagatgaaaagaaaacaaaaattaatccccctcctggaaccttatactgattcacttagtggttggttgctgctctaaggtctgtctccttttccccccattaataatcataatcaaatagccattattggtttttggcaacagggtaattactttctcgtccttattttctgggacagtcggtctgttcatgagaaagggcggttgtgcaatcgttctccctacgtcaactctgagaagtcggttgtgccctgcagatattgatgtcagcctgtttgctctctggtggagtgaaagaagaaaataattttattgtgctatttttagacttcaatttagattgcagcctttatgatactttgtgtactggggatagaactgttgatgcagaattcagctgtgagatgctctaggttctacctgcccctgaaagatttttttcgcttagcctgaatgccttagaactattcagtcacattgccttcatcgtcagctgaaagctgagccatgacaatagggttaagatgcactttgtattgacagtgtttgccgatggaacttgctgccagcagtctccaagtctgcaatttgcctgtaaaggcctaatctgatcttttatatgcataaataatcagcaccatacaaattctctatgaacaaaatgagtttattgaccattcttgcttttctaaatagttttccagaggtgatagatgaaagccagacaagaaaaaaatatttctttgctggatttccttctgaacaaacaagaaggacccaggtacctgggactccaggtatctttttctcagctgggatggaaggccatggactaaagcagtcttcctagttctttggtattagcaatgagctggtcttaaacttcaaagggatctaagaaagtgccagtaatacctgtctgagcttgttttgtgacgtgtatgttaccacatgtattaaaattcagatcaatttgcatctaaattatatgagtgcatgtgacttggcaatccagaagctgtcaattttagtttataagaacctcaacttgggcctggtatggtggcttacacctgtaatcccagcactttaggaggctggagcaggtggatttgaggtcaggagttcaagagcagcctgaccaatatggtgaaaccccgtccctactaaaaataccaaaattagccgggtgtggtgacgtgtgcctgtagtcccagctactcaggaggctgaggcaagagaatcacttgaacctgggaggcagaggttacagcgagccgagatcgcgccactgcactccagcctgggcgacagagtgagaccctgtctcaaataaaaaagaaccgcaacttggtccttgctactccaagtgtggactgcagaccgacaacattgacatcacatgggtgtatttaaaaatgcagaatctcaggcacttccccaacctacaaatcagcacctgccttttaataatattccccaagtgattcataggcacgttaattattttttaagtcacaaatctttttaaaaatctactatgatctgaatgtttatgtctccctcaaattcatatatttggacctaatccccagtgcaatgatgttaagaagtggcacctttggtggtgattaggtcacgagggtggagccctcacgagtgctgattagcgcctgtataaaagagggcccagggggctcccatattcctttcaccgtgttaggatacagcaagaaggcaccacctgcaagaaattgggaacttaccagacatcaaatttgctgtccccttgatcttggactccccacccttcagaactgtgagaaataaatttcttttgtttataagccacccagtttatggcatttttattatagcaactggaacagactaagacagaatcccataaaatctgtggaacctcttcccataaaaatgtacctacacacacaatttgtatataatttcaatggtgcaagttccgtggacttcaatgacacttccattcattggaagcagagagcatgcctgagggagcccccaagtgccatgtgccccgaacaatgctgacccatactgctccttgattgtcacattgaccaggtcttcaccttcatgcccagaccttgtctatgtaagcagcaccagcctctggggtgtcccatgtcagcctcagcagtccctttcatccatgtggctctcattaaccagtccttgctatcattttccttgcctggctactctggtcttttgctttctccttccccagtttagggactggtgggtacaaacattttagaatgaccatgaagtaatatcagttagctgaaacatatctatcaagattctttcctacccaggtcaggctcataaacagatgagatggagaaaataggtcttcctgctgtttcagctgacggtccacccatgctgcaggtcctgctggaagttcacttctgtgttctggtctgtgctttgatttcttctaatctttggtgatggcataaagataacgcaatgcaggctaaagaaaaccctttaagaaatctgttcacttactggcggatttgcttatcataggcatgccctgaccttgaagagtccttgctctggtagggaagcgcatgtacatggatgattaaaataaatgcttcatgtcaaaatacaggtacagaatgctattttgctacgaaggaaagaataactagcttttccaagaggagtgagggagagcatcaaagagagctggcttttaagggataaagcagacattgcctgagataagagctatagaattgcttattgtgagaaactacagagttgggaagatcgagatgagacaggcatatgagcagaagtgagagacctggcaggaataagagctagcacttagaccctgcttgctgtagggcgtgcacaggtctaagtgctttatatcaagtacctgtgcagttctctccacagtccaacaatccttccattatggatgaggaaacacatgcgcaccgagttcagtatcttacctaacgctgtgcgactgcaagcggtggagctgggctttgaacctatgcagtctggcttctacactacacttccaggcagagggcatctccctgtgcttctgggataaaacttgcagcactgccttgtctaggactcaagaggcactgttttctcctcccctactggttctgcagtctttccaactctttccacttcatcaatgagccatgtccatcctcacatcactatgtgtctgtgtaatacttagggccactgagttatggggaaaaagagaaatggatatttcttagtattgtctggtggttctgttatctcacagttcatggaaaccagatcactacaggcacaaggcattaccatcagtctcagagaaggcctgacaccctgttatagatcagagagtcctggccaccactaaaggtcttggaaagacacggggaaggcaagcagaagcacaggtggcctgaagacatctccttccggccaagagtccccctgctcatcccccaccccacccaggcaccaccctccacagctaaaggtgtttatgctgtaggaaccctgtggtggtaggagatcgggggtgatagggctgcttggataggaaacatatgtgattgttggcactgctcttcatactgttgtcaaattcctcactgcttctgtgtacttggggtgacagtctgacagcctgtgaggtcagacaaagcccagattacttccctctcgcctttccgtctcgcacttgtgggctgccattcttaaaggagtcacgtgtgggctttctttgccagaagtaggcatggcttacatcttttgggtttgggcactggaaatgactctgcttcccctgcagacacaccctgatgaggctgcaggccactagctctgctcctttttcaacatcagaaacagaggctgaccattgctgggacatttcccttctcctgctcctaactttgtcatagacgtctcctgaactgcagttactgtgtttggaggtaacatcatccccttctttggtttgtgggtagggaataccagatacgagggagagcatccctaaggccttcacctgacaccagtcctgcctgtatgtcctgcagttctcatggtaagccatcagcctcgccaggcggcacctgtgtgccaagcagcatggcttattcattgtcttctctagaacacagaatagtttggggacatttctatgttttctttctccaacagccagatatatgtgtccacttagctgcacataattattgtctctctaactctttgacctacacatttgtcagataaaatattttgggtgagagcttgttagcagatctatttccattccactcttggcatttattagcgagccctcttcgggaagccgtatgttctgagaagtaggaagaataacgcattttacctatagtgcaatgtctggccagcgcctattctttctgggttgagctgctggaatgtattcaggggataagctgagaggcaatttacgaggagtgctctctccttgtcttgctcctgcttccctttaaaattccttggctgcccttagtgtggcatgtgtttccatttatcctatagttctcaagactcaatggagctcttgtacttctgaagggcacttactaggaagcccttctccctgacgttgagcttgcactgatcaaaacggggggccccacaaaccatcacagggatatctatgcaaagagggaatgtttagcaccaaagttaaggactgtgcatgctttggcagaattacaaaaggaaaaataagtggacaaagtatgttactttttttttattattatagggtgtgtgtggtttatttgaacatcaacaaaaacacaacagaaaggggtaaaatgtaaaggaaaagcccctttgctgtcctccaaccctgaggaagctgttagcagttttaagtgcatccttccagaaatatctccctcatgtatggacatatatgtcctttagttagttttagaaaattcaaatggaatcataacagaattttgttctgtgccttgccgtttttgcttaatttttcttggaggtctttcaactttaagaggtaaaaatggctgtatgatatgtaacaataagtgtggctgattccaattttctccatttggttgtttccagttatttctcatgctgcaatgatcatccttttttatatgtctcttttaccttgtctgaatattcctgtagagtaaatgcctagaagaggaatagctagtgaaaaggacatgtgcattttaaattttgaccaatattgctgaattactttcctaaaaaaggcagctgacgtatccctcacctttaaaacagcaaactatacaactctcagacctttgtctccgaatctgtgcatctttatattggatgcaaatcacgtgttgattggctggttttttttcttcctctgccttttttgcttattttcttttgggatatttgcttttttacttcttgactttcaaaactctttgctggttaataaatctaactcatagaccatgcaacgtgttagaaatatttttttctaatatgtcatttgtattttgactttcatttatggcacgtttgccacataagagtttaaattgatatgggtctaaatttttcactgtttttcacttttggatttcaggtttgtgtttgcttagaaagtttttccccacttcgagtcagtcaaataatgtaaaaattttcatttagctattgggcagcttagaagccaatactagactggccaaatagagtggcatgttgagacttaccctgagattttagtttttaatgcagcgaatccattgggatcacttataaaaaaggattttaccctattaatctttactagaatggatatttttaaacttaatttttttgtgcttctcattgtaggagtcctttgaatgtagcttccctgagtgttcattcagttcattagttattgagttcttatagtggatgacatcttgctagtctataggcttcatagggttttaaaaaatgaatacgaaatattcccaaacctgattaattacatacagtagaagagggcgtctgtagacaaaacccagagatcaaggcagaatgtaacaactctcttgagagtactacaaaatgccatgtggttcagagaaaggacatcacatatggttgtgaggtcagggaagaattttatattaaagtgacatgagccttgagatgggccttgatgaaatggttggatttcaatgggtgtggattttagagaaagggcataagaagtatgcatctgtaattagaaacctgtgcatttgttctgggaagagtgagaggtccactttgcaggtgtgtggagaactcacaggggtgcaatgaatgagtccagagtagggctgagggcatcttgggaaaggcctctgagaattttactctccatttgaaaaggaataagaggcattcacgtttttggagcaatactagatgggatagattggaagtatgaaatatttgaaggcagaaaggcccatcactaattgtagttttcctggagtaaagaaataaaatgcttaatgtggagtagtactggaaatggggaaaagcagacctgaacaatattctcagtttataatctgtaagagggtactggggctgaggaaaataacaagtttaaaaatgaccaagatttctagccttcatttggttcactctcttaacatgcattgagttcttgctttatgtcagcactgctctgggcgcttggactagaggagttgaggagttggcacgagacaggatccctgctctcaaggggccacagaaaacaaacaggccaggatgatggcaggtataaggaaaccatgaaaaaaaatggagaaattaagaagaagggcttgtttgatggggagccctgataactttgattctgtgtcttaaaacttcaattcatgcttcatataattatcaatttaattattgctggtggtgttctctttctctcttcaccctggtatactacggagtgatacaggaaaataaattgttattcgaattttaaataggctgcaaagttggaaagcactttaaaatctgaggctggttggcttatccacaacaggaaagggaagtatgttacactaatgatgagaatagatctttgatttctgattatatcttagtttcttcacctgtgaaatagagacctgtcctaaaacagtccttgtactttgctcacagtttggtgagtcataaatttgggaaggtcttgcctgggcatttctctgcacgggcctgcaccagggctgaacgtgcacaatggctcactggtgtggctggcaatggcaacagctgtcagctgggatcccagagggggccactgaccaggcatgtagtccctccagcatgacagtcttgggctagatagactccttgcatgacagctggcttgtctcagagcaagtgtcccagaagaaccaggtggaagctacagggcctttccctaccttgccttggaagttagatggcattactccctctctgttctattgattactccaaggaaggcaccaaggtcagcccagattcaagagaggaggatatagataccagctctttattataggaatgtcaaaagagtaaaggttatgtttttaaaacactacaatgtagcaataccatcttacctcatgaggttgtcttgaagactaaatgaagtaatatatgtacaacccttagaacagcacctgaccaaaaattattaacaatggtgatgatggtggtgctgggagtggggatggggatgaggatgatgaggatgatgatgagtagctgcactgcctttctgcaaaatgtcccttgttgtctggcaggttgagttttggaaagaatggttttacctaatttatatttcctataactatgaaggggattgtcatcggttaattgccttaatctcatcaccttctaactaacaatccagggaggggttgctatccattgtgttctaagggagacctgaatataaactaaattctattaggaagggcaagacaagaaagagagggtagtttcaaccaacaatgacctctacatgttcctcgttaaatactctttcttatattacatttcttcctttcatattatgaaatggcattggagaagaagactaaaatttccaaaagttcagtggaaaatagtattactttttctttcttagaaaatagaagacaaaggaataaaatttaaacatgtctaataacaacattttttttttctgaattggaggttaagttgcccagggaaaaacatgcctacagacacatgaggactattctaccttcatcagtttgcagtggaaaatttagtactattggccattcacatatcagatattttacaccatcatataaaaagacctggttattttctttttttctttttttatttattttattttattattattatactttaagttttaggatacatgtgcacaatgtgcaggttagttacatatgtatacatgtgccatgctggtgtgctgcacccattaactcgtcatttagcattaggtatatctcctaatgctatccctcccccctatccccaccccacaacagtccccagagtgtgatgttccccttcctgtgtccatgtgttctcattgttcaattcccacctatgagtgagaacatgcggtgtttggttttttgtccttgcaatagtttactgagaatggtgatttccaatttcatccatgtccctacaaaggacatgaactcatcattttttatggctgcatagtattccatggtgtatatgtgccacattttcttaatccagtctatcattgttggacatttgggttggttccaagtctttgctattgtgaatagtgccgcaataaacatacgtgtgcatgtgtctttatagcagcatgatttatagtcctttgggtatatacccagtaatgggatggctgggtcaaatggtatttccagttctagatccctgaggaatcgccacactgacttccacaatggttgaactagtttacagtcccaccaacagtgtaaaagtgttcctatttctccacatcctctccagtacctgtttttccctgactttttaatgattgccattctaactggtgtgagatggtatctcatcgtggttttgatttgcatttctctgatggccagtgatgatgagcgttttttcatgtgtcttttggctgcataaatgtcttcttttgagaagtgtctgttcatatccttcgtccactttttgatggggttgtttgtttttttcttgtaaatttgtttgtgttcattgtagattctggatattagccatttgtcagatgagtaggttgcgaaaattttctcccattttgtaggttgcctgttcactctgatggtagcttcttttgctgtgcagaagctctttagtttaattagatcccatttgtcaattttgtcttttgttgccattgcttttggtgttttggacatgaagtccttgcccatgcctgtgtcctgaatggtaatgcctaggttctcttctagggtttttttggttttaaggacctggttattttcaaagcttccaagaaatggagtaaaatactaatgccctttaagtgaacatattaataaatcaggaaagagagtttggttataagagctgaaactctaggcatctgtgtcagaactcagtatttactctcctaaaatggatagaaacctacaaactcactgcctaccctctcccagagcaccattccccatagaaaaccccatagaaaaacactaacatgcctgggtctcaggaggattcaagccacactcagatgatctcctagaccccattttacttccaactggcaggaatcacaggaatggtgaatatcaattctgttctcaagaacttacccccaaacactgaaattgtatctgctagctgcctcattactggttgtgaaaacatcaccaaagatcttgcacagttcaagtagctgtgagtccttctcttcaataattattactaatgatctaattgttttccctgggcagaaatagcaaagcttctgaagacttcaaaatcaaccactactctctttagcacaaacagatcaataagcaggtatcttaaaaatccaaagcttgatgaaattattatgtattatgaaattacactgtaattttaccactacatattttcctcagcactcagcatagcatcccctcaaactggcttccaaactttgacgtgtgcattctaagagaagtttggggactaccctcactcagggtgcacatactctgtcccatcaaatagcaaatagacacaaagttacagtattccagtcattatactgcagaaaattatttacagaagcagcaaacctttcttaggaaagttataagctaggctgctggctctttcctttgaatccagcctcgctcacaccgtagaagccagagcaatgttgatggatctgtcaagttcataataaaaaataacagagaactcctcaggtaactgaaggtgatttggcccacgtgtggggagaaaacagatgacagcgtggagggggcttttcagacatgaccttttcttcctccttcacattgctgaggaaggccaatcctacctaggtgcttcaagaaacaaacccctgctggcagtgaggctcggggttttgaggctagacaagcagtagcagaggagcactgcctgggagggggtcaagcagatggccaggatttcagcatctccatcctccctgttccccaaggggtaggtaattacaattgagggagactatatttctgggagcacagatgattcccccagaggctcttttccctgcaccctcctaccacctgaacccactgggacctgcacaagtgagggccttggcagtatccacttctccaaagacccaagaccagcctaagtagtaaatatgcctaaggaaataagaactcatttggaaaataaacaagactgctttggaggacaactgttaaaaaagaacggtaataaatggaatggctaaaacctgatgaagcatggttattgaagtagacatacaaggtgttcaacacacatatcaaatattccccaaagataaggaaggatgttggaaacatgacataaaacatgaagccctaaaaaggagtcaaatggaaaatcaaagaatgcaaagtttaattattttaagtttcagtaaatgcattgaatgccaagtggattcagctgaaaagccaattggcgagccagaagatctcagaaattctcagaggacaccagcaatagtcagagagagggaaaacttaaaagataaatgaaacaatatgaagaatagcaatatgaagatcaacattcatataaaaagaatcccagaaggaaggaaaacaatcaatagaaaaatggaaggaaacatatgagagaaagattgagaaccgcccagaaaaaggaaaaggtataaaacttccgaccaatgggcttagtagagtgcaaaacaggatagaaaaaaaggtgatgggcatacctcaataggttgtaataacatttatgtaaaacaatgatagagagaacattctaagagcaggtcaattattaaaggatgagaactagatcgacctcagacttctcaatagaacacataatacaagaggctatcaagtaatattttcaaaatgtggaaccaagaatattaaataaaaccattatttaaatgggactatgagacctggaaatacttaaaagatttatcatgcacaagcttcttgggaatattcttagaggaagtatttcaagaagatgaaaagtctaggaggacagtatgagtggtgtcacagagtatgcaaataaagcttagcaaattttttactgttgatgacatacagaacttacaaatagaacaaacaacaaaaatatattttaataaaaatctggaactaaaatatagcctcaaaatatatatatcaatttattgcttcagcggtgggatggggtagaatgagagtgaggtgaaaatacattaaagcacttttcttcttgtttgcgaggaagatgtagaaattgaaaagctcagcaaatttatatggaaaaacataaatatgatcttacagatacaaaggaaactaccagaagacaaaaatataatgtataatttttagaccaatagaagaagatttgatccacccaatggaaagtaggaaaagagaaaaaaaataaaatgtttaaaaagtaagggaggattcctggtctgggaaaaaaggcatggaccatttctctctgcttcttcccactaaatgcaattctaaatcctgaaaataatgcaagagctaatcaaatgagaactctgaaagaccgtgagaggacagaaatctgccttgggactgcaggactagagagaaaacatagcttcagggcttcctacatacctacttcatgaaaaaagatgacctagacctggatttcctgacctccaaccaagcaaccaaaggcagctcagctaggtttattcctgccctggatttggagccctgctaacaataagcagccaggggagagctctccttccctcctggcctccaactcccttcccctcccagagacacctggttagccaggagcccctggcaagagggaccctgccacaacagatgcccagcctgggaagtgttctcctctcccctccctgccccaccaggggcaaggggaattctgccacagtaggtacccagccagggaagtcttcttgtcctccagagtctgagactttacacccgcaccaagagacattcagttacctgacctggggggaggccccttctgccttctgccccttcagctagcaccatcagggaccagtggaagttctcctagagccagatgaatcaagcagaaaaacaacaacaacaacaacaacaacaacaacaacaaaacaaccaactccaaaacaaaactcgaaggcaaacaaatgaaaggaaccacactgcaatggcttcaaaaataaaaattccattggaaccacatatagaaaagtaggacgggacctgcatgataaagctaaacagaattccctcataaaataaaaaaattaaatggaactcaacgttttttaacataatgacccacatgcccaagatataattaggaaccattcatcataacaaaaaccaaggatatcacagcttggatgaggaaagagaatcaaccgataccagcggggagatgaatcagatgttggaattatgtgacaagaattgtgtatggtcatcatgaaaatgcttcattaaactattacaacaaatgaaaaaattagaaaaggtcagcaaagatatagaaatttttttaaaaaccaaatgtaaattatagaactaaaaatacaaaaacagaaataaaaatctcactgcataggctcaattgtagagtgaaggtgatggagaatagattcagtgaaattgagaacagattaatagaatttacccaatctgaacaagaaacaatggactgaaaaaaatgaacagagcctctgggctctgtgggacatagcaaaagatccaccatttatatcactggattctaaaaggagaaaaggagagtgaggctaaagaatatttgaagaaacaatggctaaaagctccccaaatttggtgaaagccataatcctacagattcaagaagctacacaaatccccaggagaataaataaaaataaatccacactaagacacataagaattgaatttctgaaaactaaagacaaggaaaaatccttgagaacacccagagagatttactacctatagggaaacaaaatttcaataacagtgaatttatcatctgaaactatggaagccagaaagaagtggcacaattatcaaatgataatagaaaagaactgtcaacctacaatcctacatccagcaaaattatcctttaagaatgaaaaggaaataaatatattcccagatgaagaaaactaccgtgtatttgttgttagcagattaaagattagctagatgaaattctctgggaggtacagaaaggaatatagaataaaaagagtagaaacacagtagaatatcctttacctcatgtgttttatgtattgtattatggtgattgagacaaaaattttaatgtcatctgatacttaagacaatagtattttattttattttatttttgagatagagtctcactctcacccaggctggagtgcagtggcgtgatcttggttcaccgcaacctccaattcccaggttcaagtgattcccctgtctcagcctcctgagtagctggaactacaagcatgtgccactgtgtccagctaatttttgcatttttagtagagatggggtttcaccatattggtcaggctggtctccaactcctgacctcaggtgatccacctgcctcaccctcccaaagtgctgggattacacacgtgagccaccacaccaggcaataatattttaaagtgcagaaggtagagattgaaatggaagggaggtttatttgcatacttaaagtagtaaaatattggtgccagttgatggtgatgccaccagcatattgtaatacacagagcaattattatgcaaaatatacaaagagatataatccaacatattataaataaattgagatgaaaccctaaaaataggtccataaaaatatgctcaaatgatttttggcgaaggtataaaagtaattcagtgaaggaacaatagaatttttaacaaatggagctggagctattgaacattcatagggaaaaagatgaactcaaactaagtctcatacttcatgcaaaaattaacttaaaatggatcaaggacctaattgtaaaatataaaactataaaacttttaggaaaaaaaaaggagtcagtctttgagacctaaggcaaggcaaagatttccaagactcgacattagaagcacaatcaagtaaaaggaaacattgataaactgaacctctttgaagtaaaaccttttgctctgtaaagattcatgtgaagaagatgaaaagacaaactagtgactgggagacatgtttgtaaccatatatatctgacaaagtattagtatgtacaatgtacaaagtctcaaaacccaacactaaaaaatattgagtagaaaagaagcaaaagacataaaatacacctcactgaagaggctatatggatggcaaagaagcacgtgaaaagatagtcaacatcattagaaatgagggtaatggaaattgaagctgcaatgagataacattacacacctctcagaatggcaaaaacaaaaaaaaattataacaccaaatattggtgaggatgcagagaaacaagatcactcatgcatttttgataagaatgcaaatggttggccactctgaaaaacagtttggcagtttcttataaaactaaacatgtaactatgatacaacccagcaattgtgctgttggacaatcatttcagaaaaatgaaaatttaatttcacacaaacacctatgcacagatgttcatagcatcttttattcataatatccccacactaggagtatccgagatgtctttctgtgggtgaatggttaaacacactgtggtacatacctagcatggaatactactcagcagttgaaaaggaataaacaattgatacacgaatctccagggaattatgctgagcacacacacacacacacacacacacacacacacacacacacacaaaagccaatcccttaaggtatctactttatgattctgtttatacaacgtttttgaaatgacaacattttaaaaagtagtaacagacttagtggtctccagggactagagagggggaggcatgggacgtaggcaggtgtggttataaaagagaacatggagattcatgtgataattgaattgttctgtatcttgaatgtggtaaggggtacctgaacttacataagtgataaaatcacataaaactatgaaaacacacacacaaggggaactctgacgaagatcagtggattttatcaatgtcaatactctggcatgatattgcactactgttttgcaaagtgttaccattatgaagaactgggtaaaggtacatgagatctctctatactctttcttacaattgtatgtgaatttaataaaaagaaaaagaaaaatagaaaaaaagaagggaatttttttaattgaaagacttggctaaaatcaatgagaaaacaccaataattacacagtaatgagagtacatgggttaaaaccagcaatccaaagataaaatcttagaatgggttttatacaattcagcaaatgttttatgttggagtcattttaaaaatatgcaaataaactgaaaacatagaatggtaaaaagataagctgggtaaatttgaattaaaagagtgttggaataacaattttagtatcagacaatttaggctttaaggctaaaatatcatcaacaatgaaaattgtcacatacagaaaagaagcagttagaaaatttaatgattggaacccctaggtatctaacatcataatctgagactcataaaacaaccaataaaactatgggaggaaatagaagaatcaagaattatagttggaaatttaaaacactcatttcagaaactcatagatgaagcaaacaacaagaaaaacaaatatataaagattttaataacataatatgccttcactttccattcagtgaacacaatgctcattctttttggacatatatggaaaatttacaaaaatcaatgaagtactaggtgataggagaaccctcagataattttatagagttaattctcaaccagtatgcaataaaattagatattaatctaacaaaaagggaggtgaaaaaaatttattctatttagaaacgaaaaaacatatcactatacagcccttgaattaacaagggaaccattaggaaagataatgaaaaggctgtctgtcaaatctgctggacaacaatgctgtaaattctaaaaatatatataatgcaattacagaaaaatatttcaaattgatagattttgtgataggcccacaagtgtttgttaaattattttccatatttttctgtatatttaaaatatttaataattaaaaatttaaataaacatatattggacatggttaaaatattactcagacaaaaattaagagctttaaatatgcttattataacattaaaatttcaaaagaggaaaatatgaaaaagaaaacaaatgagttaagtgtttacagaagctaataaaaagtaagataataaatccaaagaaatggggaaaatggaaatataagataagaacaaatcaatgaaatagtgaacaaagatcaatatgaagattaacaaaggaaaaattagttctttgaaaggagtaataagatagcaaacctttgataagactgaataagataaatcagaaaaagaataagtaaaacacatactgaaaaggagatctacgaacaagtaattttttaaatactttttaaaaatatgaacaaacatgtaacaatacatttgaaattcattaaaaaaggattaatttatataaaggatataaaatattaaaatggcagaagaataaatggaaaacttgaatagaatgtaaaagttaaagatactgaagtggtaataaaatgtctccttcaacaaaacccttgggaaaatgatagggaacccgagaaagaagcaaataggcaatgaatatgaaaagataattcacagaatagtggagaaatacaacttcaaacgagatcccaccttttacctattagattggctaaaattaggaaaatgaaaaataccaaagtgcccccaactgaggactgttgtttctagtatggaccagaccccattcctgcactcctgctccagtgcatcgctcagaaaatctattccctccctccctcgctccatctctccctccctccctccctgcctccctccctccctcccttccttccttccttccttctttccttccttccttcttcccttccttccatccttcttcctttccttccttccttccttccttcttcccttccttccatccttcttctcttctttccttccttccttccctctctccctccctcactccctccctccatccttccttccttccttccctctctccctccctccctccctcctcccttcctcctttcctccttttcttattatattttctttctctttttgttattttatattggcatagacataaaatacatacatcatgaatgtactgcttgatgatttttcactcatggaatcctcaccatcaagaaataaaatacttcttgtacacaagaagtcccattatgcctctcacattatgagccaccatgcctagcctattttacttttatttttgaagaaaattcaaataattgcatagttctagattgccagttaatttctttcagcattttaaagatgttttattgtctcctagctttcaccacttctgtcatccatatttttgttcctttgaagttaatgtgcctttcttttctctcactgcttttaagagttttgttctgtctttgattttagaagtttcattatgatgggcctaggtatgtgttttctttgatcttttcctattttgagttgtagtgtttcctaaatgtgtggtttgatgttctttgtcaatacatgatattcttagctgataatctcttctaatattacttctgcctaattccttctttatccttccgatctcatcatttatggacatgggctttgagagaatgttaattaatatgtccaaaaaaatagggaacatgatggagaattttgacagagaactggaatatataaaagaaaatcaaattaaaagtatctgaaacttagaacacaatagtggattgaacagcagattcgagacagtagaaggaagggttattaaatggaagataggtctgtagaaaatatctagcatgaagctgtctggatgggagaaaatatatattttcattgaaaattcctgattttggttttccaaaaaaaagtcaaaattttgtcaacattgaatttacattcctcatagtttcagtgtgcctgagctgatgagttgttgatgtgctccatatacagggatatgtactcccaactagccacagtcactcctttctattatcttctaccactgaggctgaatattagttgtcatggtttatatcatcccttccctgctccacacatttacattgcgtgctgggcatgctatatatttgagttcatgatccatgttgaagatgccataaagacccaaaaatgtgaaagagacttggaaccgggtaacaggcagaggttggaactgtttggagggctcagaagaagacaggaagatgtgggaaagtttgtaacttcctagagacttgttgaatggctttgaccaaaatgctgatagtgatatggataatgaagtccaggctgaggtggtatcagatggagatgaggaacttgttgggaactggagcaaaggggactcttgttatgttttagcaaagagatgggcagcattttgccccagccctagagatctcgggaactttgaacttgagagaggtgatttagggtatctagtggaagaaatgtctaagcagcaaagcattcaagacgagacttgggtgctcttaaagcagtgcatttttatgcattcatgaagagatgatgtgtaattggaacttatgtttaaaagggaagcagggcataaaattttagaaaattcacagcctgatgatacagtagaaaagaaaattccattttctgaggagaaattcaagctggctgcagaaatttgcataagtaacaagcaaccaaatgttaatccccaagacaatggagaaaatgtctctagggcatgtcagaggtcttcatggcagcccctcccattacaggcctggagacctaggaggaaaatatggttttgtaggctgtgcccagggccttgcagctttgtgcagtctcaggacttggtgccctctgtcccacacatggctaaaaggggccaacgtacagctcagcctgttgcttcagagggtccaagccccaagccttggaggcttacatgtggtgttggacctgtgagtgcacagaagtcaagaattgaggtttgggaacctctgcctagatttcagaggatgtatggaaacgcctggatttccaggcagaagtttgctgcaggggtggagcactcatggagaacctctgctagagcagtgtggaagggaaatgtggggtcagagcccccacactgagtccccactggggcactgcctagtggagctgtgagaagagggccaccaaccttcagaccccagaatggtagatccactgatagcttgcaccatgcacctggaaaagctgcagacactcagtgctagcccgtgaaagcagccaggagggggactgtaccctgcaaagccataggggtggagctgtccaagaccataggaacccatctcttgcatcagcatgacctggttctgagacatggagtcaaaggagatcatttcagatctttaagatttgactgcccagctggaatttggagttgcgtggggcctgtagcccctttgttttggccagtttctcccatttgggatggagcatttatccaatgcctgtacccctaatgtatcttggaagtaactaacttgcttttgattttacaggctcctaggtggaagagacttactttgtctcagattagattttggacttggaattttgggctaatgctggaatggattaaaactttggggtactgttggaaaagcatgattggttttgaaatttgaaagggacttgagatttgagaggggctggggtgaaatgatatggttaggctttgtgtccccacccaaatttcatcttgaattgtaatccccataatccccaaatgttaagggagagaccaggtgtaggtaattgaatcatgggggcagtttccccagtgctgttcccaggatagtgagttctcatgagatctggtggctttataaagggctctttccccctgcgttctgcacttctccttcctgccaccatgtgaagaaggtgccttgcttccccttcaccttctgccatgattgtcagtttcttgaggcctccccagccacactgaactgtgagtcaattaaacctctttcctttgtaaattaacgagtcttgggcagttctttttagcagaatgaaaacagactaatacaatagagtctagcaatagagtggagaacaacatagttacaagctctgccctcagggaaaagaaaacaataatcacataaataattagaattgtcataaatactctgaaataaaagtaaaaaatgctaagagatccatcatattttccagttggaaggcagggaatgtttctcccaaaaaggttatttaagccgagttctgaagataatatgatttagtcagatgaaggcctggttatatgtgtgttgatggagaatggacagagagagttgatctagcctggaagagttggacaggtctgaggattcaaagcaagtcagtagggctggtaacacatatatgagcagaaactgtgttgtattgggctgctgagtctggaaaaatacataggattttggatgcctttgcagaccaagcagctaaggaccagcacatttcatggtgacttgatggtgtgcaaatttggtccctctgatgaatcttctagttgaaataatttatcactgtgaaattaagtcatcaagtataagatgaccagctgacttttagccaggtagacaagctatcgacttggtatgatgaaacatcactgagttcatttttatgcctttgtttcctctctgtctcagctgcaagtgtccactgcttgggaaagtgtgggaagctgacttggaagcttgtcgattgcttatccagagcctacagctccaggaagccaggggaagcctgtctgtagaagatgagaggcagatggatgacttagagggagctgctcctcctattccccccaggctccactccgaggataaaaggaagacccctttgaaggtattggaagaatggaagactcacctcatcccctctctgcactgtgctggaggtgaacagaaagaggtctgtccttttcacatggcctccagaggggacccttattctaaggggtgctttgggaccatgctccaaatgggaacaataaatattgggaaggcttcccattgagcatataaacttttaaaagtttcaataactcttgaatatgattacaaaataaagtagaatttttgtagaagtttgaaatatagaagagcaaaaaaacccaacttttggcatatttagttccattttcattctaatttagtggctagaattagaatatgcagtctaatatagaattaccatatatagcatacattatatatgtcatatataatatagttattatattcactatagattatatatgccatacatgatatttaacatatatactatacattatgtattgtatgtcatatatgatgtttatatatttagaatctatatattacaatatattattatttatttgaatgaaacattcaaatatatgtatttatcccattaattgtttttaccaaaaccagatcataatgaatgtttataatgttcttctttcataaacacaactaaagtttggcctgagatatatgtattacaaattaaaatatggttttatttaactttctttttctctcccctctttcctctctcccttccccttccactcctcactttcctcctcccaccctaacaagagtcttttccataaaaaggccctaggaagtttttcctgatttcagtgccatgacctgagtcttcagagctctttgttggcccaggttcacaggtgttgtgagtgtggaaaggaaatagcatggagaggctggaggaacttgacacatggctttcaagtctcatggttgatgttagatgttagctgtgcttcctccctgggtctttgccaggactcaccactggtgggtggggaaattgccacctccccaggccagccctgccagtctcacatcccatctgcaggaccatggccttcagagagcagacatttgtgttcttagtcactgcctctggactgcttagaaaatcgatccttgatcctatatttggattccatggtcttataagaaagcaaccactgtcactctgcatccttgctggcccagtcacaggctctacgttaatttatagacatctgaactcgtttgttaacccaggtgctacaggtggaatcctccagcaaagcctggtctccaatgctctgggtagaggtgttgaaaagttaccacctgctgcaagtggatggcttgaaagaatattctggggtggagggaagtgccacctaagcaagggtttattcctgggtggaagaaactgcaggctctgggaatgagtctttgaaggtgtgtctggctttccaaagtgagctaccaggaggagctcggtgtggggcagtcagaagccatgccaggtatgtgatggagtccctgggtcattgattctgctggaggccaggactgattcagcagagatccctttctcctgtagcggagaaaaatgaatatctttcctttacccatctaaaggttctcagctgaggcacctatacagacagagtaacaagagaaaaatgtacaaatgtgcttaatataaaatttacatgacacaggagcctgcaaaaatgaagacccaaagcaacagaaaccagtgtatttttatgcttaagtttgaagaatagcagacagatgtggagaagtataattggacaaaaggagtatgatttaatggcaataaactggggggagcttagcaaggcctgtttgctcggattcttcgctgcatccctgtgtcttcagagataaggatactcctttcctttgggtataggttgtacactttaaagatgagggtgttataatctactttagaggaaggtcagataattatttatggcctgcttcaggaaagaatggctgcagaaggtcagagagactttcctgcttctgctgttcctcaaatgccaaggtgccatattttggggtagtgtattctgaaccccatcactctctccctccctttctccctccttctctgccctgcccactgagccctgagccctccaggttctctggggacagactttcccaggcataggaggattcgttttcctgcatagcattataagcacctgtcaatttcactatctggctataagaacacagagctatatttgttgaaaaataggaagcttggggtcaaggatgaaaaaagagtggagccttatcctaggtcctgtggtggctgctgtttgggtgctccaggggtcccccagaaagcctcccagatcctctccaccaattgaaatgctgaggatgtcctacatttatttgttgctttcttgggggtgagtgggtgctttagaggggaccttacagcttttgctggctttcagtatcatttccctgttcctgtatcgcagtgctggatttattttcacagagagggcagccgttctctgaattcatgagtgccatggattttaagacaatgttttctgagaacggcaaaattatggggccagaaaacagataaattgttgccaggaaaggggaagggactcattacaagagcaacaggaaggaatttggtgcgtggaagttgggggcggttgatagaagtattctctctcttgattgtggtggtagttacgtgactgtgtgtatgtctaaatccataattttacatcagaaagagtgaattttactgtgtatgactttataacaattattttaaaaagtttttttaaatgtctacctatttaaaaaccgggctatagatgtttgagtcagtgcttttgtctcagggctatcaaaacgcagtgtggcctgctggaaagagcacaaggcttgagtctgagtctgccgtgatcctcagttaggcctcttacaggccgcatctccacatctgttaagaaaaacaccaccacgacataagaggataatgaaaactatctcatgaagctgtgatgaagatttaaataaaaggatgcatcacgaagtgagagacgcatacctagtagaggctaatttgtgttaacttttgtttctatcactctttctggcagtcacaagcatctctaaaatgctttttgcatgcataatgctagcaacagctatttgtttgtatgactcagtctccaggcttaactttcccttttgcataaggagtttgggggtgcttgagattgttacattttcctttacatgtataatatactcactttttgtcaagaagtagacatgagcaatgagttagtggaagatacccagtcagagaaaggtttaacgaaggaaaactcccgtttgtattcgggtccaggattttagactacagaaggtatggggaaatgataacaagggcctcattaggaaggtgcttttgttttcctgcctgtgttttccgggagccattgaagtctgggtactttgcatttatcctgtggctaaaatgtctattcgctttgcagctctgggttggttttgctttagaactccgtgctgggtctgaccatcctatactggtttctctactgaagcttgatgccctgaaggcagtcaatttctttttgacccaaatcttccatgaaaaattactgacttcttatagaggtccacaggaagatgagttttcaaagcctttcccgtgcttatctggccaacgtggtgatgtgcattctggtgatactttgttatcacaagggctttgtcgttgatcgacaccttcctgggtctacggtccttgccaggctgctgagcaccattcccccattttacagctgggcccattaaggtgaagcaatttgctttggactttgttacagaggaagtcagcaacagggctcagtgccaatcttagcatgaacttcctcgaattgtaagagcccttttcttttccagtttcatgacagctctcctccaagcctttctgaggactctggaaactatcagctgctgttggccctggttcgtagatgttacagaaattgaatgggggccagacaggagccagggaaatcagtgagccaggcttcctccgctgtgaagtggctcttttgtacgggctgttctggccataatacctgcgtaatttttttctttcctgtctgcaacctagcctgcggggtgctttctgtttccattgctttttctacataatgggctccctggacagtaccactctcccagacttctcatcccagctgcttcctttctgagccagtagctctcaccaaagaggccaaaagaggacctggcaatgttgagtcattgcgtgcagtatgtgagtagccagtgtgtgaacacagctgcagtgaacggaattagcaataagacacaacgcaaccaggtagcctcatttgttcagattgaggtcttcaaaaagcattttaagtttacgggtggaaaggtgtgatacctttcctcagccatcattaagggctaacactcctataacaaaaacaagttaacgagaaaaacataacacatttatttaatcagagttttacatggcacaggaggcttcagaaatgaagacccaaagacccaggaaaaactatttttatgctcagatttaatgcagaatagtcagctatgaaggaatgtgattggacaaaatgggtatgatctaatggttatagactggggggtgagcggaccagcaagccctgtctgttcagattcttcttggacgctctgcatagtatttattcctcttgggtatggggccggactcctctaaatggagggccttcaagggagaaggaagagagtgacctttctaggttttatgtttgctttgagggagaggagtcctagtttctaagactggctttgaggaagaggaattctggtttctatggcttgcatccagggagaaaaaggagtgtgagaggggagggcaagagaaggcagagagatcttgcttctgaggccttccaatctcctttggttcaaagtactcaggcataccaaagtgtcacacttaggggtatcatactgtgagccacaacataaatatctccattcctttttactcaatttcacataaaaaaagctggaaaaggtgatccacaatgaagatgtttgtcctccttgtcttaaactgatttctatatctagaaatgtctttattgggcaaaagtgcttcctcttcagccataatgggtgcagatctgattctttctcttctctgacacttactggccctgggacagctggaaactcactttgactctccaaggagcacatccctaaatgtgtgatctcttgatatctctcccacttttgaaaagaactgacaagggaaaaatgttcacttacccatcagctggttttgctctttttgtttaatgtctttggggagaaagcaaagagctaggacatgaagaaaacttctatctccactacattttgaagttttgcaaacccagccatctttcccaagagcagagcagtagaagcaaccaggatgcaaccagggttggtaattggttggttaattaaaaagtcagaaaagacagagagtcataaaagatgatgcatgcgtattttaaacctttttgtttctagctgaaaacatgccctctcatttgctattcattcgatataggaccaagtatttctgtttgtagagcctgctgattggcttttcacatgaccctcgttgtgtaaacctcacctatcatgcatcattaacagtttccacttttggtgtctttaaagtgggctggaaacgttaagagcacttgcaaaacaattctcagcctccccctctctcttaaatgttattgaatctttccaaggtttccagcttgctttttatggaaacaagcacttgttttcacattaatatttcatcgctctacatagtatttatttagattgtataattaggatatttaaatataactggcctacagaagtgaagaaacaaatgcaacttgaccaatagaaacagaactgcgcaggcttaccaggaccctgcccctggcctggccctggccactgaatttgagagagaaagcagagtccagggacaagctggcaggtggctaagagaggtgtttcttctgttcagtgagctggctcctgcttgttctctcagttaattactgtcatcagcttgtcagcctccttcacctgcccaagggaaggaaggggactcctctgatctgctggcttctccttctttcggaatatggtggaaccttgctccatgctccagggcctcacggtggccctttttccactaattaaaaatagttcagcgcttctgaggaaaacaacctcagggctcttggtgacgccgttcaccttggaaattccttgtcattcactggtgcagtctgagggatcgatagcaggatggcgtctgcctgaatggcaaggactcattttctgcgtttgtaacagaggaggtgttatccaatgaatcctcagtggtgtcctgggagtagctgagaatttttgtaatgtggaaaatcagatagcaccctgccccctcatcacagctacagtgcatttgatttccccaccactgggcagccagggcagttaaagtcaagagcttccactccccaggagcttccggtccccccataagctggtcgtgagctattcctgcccttcctaactaggagcaggcaggagatgaacactggagttccatgaagctcctgtgtgaaattatcttacctctacccaagacttactgcactgcctttttcagtgaatcgtttaatgccttccattctgaatatgctggtgtttctgctttgccagccattggtcattccacaccccctcctccacccggcccccaggtatttcctggaaccattgattatggcagagcagatcttattttgctcatatgattaggaacctcaggccaacaagcttgggtaatagtgagagaaaagaagagtggtcctttgctcttctttgtctgagtgccttttgagggaaagagtagataccaccaaccatagcatctctatctctttccagcaaactacaggggtgcctgagactttagggtgggaagcaagctctcacttggaaggggtgacatccagaaccaggattaaacgctgacatttaaaaacagtaactatggtagtcttaatagttgcacagtttacagtgaaacacttagagttcaaatttgttttactataaagtgaacatggatggagtttgggatttgattctgtcttcatagtttccctttacagttcaatatttcttagctcaaagaacacattcccttaaaaggaatccctcaaaccacttttcatttttattttcttatacaaaagggccttagggttggatgtccctatccccagaatctgaatccttttattaaagggactgtatctaggtactagccggtgccatgaatctcattttggattcaaatggattattttctcagagggttctaatgaagtctggttttattcagatttgactatcaaactcagtgtgcttaaagaaaagcagttcacatcctcttgcatgatcgtgtaatataaactgtagcttttcaaaggttgcccttcaagtaaactagaaaccacaggccaattaacatatgccaattagcaagcaatttcttcttctaacaatttgtttggttctgttaattacttttatttagagcttgtttctccgagccaccttcaaaacaagcatgcagatacttgaagacaattcattgcttgctctgacctaaagagatctaaatatttcagatgctttctttaccactgacgacatctggatgcttttaaaagaaatacgcagttactcagggagtaaccgtataatttgaatatgtggatgaatactcgtatcagatagagtaataaagatatacccttaatgtctcaattttgcctctctgcttattggcatttacacgactgtagaactcggcatttatatgaaaagacaaacaccataaaatctatgtcaaatacatgtgaaatatattctttgtgttgtgggcaggcatttgtttgaagtaaatattctttttgtagataatattttaaaaagaaagacaaaaccatatcgcattcctgaaatgtatttaccaagcatgtattttggaaaacagcccaaaggaacatatttaaggcagtgattttaatactaaactaaggaaatagtttgatgcatcagctaagctcatggatgggggtaatcacatctggtttacctcaagccagaggagaaggagtgagacttgacaatgtaacaaccattatgaggctcaagtagcgtttttggcttgtaagcatgtatgtgtgctgtgttcaattttatgttttaaatttttttaaaaaacttaaatactgctatagccccaaaatatgtaaaatatgtaaacagttggcttgatggccataagggaacacctgtccttttttttttttttttttttactgtgtagcctgtgactttcactgcaggaattaaatatttttgtggataggcaaatgtacttttgtggataggtattgctgatgccttattacacagccgaaggaaataaggatctgggtaagcagttgagtcacggcaaagagtaccgtatccaaggtggaggttcatgctagagctacaggtactaacccctcccctgcaccactgatccctagcaagggtggaagtaactgatgacttctctgcacagcccccagcacaatgtctgaaagagagacctaaagggtaactgcctgttttctcagattaatcttagaatcctagcagtacttgttacttcagtttggagatttttcttattctatttcttcacactcttacagacttcacatagcccctccctttagaataactcttgacagcattttaagaaatagactatttaaatcatcttgatccagtgactcaacccaaggtgtaacccgtctgctcaaatttgtggcaaatctccattgatgataacatttctgcaaatgtatgctctgttaggtgtggaagcttttcctaagtagaattggcagccacagtagtacctgcctacctagcaatttaacttaaacagtttccttttccaaaggtattttctattcagaatagaaataataaatatgatttcctcatcactctagatacagcctgatgagatattgttaccagatactttctcttatcaaaacaaccattattatttcataatatattccctaccctttgaatataagctccagttctaaaattttaaatgttggtatcttccacatgtcatagggaaagacttaagccttgtttatgggaactataactgagtaaaaatgtccttggagaccctggtcatggaatttgtgactgttcttctactggggcatgtggtttagacatctgctcaggctagcaactgaatgcttttctgggtatacacttttatagaaaaatgttttttgaaagtcactttaatgggaaaatcctgcccaacaaaggaaatcctattttctgtttttaaatcatgctccctgagttgggtgagcaggacagaaatttgacatcagctctaattcaacacagctgaaaatttctgcccaaacattcaggcctacgggttttgatcatgaggtgagttaagcaaattatatttgaaatattgcctggctgagtcggtgctactgaatcatacccgtgtggagggacagcagggaagatggtagggctttattggaggccaataaaagccatgtgtgtacagcagtactgtggagagccccaggaccccatggctgtccctgtggcaagaaaattatcccagataaggctgttcttggctgcactgaggcaaaggcatgtgctcagcatccagaaaggttcttcttgttggttttccttcctggctctttatacttgggtgaaatgatctcttggaagtgtagtctgaaaaacagcatagcttcttccaagttctcataagatacaggagtctccattcattacttcaataagcattactgagcaccttttatgagcaaggtaggacgacggaggcagggccaggacctcatcatctggtgagggagacaggcggttacacacacagggaggaactcctcctacccagaagtaggcagggttggagattcgggattgaagcgttcctggaagaagtggaatctgcagagttttgaaaagagggggtgttaggtgtagcaatcagaccatttacctgttataactgcttttcattctgacccacagggaaaacaattattcctagggttaaatgatgggacggatgttcagaaaggaaaaaaataataatttgaattgctgaaaggaccgcctgacaagacaaattactggagagtcttcaaagacactttgaaatgccgagcaacctgtactttaaatgatccgttcagaattgcaaatcctacagaactccatttctgctaggaatcccagtggctttgtagtgggtggtatttttaattatcaagtatcagtcttgcaggctttctcttttttgtcacatttgatttaaagtacaaatgggagcaaaagttacaaattgcacacagagagacactcagtgtgtgaaattgtggacatggttcagaatatgtttatttggaatccgctgaggtgatgggagagatgggactggatactcatgttcttatttggaaaggagacattctcgccgcctttcccatgtgcccgcttgcttttctctgcttccagcctgatattttatggacttgtggatgagaggtcctgtgcccatcattgcattccagattgtcatggctgttatcattgtcacctccactcattatgcctctgagtcctggagggagcatatccctttaataagactgaaccctccaggaattgagactttgcagagttctgagagtagaagggaagagaacaccatgtctacaggtgctggctgtttgcttctctgatttctttccatcaccactttgcccaagttacatgactctttggccaggtgcggtggctcacacctgtaatcccagcactttggggggccaaggcaggcagatcacctgagatcgggagttcgagaccagcctgaccaacatggagaaaccctgcctctactaaaaatacaaaaattagcctggcatggtggtgcatgcctgtaatcccagctactcgggaggctgaggcaggagaatcacttgaacccaggaggcagaggtcgcagtgagccgagatcacgccattgcactccagcctgggcaacaagagtgaaactgtctcaaaaaataaataaataaataaaaaataaaaggattcttcaagcctcttttcttgtatgtaaaagaaggaattgaaccttgaggatggttcctaagacccctaccaaatccagcagcattctctgcttctaggtattaagattctcagggaaggtttccgacattcttagtagccctgaaggaggcagatctttgtaaatgcatttgctgtcctgggaaacaagatggtgttaagataaggaagctccatttctcttgcagttataacccaaggcagagcctcctgattaatacacctggcttacaactcactattttggcttaaacagaaatgtcagtgcttccaaagctttcactgagacaccttctttgaaaaaaaaagtttcttttattttaaaagaagcaaacaaactctcttcaacagctccctttatttacttatttatttatgtagctttctatgaaggcttagcaaataaaacagataaaattagaaattgaggtagagttgacagtaaaaacccttttcctcctaccaggggatctctgcagaataaggcctaaagcccactggtttgagagaacatcctccagcaaggtgctttccaagcaagggaaaaactagaagtagtgaaatacacctgatgcttcctgcagagactatgccatcctgtgtgaccgtaacatataaacatcacataccaggaggacatgatcatttaaatagacagcaatgttgtccttcagtctaggcaatgggtgtgccccaaggccagtgtgagggtccgggagacaggaatctgtatatttcttacctgcctctcatgatttgagcaccttgctctgctaagtgtcccctgaagaaagagccacttctcccaaaggtggaagaaagtgaggccctgtgggtcgtattgcactcagatatgttggactcactgtggtatgtcttcctgcggaattttctaggaaatttttactgtacttgttctatacacacacacacacacacacacacacacacacacacacacaaccctcagaatatgtgcctgtactgttcaataacttacacattgaactaggagccagggatctgggattactgaccagtggcagtggtcacttgttgtaatgtctttgttgcacttcccagcctaatggccatagtgccctgcatgggtaggagttcccctccctgagagaagtctgggcattagcactttagtgagcttcaaaaatctagtcagtcaactccctaactaggtaccagtgaagatgcactacctgctagcattttgcaatagttttatccactcatgcgaggttctgtgtagggacaacccatgtaaggcccctgttttacctactatggctgcatcaaaatcaacacaaacttagtgatttaaaagaactatttatgatattcgtggatctgtagatcagaaattggggtggggcacagtgaggagggtgtctctgctccacaatgtctgggtgttagctgaatgacttgaagacaggggctgggaccatctgaaggttcagtccttcccaagtctggctgttgactagggctttagctgaggctctcatccagacatctacaaacagcccgtccatgtagctggggcttcctcacaacatggagactgggttccagggtgggtgatctgaaaagacatgtcaccttttatgacctgactttgcaagtcaggcagtgtcaccttcactatgttccatacattgaaggaatttcaagggccatccaggtttaacaggagaagtaataaactctgttttttgatggtggagaggcaatattccagaaaagtgtgttgctatgaccacacctacactctgccatacctgggcatggcatggaaatggccaaggaagttttgcctagtccctcatttatggactaagaggcaggacctgggcaactttgcacagctgatacccatggttcttttttttttttttttgagatggagtctcctctgttgccaggccggagtgcagtggtgcgatcttggcttactgcaacctccgcctcctggggtcaagcaattctcctgcctcagcctcctgagtagccgggactacaggcctgcaccaccacacccagctaatttttgtattattagcagagacagggttttaccatgttggccaggatggtctcgatctcttgaccttgtgatccacctgcctcggcctcccaaagtgctgggattacaggcgtgagccaccgcacccagccaatacccatggttctgatcccaccctgctctctctggacagacagatttctttggacagaacagaattttttaccagattgatagaaatatattaatctaccctctcatcagctgtcaattctcagtttaggtctaaactaattctattctcaagattccaagtgagagcattatagtaaaataatacatctcttaagtcatatctacccagttcacattcagtatcctctcagttcagaatcagaagctgtcagaccaacaagtagaggttcctgcacaagcctgagagagatcctgtaacttgtgccttggaaaaacagccccacgaaagaatgccagacagatcttctcttagcaaacatggtgacttgagctctctgaatttgttcccttatgctacattgtttgttttttaatctctaaaaaaactaaaaaaaatttcaggtaaattttcttcctttttcattatggtttgaattagggtttttgataactttttttccaaattttagggatgttaaaggttcaggaaagtataaagaagaaaataaaaatttcccataatactgccccacaaataataattataaatattttgatgctttgcctatttgagttatttcatcatcattaaatgctcttttattaaaaatgactttaatggctaaatacaaatctgctgtaccagaacttatttaactattccctttattgtttgacaattatttcctctttcctatacttttaatgccatcatctttgcatgaaaaatcttttatttctatttgatatatgtgataggcattcttccatgcttattacaagggcttaataaaagttatttcaatttaaatgaacaaattatttctttttgatagattccaggaaaggaaattattgggtcttaagatatgaacaatttaaagcgcttgttatgtggtctgaaattgctttccaaaatttatgccattactatcaatctgcacgtaatttaaaaaggaaaacaggcaggaattatatgtttgggggagaaatgtaaaactgtattttaaatttgtttaatttagctctgatttcatatttgttgtagccgtaagatgaagattgagtcatttgatttcgtttaagggtgttttaaattccttggttttctgttctgagtctatgctttttctctctttaagtgtgttttcagtcatatatattttcctgctaacactgtatgtttctccttcctgtatgatttgtggtttcatgactgtcaatcattttttcccagatgttctttttcttagtgtctcatttatttcatgtgtgatgacggtgttgctcattatccagtgtctacacttctgtgtaatctcctaccctcaatttcatatctttgattattttagggtcagtaaaaatatatatatatatacacacacatatatatgtatatatgtgtatgtgtgtatatatatatatatttatctgtgatttttatgtatttgctgtcttctcaccactgttatggtcaattttgagtaaaccttcaagggtgcttcacactaaagttcctttaattgtgtccatatgtccccacaattgtatgttattgccactagatttcttttctatatacacttacttgcagtaaatgcgtatagtcattctgtagcttgtagctcattcttcagtatactagacttctccaatatttacaagttatttgcagttcccattagcactattttattctaaggtagttggccattggcatgtaaattttaaagctaatatagcaaaaatcaaattatatacttggatttaattgttatttgtggaatctgattataatattatataatgcataatctccagactttttaacagtgactattaccgcacctgggcattttgttgttattgttgggtattcattgtatcttttgtagtaaacttgtagtttaagatgaatacgtatcttaatagtttcccttatgcaatttttttttttttgagacagcgtcttgctctgttgtccaggctggagtgcagtggcatgatctcagatcactgcaatctccgcctcccgggttcaagcgattcttctgcctcagcctcctgagtggctgggattacaggcgcccaccaccacgcccagctaatttttgtatttttttagagagggggtttaaccatgttggccaggctggtcttgaactcctgacctcatgatccacccgcctcagcctcccaaagtgctgggattacagctgcgagccactgcgtctggtctgcaaaaattttttagtgtatcttctaagcttttcactcttagttaaggaaatattatctttacatttaagttttaattctgagctcatttcttgcttctccccctgctttattctaagaatctctatcttctgttctcattttcttaaccttgtttattgttttcctttacaagtctgtggccttctacatgcccagcattcaactttgggccctgggatgaatgactaacccatggcccttacctcacttgagtggacacagaacttatcagaatggttgcactacaaggtgaaattattatttggggcctttgaaggaaaggctgggtttcagagaagagtggggaagaatgcatgggcaaaaagaacagcatgagagagggcaacggggttagtcctattggcaaagcttagcttgcctttaggagagttgaggggatacagctgaaccatgggctttgtgcgaggtcctatgaagacagagtttagactctgctctgtggtggtttcgtgcaggaaagtgacataaagaaggcccttttctggctcatagaaataagtttgaagggaaaaagtgagagaaggaaggagctctaagacttaattgttcattcatcaaatcgatcaatactacgtgcctgtcatgtgccagatactatgccaggggctctagggatagcagcaaaaagacatcatcatcctgctctttttttttttttttttgagacggagtctcgctctgtcgcccaggctggagtgcagtggtgcaatctcggctcactgcaagctccgcctcctgggttcacgccattctcctgcctcagcctcccaagtagctgggactacaggcgcccgccactacgcccggctaattttttgtattcttagtaaagacagggtttcaccgtgttagccaggatggtcttgatctcctgacctcgtgatccgcccgcttcggcctcccaaagtgctgcatcatcctgctcttatgcggcttaagctcccatgtatggtggtggtagcggcggctctatatgcagtatacacctcatggcacttaaattctagcattcagggactgtggtcagtgtgtaaacaaatgttcagtgccctagtcaattcctgtgtccttaaaggaatattccctttgtccttacaggttagcccagttgctcctgtgacccctctccccactttttttagaatccctattaaattttgaatttgcccattgatacagctggaagattcagggtttctgtttgataaatccacttcgggacttgacatactggaagcagcactttgaaatgaccagacaagggaggtggcctgtggaactgcatagacaaagctcattctttggtcataacttgaaatcagttcacaacttctcaagtttgatacccaggggagacacaggccctagcacaattctgactgaggtgcagaaactgggagagctctttccaggaagcttccctttgtgttctgtctgtgtccacggcactaacaagtgatcttgttttccccctctcgccaggaatcttacatcctttctgcagaacttggagaaaagtgtgaagacataggcaagaagctattgtacttggaagatcaacttcacacagcaatccacagtcatgatgaagatctcattcatatccttttcatcttgcagatgaagcaaggcaaagttattttataaaagagagtctttaaagggtagtttttcatgcctcagaagatggcaacaaatagatgctgccgttatttatagcacagagcacctcagatctctgagattatcaggtatgaaatgttttcgctctagttttcagtaagcaattcagtattttttttcttttttcttttttttttttttttttttttgatgccttggctggagtgcaatggtgtgatctcagctcactgcagcctccacctcctgggttcaaacggttctcctgcctcagcctcctgagtagctgggattacaggtgcccgccagcacgcccagctaatttttttgtatttttagtagagagagggttttgccatgtttgccaggctggtctagaactcctgacctcaagtgatccgcccgccttggcctcccaaagtgctgggattacaggcttgggccaccgcatccggccgcaattcagtatttttaattgctaaatccaaccactaacaattaagttcagacatgcacacgaggaagccaaagaaaatgcagtttcaagtgagtgtaaatccagctctgtgtggccctggtgaaggaaaacagttgactggctcgttttcctacgagctttttcaataccaagcgtcctaaatccacctctgaatttctttatctagcaactctacctagtaatgggcagtacagtaacaaagcaaacaggactttagattatttcctttcaacttctaaccatatacacatggttgaactcaatttagtcctttctgcttcccggtgatagaatttcttgcaccagacttaatatccttggtgtcacacctacaaacaccttcatcaaaactagctgctagtgagcaagatggcctgagaacatactggcttaggtatgcatccgtatgaagcactccattctgtgttgatctgtggacgcagtttgtcactggttagttctgctgagagcaaatgggtgggtacccagtggatgaagggatatgcagccctatatcctttcagttatttaatcacaagttgacacttctctagctaaaaacacttccctattcattcatctttctattcagtagaatgtcttttattagatgcgtttgtggcaaatttagctattttggtctttgcttgtcattgctgtcttactttttagaattccagtttttaccatactttatgggctgtgtgtgtgtgtgtgtgtgtgtgtgtgtgtgtgtgtataagtagctttgaatggccaaggattaatcttagtcataaatactcagatctgtgatttaatgatagagccaatgctacacttccttccttgttataactttacatttttgaccttgtttattaagctgggcagaaaatttcttcaacttaatctgcaatgattttcacattgctttaggtagattttgaaaagcagatattctgttcttcataatttcctgcctcttaaaaatcttgtatttaagctattatcagaaagtttcccaaagaagcatacaaacatagaataaggatggcgtttttatttattccatgttcatgggatcacaaatctttgagcacgtgtaagggggtatggcttccagcaaatgggcaggtgatggggcacagaggaggagtccgctgcagagagtcaatggatatgaaggggttcccgttccccagctcagcagatgggcagttcctaagtgggttgtacctctgtcttcaggtagcagttctgagggctgcagtcagggttctgtactggtctcttattgtgaaattttctaaaaggttagtttgattctggggaattggatcttatttttactagagctggggaggcagctctagtaaagacccctgaaaaacaaggggttaagcagtatttaaacaagatccttttagtttttccacagctgatccaagccagatttaatagcagcgggatcatggacaagtgataatgcctctttaagcattagggttcccatctgtaatgtcaggataacatacacctctaaggtttttatgggggttaaataaggctatattcataatgtacatttaatatagccatgggtaagtgcatgattttaggtagaaaagtgttatccaagtccatttgtctcacagttcttagtatattagtcacaagctccaagaacatcccatttatacacaaaaagtttcaaggtagattgattcaactattttgtaataaatgcttgaaggaatcaatcagtaactccacagtggcttagatgaaaagtcatttaattataataaaacttctataaatggatagggattattatccctacttgacagtcgaaaaaaacaaggcttgggaacaacttggctgacacaaggtcatttctgatgagtagcagaacttttcagccacaagccccatggtcctattaggcccatcagaaagcagccattgcctataagtaaatggacaggtctgttccatggaacacacaattactttaaatttttaagctataataaaagtatttcactgatgttatgttttttgagaatttcactgatctctgttgtgataagcggagaagcactggtttaggtactgaagtcagctctctccctccctccaataaaatctacaccacgatatggatttcaacccagacttttcttcgaatcccagtcttctgtcactgattgagaataaatacacagaaggtgtctttgaattcatacaaagcagcacttaaatttgagtggaacattatgccctaagatagaaagacatgtttacaaggctctacggaggctcccctacacaaaccaaagccgttgtttcaaattagagcttatctagagattcagagaggctgccagaatcctccagccaaactctgactggtttgtatttccatgtttgatcaaagatacaatgctgagtgtggctgaaagaaacagcttgttgtcaacaatacagaaggattgcacagtgagtgcagtgttttgtttgattccattaagcatgctgccagagcctactgtgcatgtgaggtacccaattaatctaagtccacattggaatgtgattaatgtaagcccacattggacgtccacgtctgaaagattcaagccacagatgctaccatggatgtttattctttgtgtggaaaacactcagtttttgcataaaccatgtgtgtacataagggctattagtgtggcatcaacagaggttaggggtgggcagtgggactgagtttccaaataggtccttgtggcctcctgatttaatatccgagttatgggcagcctcatgggtgtctcctacttttattaagaagacactgattgaccaatccaaggagggagaggaaagagcctttgcctcttcatcgtcctgacaggctcaccaaattacatgtagcatgaccaaagaacaacacaaacccatcagctactgtggcagcgagtgtctgaaaacagtctgcaccattaattgggagagctggtggtggagaggtggctcacgcctagtgcctgagaaggggacaggaggtgtgacttcacggccagtcaagtcgggacttccaggagcattagtgtgagtctctagtgcattttcacagtcatctggggtcctaattagcagtatgttttgaaaaacattttgactgtgtgcaaggtctcctccacgctcaaaatattgtaaccaaaagccttccaaattaaggtggatttttaagcaggaattttctcattttcttcctccttcaatttctacaacacactttcaaaggaaaagaagtacacctgtgatatgtacatttttggcatatttcaaagccctcagggcagttctgcctggccctcagtcatagacgaggtagagacactctgattctatttgtggttgtatttacatccatgtctgggaagcccttatggaattctgtttctctggttatttggagtgcatggtgcccaattgtttagttttggtggtgcttgagggcatgctctaaatacatccaggtttgtgcacaaataccacagctccgtaggacccttgtttagttcaactttccaaattttagcgtgaacagacaaaggtaacgagagttttgggagactgtcctgcctccttggagatgttttccacttctagagctgaggggctggaagagggatcatgaggatcatggagctccactcatgtagtttatagataagaaaatccagagcctggccaggtgcggtggcttacatcttaatcccggcactttgggaggctgaggtggatggattgtttgagaccaggagtttgagaccagcctaggcaacatggcaaaaccctgtctctattaaaaatacaaaaattaactgggcgtgatggtgcacacctgtagtcccagctactccagaggcgtgagaattgcttgaacccaggaggtggaggtcgcagtgagctgagactgcacactactacactccagcctgggcaacaaagtaagacactgccaaagaaggagggagggaaggaaggaaggaaggagggagagaggagagagagagagagaacaggaaggaaggaagggagaaaggagagacagagagagaggaaggaaggaagggagaaagagagagagaaagagaggaaggaaggaaggagaaagaaagataaagaaagaaaaagaaagaaaagaggaaggaaggaaggagagagggaaggagagaaaaggaaaggaaaaggaaaagaggaaagaaaaggaaagggaagggagagggaaaggagaagggaaaggaagagaagggaagaagggaagggagaagagacaagggaaaggagaagggaaaagggaaaggagaagggaagggagaagggaagggagcaaaagggaaggaaagaaagaaaggaaaggaaaggaaagaaagaaaaggaaaggaaagaaagaaaacccagagcctaagatgacagtatctaaagctttgtgtgtgatgggctggtgcagatatagtagtcacagttctttccttaggctctcacttggctttggagtgggacggggaagagttggagccccacagtgggtgggggagggcaagggcgtagacacagggctgtcctgctgctggctgagcacgaggtgggcttggcacaaggtaggcaacacagagtttctgtgtacaagcatcaaatcctttgaatcaatcttgggtatagtcaaggttttcctctttgctcttgaaagactgtgatcctcatggcttttccatgaggatctccagtaccaaattctccagcactatgggctgtagtggctacccaggaaggaatggtggtaagaagaaataatgtagcaaaagagtagcactttcaaaggataagagagtcccttatctgaaatgcttgggactagaagtgttttgaagttctgctttttttcagattgtggaatatttgcattatacttaccagtggagcataccaaatccaaaaatcccaaatctgaaatgttccaatgagcatttcctttgaacatcacgtcagtgctcagaaggttttggattctggagcatttcagattttggatttggggatttgggatgctcaacttgttctattgtttctttttgaatcaaactaggtcagatgaaccgaagacctaaatttgactaagccattgaaatgcttacttggacacttcattaacagtaaaacactcaattgttttcttataaaaacagaagactccatctaaagaattccaggatcttttgcaaatggtaccccaaatgacaatattgccttatcatctattcctgaatgcacctgtcctgctgtcttactcctttgctaattgcaaacacagacattttaacaagttacatgcttacctaccatgtggatagtctattccaaggaggtacagaatggccttgggttccaaaggtgttttaaagatgttaggatgttcatccctgcgacgttgttataatgaatctgctaattttcttagataccacaggtacttggtggcatatctgttatggggggcagatacttagctactcaggtccctaaggcacaggcttcataagaattattctgcttttttgaggcctattcttgcaattaggtgcagttagcacctctattctagttttggaatttgcctaaattgtcaatagttttggaatttgcctaaaatttctatttgcctaaaattccaattgtttttcagctctttgacaacaattaaggtcaaggtcaagtgtttcaatgggcttcaactatgagcttattggattagtgatcagaaaatctgattatagctactacttagccttaagatcttgcttagatgtatcaaatgagatagtttatttttctgtaagtcagtgagaaaatgttgtaaaacacttttttttaatttatggttcaatatgaatgccagacttatatctgttttctactcagcatcttctcttggctaataagcttgtccaacttaccctgtctaaaatccaattccctccctcctctaccaaaaacaccaaacgctcattcctagtcctcttcttctcaataaaatacagatgtgtttttctaattatttgctcagataaaaaatcagggagtccttcttaatgccttttccattttccatatcccacatccagtctgtcaccatatatatgctcttggttgtatctgcaaaacacatccagaatctagccacctctcctacctccactgccactgccctgttcaagccatgattgtttcttgcctgaactattgcagtggtctcctaactggtcactctgtgcttccgcccttatcctctttaatccattctcagcacagcagccaaactgacgatgtcacccctctgctccaggtcctcccacggctcctcccatctcactgtgaacacagcacagctggacataactaccaacatggcgcctgtttactttctgctgcactcataggtatttatgccatctatgatggcagtgactttgattcactcctgtgctgctgtgtccataacatttgttaacacacagtagaaactcaggaaacatttgaatgtttgaatgagtgaaagaaagaaagaggatctctctctgcactttcatttctcccaactgcttaacagggaaatgtttccagtggtttattttataaacatattgatttaaatatgaaagcaatgcatttccattgtgaatatcacagaagtatggaaaataaagctgtttccctcccagtgcctctacctagaggtcactactttgtcctcctgaagcctttttctagagctataaatggcttatctcaatagtcttgtttgcaaaattaggatcattccatataaattattctacaatatgctttcccccaccccattctgcatttttttttctggggacttttccatttcagtacatccaaaatgctttgtaactcacattttgtaagggatcctacaggcaggttggttagagagaagtactttgcaaataaaatgtgattttaattattattttgatgattattacccctacaggctcattaaatgaggtttctaggatttctggtgtctaaccctgctttgacagagcctaaggtatttgttacatctgttatattaagaaataaaccttaaatctcatgatgactcacaaaagacactatactctcacaaggaaaaaatatttctttgaaccctaaccctatgaggaaggtacaagattcagtattttacaagtagaataattaagcattgattgatttgctgagcttaccatagctagggagttatctcctcaaatgctggttttctgacaccttctgtgttagcgattgttgtatgagcggctgatgtgttaatttagttatagactaacttcatcctttgcttttgtaacttctcactccttatttttaaccttttcagtgggttctgccagttccagaagatttatcagtttttaaagaaacatcacattttggttcaagttctctctctaaaaacctttgtcatcatttagcacattacaaatagtggcatgtagtcccaggctgcatgtcaaaggtaattaaatctcaggagttggtcaggattcataaactgcttaagctccatgcctgctcttcacagatgtggcatgactttgtgattaaattccagtttggtattcagtctgggaccacttatgcttttgcttgtataaagtggtatatgttttcaataaagattagcggaactatgtaagaaatggaagcaaccaaagcctcttaaaaaaaaaaaagaaagaaaggtaaatattggattctttcctcccctgatcaccctcctacccaactcccattttcctcaagttcaaatgcaaggagagaaccctagcagtaaatgcatgtaaattacgcaaatgatagagtctggggaagcttcaagagggtgaaaaactgccagaacttttcagggtggttcagcacgtttgcttagaagagattcatttccatcattcccaggatcctctggagttccttgttcaaaaaagaaaggaggctgggcatggtggctcatgcctgtaatcccagcactttgggaggccaaggcaggcagactgcctgaggtcaggggttcaagaccaatgtggccaacatggtgaaaccccatttctactaaaaatacaaaaattagcatggtggcacacacctataatcccagctatttgggaggctgaggcaggagaatcacttgaatcccggagacggaggttgcagtgagccgagatcgcaccactgcactccagcctgggctacaagtgcaaaactctgtctcaaaaaaagaaaaaagaaagggaaagctttccacagtggttcgtaagggtgtctttttctctttttgttgcattgacattgcactttgggaaaaccacattgtagtgtaggtggtctgtaaacagtcagactccctttcccaaagctccccattccctccatgtctgatctagtactgttgtagtggtctggcttcatgtgatgacttcagcatatttaggtggtgtcaacatatttctactttctcccttggtccctgttggatgaaagtggatcaatttgcttgttaaacccaacgctcaccctgcatttgccttagagttgttctgactacaagtaaaaagggaaaggatgaactggatgacgtatggacagaaaaaccactgttggttaaacattcccctgcttgaacacccagacgagaacccgtttcttaagagaactacagccttatctattactgtttgctaagttctctatgattgttttctccatgtgagaaataactttataggttagaaaactaagagcccaacagcttaagaaaagaaaagaaccaaccaaccaacccagaaaaagcaagctctctttgccctttgatggactctatcaagtcttgagtgcccttcctagttcctgggaatatactaacagataacagcaggttagagcctggagaggtttcaaagtcatcatttcaagagaaaactgaggctttaactggtttgcatagggtgacaaggctggttaatggtgaacagagaccatgttaataactgagtctgctccagtatgcctctccctacttatggcaaccccaaatcctgtatttctcaaggagattttgatttcaagcattctaccctttccactgtcaagagtatttgcatctgacagttgccccaccccaacttggggcttagccggagttatggtcacgccaactgcatatcattgagcatctgagttgaccatctgtctctgctaagagctgactcttccatttcctttttaagacctgacttgagagagtcaatacctcaggcattgtagaaattgcggtcattctaaaatataaaagtgtaaagtttatattaatatttaattcatacaattggaaagaactttaaactgcaattaggtgtcagtacccatctgcttccagcaggctcacacgctcttcgatccctcggaggccgcagagggccacgatcaccttcgaatgtgctccttaacaatgtgcccacagtctctcaggagggagctccagatggtgaaggaaactctgcaggccatgatcctgcagctccagccagcaaaggaggcgggagaaagagaagctgcagcttcctgcatgacagctggtgtccacgaagcacaagcctgaggagtgacgggatgggggagggaggtgggccaccatgtttggacccggggggctgctcttccctcccccgccatagctaagatgcctgaatcaattacggagatacagagccttgaggtctttcagtggaaaggtggttcatgttcattctcatcagtgtgaaactgaggagtctgcaatttggaatatggagagagagactgatttgctgaatttccttctaaatgtcactcaaaaatttcttttccatgtcattcttgggaatgtcttccacaggatttgagaatagtttcatctcagcccccattagagagaagttggggtgaattctggaaaaatgtctctttttcctgtgccatttgccttctgctgcaacgaaaatatttcctgattcaagattctataaaaaggaaaccaagcataagactctgtcatcatacctgttacacgttcctacaggtgcacaatctaagagagctaattaacctcagagtctggagttaacagcttttcaccttacttctcctgtgatctaatattatcttagaaaaattaatatgcaatttccaaaagatattttggtaagacaacaacctcccagtgatatgccacctttcaattttccttttgtggcaatgattgcatctgaagaaaggatccctgagagtctctgtttcatcaggacattctgaaatttacccacagtgaggctgtggatggatcaggggacctgtataaaatgtttgagcctgttccattttcccgtggaacctgtttcactcaatgccaggcagtgcagcatttaggaaagcagtgcagtactcagtaaggcagtgcagtactcagtaacacaatacagtactcaggcagtgcagtactcagtaagacagtgcagtgctcagtaaggcagtgcaatactcagtaacagtgtagtactcagtaacagtgaagtactcagtaatacagtacagtattcagtaaggcagtgaagtactcagtaatacaatacagtactcagttaggcagtgcagtactcaggaatgcagcacagtactcaggcagtgcaatactcagtgcggtactcagtaacacagtgcagtactcagtaacagtgcagtactcagtaacagtgcagtactcagtaaggcagtgcagtactcagtaacacagtgcagtactcagtaatacagtacagtactcagtaaggcagtatggtactcagtaaagcaatgcaatgctcagtaacacagtgcagtgctcaataaggcagtgcagtgctcagtaaggcagtgaattgcttagtaacacagtgtagtgctcagtaggacagcatagtactcagtaacacagggcagctagtactcagtactataagtactgagtacttatataggcaatgtagtactcagtaaatcagtgcagtactcagtaatgcaagggcatttcaggctcctgctgggctgcttctttggcccagctgggactcctattgagacagctgcaaaacaggctgatttcaattaggcagcacttcccaaagtgcactgaggaaggtggccccaagagaagctctctaaacaaaggagtaccctctctggtcaagtacctttggtaaatacaccataccataatatctgcttggagaaccacaatgcacattagcatattagtctgagagagaacttatagtaaggaaactcacttgattttatctaacctcaaactttccaagtttaatggatcgtgaatttttttcatgtaactcctattcatatcccatagatctagtattgtacagcactgcattctctgaggaagtcccagtccaaactctgatttacatcactttagaaaccacactcacacttttgcagagtgttgagcttaataactacctgccacagattggtaaatttaatccagtggttgttctgtttgtgcttctgttctcatttatgtgtttagggatagtgaggttcctgccttcactaggatccacggatatgagaccatttttgtcatttcctgaagtcacactggcgtttccagaaggcatctggtgctttgctcagccttccatgctgtgcagcacttctgtcctcagtcaaggagatggccatgcttaagccagcaattggctggggtccaggaaacaaagcaaaagcacaatatgtgaatgtgctgattgtgttccctatggctttatctcgagcaaaatacactctacatattttaataataagtataattagcttgttcctggacttcattttcaatgatgaaccaaattcctgaattatttataattgtgtctaaagaaaattatgaactggtcacatggcacttggaatccttgagttaattccagtgaagcaaaacttgggaagagtcaggattggccacattgccaataacaaattcctacttcgacatatgtcttttcaaaaagcctcccagacacaagacatcttaaccgtcactagcccaagtgttttgtattactcagacaccatcatgaaataattctgtgaggtcatgatgtatttgaaaattctgcaagttaataactgccttgaattgtttgaacccgaaataagggttctttggtacctctagtagatagtgtgttcatttccctgctgcaaattttgaagtatttgggcaggtgagtcatgttttaaccacaagccataactcatctgttgtctttgcttggtcttagagtatcattcagaaagtccgctaagggccagcgtgcttcttctggctacacaaccttctcaggacaagcccactgtcttaagccactttgaccctgggagacacaggactgtgtatcctcaatcatactatacagcagtttttgtcaggggaacataaaaatatccaagagaggttagggcttagatttaaaagcatcaaaacaacaacaatggaaatttatgttggcgatagccaagaccacaagcaaaagcacatactggaaatgatgagttagaatctgatttgactgggatgttttatgagaatgtaagtgtgatattatactgtctgccttgctggaatgctggctttcaaatggtcacccatttttctttcactggcctgagttaggacatgctatcagtaatagtcccagttccatccaactttctgaaatttcatttttttttttgagatggagtctctctctgtcacccaagttggagtgcagtggccccgcaatctcggctcactgcaacctctacctcccaggttcaagctattctactgcctcagcctcccaagtagctggggttacaggcatttgccaccgggccctgatgatttttgtatttttagtagagacagggcttcaccatgttggctaggagggtctcaaactcctgacctcaggcgatccacccccacctcggcttcccaaagtgctgagattacaggtgtgagccaccgcacccggccaactttctgaaatttcaaaactgaattgatccttctccaaattagtatatactattggaaacttgtctttccctgcagtaaggctggtttccccaccccagaaacatgtaacggttggtaccatgctaagcccttgccatgctaagccctttacagtcatatcctataatccccatatcaaccttataaggaaggtgtttgtagatgatgcaactgagccttaagaggactaattccctttttctaaggcacagagctggtaaaatgtgaagtaatagtgaacctaacagtcagagacaggcagcatgctcttaactagtgctcttcctaaagttcctttaatgtccttttgagattttgagccatggaacttacttgttcacctggctaagaactcatggccactgtggaaatcttggttagggagtcaaagaaactgagcctggggcaaacgaggcttcccacactgccaggggagcctcactgtgaagtctaggctcagacaggcatcaacaaacctattcaccccaccatcatcctgatctaaccattccccagtcatcccaggaaaaccactcacagcctgacactgggctgactttcttgaagatcctcatccaattggtgtttttcagaagtgttccaatattatgaattctgtgttgtggagaaaagcaaccatgcatttactggtcaatgccttcttgtatatgtaattcaatacttttacttttaatatcctcaccttatctaatctttgaattttgtcatgtaatttattgcttcattaaggttactttttgttatacaaaataaaagctgatatccaaggcatggtgcatcttgatgattttttgtcctttgaagtatggatgatagaaaaatgtatcaggtttattcatctcatctttctgttacaggatgattaattgtacagttacatcacacgaaacatttataataaagtcatgctttagaattgcacgtgtttactttcagatatcagcaaatcagaaaatcatgaaatattctattcccagactcctagattaaggaaattgaaactaacattaccactagagttgggaacaagacaaagatacccacaataccactgaaaatattttactggagatgctggctaatttagttaggcaagaaaaatatagaagggttacaatttggaaaagaaaaaataaagctttaatattcacagatgatgttattgtaagctgaaaacccaaaagaatcaacagaaaaacgtttacacaattaacacaatcaaaatcaacaggcttcatatatgcagacaataattggaagactgagtataagaaaagaccccattttcaatagcaacaaaaagcaagagaaaatggctaagaatatatttatataagaaatgcataagccttagatgaaggaaattttaagatgaaggacatgagacaagaattgaatacatggaaagacacattaagcttttggataggaaggcatcataaatttaaaaatttccatcagtcaatttataaatatttcctgatcccaaatgggacaatagatgtctagttagcccccatatgcattctcccattcatccttttagccgttctgggtctagacacttaaactgcagaggtacatgtgtgcagaattgcaggtgtacatttgtagcattgtttgtaaggacaaaaatgagaacaaccgacattttcatttgcatcagttggagactggttaaataaattattgtacattcacactatggaatgctttacagttgctaaaaaaaaatctttgtgtacatatgtgcttgtctctgcagagctatcactggaaagggatccaggcgacttagtggaaaaaaaatgggaaggagacttacttttctgctctatactattcttttaaacatttggtaccgtgtgcatatatttacctattcaaaaataaaatcatttctttaagaaaaaagtcataattaatgagcctgagagtaggccctcaagtagggcagggcacacaggctgttgaatggttgcccaggtctccttgcggcttatgcaggattcatctgctatttatttacctactgtgtgcccagcactgtgctagggtatgcacctgatattggtgataaaaaggcaagtatgaactatgacataatgtgatcagatgattttaagttgagaaggaaattgatatggttaagctttgtgtccccacccaaatcttatcttgaattacaatcctcataacccccataatctccgtgtgtcaagggaaagaccaggtggaggtaattgattcatgggggcagtttccccagtgctgttctcatgatagtgagtgagttctgataacatttgatggttttataaggggctcttcccccttcactcagcacttctccttcctgccgccttttgaagaaggtgccttgcttcccctttgccttccgccattattgtaagtttcctgaggcctccccagccatgctgaactgtgattcatttaaaactctttcctttataaattacccagtctcaggcagttctttatggcagtatgaaaacggactaatacagaaatgaatcattatcattgaaacttttatcactttaaagacaagagattgattttgttctggccattttcttgaacaactacatctgggggtgtagtactgaaaattcagcactctctggaacccacgttctctcccttctgggttagggaataacatcaagtctccttcctttgagccctggatcaaagagacctgataggtgattgattgtgtgggcagaatggagacttgggaaatggagtcacattctgcccttcattgtccctcaggaactacttatgccctcattgttttatccaattttaggtgtgcttttaaatgtacatatcacatacacttttccactgtggaaaagacacaaccacaaactccagcatgaagaacag

(19) United States
(12) Patent Application Publication
(10) Pub. No.: US 2016/0120916 A1
(43) Pub. Date: May 5, 2016

(54) COMPOSITIONS AND METHODS COMPRISING BACTERIA FOR IMPROVING BEHAVIOR IN NEURODEVELOPMENTAL DISORDERS

(71) Applicant: California Institute of Technology, Pasadena, CA (US)

(72) Inventors: Elaine Hsiao, Rowland Heights, CA (US); Wei-Li Wu, Pasadena, CA (US); Sarkis K. Mazmanian, Porter Ranch, CA (US); Paul H. Patterson, Altadena, CA (US)

(21) Appl. No.: 14/925,240

(22) Filed: Oct. 28, 2015

Related U.S. Application Data
(60) Provisional application No. 62/072,917, filed on Oct. 30, 2014.

(51) Int. Cl. A61K 35/741; G01N 33/68; C12Q 1/68; A61K 9/00

(52) U.S. Cl. CPC A61K 35/741; A61K 9/0053; G01N 33/6893; C12Q 1/6883; G01N 2800/30; G01N 2333/47; G01N 2333/4703; C12Q 2600/158; C12Q 2600/156

(57) ABSTRACT

Some embodiments include probiotics for use in treatment of one or more autism spectrum disorder (ASD) or epilepsy in a subject in need thereof. The probiotics can comprise Bacteroides bacteria (e.g., B. fragilis). In some embodiments, the subject is identified in need of treatment ASD or epilepsy based a combination of behavioral symptoms and optional genetic markers. Upon treatment, one or more ASD-related behaviors are improved in the subject.

### CROSS-REFERENCE TO RELATED APPLICATIONS

The present application claims the benefit of U.S. Provisional Application No. 62/072,917, filed Oct. 30, 2014, which is hereby incorporated by reference in its entirety.

### STATEMENT REGARDING FEDERALLY SPONSORED R&D

This invention was made with government support under Grant No. W81XWH-11-1-0515 awarded by the Army under Grant No. GM099535 & NS074374 & MH090749 awarded by the National Institutes of Health. The government has certain rights in the invention.

### REFERENCE TO SEQUENCE LISTING

This application is filed with an electronic sequence listing entitled CALTE113ASEQUENCE.TXT, created on Oct. 23, 2015 which is 556,367 bytes in size. The information in the electronic sequence listing is hereby incorporated by reference in its entirety.

### BACKGROUND

Autism spectrum disorder (ASD) can be a devastating illness involving repetitive behaviors and deficits in social interaction and communication. There are an estimated 1 million people with ASD in the United States. Alarmingly, the diagnosis of ASD has increased over the past several decades, making it a highly visible public health concern. There is growing support for contributions by both genetic and environmental risk factors in ASD.

Field

Some embodiments described herein relate generally to probiotic compositions, which can be used to treat autism spectrum disorder (ASD) symptoms and/or epilepsy symptoms.

### SUMMARY

In accordance with some embodiments described herein, methods for improving behavioral performance in a subject having autism, epilepsy, or cortical dysplasia focal epilepsy are provided. The method can comprise administering an effective amount of one or more Bacteroidies bacteria to the subject having autism spectrum disorder (ASD), epilepsy, or cortical dysplasia focal epilepsy. In some embodiments, the method further comprises detecting in a sample of the subject a presence or absence of a loss-of-function of at least one of: contactin associated protein-like 2 (CNTNAP2); and disrupted in schizophrenia 1 (Disc1). The method can further comprise administering the effective amount of one or more Bacteroidies bacteria to the subject if the subject has a loss-of-function of at least one of CNTNAP2 and Disc1. In some embodiments, the effective amount of one or more Bacteroidies bacteria comprises B. fragilis. In some embodiments, the effective amount of one or more Bacteroidies bacteria comprises B. fragilis, B. thetaiotaomicron, B. vulgatus, or a mixture of two or three of the listed bacteria. In some embodiments, the improved behavioral performance comprises at least one of language comprehension, language production, sociability, communication, sensorimotor gating, anxiety, or repetitive behavior. In some embodiments, the improved behavioral performance comprises at least one of anxiety, sensorimotor gating, and sociability. In some embodiments, a sole active ingredient administered to the subject in the method consists essentially of one or more Bacteroidies bacteria. In some embodiments, the effective amount of one or more Bacteroidies bacteria is in a composition substantially free of bacteria other than the one or more Bacteroidies bacteria. In some embodiments, detecting a presence or absence of a loss-of-function of CNTNAP2 or Disc1 comprises detecting an allele of a gene encoding CNTNAP2 protein or Disc1 protein. In some embodiments, detecting a presence or absence of a loss-of-function of CNTNAP2 or Disc1 comprises detecting a presence or absence of a CNTNAP2 or Disc1 polypeptide. In some embodiments, detecting a presence or absence of a loss-of-function of CNTNAP2 or Disc1 is performed on a sample of the subject. In some embodiments, detecting a presence or absence of a loss-of-function of CNTNAP2 or Disc1 is performed on the subject in vivo. In some embodiments, detecting a presence or absence of a loss-of-function of CNTNAP2 or Disc1 comprises detecting a deletion of at least a portion of a gene encoding CNTNAP2 or Disc1. In some embodiments, the subject suffers from anxiety, autism spectrum disorder (ASD), or a pathological condition with one or more of the symptoms of ASD. In some embodiments, the effective amount of one or more Bacteroidies bacteria is administered orally to the subject. In some embodiments, the method further comprises identifying the subject as having at least one of ASD, epilepsy, or cortical dysplasia focal epilepsy. In some embodiments, the effective amount of one or more Bacteroidies bacteria comprises at least about 10⁷ colony forming units (cfu), for example at least about 10⁷ cfu, at least about 10⁸ cfu, at least about 10⁹ cfu, at least about 10¹⁰ cfu, at least about 10¹¹ cfu, or at least about 10¹² cfu.

In accordance with some embodiments described herein, a kit is provided. The kit can comprise a composition comprising one or more Bacteroidies bacteria, in which the composition is suitable for administration to a human subject. The kit can comprise at least one of a nucleic acid substantially complementary to a gene encoding contactin associated protein-like 2 (CNTNAP2), or a gene encoding disrupted in schizophrenia 1 (Disc1); or an antibody that binds specifically to a CNTNAP2, or Disc1 polypeptide. In some embodiments, the composition is suitable for oral administration. In some embodiments, the one or more Bacteroidies bacteria comprises B. fragilis. In some embodiments, the composition consists essentially of one or more Bacteroidies bacteria. In some embodiments, the composition is substantially free of bacteria other than Bacteroidies fragilis, B. thetaiotaomicron, and B. vulgatus. In some embodiments, the composition is substantially free of bacteria other than Bacteroidies fragilis. In some embodiments, the composition comprises at least about 10⁷ colony forming units (cfu) of Bacteroidies bacteria, for example at least about 10⁷ cfu, at least about 10⁸ cfu, at least about 10⁹ cfu, at least about 10¹⁰ cfu, at least about 10¹¹ cfu, or at least about 10¹² cfu of Bacteroidies bacteria.

### BRIEF DESCRIPTION OF THE DRAWINGS

FIGS. 1A-F are a series of graphs depicting that several ASD-relevant behaviors in BTBR and CNTNAP2 mice are corrected by B. fragilis in accordance with some embodiments described herein. Shown are graphs depicting data for B-fragilis treated and control mice assessed for: open field entries (FIG. 1A); marble burying (FIG. 1B); prepulse inhibition (PPI) (FIG. 1C); open field distance traveled (cm) (FIG. 1D); open field velocity (cm/s) (FIG. 1E); and grooming test (FIG. 1F) in accordance with some embodiments described herein.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

It is contemplated herein that both genetic and environmental risk factors can contribute to Autism spectrum disorder (ASD). In accordance with various embodiments described herein, methods, uses, comparisons, and kits are provided for treating or preventing ASD symptoms in subjects with particular risk factors or combinations of symptoms. It is further contemplated that subjects with certain genetic markers and/or combinations of symptoms can be identified as in need of treatment for ASD and/or epilepsy symptoms, and further as candidates for treatment with probiotics in accordance with some embodiments described herein. A subject identified as having a mutation in one or more of contactin associated protein-like 2 (CNTNAP2) or disrupted in schizophrenia 1 (DISC1) can be at risk for a neurodevelopmental disorder, for example ASD or epilipsy. An effective amount of a probiotic comprising Bacteroides bacteria can be administered to the subject so as to treat, or prevent ASD symptoms and/or epilepsy symptoms.

A substantial environmental risk for ASD is maternal infection, as validated by large epidemiological studies showing that rates of ASD are higher in children born following a severe infection during pregnancy. Similar associations have been found with elevated cytokines and chemokines in maternal serum or amniotic fluid during pregnancy. Analysis of spinal fluid and brains from autistic subjects reveals activated microglia and elevated pro-inflammatory cytokines compared to controls, as well as dysregulation of immune-related genes in the brain and periphery. Furthermore, a significant proportion of subjects with ASD suffer from gastrointestinal (GI) abnormalities including constipation, abdominal pain, immune activation and intestinal barrier dysfunction (“leaky gut”). Consistent with these non-neurologic features, recent studies have shown that the composition of gut bacteria (the intestinal microbiome) is altered in children with ASD compared to controls, a feature exacerbated in those with GI complications. Collectively, mounting evidence suggests a potential gut-immune-brain connection in ASD.

It described herein that activation of the maternal immune system of mice and non-human primates results in offspring that display core behavioral deficits and neuronal abnormalities found in ASD. Without being limited by any theory, it is contemplated that the microbiomes of mice are altered following maternal immune activation (MIA). In accordance with some embodiments described herein, a probiotic from the human microbiome, Bacteroides fragilis, can ameliorate immune activation, ASD-related behavioral deficits and/or cortical dysplasia focal epilepsy in mouse models. Without being limited by any theory, it is contemplated that gut bacteria play a substantial role in modulating gene-environment interactions relevant to ASD.

Although there are several mouse models of ASD based on candidate human genes, the contactin associated protein-like 2 (CNTNAP2) knockout mouse is of particular interest because association, linkage, gene expression and imaging data support the role of common and rare variants of this gene in ASD. Notably, the same CNTNAP2 variant that increases risk for language deficits in ASD leads to abnormal functional brain connectivity in human subjects. The CNTNAP2 mouse also models behaviors characteristic of cortical dysplasia focal epilepsy. The BTBR T⁺ tfl J model is complementary to CNTNAP2−/− mice as it is an inbred strain that displays the three core behaviors of ASD. Further, BTBR mice have been shown to have elevation of innate immune responses. The BTBR model includes loss of function in the DISC1 gene.

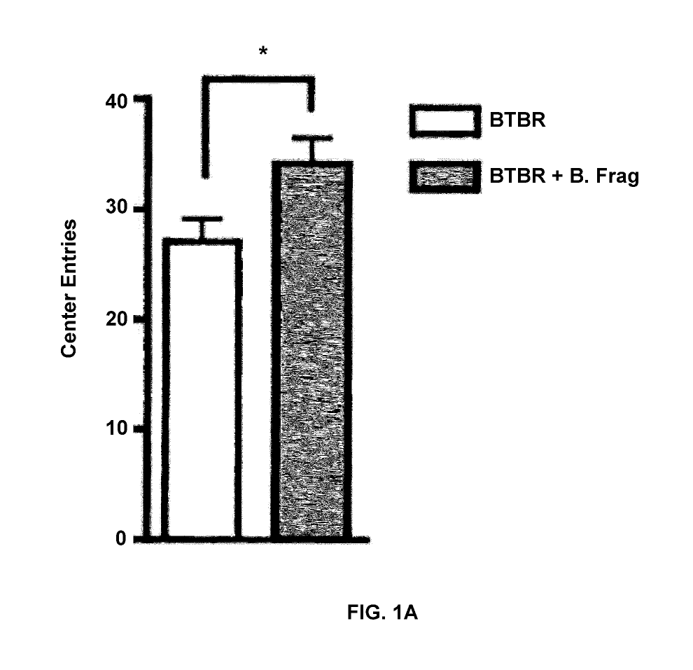

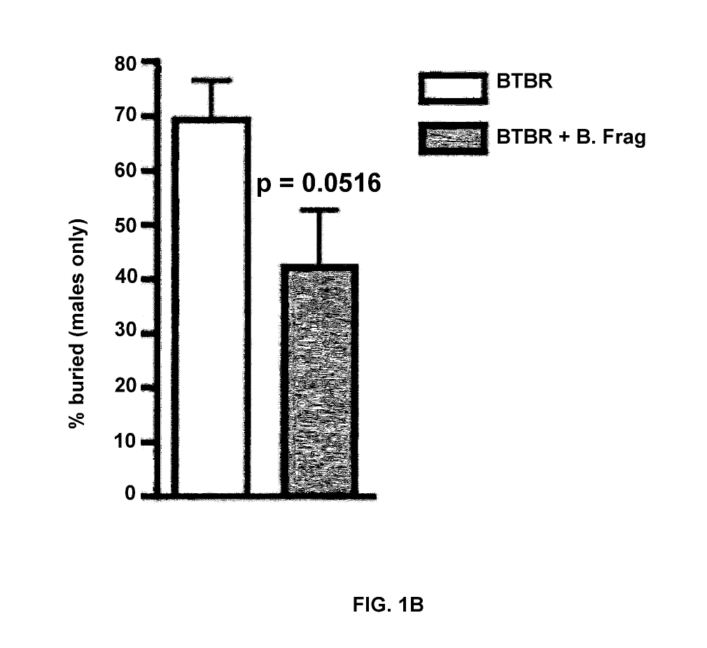

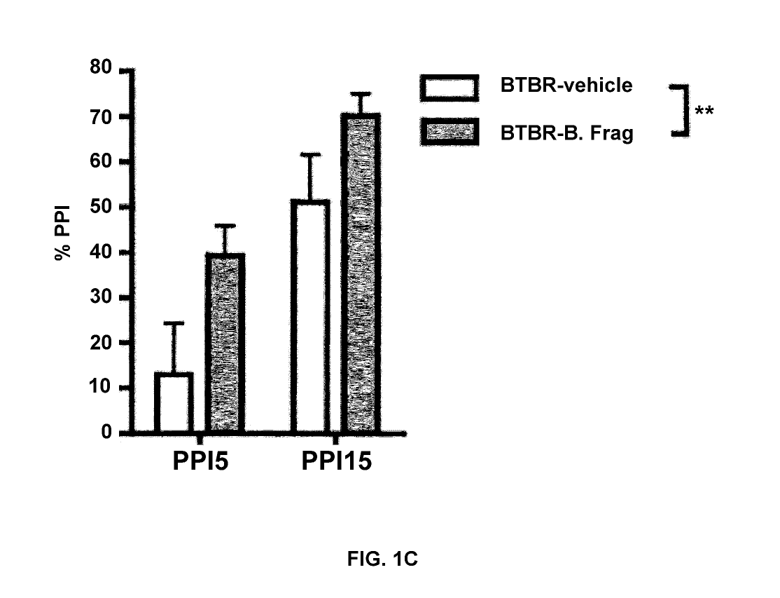

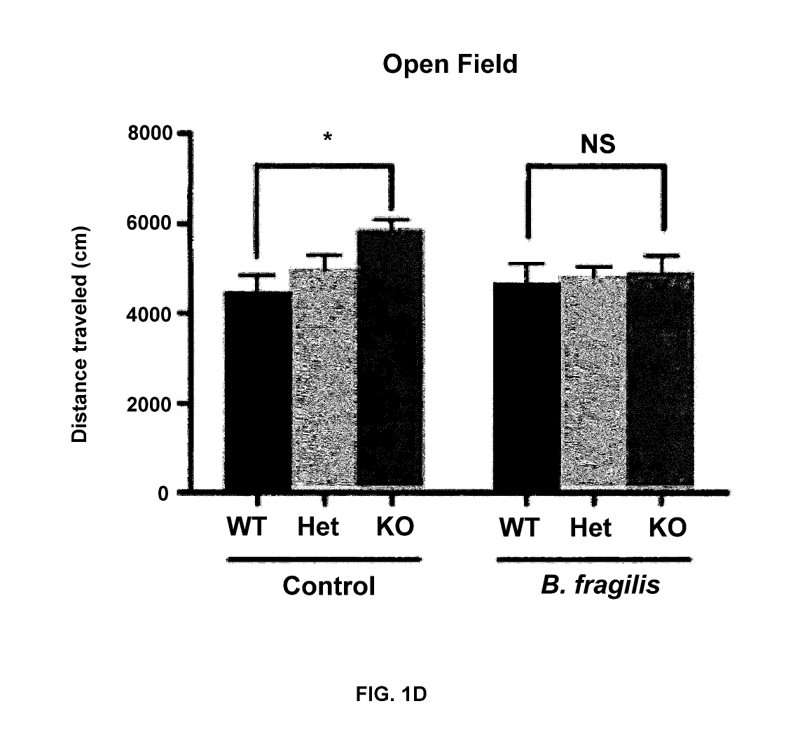

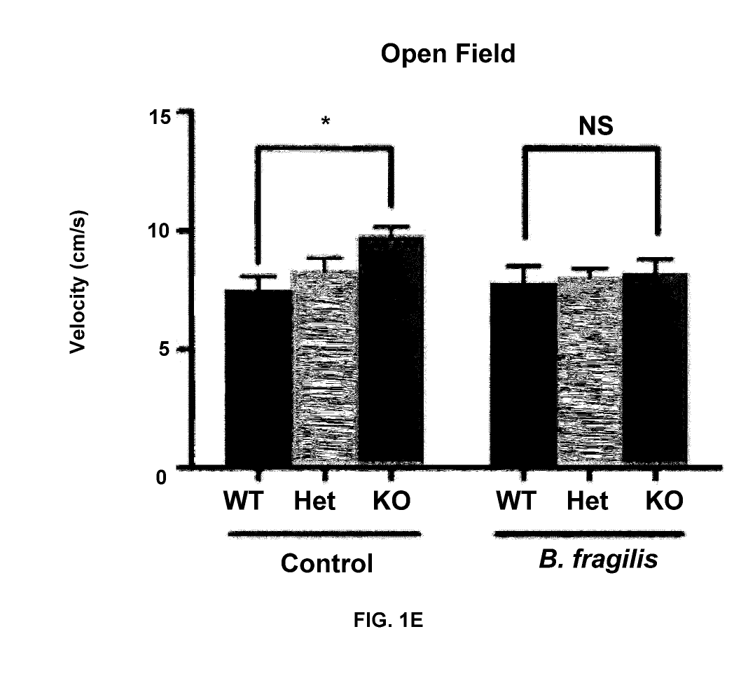

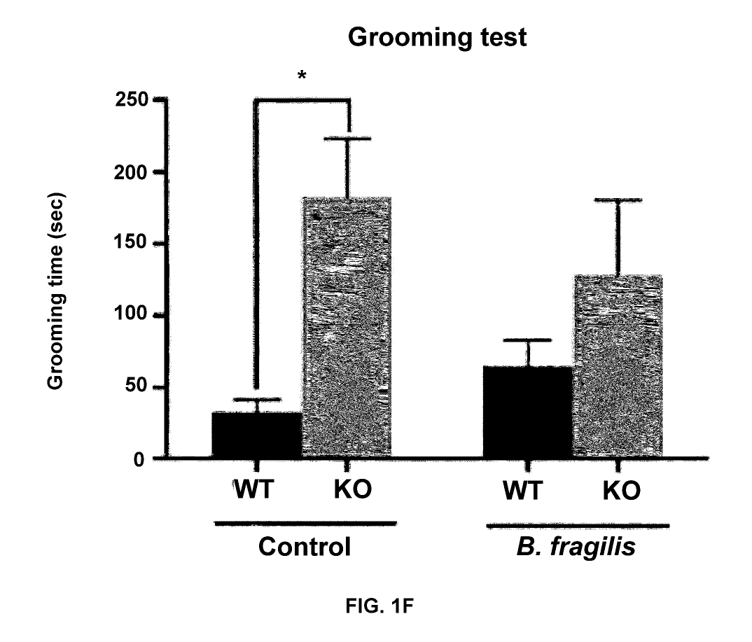

B. fragilis treatment was tested in CNTNAP2 and BTBR mice. It is shown herein that B. fragilis treatment corrects hyperactivity and increased self-grooming (a repetitive behavior) in CNTNAP2−/− mice (FIG. 1D-F). Moreover, B. fragilis ameliorates anxiety, repetitive behaviors and the startle response in BTBR mice (FIG. 1A-C).

As shown in FIGS. 1A-C, administering the Bacteroides bacterium B. fragilis to BTBR autism model mice ameliorates anxiety-like behavior (as measured by center of field entries; see FIG. 1A), ameliorates repetitive behavior (as measured by marble burying; see FIG. 1B), and ameliorates deficiencies in sensorimotor gating (as measured by a prepulse inhibition test; see FIG. 1C). Accordingly, in some embodiments disclosed herein, methods and uses comprising the administration of Bacteroides bacterium to a human subject having symptoms and/or genetic background corresponding with the BTBR model can treat and/or prevent ASD symptoms. Corresponding to the behaviors improved in the BTBR model, in some embodiments, the subject is identified as having deficiencies in sensorimotor gating, impaired sociability, and/or anxiety, and is administered an effective amount of a probiotic comprising, consisting of, or consisting essentially of Bacteroides bacteria, thus treating or improving the indicated behavior. In some embodiments, the subject is identified as having a mutation corresponding to a BTBR genetic background, for example a mutation in DISC1, and is and is administered an effective amount of a probiotic comprising, consisting of, or consisting essentially of Bacteroides bacteria, thus treating or improving ASD behavior.

As shown in FIGS. 1D-F, administering the Bacteroides bacterium B. fragilis to CNTNAP2 autism model mice results in amelioration of hyperactive behaviors, as measured by open field distance traveled (FIG. 1D) and open field velocity (FIG. 1E), and amelioration of repetitive behavior (as measured by a self-grooming test; see FIG. 1F). Accordingly, in some embodiments described herein, methods and uses comprising the administration of a probiotic comprising, consisting of, or consisting essentially of Bacteroides bacteria to a human subject having symptoms and/or genetic background corresponding to the CNTNAP2 model can treat and/or prevent ASD symptoms and/or epiliepsy (e.g. cortical dysplasia focal epilepsy symptoms). Corresponding to the behaviors improved in the CNTNAP2 model, in some embodiments, the subject is identified as having at least one of the following behavioral deficiencies: deficient communication behavior (for example, deficient language production and/or comprehension, deficient sociability, and or deficient communication), deficient sensorimotor gating behavior, increased anxiety behavior, hyperactive behavior and/or repetitive behavior. The subject can be administered an effective amount of a probiotic comprising, consisting of, or consisting essentially of Bacteroides bacteria, thus treating or improving the indicated behavior. In some embodiments, the subject is identified as having a mutation in CNTNAP2, and is administered an effective amount of a probiotic comprising, consisting of, or consisting essentially of Bacteroides bacteria so as to improve or prevent at least one behavior for which CNTNAP2 is a risk factor. Optionally, the subject is identified as homozygous for a loss-of-function mutation in CNTNAP. Optionally, the subject is identified as heterozygous for a loss-of-function mutation in CNTNAP. Optionally, the probiotic further comprises Enterococcus bacteria.

The Homo sapiens Contactin-associated protein-like 2 (CNTNAP2) gene encodes a product that is identified as a member of the neurexin family which functions in the vertebrate nervous system as cell adhesion molecules and receptors. The Homo sapiens CNTNAP2 gene is identified as spanning over 2 Mb of DNA at cytogenetic location 7q35 on chromosome 7. A partial coding sequence (CDS) for the Homo sapiens CNTNAP2 gene, available as Genbank accession no. AH010723, and a corresponding reference sequence is provided as SEQ ID NO: 1. In some embodiments, subject are identified as having a loss-of-function mutation in the CNTNAP2 gene. As used herein, “loss-of-function” mutation is used broadly, and encompasses hypomorphic mutations, null mutations, and dominant negative mutations. Non-limiting examples of “loss-of-function mutations” in CNTNAP2 in accordance with some embodiments described herein include a point mutation that interferes with the function of CNTNAP2 gene product, a mutation is a regulatory element such as a promoter or enhancer that decreases or eliminates CNTNAP2 expression, a deletion of all or part of CNTNAP2, or any combination thereof.

The Homo sapiens “disrupted in schizophrenia 1” (DISC1) gene, has been identified as encoding a protein with multiple coiled coil motifs which is located in the nucleus, cytoplasm and mitochondria. The protein encoded by DISC1 is involved as being involved in in neurite outgrowth and cortical development through its interaction with other proteins. DISC1 is associated with familial behavioral disorders, including schizophrenia, depression, and bipolar disorder, and encodes a protein implicated in a number of cellular functions, including those of neurons, and is mutated in the BTBR model. The Homo sapiens DISC1 gene is identified as residing at cytogenetic location 1q42.1 on chromosome 1. The sequence of DISC1 is available as Genbank accession no. NG_011681. A reference sequence of DISC1 is provided as SEQ ID NO: 2. Non-limiting examples of “loss-of-function mutations” in DISC1 in accordance with embodiments herein include a point mutation that interferes with the function of DISC1 gene product, a mutation is a regulatory element such as a promoter or enhancer that decreases or eliminates DISC1 expression, or a deletion of all or part of DISC1, chromosomal translocations disrupting the DISC1 locus, such as t(1;11)(q42.1;q14.3), and any combination thereof.

The sequences, or the presence of polymorphisms in any of the genes disclosed herein can be ascertained using methods known to one of skill in the art, for example polymerase chain reaction, nucleic acid sequencing, microarray analysis, affinity for a nucleic acid probe, in situ hybridization, and the like. In some embodiments, the presence or absence of polymorphisms in any of the identified genes can be ascertained by identifying a presence or absence of wild-type gene product, for example using an antibody specific for the wild-type gene product, or an antibody specific for a mutant isoform of the gene product.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, N.Y. 1994); Sambrook et al., Molecular Cloning, A Laboratory Manual, Cold Springs Harbor Press (Cold Springs Harbor, N.Y. 1989). For purposes of the present disclosure, the following terms are defined below.

As used herein, the term “subject” is a vertebrate, such as a mammal. The term “mammal” is defined as an individual belonging to the class Mammalia and includes, without limitation, humans, domestic and farm animals, and zoo, sports, or pet animals, such as sheep, dogs, horses, cats or cows. In some embodiments, the subject is human. In some embodiments, the subject is a non-human primate.

As used herein, the term “condition/disorder/symptom” or “behavioral abnormality” refers to a symptom expressed by a subject including but not limited to anxiety, Fragile X, Rett syndrome, tuberous sclerosis, obsessive compulsive disorder, attention deficit disorder, schizophrenia, epilepsy (e.g., cortical dysplasia focal epilepsy), autistic disorder (classic autism), Asperger's disorder (Asperger syndrome), pervasive developmental disorder not otherwise specified (PDD-NOS), childhood disintegrative disorder (CDD), or a pathological condition with one or more of the symptoms of ASD.

As used herein, the term “subject in need of the treatment” refers to a subject expressing or suffering from one or more of the behavioral disorder/symptoms mentioned above. In some embodiments, the subject in need of treatment suffers from at least one of schizophrenia, ASD, epilepsy (e.g., cortical dysplasia focal epilepsy), or a gastrointestinal or immunological pathology associated with ASD or epilepsy (for example leaky gut syndrome). An appropriately qualified person is able to identify such an individual in need of treatment using standard behavioral testing protocols/guidelines. The same behavioral testing protocols/guidelines can also be used to determine whether there is improvement to the individual's disorder and/or symptoms. As used herein “sensorimotor gating” refers to ability to filter out irrelevant and/or intrusive sensory stimuli. As such, subjects deficient in sensorimotor gating behavior can have impaired ability to filter out stimuli, and/or can have difficulty coping with intensely stimulating environments. By way of example, sensorimotor gating behavior can be assessed with a prepulse inhibition (PPI) test. In accordance with some embodiments described herein a subject is identified as being in need of improved sensorimotor gating, for example in need of improving filtering out of irrelevant sensory stimuli such as background noise, background light, and the like. As used herein, “communication behavior” refers to communication, language comprehension and production, and sociability, including vocal and non-vocal social communication. In some embodiments, a subject is identified as deficient in communication behavior based on impaired sociability. In some embodiments, a subject is identified as deficient in communication behavior based on impaired language comprehension and/or production.

As used herein, the term “improvement in behavioral performance” refers to prevention or reduction in the severity or frequency, to whatever extent, of one or more of the behavioral disorders, symptoms and/or abnormalities expressed by individual suffering from ASD, schizophrenia, or a pathological condition with one or more of the symptoms of ASD or schizophrenia. Non-limiting examples of the behavioral symptoms include impaired communication, impaired sociability, impaired language comprehension and/or production, impaired sensorimotor gating behavior, repetitive behavior, and increased anxiety. The improvement is either observed by the individual taking the treatment themselves or by another person (medical or otherwise). In some embodiments, a probiotic comprising an effective amount of Bacteroides and/or Enterococcus bacteria as described herein is administered the subject. In some embodiments, sensorimotor gating behavior is improved in the subject after administration of the probiotic. In some embodiments, communication behavior is improved in the subject after administration of the probiotic. Examples of communication behaviors that can be improved include communication, sociability, and language comprehension and/or production. In some embodiments, anxiety behavior is improved in the subject after administration of the probiotic. In some embodiments, repetitive behavior is improved in the subject after administration of the probiotic. In some embodiments, sensorimotor gating behavior and communication behavior are improved in the subject after administration of the probiotic.

As used herein, the term “treatment” refers to a clinical intervention made in response to a disease, disorder or physiological condition manifested by a subject, particularly a subject suffering from ASD, schizophrenia, or a pathological condition with one or more of the symptoms of ASD or schizophrenia. The aim of treatment may include, but is not limited to, one or more of the alleviation or prevention of symptoms, slowing or stopping the progression or worsening of a disease, disorder, or condition and the remission of the disease, disorder or condition. In some embodiments, “treatment” refers to both therapeutic treatment and prophylactic or preventative measures. Those in need of treatment include those already affected by a disease or disorder or undesired physiological condition as well as those in which the disease or disorder or undesired physiological condition is to be prevented. For example, in some embodiments treatment may improve behavioral performance of the subject, including ASD-related behaviors such as sensorimotor gating behavior deficiencies and/or communication behavior deficiencies. As used herein, the term “prevention” refers to any activity that reduces the burden of the individual later expressing those behavioral symptoms. This takes place at primary, secondary and tertiary prevention levels, wherein: a) primary prevention avoids the development of symptoms/disorder/condition; b) secondary prevention activities are aimed at early stages of the condition/disorder/symptom treatment, thereby increasing opportunities for interventions to prevent progression of the condition/disorder/symptom and emergence of symptoms; and c) tertiary prevention reduces the negative impact of an already established condition/disorder/symptom by, for example, restoring function and/or reducing any condition/disorder/symptom or related complications.

“Pharmaceutically acceptable” carriers are ones which are nontoxic to the cell or mammal being exposed thereto at the dosages and concentrations employed. “Pharmaceutically acceptable” carriers in accordance with methods and uses and compositions and kits herein can comprise, but not limited to, organic or inorganic, solid or liquid excipients which is suitable for the selected mode of application such as oral application or injection, and administered in the form of a conventional pharmaceutical preparation, such as solid such as tablets, granules, powders, capsules, and liquid such as solution, emulsion, suspension and the like. Often the physiologically acceptable carrier is an aqueous pH buffered solution such as phosphate buffer or citrate buffer. The physiologically acceptable carrier may also comprise one or more of the following: antioxidants including ascorbic acid, low molecular weight (less than about 10 residues) polypeptides, proteins, such as serum albumin, gelatin, immunoglobulins; hydrophilic polymers such as polyvinylpyrrolidone, amino acids, carbohydrates including glucose, mannose, or dextrins, chelating agents such as EDTA, sugar alcohols such as mannitol or sorbitol, salt-forming counterions such as sodium, and nonionic surfactants such as TWEEN™ surfactant, polyethylene glycol (PEG), and PLURONICS™ surfactant. Auxiliary, stabilizer, emulsifier, lubricant, binder, pH adjustor controller, isotonic agent and other conventional additives may also be added to the carriers.

The pharmaceutically acceptable or appropriate carrier in accordance with methods and uses and compositions and kits herein may include other compounds known to be beneficial to an impaired situation of the GI tract, (e.g., antioxidants, such as Vitamin C, Vitamin E, Selenium or Zinc); or a food composition. The food composition can be, but is not limited to, milk, yoghurt, curd, cheese, fermented milks, milk based fermented products, ice-creams, fermented cereal based products, milk based powders, infant formulae, tablets, liquid bacterial suspensions, dried oral supplement, or wet oral supplement.

As used herein, the term “probiotic” refers to live microorganisms, which, when administered in adequate amounts, confer a health benefit on the host. The probiotics in accordance with methods and uses and compositions and kits herein may be available in foods and dietary supplements (for example, but not limited to capsules, tablets, powders, and liquids). Non-limiting examples of foods containing probiotic include dairy products such as yogurt, fermented and unfermented milk, smoothies, butter, cream, hummus, kombucha, salad dressing, miso, tempeh, nutrition bars, and some juices and soy beverages. In some embodiments, the probiotic comprises a single microorganism. In some embodiments, the probiotic comprises a combination of microorganisms. In some embodiments, the probiotic comprises a single composition. In some embodiments, the probiotic comprises two or more compositions, which can be used together, for example administered simultaneously or administered sequentially. It is noted that a probiotic can serve as the “active ingredient” or a composition or compositions for use in administration to a subject. That is, the method, use, and/or composition or compositions (either individually or in the aggregate) can comprise an effective amount of probiotic to improve at least one behavior in a subject. In some embodiments, the probiotic is the sole active ingredient for administration to the subject. In some embodiments, the “sole active ingredient” probiotic for administration to the subject can be provided in a composition or in a method or use that is substantially free of or free of bacteria other than the probiotic, antibiotics, and drugs. Even if the probiotic is the “sole” active ingredient, the composition or composition comprising the probiotic may comprise additional substances (such as buffers, bacterial feedstock, excipients, flavors, and/or food) that do not substantially affect the behavior of the subject, but may be useful for the function of the probiotic or its administration.

In some embodiments, the probiotic is comprised in a composition or compositions that are substantially free of bacteria (other than the probiotic) and/or drugs or antibiotics. By “substantially free” or “substantially absent”, it is understood that while a bacteria other than the probiotic, drug, and/or antibiotic may be present in trace amounts, the bacteria other than the probiotic, drug, and/or antibiotic have no appreciable effect on the subject.

As used herein “effective amount” of probiotic refers to a quantity sufficient to achieve a clinically significant change in a behavior of a subject.

As used herein, the term “neutraceutical” refers to a food stuff (as a fortified food or a dietary supplement) that provides health benefits. Nutraceutical foods are not subject to the same testing and regulations as pharmaceutical drugs.

Probiotics for Treatment of ASD and/or Epilepsy

Without being limited by any theory, it is contemplated that the BTBR mouse model displays both neuropathological and behavioral features of ASD, for example impaired sensorimotor gating, elevated anxiety, and impair sociability. Without being limited by any theory, it is contemplated that the CTNAP2 mouse model displays both neuropathological and behavioral features of ASD and cortical dysplasia epilepsy, for example, impaired communication behavior (e.g. sociability, language comprehension and/or production, and communication), sensorimotor gating, and anxiety. It is demonstrated herein that treatment of BTBR mice and CNTNAP2 offspring with the human commensal bacterium Bacteroides fragilis corrects particular behavioral deficits. Accordingly, some embodiments include a probiotic treatment for symptoms of ASD and/or epilepsy. It is further contemplated that in accordance with some embodiments described herein, Bacteroides bacteria, and combinations of Bacteroidies bacteria and Enterococcus bacteria (which has been shown to affect behavior symptoms associated with an MIA mouse model of ASD) are useful in treating or preventing symptoms of ASD and/or epilepsy (e.g. cortical dysplasia focal epilepsy).

In some embodiments, the subject is in need of improvement in sensorimotor gating behavior, anxiety behavior, and sociability behavior (it is noted that these behaviors are in line with the behaviors of the BTBR mouse model). An effective amount of a probiotic comprising, consisting of, or consisting essentially of at least one of the following is provided for administration to the subject (or is for use in treating the subject): (a) Bacteroidies bacteria (e.g., B. fragilis, B. thetaiotaomicron or B. vulgatus); (b) Bacteroidies bacteria (e.g., B. fragilis, B. thetaiotaomicron or B. vulgatus) and Enterococcus bacteria (e.g., E. faecilis, E. faecium, E. hirae, E. avium, E. durans, E. gallinarum, or E. casseliflavus); (c) B. fragilis; (d) B. thetaiotaomicron; (e) B. vulgatus; (g) B. fragilis and B. thetaiotaomicron; (h) B. fragilis and B. vulgatus; (i) B. thetaiotaomicron and B. vulgatus; (j) B. fragilis, B. thetaiotaomicron and B. vulgatus; (k) B. fragilis and Enterococcus bacteria (e.g., E. faecilis, E. faecium, E. hirae, E. avium, E. durans, E. gallinarum, or E. casseliflavus); (l) B. thetaiotaomicron and Enterococcus bacteria (e.g., E. faecilis, E. faecium, E. hirae, E. avium, E. durans, E. gallinarum, or E. casseliflavus); (m) B. vulgatus and Enterococcus bacteria (e.g., E. faecilis, E. faecium, E. hirae, E. avium, E. durans, E. gallinarum, or E. casseliflavus); (n) B. fragilis and B. thetaiotaomicron and Enterococcus bacteria (e.g., E. faecilis, E. faecium, E. hirae, E. avium, E. durans, E. gallinarum, or E. casseliflavus); (o) B. fragilis and B. vulgatus and Enterococcus bacteria (e.g., E. faecilis, E. faecium, E. hirae, E. avium, E. durans, E. gallinarum, or E. casseliflavus); (p) B. thetaiotaomicron and B. vulgatus and Enterococcus bacteria (e.g., E. faecilis, E. faecium, E. hirae, E. avium, E. durans, E. gallinarum, or E. casseliflavus); (q) B. fragilis, B. thetaiotaomicron and B. vulgatus and Enterococcus bacteria (e.g., E. faecilis, E. faecium, E. hirae, E. avium, E. durans, E. gallinarum, or E. casseliflavus); (r) B. fragilis and E. faecilis; (s) B. thetaiotaomicron and E. faecilis; (t) B. vulgatus and E. faecilis; (u) B. fragilis and B. thetaiotaomicron and E. faecilis; (v) B. fragilis and B. vulgatus and E. faecilis; (w) B. thetaiotaomicron and B. vulgatus and E. faecilis; or (x) B. fragilis, B. thetaiotaomicron, B. vulgatus and E. faecilis. Following administration of the bacteria, the sensorimotor gating behavior, anxiety behavior, and sociability behavior can be improved. Optionally, a sample from the subject is identified as having a loss-of-function mutation in a gene associated with the BTBR model, for example a loss-of-function mutation in DISC1. Optionally, a loss-of-function mutation in DISC1 is determined to be indicative that a subject is at risk of developing, or in need of treatment for, at least one of sensorimotor gating behavior, anxiety behavior, and sociability behavior. Optionally, the subject is administered no other bacteria, or substantially no other bacteria apart from the identified bacteria of the probiotic, and as such the probiotic for use in treatment of the subject is in a composition or compositions free or substantially free of other bacteria. Optionally, the subject is administered no antibiotics, or is administered substantially no antibiotics, and as such the probiotic for administration to the subject is in a composition or compositions free or substantially free of antibiotics. Optionally, the subject is administered no drugs, or is administered substantially no drugs, and as such the probiotic for administration to the subject is in a composition or compositions free or substantially free of drugs. Optionally, the subject is administered no pharmaceutically active ingredients, or is administered substantially no pharmaceutically active ingredients, and as such the probiotic for administration to the subject is in a composition or compositions free or substantially free of pharmaceutically active ingredients. Optionally, the B. fragilis comprises wild-type B. fragilis, mutant B. fragilis lacking polysaccharide A (dPSA), or a combination of wild-type B. fragilis and dPSA B. fragilis. Optionally, the B. fragilis comprises wild-type B. fragilis, mutant B. fragilis lacking polysaccharide A (dPSA), or a combination of wild-type B. fragilis and dPSA B. fragilis. In some embodiments, the subject in need of improvement in sensorimotor gating behavior, anxiety behavior, and sociability behavior has ASD. In some embodiments, the method further comprises determining whether or not the subject has ASD, as described herein.

In some embodiments, the subject is in need of improvement in communication behavior (including language comprehension and/or production, sociability, and communication), sensorimotor gating, anxiety, and/or repetitive behavior (it is noted that these behaviors are in line with the behaviors of the CNTNAP2 mouse model). An effective amount of a probiotic comprising, consisting of, or consisting essentially of at least one of the following is provided for administration to the subject (or is for use in treating the subject): (a) Bacteroidies bacteria (e.g., B. fragilis, B. thetaiotaomicron or B. vulgatus); (b) Bacteroidies bacteria (e.g., B. fragilis, B. thetaiotaomicron or B. vulgatus) and Enterococcus bacteria (e.g., E. faecilis, E. faecium, E. hirae, E. avium, E. durans, E. gallinarum, or E. casseliflavus); (c) B. fragilis; (d) B. thetaiotaomicron; (e) B. vulgatus; (g) B. fragilis and B. thetaiotaomicron; (h) B. fragilis and B. vulgatus; (i) B. thetaiotaomicron and B. vulgatus; (j) B. fragilis, B. thetaiotaomicron and B. vulgatus; (k) B. fragilis and Enterococcus bacteria (e.g., E. faecilis, E. faecium, E. hirae, E. avium, E. durans, E. gallinarum, or E. casseliflavus); (l) B. thetaiotaomicron and Enterococcus bacteria (e.g., E. faecilis, E. faecium, E. hirae, E. avium, E. durans, E. gallinarum, or E. casseliflavus); (m) B. vulgatus and Enterococcus bacteria (e.g., E. faecilis, E. faecium, E. hirae, E. avium, E. durans, E. gallinarum, or E. casseliflavus); (n) B. fragilis and B. thetaiotaomicron and Enterococcus bacteria (e.g., E. faecilis, E. faecium, E. hirae, E. avium, E. durans, E. gallinarum, or E. casseliflavus); (o) B. fragilis and B. vulgatus and Enterococcus bacteria (e.g., E. faecilis, E. faecium, E. hirae, E. avium, E. durans, E. gallinarum, or E. casseliflavus); (p) B. thetaiotaomicron and B. vulgatus and Enterococcus bacteria (e.g., E. faecilis, E. faecium, E. hirae, E. avium, E. durans, E. gallinarum, or E. casseliflavus); (q) B. fragilis, B. thetaiotaomicron and B. vulgatus and Enterococcus bacteria (e.g., E. faecilis, E. faecium, E. hirae, E. avium, E. durans, E. gallinarum, or E. casseliflavus); (r) B. fragilis and E. faecilis; (s) B. thetaiotaomicron and E. faecilis; (t) B. vulgatus and E. faecilis; (u) B. fragilis and B. thetaiotaomicron and E. faecilis; (v) B. fragilis and B. vulgatus and E. faecilis; (w) B. thetaiotaomicron and B. vulgatus and E. faecilis; or (x) B. fragilis, B. thetaiotaomicron, B. vulgatus and E. faecilis. Following administration of the bacteria, the communication behavior, impaired sensorimotor gating, anxiety, and/or repetitive behavior can be improved. Optionally, a sample from the subject is identified as having a loss-of-function mutation in a gene associated with the CNTNAP2 model, for example a loss-of-function mutation in CNTNAP2. Optionally, a loss-of-function mutation in CNTNAP2 is determined to be indicative that a subject is at risk of developing, or in need of treatment for, at least one of sensorimotor gating behavior, anxiety behavior, and sociability behavior. Optionally, the subject is administered no other bacteria, or substantially no other bacteria apart from the identified bacteria of the probiotic, and as such the probiotic for use in treatment of the subject is in a composition or compositions free or substantially free of other bacteria. Optionally, the subject is administered no antibiotics, or is administered substantially no antibiotics, and as such the probiotic for administration to the subject is in a composition or compositions free or substantially free of antibiotics. Optionally, the subject is administered no drugs, or is administered substantially no drugs, and as such the probiotic for administration to the subject is in a composition or compositions free or substantially free of drugs. Optionally, the subject is administered no pharmaceutically active ingredients, or is administered substantially no pharmaceutically active ingredients, and as such the probiotic for administration to the subject is in a composition or compositions free or substantially free of pharmaceutically active ingredients. Optionally, the B. fragilis comprises wild-type B. fragilis, mutant B. fragilis lacking polysaccharide A (dPSA), or a combination of wild-type B. fragilis and dPSA B. fragilis. In some embodiments, the communication behavior to be improved (and that is improved after administration of the probiotic) comprises at least one of language comprehension and/or production, sociability, communication, and sensorimotor gating). In some embodiments, the subject in need of improvement in communication behavior, sensorimotor gating, anxiety, and/or repetitive behavior has ASD or epilepsy. In some embodiments, the subject in need of improvement in communication behavior, sensorimotor gating, anxiety, and/or repetitive behavior has ASD. In some embodiments, the subject in need of improvement in communication behavior, sensorimotor gating, anxiety, and/or repetitive behavior has epilepsy. In some embodiments, the method further comprises whether or not the subject has ASD or epilepsy (e.g., cortical dysplasia focal epilepsy). In some embodiments, the method further comprises whether or not the subject has ASD. In some embodiments, the method further comprises whether or not the subject has epilepsy (e.g., cortical dysplasia focal epilepsy).

In some embodiments, the probiotic comprises any of the above-disclosed bacterial species or combinations of bacterial species, and is provided for administration to the subject (or is for administration to the subject) in a single probiotic composition. In some embodiments, the probiotic comprises any of the above-referenced bacterial species or combinations of bacterial species, and is administered to the subject (or is for administration to the subject) in two or more different probiotic compositions. For example, a probiotic of “bacteria A and bacteria B” can be administered either in a single composition comprising bacteria A and bacteria B, or in a first composition comprising bacteria A in conjunction with a second composition comprising bacteria B. In some embodiments, first and second compositions are administered simultaneously. In some embodiments, the first and second compositions are administered separately.

In some embodiments, a probiotic comprising a combination of Bacteroides bacteria as described herein is provided as a first composition comprising a first Bacteroides bacterium or combination of Bacteroides bacteria, and a second composition comprising a second Bacteroides bacterium or combination of Bacteroides bacteria as described herein. In some embodiments, a probiotic comprising a combination of Enterococcus bacteria and Bacteroides bacteria as described herein is provided as a first composition comprising the Enterococcus bacteria, and a second composition comprising the Bacteroides bacteria or combination of Bacteroides bacteria as described herein. In some embodiments, the Enterococcus bacteria and a first Bacteroides bacteria (or combination of Bacteroides bacteria) is administered in a first composition, and the Enterococcus bacteria and a second Bacteroides bacteria (or combination of Bacteroides bacteria) that is different from the first is administered in a second composition. In some embodiments, the Enterococcus bacteria and a first Bacteroides bacteria (or combination of Bacteroides bacteria) is administered in a first composition, and a second Bacteroides bacteria (or combination of Bacteroides bacteria) that is different from the first is administered in a second composition.

In accordance with any of the embodiments described above, optionally, each composition, use or method is free of, or is substantially free of bacteria other than the identified bacteria of the probiotic. In accordance with any of the embodiments above, optionally, each composition is free of, or is substantially free of antibiotics. In accordance with any of the embodiments above, optionally, each composition is free of, or is substantially free of bacteria other than the probiotic and antibiotics.

In accordance with embodiments described herein, the probiotics of the methods, uses, and compositions described herein can be for any suitable route of administration. For example, the probiotic can be administered to the subject via oral administration, rectum administration, transdermal administration, intranasal administration or inhalation. In some embodiments, the probiotic is administered to the subject orally.

In some embodiments, the effective amount of bacteria in the probiotic composition, use, or method includes at least about 10⁴ colony forming units (cfu), for example at least about 10⁴, 10⁵, 10⁶, 10⁷, 10⁸, 10⁹, 10¹⁰, 10¹¹, 10¹², or 10¹³ cfu, including ranges between any of the listed values, for example 10⁴-10⁸ cfu, 10⁴-10⁹ cfu, 10⁴-10¹⁰ cfu, 10⁴-10¹¹ cfu, 10⁴-10¹² cfu, 10⁴-10¹² cfu, 10⁵-10⁸ cfu, 10⁵-10⁹ cfu, 10⁵-10¹⁰ cfu,10⁵-10¹¹ cfu, 10⁵-10¹² cfu, 10⁵-10¹² cfu, 10⁶-10⁸ cfu, 10⁶-10⁹ cfu, 10⁶-b 10¹⁰ cfu, 10⁶-10¹¹ cfu, 10⁶-10¹² cfu, 10⁶-10¹² cfu, 10⁷-10⁸ cfu, 10⁷-10⁹ cfu, 10⁷-10¹⁰ cfu, 10⁷-10¹¹ cfu, 10⁷-10¹² cfu, 10⁷-10¹² cfu,-10⁹ cfu, 10⁸-b 10¹⁰cfu, 10⁸-10¹¹ cfu, 10⁸-10¹² cfu, or 10⁸-10¹² cfu. In some embodiments, the effective amount of bacteria comprises a log phase quantity (at 37° C.) of bacteria in a composition for administration to the subject. In some embodiments, the effective amount of bacteria comprises a stationary phase quantity (at 37° C.) of bacteria in a composition for administration to the subject.

Methods of Treating and/or Preventing ASD Symptoms and/or Epilepsy Symptoms

In some embodiments, methods of treating ASD and/or epilepsy symptoms are provided. A subject can be identified as in need of improving sensorimotor gating behavior, anxiety behavior, communication behavior (including language comprehension and/or production, sociability, and/or, communication), and/or repetitive behavior. Optionally, the subject (or a sample from the subject) can be determined to possess a loss-of-function mutation in the DISC1, and/or CTNAP2 gene. Optionally, a loss-of-function mutation in the DISC1, and/or CTNAP2 gene can be determined to be indicative that the subject is in need of improving sensorimotor gating behavior, anxiety behavior, communication behavior, and/or repetitive behavior. The subject can be administered a probiotic comprising, consisting essentially of, or consisting of an effective amount of Bacteroides bacteria, or a combination of Bacteroides and Enterococcus bacteria as described herein. The sensorimotor gating behavior, anxiety behavior, communication behavior and/or repetitive behavior can be improved. In some embodiments, the communication behavior that is improved includes at least one of language comprehension and/or production, sociability, or communication. In some embodiments, the method further comprises determining whether or not the subject has ASD. In some embodiments, the method further comprises determining whether or not the subject has epilepsy (e.g., cortical dysplasia focal epilepsy). In some embodiments, ASD symptoms are treated. In some embodiments, epilepsy symptoms are treated. In some embodiments, ASD and epilepsy symptoms are treated. In some embodiments, the epilepsy comprises cortical dysplasia focal epilepsy.

In some embodiments, methods of treating ASD symptoms are provided. A subject can be identified as in need of improving sensorimotor gating behavior, anxiety behavior, and sociability behavior. It is noted that such behaviors are in accordance with the BTBR model. Optionally, the subject (or a sample from the subject) can be determined to possess a loss-of-function mutation in the DISC1 gene. Optionally, a loss-of-function mutation in the DISC1 gene can be determined to be indicative that the subject is in need of improving sensorimotor gating behavior, anxiety behavior, communication behavior, and/or repetitive behavior. The subject can be administered a probiotic comprising, consisting essentially of, or consisting of an effective amount of Bacteroides bacteria, or a combination of Bacteroides and Enterococcus bacteria as described herein. The sensorimotor gating behavior, anxiety behavior, and sociability behavior can be improved. In some embodiments, the method further comprises determining whether or not the subject has ASD. In some embodiments, the epilepsy symptoms are treated.

In some embodiments, methods of treating epilepsy symptoms are provided. A subject can be identified as in need of improving communication behavior (including language comprehension and/or production, sociability, and/or, communication), sensorimotor gating, anxiety, and/or repetitive behavior (it is noted that these behaviors are in accordance with the CTNAP2 model). Optionally, the subject (or a sample from the subject) can be determined to possess a loss-of-function mutation in the CTNAP2 gene. Optionally, a loss-of-function mutation in the CTNAP2 gene can be determined to be indicative that the subject is in need of improving communication behavior, sensorimotor gating behavior, anxiety behavior, and/or repetitive behavior. The subject can be administered a probiotic comprising, consisting essentially of, or consisting of an effective amount of Bacteroides bacteria, or a combination of Bacteroides and Enterococcus bacteria as described herein. The sensorimotor gating behavior, anxiety behavior, communication behavior and/or repetitive behavior can be improved. In some embodiments, the communication behavior that is improved includes at least one of language comprehension and/or production, sociability, or communication. In some embodiments, the epilepsy comprises cortical dysplasia focal epilepsy. In some embodiments, the method further comprises determining whether or not the subject has epilepsy (e.g., cortical dysplasia focal epilepsy).

In some embodiments, methods of treating ASD symptoms are provided. A subject can be identified as in need of improving communication behavior (including language comprehension and/or production, sociability, and/or, communication), sensorimotor gating, anxiety, and/or repetitive behavior (it is noted that these behaviors are in accordance with the CTNAP2 model). Optionally, the subject (or a sample from the subject) can be determined to possess a loss-of-function mutation in the CTNAP2 gene. Optionally, a loss-of-function mutation in the CTNAP2 gene can be determined to be indicative that the subject is in need of improving communication behavior, sensorimotor gating behavior, anxiety behavior, and/or repetitive behavior. The subject can be administered a probiotic comprising, consisting essentially of, or consisting of an effective amount of Bacteroides bacteria, or a combination of Bacteroides and Enterococcus bacteria as described herein. The sensorimotor gating behavior, anxiety behavior, communication behavior and/or repetitive behavior can be improved. In some embodiments, the communication behavior that is improved includes at least one of language comprehension and/or production, sociability, or communication. In some embodiments, the method further comprises determining whether or not the subject has ASD. In some embodiments, ASD symptoms are treated.

In some embodiments, methods of preventing ASD and/or epilepsy symptoms in a subject at risk of developing these symptoms are provided. A subject can be identified as at risk of developing impaired sensorimotor gating behavior, anxiety behavior, communication behavior (including language comprehension and/or production, sociability, and/or, communication), and/or repetitive behavior. Optionally, the subject (or a sample from the subject) can be determined to possess a loss-of-function mutation in the DISC1, and/or CTNAP2 gene. Optionally, a loss-of-function mutation in the DISC1, and/or CTNAP2 gene can be determined to be indicative that the subject is at risk of developing impaired sensorimotor gating behavior, anxiety behavior, communication behavior, and/or repetitive behavior. The subject can be administered a probiotic comprising, consisting essentially of, or consisting of an effective amount of Bacteroides bacteria, or a combination of Bacteroides and Enterococcus bacteria as described herein. The subject can develop with minimized deficiencies or no discernable deficiencies in sensorimotor gating behavior, anxiety behavior, communication behavior and/or repetitive behavior. In some embodiments, the communication behavior that develops without discernable deficiencies includes at least one of language comprehension and/or production, sociability, or communication. In some embodiments, ASD symptoms are treated. In some embodiments, epilepsy symptoms are treated. In some embodiments, ASD and epilepsy symptoms are treated. In some embodiments, the epilepsy comprises cortical dysplasia focal epilepsy. Optionally, the at-risk subject is an infant or child.

In some embodiments, methods of preventing ASD in a subject at risk of developing these symptoms are provided. A subject can be identified as at risk for developing impaired sensorimotor gating behavior, anxiety behavior, and sociability behavior. It is noted that such behaviors are in accordance with the BTBR model. Optionally, the subject (or a sample from the subject) can be determined to possess a loss-of-function mutation in the DISC1, and/or CTNAP2 gene. Optionally, a loss-of-function mutation in the DISC1, and/or CTNAP2 gene can be determined to be indicative that the subject at risk for developing impaired sensorimotor gating behavior, anxiety behavior, communication behavior, and/or repetitive behavior. The subject can be administered a probiotic comprising, consisting essentially of, or consisting of an effective amount of Bacteroides bacteria, or a combination of Bacteroides and Enterococcus bacteria as described herein. The subject can develop with minimized deficiencies or no discernable deficiencies in sensorimotor gating behavior, anxiety behavior, and sociability behavior. Optionally, the at-risk subject is an infant or child.

In some embodiments, methods of preventing epilepsy symptoms are provided. A subject can be identified as at risk for developing impaired communication behavior (including language comprehension and/or production, sociability, and/or communication), sensorimotor gating, anxiety behavior, and/or repetitive behavior (it is noted that these behaviors are in accordance with the CTNAP2 model). Optionally, the subject (or a sample from the subject) can be determined to possess a loss-of-function mutation in the CTNAP2 gene. Optionally, a loss-of-function mutation in the CTNAP2 gene can be determined to be indicative that the subject is at risk of developing impaired communication behavior, sensorimotor gating behavior, anxiety behavior, and/or repetitive behavior. The subject can be administered a probiotic comprising, consisting essentially of, or consisting of an effective amount of Bacteroides bacteria, or a combination of Bacteroides and Enterococcus bacteria as described herein. The subject can develop with minimized deficiencies or no discernable deficiencies in sensorimotor gating behavior, anxiety behavior, communication behavior and/or repetitive behavior. In some embodiments, the communication behavior includes at least one of language comprehension and/or production, sociability, or communication. In some embodiments, the epilepsy comprises cortical dysplasia focal epilepsy. Optionally, the at-risk subject is an infant or child.

In some embodiments, methods of preventing ASD symptoms are provided. A subject can be identified as at risk of developing impaired communication behavior (including language comprehension and/or production, sociability, and/or communication), sensorimotor gating, anxiety, and/or repetitive behavior (it is noted that these behaviors are in accordance with the CTNAP2 model). Optionally, the subject (or a sample from the subject) can be determined to possess a loss-of-function mutation in the CTNAP2 gene. Optionally, a loss-of-function mutation in the CTNAP2 gene can be determined to be indicative that the subject is at risk of developing impaired communication behavior, sensorimotor gating behavior, anxiety behavior, and/or repetitive behavior. The subject can be administered a probiotic comprising, consisting essentially of, or consisting of an effective amount of Bacteroides bacteria, or a combination of Bacteroides and Enterococcus bacteria as described herein. The subject can develop with minimized deficiencies or no discernable deficiencies in sensorimotor gating behavior, anxiety behavior, communication behavior and/or repetitive behavior. In some embodiments, the communication behavior that is improved includes at least one of language comprehension and/or production, sociability, or communication.

In some embodiments, the probiotic comprising, consisting essentially of, or consisting of Bacteroides bacteria, or a combination of Enterococcus bacteria and Bacteroides bacteria of any of the methods described herein is selected from the group consisting of (a) Bacteroidies bacteria (e.g., B. fragilis, B. thetaiotaomicron or B. vulgatus); (b) Bacteroidies bacteria (e.g., B. fragilis, B. thetaiotaomicron or B. vulgatus) and Enterococcus bacteria (e.g., E. faecilis, E. faecium, E. hirae, E. avium, E. durans, E. gallinarum, or E. casseliflavus); (c) B. fragilis; (d) B. thetaiotaomicron; (e) B. vulgatus; (g) B. fragilis and B. thetaiotaomicron; (h) B. fragilis and B. vulgatus; (i) B. thetaiotaomicron and B. vulgatus; (j) B. fragilis, B. thetaiotaomicron and B. vulgatus; (k) B. fragilis and Enterococcus bacteria (e.g., E. faecilis, E. faecium, E. hirae, E. avium, E. durans, E. gallinarum, or E. casseliflavus); (l) B. thetaiotaomicron and Enterococcus bacteria (e.g., E. faecilis, E. faecium, E. hirae, E. avium, E. durans, E. gallinarum, or E. casseliflavus); (m) B. vulgatus and Enterococcus bacteria (e.g., E. faecilis, E. faecium, E. hirae, E. avium, E. durans, E. gallinarum, or E. casseliflavus); (n) B. fragilis and B. thetaiotaomicron and Enterococcus bacteria (e.g., E. faecilis, E. faecium, E. hirae, E. avium, E. durans, E. gallinarum, or E. casseliflavus); (o) B. fragilis and B. vulgatus and Enterococcus bacteria (e.g., E. faecilis, E. faecium, E. hirae, E. avium, E. durans, E. gallinarum, or E. casseliflavus); (p) B. thetaiotaomicron and B. vulgatus and Enterococcus bacteria (e.g., E. faecilis, E. faecium, E. hirae, E. avium, E. durans, E. gallinarum, or E. casseliflavus); (q) B. fragilis, B. thetaiotaomicron and B. vulgatus and Enterococcus bacteria (e.g., E. faecilis, E. faecium, E. hirae, E. avium, E. durans, E. gallinarum, or E. casseliflavus); (r) B. fragilis and E. faecilis; (s) B. thetaiotaomicron and E. faecilis; (t) B. vulgatus and E. faecilis; (u) B. fragilis and B. thetaiotaomicron and E. faecilis; (v) B. fragilis and B. vulgatus and E. faecilis; (w) B. thetaiotaomicron and B. vulgatus and E. faecilis; or (x) B. fragilis, B. thetaiotaomicron, B. vulgatus and E. faecilis. Optionally, the subject is administered no other bacteria, or substantially no other bacteria apart from the identified bacteria of the probiotic, and as such the probiotic for use in treatment of the subject is in a composition or compositions free or substantially free of other bacteria. Optionally, the subject is administered no antibiotics, or is administered substantially no antibiotics, and as such the probiotic for administration to the subject is in a composition or compositions free or substantially free of antibiotics. Optionally, the subject is administered no drugs, or is administered substantially no drugs, and as such the probiotic for administration to the subject is in a composition or compositions free or substantially free of drugs. Optionally, the subject is administered no pharmaceutically active ingredients, or is administered substantially no pharmaceutically active ingredients, and as such the probiotic for administration to the subject is in a composition or compositions free or substantially free of pharmaceutically active ingredients.

In some embodiments as described above, the method further comprises determining that the subject is in need of improving a behavior. In some embodiments, for example uses, methods, and or compositions directed to infants and/or children, a subject at risk for an ASD behavior is identified based on maternal immune activation and/or other risk factors. In some embodiments, the subject is diagnosed as having ASD based on the level of an ASD-related metabolite or combination of metabolites in the gut, in a bodily fluid (for example, blood and urine), or any combination thereof. Methods of diagnosing ASD based on levels of metabolite in a subject are described in detail in US Pub. No. 2014/0065132, hereby incorporated by reference in its entirety. In some embodiments, the subject is determined to have a lesion or developmental deficiency in a region of the brain associated with speech production, speech recognition, impulse control, and/or socialization, for example regions of the cerebral cortex, the corpus colosum, Broca's area, and/or Wernicke's area. In some embodiments, an ASD behavior, for example a deficient communication, vocalization, sensorimotor, anxiety, and/or repetitive behavior, or a combination of two or more of these is identified using standard diagnostic criteria, for example in the Diagnostic and Statistical Manual of Mental Disorders, Fourth Edition (DSM-4) or Fifth Edition (DSM-5). In some embodiments, the presence or absence of ASD in the subject is determined using a behavioral test, for example at least one of the Autism Behavior Checklist (ABC), Autism diagnostic Interview-Revised (ADI-R), childhood autism Rating Scale (CARS), and/or Pre-Linguistic Autism Diagnostic Observation Schedule (PL-ADOS). The behavioral test can include, but is not limited to, detecting the presence and/or extent of 1) preoccupation with one or more stereotyped and restricted patterns of interest that is abnormal in either intensity or focus, 2) inflexible adherence to specific, nonfunctional routines or rituals, c) stereotyped and repetitive motor mannerisms (such as hand flapping, finger flapping etc.), and/or d) persistent preoccupation with parts of objects. Non-limiting examples of behavior that can be included in a behavioral test and suggest a need for improving behavioral performance in the subject under the test include: a) sensory behaviors, including poor use of visual discrimination when learning, seems not to hear, so that a hearing loss is suspected, sometimes shows no “startle response” to loud noise”, sometimes painful stimuli such as bruises, cuts, and injections evoke no reaction, often will not blink when bright light is directed toward eyes, covers ears at many sounds, squints, frowns, or covers eyes when in the presence of natural light, frequently has no visual reaction to a “new” person, stares into space for long periods of time; b) relating behaviors: frequently does not attend to social/environmental stimuli, has no social smile, does not reach out when reached for, non-responsive to other people's facial expressions/feelings, actively avoids eye contact, resists being touched or held, is flaccid when held in arms, is stiff and hard to held, does not imitate other children at play, has not developed any friendships, often frightened or very anxious, “looks through” people; c) body and object use behaviors: whirls self for long periods of time, does not use toys appropriately, insists on keeping certain objects with him/her, rocks self for long periods of time, does a lot of lunging and darting, flaps hands, walks on toes, hurts self by banging head, biting hand, twirls, spins, and bangs objects a lot, feel, smell, and/or taste objects in the environment, gets involved in complicated “rituals” such as lining things up, is very destructive; and d) language behaviors: does not follow simple commands given once, has pronoun reversal, speech is atonal, does not respond to own name when called out among two others, seldom says “yes” or “I”, does not follow simple commands involving prepositions, gets desired objects by gesturing, repeats phrases over and over, cannot point to more than five named objects, uses 0-5 spontaneous words per day to communicate wants and needs, repeats sounds or words over and over, echoes questions or statements made by others, uses at least 15 but less than 30 spontaneous phrases daily to communicate, learns a simple task but “forgets” quickly, strong reactions to changes in routine/environment, has “special abilities” in one area of development, which seems to rule out mental retardation, severe temper tantrums and/or frequent minor tantrums, hurts others by biting, hitting, and/or kicking, does not wait for needs to be met, difficulties with toileting, does not dress self without frequent help, frequently unaware of surroundings, and may be oblivious to dangerous situations, prefers to manipulate and be occupied with inanimate things, and/or a developmental delay identified at or before 30 months of age. One of ordinary skill in the art would appreciate that the attending physician would know how to identify a subject in need of treatment disclosed herein.

In some embodiments as described above, the method comprises administering the effective amount of probiotic in a single administration of one or more compositions. In some embodiments as described above, the method comprises administering the effective amount of the probiotic across two or more administrations of a single composition as described herein. For example, the compositions can be administered about 1 minute, 2, 3, 4, 5, 6, 7, 8, 9, 10, 15, 20, 25, 30, 35, 40, 45, 50, 55 minutes, 1 hour, 2, 3, 4, 5, 6, 7, 8, 9, 10, 11, 12, 13, 14, 15, 16, 17, 18, 19, 20, 21, 22, 23 hours, 1 day, 2, 3, 4, 5, 6, 7, 8, 9, or 10 days apart, including ranges between any two of the listed values, for example 1 minute-10 minutes, 1 minute to 30 minutes, 1 minute to 1 hour, 1 minute-2 hours, 1 minute-4 hours, 1 minute-12 hours, 1 minute-18 hours, 1 minute-1 day, 10 minutes to 30 minutes, 10 minutes to 1 hour, 10 minutes-2 hours, 10 minutes-4 hours, 10 minute-12 hours, 10 minutes-18 hours, 10minutes-1 day, 30 minutes to 1 hour, 30 minutes-2 hours, 30 minutes-4 hours, 30 minute-12 hours, 30 minutes-18 hours, 30 minutes-1 day, 30 minutes-2 days, 1 hour-2 hours, 1 hour-4 hours, 1 hour-12 hours, 1 hour-18 hours, 1 hour-1 day, 4 hours-12 hours, 4 hours-18 hours, 4 hours-1 day, 1 day-2 days, 1 day-3 days, 1 day-4 days, 1 day-5 days, 1 day-7 days, 1 day-10 days, 2 days-3 days, 2 days-4 days, 2 days-5 days, 2 days-7 days, 2 days-10 days, or 5 days to 10 days. In some embodiments as described above, the method comprises administering the effective amount of two or more different compositions as described herein across two or more administrations of a single composition. For example, the second composition can be administered about 1 minute, 2, 3, 4, 5, 6, 7, 8, 9, 10, 15, 20, 25, 30, 35, 40, 45, 50, 55 minutes, 1 hour, 2, 3, 4, 5, 6, 7, 8, 9, 10, 11, 12, 13, 14, 15, 16, 17, 18, 19, 20, 21, 22, 23 hours, 1 day, 2, 3, 4, 5, 6, 7, 8, 9, or 10 days after the first composition, including ranges between any two of the listed values, for example 1 minute-10 minutes, 1 minute to 30 minutes, 1 minute to 1 hour, 1 minute-2 hours, 1 minute-4 hours, 1 minute-12 hours, 1 minute-18 hours, 1 minute-1 day, 10 minutes to 30 minutes, 10 minutes to 1 hour, 10 minutes-2 hours, 10 minutes-4 hours, 10 minute-12 hours, 10 minutes-18 hours, 10 minutes-1 day, 30 minutes to 1 hour, 30 minutes-2 hours, 30 minutes-4 hours, 30 minute-12 hours, 30 minutes-18 hours, 30 minutes-1 day, 30 minutes-2 days, 1 hour-2 hours, 1 hour-4 hours, 1 hour-12 hours, 1 hour-18 hours, 1 hour-1 day, 4 hours-12 hours, 4 hours-18 hours, 4 hours-1 day, 1 day-2 days, 1 day-3 days, 1 day-4 days, 1 day-5 days, 1 day-7 days, 1 day-10 days, 2 days-3 days, 2 days-4 days, 2 days-5 days, 2 days-7 days, 2 days-10 days, or 5 days to 10 days. In some embodiments, the probiotic is administered in a slow release formulation (for example a slow-release capsule or implant) for any of the durations described above.

In some embodiments, the probiotic is administered to the subject until an improvement in behavioral performance is observed. Optionally, the probiotic is administered to the subject after an improvement in behavioral performance is observed, for example to solidify or maintain the improved behavioral performance.

### EXAMPLES

Some aspects of the embodiments discussed above are disclosed in further detail in the following examples, which are not in any way intended to limit the scope of the present disclosure.

### Example 1

### Correction of ASD-Relevant Behaviors in BTBR Mice by B. fragilis

Adult BTBR mice were given applesauce containing B. fragilis or applesauce alone for 1 week post-weaning. The single mice were placed in the open field and the number of entries into a center square during the first 10 minutes was recorded (FIG. 1A). Similar findings were made for duration in the center of the field (not shown). No group difference was found for total distance traveled. Treatment with B. fragilis prevents or treats this abnormality. *p<0.05; n=30-35 animals per trial, replicated two times.

When placed in a cage with multiple marbles arranged on top of the bedding, control BTBR male mice compulsively bury them, while B. fragilis treated BTBR mice are reduced in this repetitive/stereotyped behavior (FIG. 1B).

Prepulse inhibition (PPI) as measured at 5 and 15 db above background in BTBR mice was observed (FIG. 1C). Treatment with B. fragilis normalized this measure of sensorimotor gating. *p<0.05 **p<0.01. n=15-20 animals per trial.

Thus, treatment of BTBR autism model adult mice with Bacteroides bacteria in accordance with some embodiments herein improves behavioral deficiencies associated with ASD.

### Example 2

### Correction of ASD-Relevant Behaviors in CNTNAP2 Mice by B. fragilis

Wild-type mice (C57BU6), Heterozygous (Her) mice (CNTNAP2+/−) and knockout (KO) (CNTNAP2−/−) mice were tested for a variety of ASD-associated behaviors. Tested were adult controls given applesauce alone for three weeks post-weaning, and adult mice that had been given applesauce containing B. fragilis for three weeks post-weaning. As reported by Penagarikano et al. (2011), the mutant mice are hyperactive. Hyperactivity in the open field was measured (FIGS. 1D and 1E). Notably, B. fragilis feeding in applesauce was able to prevent hyperactivity, as measured by distance traveled (FIG. 1D) and average velocity (FIG. 1E). Similarly, CNTNAP2−/− display repetitive behavior as assessed by Penagarikano et al. (2011), which can be measured by self-grooming. Upon measurement of self-grooming (FIG. 1F), B. fragilis treatment ameliorates this ASD-like self-grooming behavior. *p<0.05 NS=not significant. N=4-11 mice, replicated 2 times.

Thus, treatment of CNTNAP2 autism model adult mice with Bacteroides bacteria in accordance with some embodiments herein improves behavioral deficiencies associated with ASD.

### Example 3

### Correction of ASD-Relevant Behaviors by B. fragilis

A human adult subject is identified as having a combination of sensorimotor gating behavior, anxiety behavior, and sociability behaviors. The subject eats a cereal bar comprising an effective amount of a probiotic consisting essentially of B. fragilis weekly for three weeks. After about three weeks of eating the cereal bar, the subject is expected to exhibit increased response to auditory stimuli, reduced anxiety, and more frequent social interaction.

### Example 4

### Prevention of ASD-Relevant Behaviors by Bacteroides

A human child subject is determined to be heterozygous for a loss-of-function mutation in the DISC1 gene, and is thus determined to be at risk for ASD behaviors, including impaired communication behavior (language comprehension and/or production, sociability, communication), impaired sensorimotor gating, anxiety, and repetitive behavior. The subject drinks a yogurt drink comprising an effective amount of a probiotic consisting essentially of B. thetaiotaomicron once every four days for six months. The subject is expected to develop with minimal impairment in communication behavior, sensorimotor gating behavior, anxiety, and repetitive behavior.

### Example 5

### Correction of Epilepsy-Relevant Behaviors by B. fragilis and E. faecilis

A human adolescent subject is identified as having impaired sensorimotor gating, impaired language comprehension and production, and epileptic seizures characteristic of cortical dysplasia focal epilepsy. The subject is determined to be in need of treatment for epilepsy. The subject takes a gel capsule comprising a probiotic consisting essentially of B. fragilis and E. faecilis daily until symptoms are expected to improve.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one of skill in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those of skill in the art. The various aspects and embodiments disclosed herein are for purposes of illustration and are not intended to be limiting, with the true scope and spirit being indicated by the following claims.

What is claimed is:

1. A method for improving behavioral performance in a subject having autism spectrum disorder (ASD), epilepsy, or cortical dysplasia focal epilepsy, the method comprising administering an effective amount of one or more Bacteroidies bacteria to the subject.

2. The method of claim 1, further comprising:
detecting in a sample of the subject a presence or absence of a loss-of-function of at least one of:
contactin associated protein-like 2 (CNTNAP2); and
disrupted in schizophrenia 1 (Disc1); and
administering the effective amount of one or more Bacteroidies bacteria to the subject if the subject has a loss-of-function of at least one of CNTNAP2 and Disc1.

3. The method of claim 1, wherein the effective amount of one or more Bacteroidies bacteria comprises B. fragilis.

4. The method of claim 1, wherein the effective amount of one or more Bacteroidies bacteria comprises B. fragilis, B. thetaiotaomicron, B. vulgatus, or a mixture of two or three of the listed bacteria.

5. The method of claim 1, wherein the improved behavioral performance comprises at least one of language comprehension, language production, sociability, communication, sensorimotor gating, anxiety, or repetitive behavior.

6. The method of claim 1, wherein the improved behavioral performance comprises at least one of anxiety, sensorimotor gating, and sociability.

7. The method of claim 1, wherein a sole active ingredient administered to the subject in the method consists essentially of one or more Bacteroidies bacteria.

8. The method of claim 1, wherein the effective amount of one or more Bacteroidies bacteria is in a composition substantially free of bacteria other than the one or more Bacteroidies bacteria.

9. The method of claim 1, wherein detecting a presence or absence of a loss-of-function of CNTNAP2 or Disc1 comprises detecting an allele of a gene encoding CNTNAP2 protein or Disc1 protein.

10. The method of claim 1, wherein detecting a presence or absence of a loss-of-function of CNTNAP2 or Disc1 comprises detecting a presence or absence of a CNTNAP2 or Disc1 polypeptide.

11. The method of claim 1, wherein detecting a presence or absence of a loss-of-function of CNTNAP2 or Disc1 is performed on a sample of the subject.

12. The method of claim 1, wherein detecting a presence or absence of a loss-of-function of CNTNAP2 or Disc1 is performed on the subject in vivo.

13. The method of claim 1, wherein detecting a presence or absence of a loss-of-function of CNTNAP2 or Disc1 comprises detecting a deletion of at least a portion of a gene encoding CNTNAP2 or Disc1.

14. The method of claim 1, wherein the subject suffers from anxiety, autism spectrum disorder (ASD), or a pathological condition with one or more of the symptoms of ASD.

15. The method of claim 1, wherein the effective amount of one or more Bacteroidies bacteria is administered orally to the subject.

16. A kit comprising:
a composition comprising one or more Bacteroidies bacteria, wherein the composition is suitable for administration to a human subject; and
at least one of:
a nucleic acid substantially complementary to a gene encoding contactin associated protein-like 2 (CNTNAP2), or a gene encoding disrupted in schizophrenia 1 (Disc1) ; or
an antibody that binds specifically to a CNTNAP2, or Disc1 polypeptide.

17. The kit of claim 16, wherein the composition is suitable for oral administration.

18. The kit of claim 16, wherein the one or more Bacteroidies bacteria comprises B. fragilis.

19. The kit of claim 16 wherein the composition consists essentially of one or more Bacteroidies bacteria.

20. The kit of claim 16, wherein the composition is substantially free of bacteria other than Bacteroidies fragilis, B. thetaiotaomicron, and B. vulgatus.

21. The kit of claim 16, wherein the composition is substantially free of bacteria other than Bacteroidies fragilis.
